(19) United States
(12) Patent Application Publication
(10) Pub. No.: US 2011/0071178 A1
(43) Pub. Date: Mar. 24, 2011

(54) Methods and Compounds for Modulating Cannabinoid Activity

(75) Inventors: Alexandros Makriyannis, Watertown, MA (US); Spyridon P. Nikas, Waltham, MA (US); Shakiru O. Alapafuja, Willimantic, CT (US); Vidyanand G. Shukla, Boston, MA (US)

(73) Assignee: Northeastern University, Boston, MA (US)

(21) Appl. No.: 12/761,987

(22) Filed: Apr. 16, 2010

Related U.S. Application Data
(63) Continuation of application No. PCT/US2008/080215, filed on Oct. 16, 2008.
(60) Provisional application No. 60/999,127, filed on Oct. 16, 2007.

(51) Int. Cl. A61K 31/438; C07C 309/68; A61K 31/255; C07C 49/175; A61K 31/12; C07C 271/12; A61K 31/222; C07C 69/96; A61K 31/265; C07D 211/60; A61K 31/445; C07D 497/10; C07D 213/56; A61K 31/4402; C07C 275/32; A61K 31/17; C07D 271/06; A61K 31/4245; C07D 275/06; A61K 31/428; C07D 263/34; A61K 31/421; A61P 3/00; A61P 35/00; A61P 15/08; A61P 25/00; A61P 29/00; G01N 33/53

(52) U.S. Cl. 514/278; 562/41; 514/517; 568/308; 514/678; 560/163; 514/551; 558/270; 514/512; 546/195; 514/319; 546/19; 546/332; 514/357; 564/52; 514/598; 548/131; 514/364; 548/211; 514/373; 548/236; 514/374; 435/7.8

(57) ABSTRACT

Disclosed are compounds and compositions that inhibit the action of monoacylglycerol lipase (MGL) and fatty acid amide hydrolase (FAAH), methods of inhibiting MGL and FAAH, methods of modulating cannabinoid receptors, and methods of treating various disorders related to the modulation of cannabinoid receptors.

### PRIORITY STATEMENT

This application is a continuation of International Application No. PCT/US2008080215 filed Oct. 16, 2008, which claims the benefit of priority of U.S. Provisional Appln. No. 60/999,127, filed Oct. 16, 2007, the entire disclosures of which are incorporated into this application by reference.

### FIELD OF THE INVENTION

The present disclosure is in the field of medicinal chemistry. More specifically, this disclosure relates to the use of certain chemical compounds in methods for treating pain, inflammation, neuropathy, neurodegenerative disease, anxiety disorder, motor function disorder, fertility disorder, appetite disorder, metabolic disorder, movement disorder, and cancer.

### BACKGROUND

Presently, two G_{i/o} protein coupled cannabinoid receptors have been characterized in mammals and other organisms: CB1, a central receptor found in the mammalian brain and a number of other sites in peripheral tissues; and CB2, a peripheral receptor found principally in cells related to the immune system. Compounds known as cannabinergic ligands bind to CB1 and/or CB2 receptors in a subject. In vitro methods for assaying the ability of a compound to bind to CB1 and/or CB2 receptors are known and results from these assays correlate with, and predict, the in vivo ability of that compound to bind to, and thereby modulate, CB1 and/or CB2 receptors.

Despite having a rapid onset of action, the magnitude and duration of in vivo CB1 and/or CB2 receptor modulation by cannabinergic ligands are relatively short, because of a rapid inactivation process comprising hydrolysis of that cannabinergic ligand. For example, anandamide is inactivated by fatty acid amide hydrolase (FAAH)-mediated hydrolysis. Although FAAH has also been shown to catalyze hydrolysis of 2-arachidonoylglycerol in vitro, a distinct enzyme, monoacylglycerol lipase (also known as MGL, MAG lipase, or MAGL) plays the predominant role in catalyzing 2-arachidonoylglycerol hydrolysis in vivo. MGL is a serine hydrolase that converts 2- and 1-monoglycerides to fatty acid and glycerol and is a key enzyme responsible for the termination of endocannabinoid signaling. A need exists for compounds that inhibit the hydrolytic activity of MGL and FAAH, thereby maintaining or increasing the magnitude and duration of cannabinoid receptor modulation.

### SUMMARY OF THE INVENTION

It has been discovered that certain chemical compounds can inhibit MGL. This discovery has been exploited to develop the present application, which includes novel compounds and therapeutic compositions for inhibiting MGL, or MGL and FAAH, methods for modulating cannabinoid receptors, and methods for treating various disorders in a subject.

One aspect of the application is directed to a method of modulating cannabinoid receptors in a biological sample. In this method, the level of a cannabinergic ligand in the biological sample is measured. Then, the biological sample is contacted with a compound of Formula (I), thereby inhibiting an enzyme that hydrolyzes the cannabinergic ligand. The level of the cannabinergic ligand in the contacted sample is then measured, the cannabinoid receptors being modulated if the level of the cannabinergic ligand in the contacted sample is the same or greater than the level of the cannabinergic ligand in the uncontacted sample.

In the compound having Formula (I), R—X—Y, Y is selected from the group consisting of

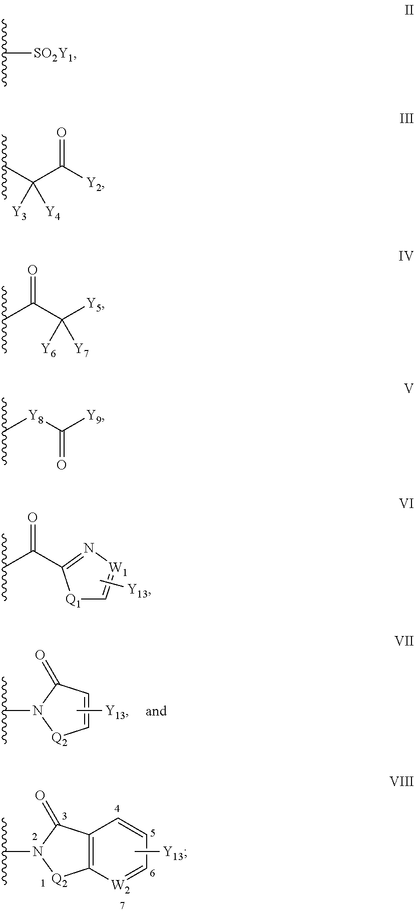

wherein: Y₁ is —F, —Cl, —O-alkyl, —O-cycloalkyl, —O-heterocyclic, —O-aryl, —O-heteroaryl, or —O-adamantyl;
Y₂ is —H, —OH, —NH₂, —OMe, —OEt, —CF₃, —C≡CH, —CH₂—C≡CH, —CH═CH₂, fluoroalkyl, —C₁₋₅-alkyl, -aryl, -alkyl-aryl, -aryl-alkyl, -aryl-alkyl-Y₁₄, -aryl-heteroaryl, -aryl-aryl, -heteroaryl, -heteroaryl-alkyl, -heteroaryl-alkyl-Y₁₄, -heteroaryl-aryl, -heteroaryl-heteroaryl, -cycloalkyl, -cycloalkyl-alkyl, -cycloalkyl-alkyl-Y₁₄, -heterocyclic, -heterocyclic-alkyl, -heterocyclic-alkyl-Y₁₄, -adamantyl, —C₁₋₅-alkyl-Y₁₄, -aryl-Y₁₄, -heteroaryl-Y₁₄, -cycloalkyl-Y₁₄, -heterocyclic-Y₁₄, or -adamantyl-Y₁₄;
Y₃ and Y₄ are each independently —F, —Cl, or —OH; or Y3 and Y4 taken together form a ketone;
Y₅ is —F, —CONH₂, —SO₂NH₂, —COOH, —COOMe, —COOEt, —CF₃, —C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₆ and Y₇ are each independently —F, —Cl, or —OH;
Y₈ is NH, O, or heterocycle;
Y₉ is —OY₁₀, —N(Y₁₁)Y₁₂, or heterocycle;
Y₁₀ is alkyl, aryl, benzyl, difluorophenyl, fluorophenyl, heteroaryl, cycloalkyl, adamantyl, heterocyclic, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₁₁ is —H, -alkyl, -aryl, or -alkyl-aryl;
Y₁₂ is alkyl, aryl, heteroaryl, cycloalkyl, adamantyl, heterocyclic, —C₁₋₅-alkyl-aryl, —C₁₋₅-alkyl-heteroaryl, -aryl-(Y₁₄)₁₋₄; -heteroaryl-Y₁₄, -cycloalkyl-Y₁₄, -adamantyl-Y₁₄; or -heterocyclic-Y₁₄; or Y₁₁ and Y₁₂ when taken together along with the N to which they are bonded form a 5- or 6-membered saturated heterocylic ring, the ring containing up to one additional heteroatom selected from the group consisting of N, O, and S;
Y₁₃ is —H, —OH, —SH, —NH₂, —CN, —N₃, —NCS, —NCO, —CONH₂, —SO₂NH₂; —COOH, —COOMe, —COOEt, —NO₂, —CF₃, —SO₃H, —P(O)(OH)₂, —CH₂—C≡CH, —CH₂—C≡CH, —CH═CH₂, fluoroalkyl, —C₁₋₆-alkyl, aryl, heteroaryl, cycloalkyl, adamantyl, heterocyclic, —C₁₋₆-alkyl-Y₁₄; -aryl-Y₁₄, heteroaryl Y₁₄ cycloalkyl-Y₁₄, -adamantyl-Y₁₄, or -heterocyclic-Y₁₀;
Y₁₄ is —H, —F, —Cl, Br, —I, —OH, —OMe, —OEt, —OPh, —OBn, —SH, —NH₂, —CN, —N₃, —NCS, —NCO, —CONH₂, —SO₂NH₂, —COOH, —COOMe, —COOEt, —NO₂, -alkyl, —CF₃, —SO₃H, —P(O)(OH)₂, —CH₂—C≡CH, —CH═CH₂, or —NHCOCH₃, or —CH₂OH;
W₁ is CH or N if Y₁₃ is not bonded to W₁, or W₁ is C if Y₁₃ is bonded to W₁;
W₂ is CH or N if W₂ is not bonded to Y₁₃, or W₂ is C if W₂ is bonded to Y₁₃; if W₂ is N then it can occupy position 4, 5, 6, or 7 in VIII;
Q₁ is —CH₂, —O, —S, or —NH if Q₁ is not bonded to Y₁₃; Q₁ is —CH or —N if Q₁ is bonded to Y₁₃;
Q₂ is —SO₂, —C(O), or —S(O);

wherein: X is —(CH₂)ₙ—, —(CH₂)ⱼ-A-(CH₂)ₖ—, cycloalkyl, or heterocycle; A is —CH═CH—, —C≡C—, C═O, O, S, or NH; n is an integer from 0 to 15; j is an integer from 0 to 10; and k is an integer from 0 to 10; and wherein R is selected from the group consisting of

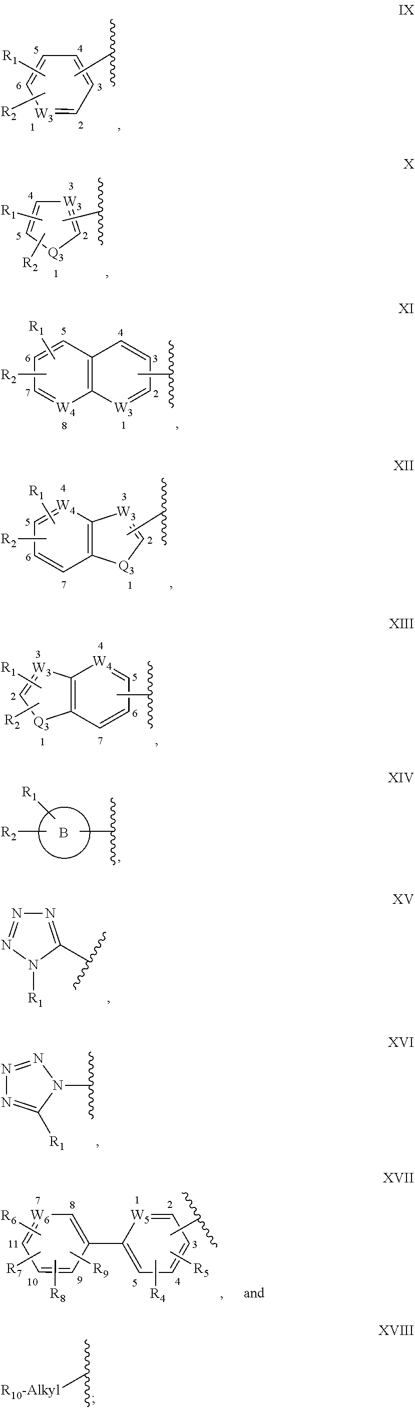

wherein: W₃ is CH, O, or N if W₃ is not bonded to X or R₁ or R₂; W₃ is C if W₃ is bonded to X or R₁ or R₂; if W₃ is N then it can occupy position 1, 2, 3, 4, 5 or 6 in IX, position 2, 3, 4 or 5 in X, position 1, 2, 3 or 4 in XI, position 2 or 3 in XII, and position 2 or 3 in XIII;
W₄ is CH or N if W₄ is not bonded to X or R₁ or R₂; W₄ is C if W₄ is bonded to X or R₁ or R₂; if W₄ is N then it can occupy position 5, 6, 7 or 8 in XI, position 4, 5, 6 or 7 in XII and position 4, 5, 6 or 7 in XIII;
W₅ is CH or N if W₅ is not bonded to X or R₄ or R₅; W₅ is C if W₅ is bonded to X or R₄ or R₅; if W₅ is N then it can occupy position 1, 2, 3, 4 or 5 in XVII;
W₆ is CH or N if W₆ is not bonded to R₆ or R₇ or R₈ or R₉; W₆ is C if W₆ is bonded to R₆ or R₇ or R₈ or R₉; if W₆ is N then it can occupy position 7, 8, 9, 10 or 11 in XVII;
Q₃ is CH₂, O, S or NH if Q₃ is not bonded to X or R₁ or R₂; Q₃ is CH or N if Q₃ is bonded to X or R₁ or R₂;
B is adamantyl or heteroadamantyl;
R₁ and R₂ are each independently —H, —F, —Cl, —Br, —I, —OH, —SH, —NH₂, —CN, —N₃, —NCS, —NCO, —CONH₂, —SO₂NH₂, —COOH, —NO₂, —CHO, —CF₃, —SO₃H, —SO₂Cl, —SO₂F, —O—P(O)(OH)₂, —O—P(O)(O-alkyl)₂, —O—P(O)(OH)(O-alkyl), —P(O)(O-alkyl)₂, —P(O)(OH)(O-alkyl), —Sn(alkyl)₃, —Si(alkyl)₃, —C≡CH, —CH₂—C≡CH, —CH═CH₂, -alkyl-R₃, -cycloalkyl-R₃, -heterocyclic-R₃, -aryl-R₃, -heteroaryl-R₃, -alkyl-cycloalkyl-R₃, -alkyl-heterocyclic-R₃, -alkyl-aryl-R₃, -alkyl-heteroaryl-R₃, —Z-alkyl-R₃, Z-cycloalkyl-R₃, —Z-heterocyclic-R₃, —Z-aryl-R₃, —Z-heteroaryl-R₃, —Z-alkyl-cycloalkyl-R₃, —Z-alkyl-heterocyclic-R₃, —Z-alkyl-aryl-R₃, —Z-alkyl-heteroaryl-R₃, -aryl-Z-alkyl-R₃, -aryl-Z-cycloalkyl-R₃, -aryl-Z-heterocyclic-R₃, -aryl-Z-aryl-R₃, -aryl-Z-heteroaryl-R₃, -aryl-Z-alkyl-cycloalkyl-R₃, -aryl-Z-alkyl-heterocyclic-R₃, -aryl-Z-alkyl-aryl-R₃, -aryl-Z-alkyl-heteroaryl-R₃, —CH(alkyl-R₃)₂, —C(alkyl-R₃)₃, —N(alkyl-R₃)₂, —C(O)N(alkyl-R₃)₂, —SO₂N(alkyl-R₃)₂, or adamantyl;
Z is —O, —S, —NH, —C(O), —C(O)O, —OC(O), —C(O)NH, —NHC(O), —SO, —SO₂, —SO₂NH, —NHSO₂, —SO₂O, or —OSO₂;
R₃ is —H, —F, —Cl, —Br, —I, -Me, -Et, —OH, —OAc, —SH, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, —C≡CH, —CH₂—C≡CH, or —CH═CH₂;
R₄, R₅, R₆, R₇, R₈, and R₉ are each independently —H, —F, —Cl, —Br, —I, —OH, —OMe, —OEt, —OCH₂OCH₃, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, -alkyl, or -alkyl-R₃; and
R₁₀ is —H, —F, —Cl, —Br, —I, —OH, —OMe, —OEt, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, —C≡CH, —CH₂—C≡CH, or —CH═CH₂; and

wherein: if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0, then R can not be IX, X, XI, XII, XIII, or XVIII when one of R₁ or R₂ is H;

if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0-3, and R is XVII, then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂;

if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0; then R can not be IX, X, XI, XII, XIII, or XVIII when one of R₁ or R₂ is H; and

if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0-3, and R is XVII; then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂.

In some embodiments, the enzyme inhibited by the compound of Formula (I) is MGL and/or FAAH.

In certain embodiments, the cannabinergic ligand is 2-arachidonoylglycerol or anandamide.

In some embodiments, the CB1 receptor or the CB2 receptor is modulated.

In still further embodiments, the compound having formula R—X—Y in the method of modulation is a compound listed in Tables 1 and 2 in the below Examples.

A further aspect of the disclosure is directed to a method of treating a neuropathy in a subject. In this method, a therapeutically effective amount of a compound of Formula (I) is administered to the subject. The administration of the compound treats the neuropathy of the subject. In some embodiments, the neuropathy is inflammation, pain, neuropathic pain, neuropathic low back pain, complex regional pain syndrome, post trigeminal neuralgia, causalgia, toxic neuropathy, reflex sympathetic dystrophy, diabetic neuropathy, chronic neuropathy caused by chemotherapeutic agents, central pain, peripheral pain, pellagric neuropathy, alcoholic neuropathy, Beriberi neuropathy, or burning feet syndrome. In particular embodiments, the compound of Formula (I) is a compound listed in Tables 1 and 2, below.

In yet other embodiments, the neuropathy is a neurodegenerative disease. In particular embodiments, the neurodegenerative disease is multiple sclerosis, Parkinson's disease, Huntington's chorea, Alzheimer's disease, amyotrophic lateral sclerosis, memory disorder, mood disorder, sleep disorder, gastrointestinal motility disorder, irritable bowel syndrome, diarrhea, cardiovascular disease, hypertension, osteoporosis, osteoarthritis, emesis, epilepsy, a mental disorder, schizophrenia, depression, glaucoma, cachexia, insomnia, traumatic brain injury, spinal cord injury, seizures, excitotoxin exposure, ischemia, or AIDS wasting syndrome.

An additional aspect of the application is directed to a method of treating a motor function disorder in a subject. The method comprises administering to the subject a therapeutically effective amount of a compound of Formula (I). The administration of the compound treats the motor function disorder of the subject. In one embodiment, the motor function disorder is Tourette's syndrome. In particular embodiments, the compound of Formula (I) is a compound listed in Tables 1 and 2, below.

Another aspect of the application is directed to a method of treating an anxiety disorder in a subject. The method comprises administering to the subject a therapeutically effective amount of a compound of Formula (I). The administration of the compound treats the anxiety disorder of the subject. In certain embodiments, the anxiety disorder is panic disorder, acute stress disorder, post-traumatic stress disorder, substance-induced anxiety disorder, obsessive compulsive disorder, agoraphobia, specific phobia, or social phobia. In particular embodiments, the compound of Formula (I) is a compound listed in Tables 1 and 2, below.

An additional aspect of the disclosure is directed to a method of treating a fertility disorder in a subject. The method comprises administering to the subject a therapeutically effective amount of a compound of Formula (I). The administration of the compound treats the fertility disorder of the subject. In particular embodiments, the compound of Formula (I) is a compound listed in Tables 1 and 2, below.

In yet another aspect, the disclosure is directed to a method of treating an appetite disorder in a subject. The method comprises administering to the subject a therapeutically effective amount of a compound of Formula (I). The administration of the compound treats the appetite disorder, the metabolic disorder, or the movement disorder of the subject. In particular embodiments, the compound of Formula (I) is a compound listed in Tables 1 and 2, below.

In another aspect, the disclosure is directed to a method of treating a metabolic disorder in a subject. The method comprises administering to the subject a therapeutically effective amount of a compound of Formula (I). The administration of the compound treats the metabolic disorder of the subject. In particular embodiments, the compound of Formula (I) is a compound listed in Tables 1 and 2, below.

In still another aspect, the disclosure is directed to a method of treating a movement disorder in a subject. The method comprises administering to the subject a therapeutically effective amount of a compound of Formula (I). The administration of the compound treats the movement disorder of the subject. In particular embodiments, the compound of Formula (I) is a compound listed in Tables 1 and 2, below.

Another aspect of the disclosure is directed to a method of treating cancer in a subject. The method comprising administering to the subject a therapeutically effective amount of a compound of Formula (I). The administration of the compound treats the cancer of the subject. In particular embodiments, the compound of Formula (I) is a compound listed in Tables 1 and 2, below.

Another aspect of the disclosure is directed to sulfonyl chlorides. In certain embodiments, the sulfonyl chloride is selected from the group consisting of

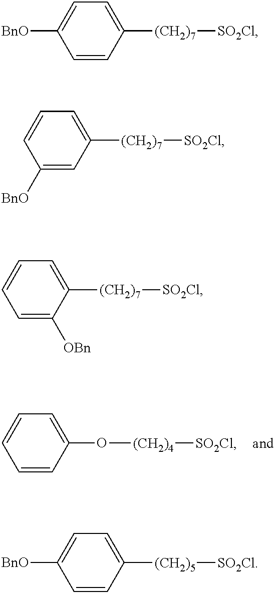

Still another aspect of the disclosure is directed to sulfonyl fluorides. In some embodiments, the sulfonyl fluoride is selected from the group consisting of

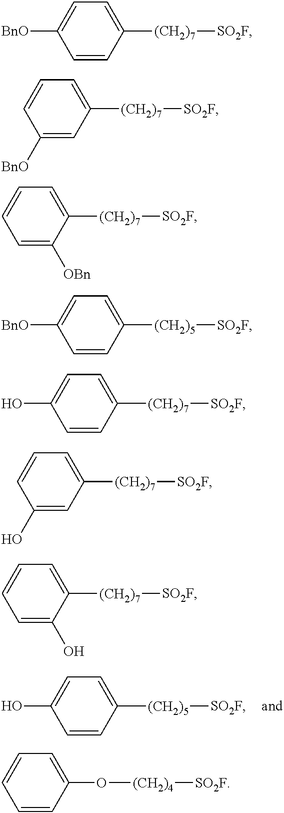

Yet another aspect of the disclosure is directed to trifluoromethyl ketones. In particular embodiments, the trifluoromethyl ketone is selected from the group consisting of

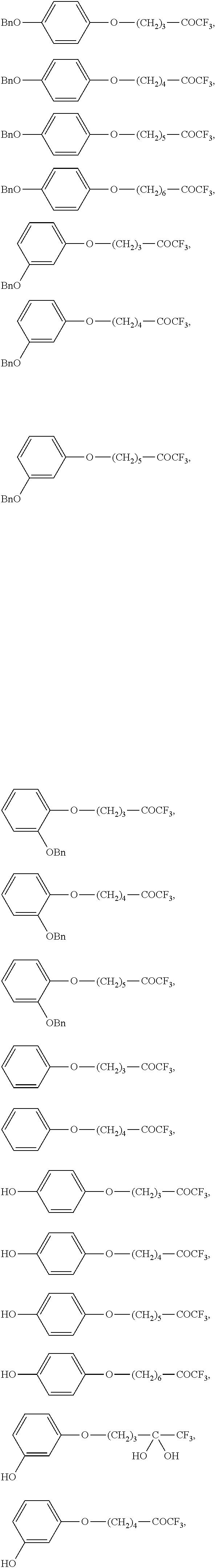

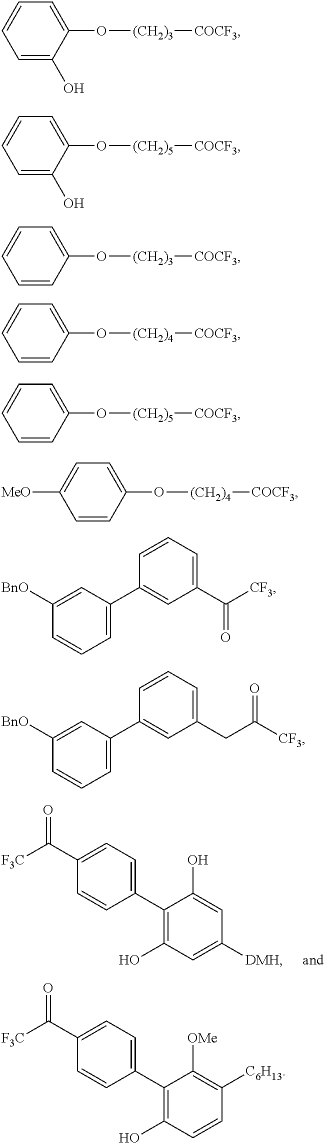

A further aspect of the disclosure is directed to carbamates. In some embodiments, the carbamate is selected from the group consisting of

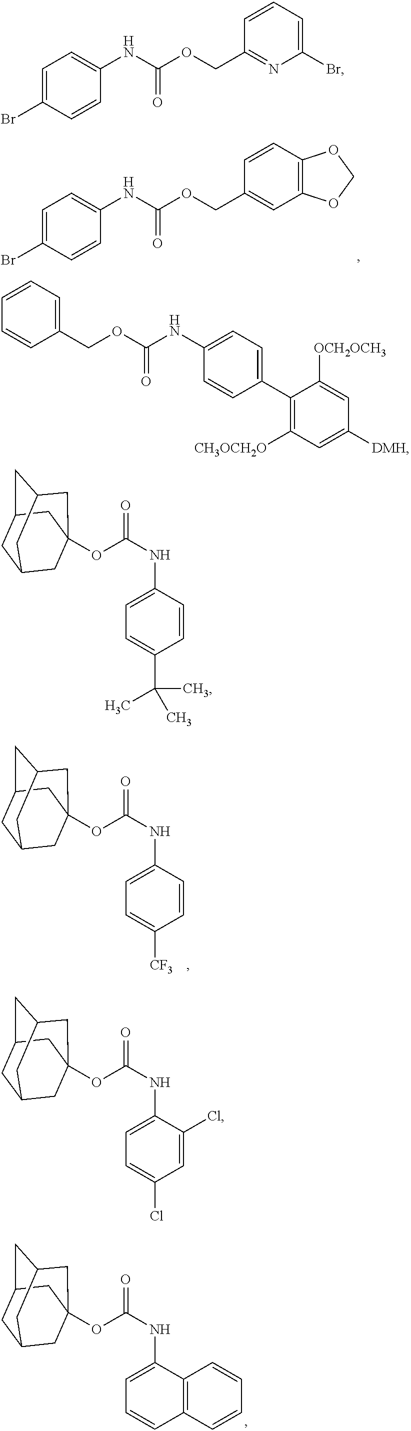

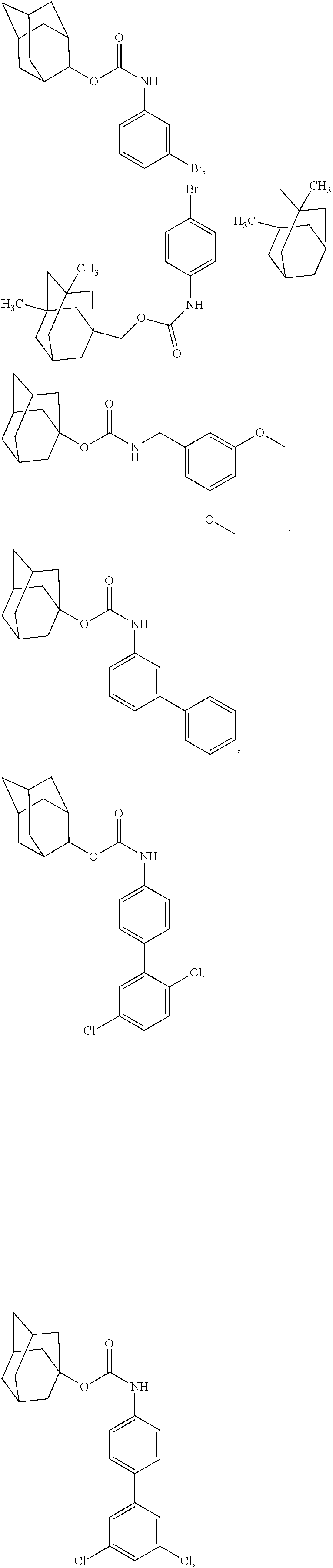

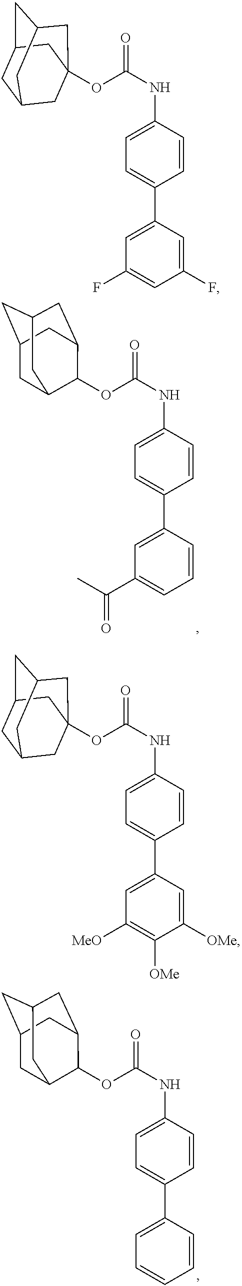

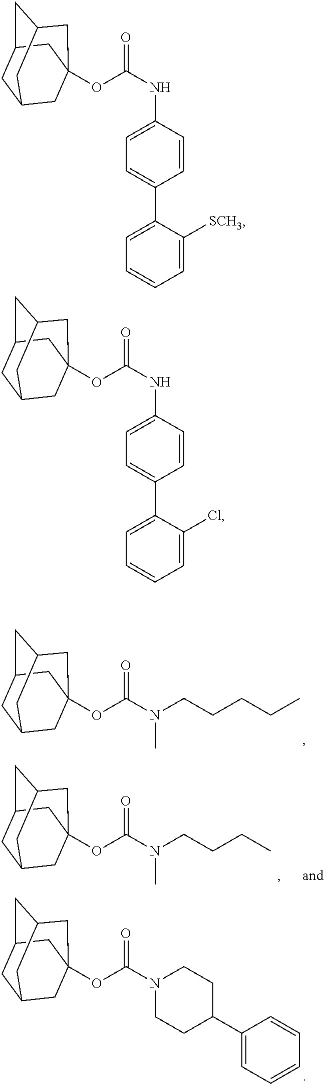

In another aspect, the disclosure is directed to ureas. In certain embodiments, the urea is selected from the group consisting of

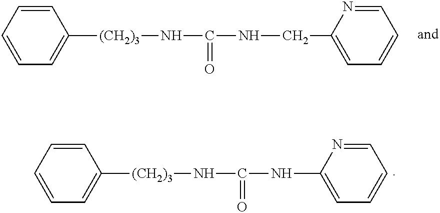

In a further aspect, the disclosure is directed to α-Keto-oxadiazoles. In certain embodiments, the α-Keto-oxadiazole is selected from the group consisting of

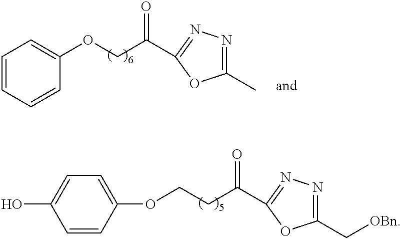

In another aspect, the disclosure is directed to saccharin analogs. In some embodiments, the saccharin analog is selected from the group consisting of

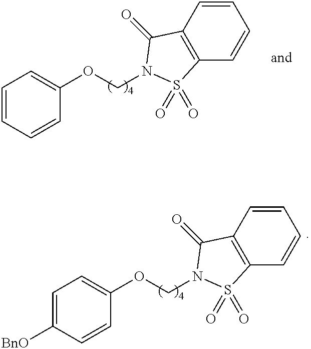

### DETAILED DESCRIPTION

This application relates to compounds, and enantiomers, diastereomers, tautomers, pharmaceutically-acceptable salts, and solvates of those compounds, that inhibit MGL or MGL and FAAH, to methods for modulating cannabinoid receptors, to methods for inhibiting MGL and FAAH, to processes for the preparation of these compounds and their enantiomers, diastereomers, tautomers or pharmaceutically-acceptable salts or solvates, to pharmaceutical compositions comprising these compounds and their enantiomers, diastereomers, tautomers, and pharmaceutically-acceptable salts or solvates, and to methods for treating inflammation, pain, neuropathy, central nervous system disorders, and neurodegenerative disorders.

Throughout this application, various patents, patent applications and publications are referenced. The disclosures of these patents, patent applications and publications in their entireties are incorporated into this application by reference in order to more fully describe the state of the art as known to those skilled therein as of the date of this application. This disclosure will govern in the instance that there is any inconsistency between the patents, patent applications and publications and this disclosure.

### 1. Definitions

The compounds of this disclosure include any and all possible isomers, stereoisomers, enantiomers, diastereomers, tautomers, pharmaceutically-acceptable salts, and solvates thereof. Thus, the terms “compound” and “compounds” as used in this disclosure refer to the compounds of this disclosure and any and all possible isomers, stereoisomers, enantiomers, diastereomers, tautomers, pharmaceutically-acceptable salts, and solvates thereof.

In general, the compositions of the disclosure can be alternately formulated to comprise, consist of, or consist essentially of, any appropriate components disclosed in this application. The compositions of the disclosure can additionally, or alternatively, be formulated so as to be devoid, or substantially free, of any components, materials, ingredients, adjuvants or species used in the prior art compositions or that are otherwise not necessary to the achievement of the function and/or objectives of the present disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs. The initial definition provided for a group or term provided in this disclosure applies to that group or term throughout the present disclosure individually or as part of another group, unless otherwise indicated.

The articles “a” and “an” are used in this disclosure to refer to one or more than one (i.e., to at least one) of the grammatical object of the article. By way of example, “an element” means one element or more than one element.

The term “or” is used in this disclosure to mean, and is used interchangeably with, the term “and/or,” unless indicated otherwise.

The term “about” is used in this disclosure to mean a value − or +20% of a given numerical value. Thus, “about 60%” means a value between 60-20% of 60 and 60+20% of 60 (i.e., between 48% and 72%).

Unless otherwise specifically defined, “alcohol” refers to the general formula alkyl—OH and includes primary, secondary and tertiary variations.

Unless otherwise specifically defined, the terms “alkyl” and “alk” refer to a straight or branched chain alkane (hydrocarbon) radical containing from 1 to 15 carbon atoms. Exemplary “alkyl” groups include, but are not limited to, methyl (“Me”), ethyl (“Et”), propyl, isopropyl, n-butyl, t-butyl, sec-butyl, isobutyl, pentyl, hexyl, isohexyl, heptyl, 4,4-dimethylpentyl, 1,1-dimethylpentyl, 1,2-dimethylheptyl, octyl, 2,2,4-trimethylpentyl, nonyl, decyl, undecyl, dodecyl, and the like. The alkyl group may be optionally substituted with one or more substituents, e.g., 1 to 5 substituents, at any available point of attachment. Exemplary substituents include, but are not limited to, one or more of the following groups: hydrogen, halogen (e.g., a single halogen substituent or multiple halo substituents forming, in the latter case, groups such as CF₃), cyano, nitro, CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}Rₑ, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each Rₐ is hydrogen, alkyl, cycloalkyl, alkenyl, cycloalkenyl, alkynyl, heterocycle, or aryl; R_{b}, R_{c} and R_{d} are each independently hydrogen, alkyl, cycloalkyl, heterocycle, aryl, or said R_{b} and R_{c}, together with the N to which they are bonded optionally form a heterocycle or substituted heterocycle; and each Rₑ is alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle and aryl can themselves be optionally substituted. The term “C₁-Cₙ-alkyl” refers to a straight or branched chain alkane (hydrocarbon) radical containing from 1 to n carbon atoms. For example, the term “C₁-C₅-alkyl” refers to a straight or branched chain alkane (hydrocarbon) radical containing from 1 to 5 carbon atoms, such as methyl, ethyl, propyl, isopropyl, n-butyl, t-butyl, isobutyl, etc.

Unless otherwise specifically defined, the term “alkenyl” refers to a straight or branched chain hydrocarbon radical containing from 2 to 15 carbon atoms and at least one carbon-carbon double bond. Exemplary such groups include, but are not limited to, ethenyl (also called “vinyl”), allyl, propenyl, crotyl, 2-isopentenyl, allenyl, butenyl, butadienyl, pentenyl, pentadienyl, 3(1,4-pentadienyl), hexenyl and hexadienyl. The alkenyl group may be optionally substituted with one or more substituents, e.g., 1 to 5 substituents, at any available point of attachment. Exemplary substituents include, but are not limited to, alkyl or substituted alkyl, as well as those groups recited above as exemplary alkyl substituents. The exemplary substituents can themselves be optionally substituted.

Unless otherwise specifically defined, the term “alkynyl” refers to a straight or branched chain hydrocarbon radical containing from 2 to 15 carbon atoms and at least one carbon-carbon triple bond. Exemplary such groups include, but are not limited to, ethynyl, propynyl and butynyl. The alkynyl group may be optionally substituted with one or more substituents, e.g., 1 to 5 substituents, at any available point of attachment. Exemplary substituents include, but are not limited to, alkyl or substituted alkyl, as well as those groups recited above as exemplary alkyl substituents. The exemplary substituents can themselves be optionally substituted.

Unless otherwise specifically defined, the term “aryl” refers to cyclic, aromatic hydrocarbon groups that have 1 to 5 aromatic rings, including monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The aryl group may be optionally substituted by one or more substituents, e.g., 1 to 5 substituents, at any point of attachment. Exemplary substituents include, but are not limited to, nitro, cycloalkyl or substituted cycloalkyl, cycloalkenyl or substituted cycloalkenyl, cyano, alkyl, fused cyclic groups, fused cycloalkyl, fused cycloalkenyl, fused heterocycle, and fused aryl, and those groups recited above as exemplary alkyl substituents. The substituents can themselves be optionally substituted.

Unless otherwise specifically defined, the term “cycloalkyl” refers to a fully saturated cyclic hydrocarbon group containing from 1 to 4 rings and 3 to 8 carbons per ring. Exemplary such groups include, but are not limited to, cyclopropyl, cyclobutyl, cyclopentyl, cyclohexyl, cycloheptyl, adamantyl, etc. The cycloalkyl group may be optionally substituted with one or more substituents, e.g., 1 to 5 substituents, at any available point of attachment. Exemplary substituents include, but are not limited to, nitro, cyano, alkyl, spiro-attached or fused cyclic substituents, spiro-attached cycloalkyl, spiro-attached cycloalkenyl, spiro-attached heterocycle, fused cycloalkyl, fused cycloalkenyl, fused heterocycle, fused aryl, and those groups recited above as exemplary alkyl substituents. The substituents can themselves be optionally substituted.

Unless otherwise specifically defined, the term “adamantyl” includes, but is not limited to, 1-adamantyl, 2-adamantyl, and 3-adamantyl. The adamantyl group may be optionally substituted with the groups recited as exemplary cycloalkyl substituents.

Unless otherwise specifically defined, the term “cycloalkenyl” refers to a partially unsaturated cyclic hydrocarbon group containing 1 to 4 rings and 3 to 8 carbons per ring. Exemplary such groups include, but are not limited to, cyclobutenyl, cyclopentenyl, cyclohexenyl, etc. The cycloalkenyl group may be optionally substituted with one more substituents, e.g., 1 to 5 substituents, at any available point of attachment. Exemplary substituents include, but are not limited to, nitro, cyano, alkyl or substituted alkyl, spiro-attached or fused cyclic substituents, spiro-attached cycloalkyl, spiro-attached cycloalkenyl, spiro-attached heterocycle (excluding heteroaryl), fused cycloalkyl, fused cycloalkenyl, fused heterocycle, fused aryl, and those groups recited above as exemplary alkyl substituents. The substituents can themselves be optionally substituted.

Unless otherwise specifically defined, the terms “heterocycle” and “heterocyclic” refer to fully saturated, or partially or fully unsaturated, including aromatic (i.e., “heteroaryl”) cyclic groups (for example, 4 to 7 membered monocyclic, 7 to 11 membered bicyclic, or 8 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached to the remainder of the molecule at any heteroatom or carbon atom of the ring or ring system. Exemplary monocyclic heterocyclic groups include, but are not limited to, azetidinyl, pyrrolidinyl, pyrrolyl, pyrazolyl, oxetanyl, dioxanyl, dioxolanyl, oxathiolanyl, pyrazolinyl, imidazolyl, imidazolinyl, imidazolidinyl, oxazolyl, oxazolidinyl, isoxazolinyl, isoxazolyl, thietanyl, azetidine, diazetidine, thiolanyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, purinyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include, but are not limited to, indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include, but are not limited to, carbazolyl, benzidolyl, phenanthrolinyl, acridinyl, phenanthridinyl, xanthenyl and the like.

A heterocyclic group may be optionally substituted with one or more substituents, e.g., 1 to 5 substituents, at any available point of attachment. Exemplary substituents include, but are not limited to, cycloalkyl or substituted cycloalkyl, cycloalkenyl or substituted cycloalkenyl, nitro, oxo (i.e., ═O), cyano, alkyl or substituted alkyl, spiro-attached or fused cyclic substituents at any available point or points of attachment, spiro-attached cycloalkyl, spiro-attached cycloalkenyl, spiro-attached heterocycle (excluding heteroaryl), fused cycloalkyl, fused cycloalkenyl, fused heterocycle, fused aryl, and those groups recited above as exemplary alkyl substituents. The substituents can themselves be optionally substituted.

Unless otherwise indicated, any heteroatom with unsatisfied valences is assumed to have hydrogen atoms sufficient to satisfy the valences.

The term “heating” includes, but is not limited to, warming by conventional heating (e.g., electric heating, steam heating, gas heating, etc.) as well as microwave heating.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The phrase “pharmaceutically acceptable” is employed in this disclosure to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salt(s)”, as employed in this disclosure, denotes acidic and/or basic salts formed with inorganic and/or organic acids and bases.

The term “treating” with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwised indicated.

The terms “effective amount” and “therapeutically effective amount” as used in this disclosure refer to an amount of a compound that, when administered to a subject, is capable of reducing a symptom of a disorder in a subject. The actual amount which comprises the “effective amount” or “therapeutically effective amount” will vary depending on a number of conditions including, but not limited to, the particular disorder being treated, the severity of the disorder, the size and health of the patient, and the route of administration. A skilled medical practitioner can readily determine the appropriate amount using methods known in the medical arts.

As used in this disclosure, the term “subject” includes, without limitation, a human or an animal Exemplary animals include, but are not limited to, mammals such as mouse, rat, guinea pig, dog, cat, horse, cow, pig, monkey, chimpanzee, baboon, or rhesus monkey.

The term “administer”, “administering”, or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a compound of Formula (I).

The term “halogen” as used in this disclosure refers to fluorine, chlorine, bromine, and iodine.

The terms “isolated” and “purified” as used in this disclosure refer to a component separated from other components of a reaction mixture or a natural source. In certain embodiments, the isolate contains at least about 50%, at least about 55%, at least about 60%, at least about 65%, at least about 70%, at least about 75%, at least about 80%, at least about 85%, at least about 90%, at least about 95%, or at least about 98% of the compound or pharmaceutically acceptable salt of the compound by weight of the isolate.

The term “tautomer” as used in this disclosure refers to compounds produced by the phenomenon wherein a proton of one atom of a molecule shifts to another atom. (March, Advanced Organic Chemistry: Reactions, Mechanisms and Structures, 4th Ed., John Wiley & Sons, pages 69-74 (1992)).

The following abbreviations are used in this disclosure and have the following definitions: MeCN is acetonitrile; DMF is dimethylformamide; DMSO is dimethylsulfoxide; HPLC is high-performance liquid chromatograpy; THF is tetrahydrofuran; EDTA; Tris is tris(hydroxymethyl)aminomethane; TBSC1 is t-butyldimethylsilyl Chloride; TBAF is tetra-n-butylammonium fluoride; “h” is hour or hours; and “RT” is RT.

### 2. MGL Inhibitory Compounds

Certain chemical compounds have been found to inhibit the inactivation of cannabinergic ligands by MGL. These compounds may not bind to, or may have lesser affinity for, the CB1 And/or CB2 cannabinoid receptors. Thus, the physiological action for such compounds and may not be the direct modulation of the CB1 and/or CB2 receptors.

Inhibition of MGL in a subject slows the normal degradation and inactivation of endogenous cannabinoid ligands by MGL hydrolysis. This inhibition allows maintained or higher levels of those endogenous cannabinergic ligands to remain present in the subject. The maintained or higher levels of endocannabinoid ligands provide increased stimulation of the cannabinoid CB1 and CB2 receptors. The increased stimulation of the cannabinoid receptors allows the receptors to produce physiological effects at a maintained or increased level. Thus, a compound that inhibits the inactivation of endogenous cannabinoid ligands by MGL increases the levels of endocannabinoids, thereby enhancing the activation of cannabinoid receptors. The compound does not directly modulate the cannabinoid receptors but instead indirectly stimulates the cannabinoid receptors by increasing the in vivo levels of endocannabinoid ligands.

The inhibition of MGL also enhances the effects of exogenous cannabinergic ligands and allows them to stimulate cannabinoid receptors at lower concentrations as compared to systems in which MGL action is not inhibited. Thus, inhibition of MGL also enhances the effects and duration of action of exogenous cannabinergic ligands.

Examples of cannabinergic ligands that bind to CB1 and/or CB2 include, but are not limited to, N-arachidonoyl ethanolamine (also known as anandamide or AEA) and 2-arachidonoylglycerol (2-AG) (both endogenous ligands for the cannabinoid CB1 and CB2 receptors), (−)-Δ⁹-tetrahydrocannabinol (the principal bioactive constituent of cannabis and exogenous ligand for the cannabinoid CB1 and CB2 receptors) and other synthetic cannabinergic analogs.

Marijuana-like cannabinoids, in addition to acting at cannabinoid receptors, also affect cellular membranes, and are known to cause undesirable side effects such as drowsiness, impairment of monoamide oxidase function, and impairment of non-receptor mediated brain function. Thus, the addictive and psychotropic properties of some cannabinoids limit their therapeutic value. Compounds that inhibit MGL activity provide an alternative mechanism for stimulating cannabinoid receptors and provide desirable pharmacological properties without the undesirable properties associated with increased concentrations of cannabinoids.

The present disclosure provides novel chemical compounds of Formula (I), R—X—Y, that inhibit MGL or that jointly inhibit both FAAH and MGL, wherein Y is selected from the group consisting of:

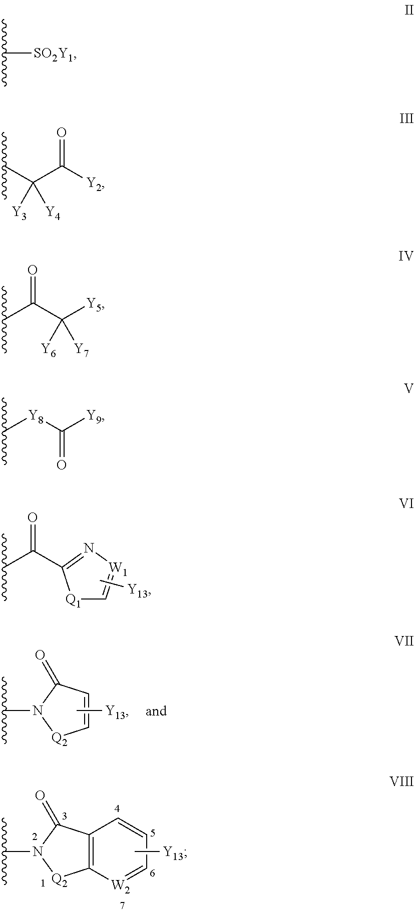

wherein: Y₁ is —F, —Cl, —O-alkyl, —O-cycloalkyl, —O-heterocyclic, —O-aryl, —O-heteroaryl, or —O-adamantyl;
Y₂ is —H, —OH, —NH₂, —OMe, —OEt, —CF₃, —C≡CH, —CH₂—C≡CH, —CH═CH₂, fluoroalkyl, —C₁₋₅-alkyl, -aryl, -alkyl-aryl, -aryl-alkyl, -aryl-alkyl-Y₁₄, -aryl-heteroaryl, -aryl-aryl, -heteroaryl, -heteroaryl-alkyl, -heteroaryl-alkyl-Y₁₄, -heteroaryl-aryl, -heteroaryl-heteroaryl, -cycloalkyl, -cycloalkyl-alkyl, -cycloalkyl-alkyl-Y₁₄, -heterocyclic, -heterocyclic-alkyl, -heterocyclic-alkyl-Y₁₄, -adamantyl, —C₁₋₅-alkyl-Y₁₄, -aryl-Y₁₄, -heteroaryl-Y₁₄, -cycloalkyl-Y₁₄, -heterocyclic-Y₁₄, or -adamantyl-Y₁₄;
Y₃ and Y₄ are each independently —F, —Cl, or —OH; or Y3 and Y4 taken together form a ketone;
Y₅ is —F, —CONH₂, —SO₂NH₂, —COOH, —COOMe, —COOEt, —CF₃, —C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₆ and Y₇ are each independently —F, —Cl, or —OH;
Y₈ is NH, O, or heterocycle;
Y₉ is —OY₁₀, —N(Y₁₁)Y₁₂, or heterocycle;
Y₁₀ is alkyl, aryl, benzyl, difluorophenyl, fluorophenyl, heteroaryl, cycloalkyl, adamantyl, heterocyclic, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₁₁ is —H, -alkyl, -aryl, or -alkyl-aryl;
Y₁₂ is alkyl, aryl, heteroaryl, cycloalkyl, adamantyl, heterocyclic, —C₁₋₅-alkyl-aryl, —C₁₋₅-alkyl-heteroaryl, -aryl-(Y₁₄)₁₋₄; -heteroaryl-Y₁₄, -cycloalkyl-Y₁₄, -adamantyl-Y₁₄; or -heterocyclic-Y₁₄; or Y₁₁ and Y₁₂ when taken together along with the N to which they are bonded form a 5- or 6-membered saturated heterocylic ring, the ring containing up to one additional heteroatom selected from the group consisting of N, O, and S;
Y₁₃ is —H, —OH, —SH, —NH₂, —CN, —N₃, —NCS, —NCO, —CONH₂, —SO₂NH₂; —COOH, —COOMe, —COOEt, —NO₂, —CF₃, —SO₃H, —P(O)(OH)₂, —CH₂—C≡CH, —CH═CH₂, fluoroalkyl, —C₁₋₆-alkyl, aryl, heteroaryl, cycloalkyl, adamantyl, heterocyclic, —C₁₋₆-alkyl-Y₁₄; -aryl-Y₁₄, heteroaryl Y₁₄ cycloalkyl-Y₁₄, -adamantyl-Y₁₄, or -heterocyclic-Y₁₀;
Y₁₄ is —H, —F, —Cl, Br, —I, —OH, —OMe, —OEt, —OPh, —OBn, —SH, —NH₂, —CN, —N₃, —NCS, —NCO, —CONH₂, —SO₂NH₂, —COOH, —COOMe, —COOEt, —NO₂, -alkyl, —CF₃, —SO₃H, —P(O)(OH)₂, —CH₂—C≡CH, —CH═CH₂, or —NHCOCH₃, or —CH₂OH;
W₁ is CH or N if Y₁₃ is not bonded to W₁, or W₁ is C if Y₁₃ is bonded to W₁;
W₂ is CH or N if W₂ is not bonded to Y₁₃, or W₂ is C if W₂ is bonded to Y₁₃; if W₂ is N then it can occupy position 4, 5, 6, or 7 in VIII;
Q₁ is —CH₂, —O, —S, or —NH if Q₁ is not bonded to Y₁₃; Q₁ is —CH or —N if Q₁ is bonded to Y₁₃;
Q₂ is —SO₂, —C(O), or —S(O);

wherein: X is —(CH₂)ₙ—, —(CH₂)ⱼ-A-(CH₂)ₖ—, cycloalkyl, or heterocycle; A is —CH═CH—, —C≡C—, C═O, O, S, or NH; n is an integer from 0 to 15; j is an integer from 0 to 10; and k is an integer from 0 to 10; and wherein: R is selected from the group consisting of:

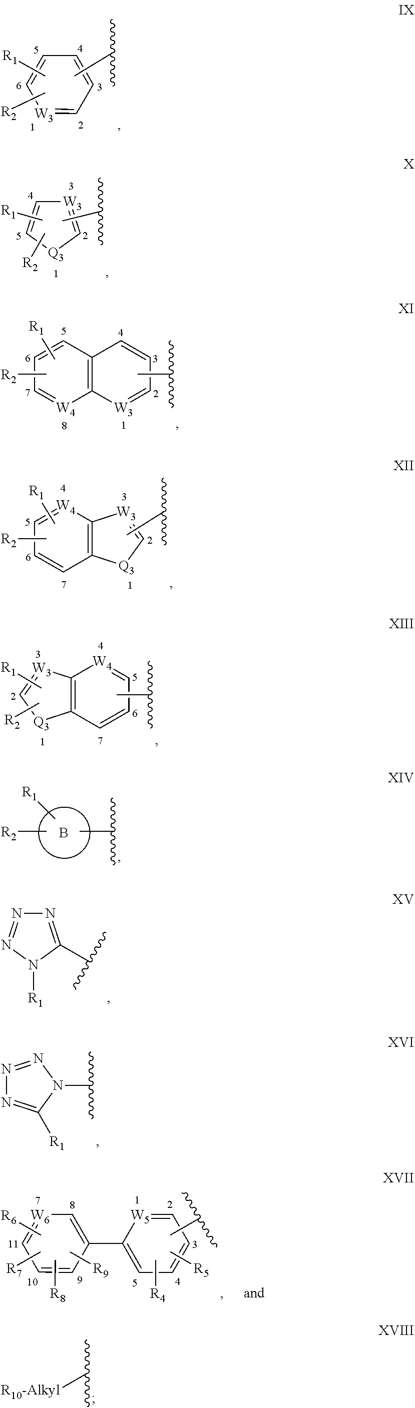

wherein: W₃ is CH, O, or N if W₃ is not bonded to X or R₁ or R₂; W₃ is C if W₃ is bonded to X or R₁ or R₂; if W₃ is N then it can occupy position 1, 2, 3, 4, 5 or 6 in IX, position 2, 3, 4 or 5 in X, position 1, 2, 3 or 4 in XI, position 2 or 3 in XII, and position 2 or 3 in XIII;
W₄ is CH or N if W₄ is not bonded to X or R₁ or R₂; W₄ is C if W₄ is bonded to X or R₁ or R₂; if W₄ is N then it can occupy position 5, 6, 7 or 8 in XI, position 4, 5, 6 or 7 in XII and position 4, 5, 6 or 7 in XIII;
W₅ is CH or N if W₅ is not bonded to X or R₄ or R₅; W₅ is C if W₅ is bonded to X or R₄ or R₅; if W₅ is N then it can occupy position 1, 2, 3, 4 or 5 in XVII;
W₆ is CH or N if W₆ is not bonded to R₆ or R₇ or R₈ or R₉; W₆ is C if W₆ is bonded to R₆ or R₇ or R₈ or R₉; if W₆ is N then it can occupy position 7, 8, 9, 10 or 11 in XVII;
Q₃ is CH₂, O, S or NH if Q₃ is not bonded to X or R₁ or R₂; Q₃ is CH or N if Q₃ is bonded to X or R₁ or R₂;
B is adamantyl or heteroadamantyl;
R₁ and R₂ are each independently —H, —F, —Cl, —Br, —I, —OH, —SH, —NH₂, —CN, —N₃, —NCS, —NCO, —CONH₂, —SO₂NH₂, —COOH, —NO₂, —CHO, —CF₃, —SO₃H, —SO₂Cl, —SO₂F, —O—P(O)(OH)₂, —O—P(O)(O-alkyl)₂, —O—P(O)(OH)(O-alkyl), —P(O)(O-alkyl)₂, —P(O)(OH)(O-alkyl), —Sn(alkyl)₃, —Si(alkyl)₃, —C≡CH, —CH₂—C≡CH, —CH═CH₂, -alkyl-R₃, -cycloalkyl-R₃, -heterocyclic-R₃, -aryl-R₃, -heteroaryl-R₃, -alkyl-cycloalkyl-R₃, -alkyl-heterocyclic-R₃, -alkyl-aryl-R₃, -alkyl-heteroaryl-R₃, —Z-alkyl-R₃, Z-cycloalkyl-R₃, —Z-heterocyclic-R₃, —Z-aryl-R₃, —Z-heteroaryl-R₃, —Z-alkyl-cycloalkyl-R₃, —Z-alkyl-heterocyclic-R₃, —Z-alkyl-aryl-R₃, —Z-alkyl-heteroaryl-R₃, -aryl-Z-alkyl-R₃, -aryl-Z-cycloalkyl-R₃, -aryl-Z-heterocyclic-R₃, -aryl-Z-aryl-R₃, -aryl-Z-heteroaryl-R₃, -aryl-Z-alkyl-cycloalkyl-R₃, -aryl-Z-alkyl-heterocyclic-R₃, -aryl-Z-alkyl-aryl-R₃, -aryl-Z-alkyl-heteroaryl-R₃, —CH(alkyl-R₃)₂, —C(alkyl-R₃)₃, —N(alkyl-R₃)₂, —C(O)N(alkyl-R₃)₂, —SO₂N(alkyl-R₃)₂, or adamantyl;
Z is —O, —S, —NH, —C(O), —C(O)O, —OC(O), —C(O)NH, —NHC(O), —SO, —SO₂, —SO₂NH, —NHSO₂, —SO₂O or —OSO₂;
R₃ is —H, —F, —Cl, —Br, —I, -Me, -Et, —OH, —OAc, —SH, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, —C≡CH, —CH₂—C≡CH, or —CH═CH₂;
R₄, R₅, R₆, R₇, R₈, and R₉ are each independently —H, —F, —Cl, —Br, —I, —OH, —OMe, —OEt, —OCH₂OCH₃, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, -alkyl, or -alkyl-R₃; and
R₁₀ is —H, —F, —Cl, —Br, —I, —OH, —OMe, —OEt, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —O Si(alkyl)₃, —C≡CH, —CH₂—C≡CH or —CH═CH₂; and

wherein: if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0, then R can not be IX, X, XI, XII, XIII, or XVIII when one of R₁ or R₂ is H;

if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0-3, and R is XVII, then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂;

if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0; then R can not be IX, X, XI, XII, XIII, or XVIII when one of R₁ or R₂ is H; and

if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0-3, and R is XVII; then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂.

The compounds of Formula (I) can also form salts which are also within the scope of this disclosure. Reference to a compound of the present disclosure is understood to include reference to salts thereof, unless otherwise indicated. The compounds of Formula (I) may form pharmaceutically acceptable (i.e., non-toxic, physiologically acceptable) salts as well as other salts that are also useful, e.g., in isolation or purification steps which can be employed during preparation.

The compounds of Formula (I) which contain a basic moiety, such as, but not limited to, an amine or a pyridine or imidazole ring, can form salts with a variety of organic and inorganic acids. Exemplary acid addition salts include, but are not limited to, acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of Formula (I) which contain an acidic moiety, such as, but not limited to, a carboxylic acid, can form salts with a variety of organic and inorganic bases. Exemplary basic salts include, but are not limited to, ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups can be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and the like.

The compounds of the present disclosure can have unnatural ratios of atomic isotopes at one or more of their atoms. For example, the compounds can be labeled with isotopes, such as deuterium, tritium carbon-11, carbon-14, iodine-123, iodine-125 or fluorine-18. The present disclosure encompasses all isotopic variations of the described compounds, whether radioactive or not.

Exemplary nonlimiting compounds of Formula (I) are listed in Tables 1 and 2 below. Solvates of the compounds of this disclosure, including hydrates of the compounds, as well as mixtures of the hydrate- and the keto-form of the compounds, are within the scope of this disclosure.

TABLE 1
MGL Inhibitors of Formula (I) (R—X—Y)

| Compound | | % Inhibition | IC₅₀ | Kᵢ |
|---|---|---|---|---|
| number | Structure | (Concentration) | (μM) | (μM) |
| 13.1* | 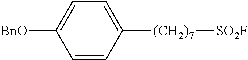 | 30% (100 μM) | 57.1 | 18.6 |
| 13.2* | 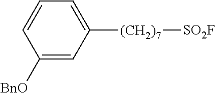 | 30% (100 μM) | | |
| 13.3* | 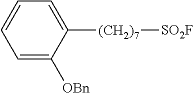 | 92% (100 μM) | 2.70 | 0.77 |
| 13.4* | 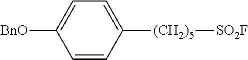 | 57% (100 μM) | 40 | 15 |
| 14.1* | 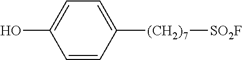 | 87% (100 μM) | 98.2 | 32 |
| 14.2* | 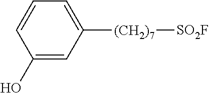 | 98% (100 μM) | 43.7 | 14.3 |
| 14.3* | 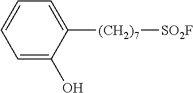 | 97% (100 μM) | 43.1 | 14.1 |
| 14.4* | 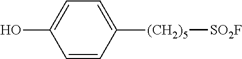 | 56% (100 μM) | 88.2 | 28.8 |
| 17* | 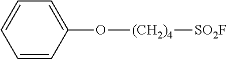 | 26% (100 μM) | 915 | 298 |
| 23.1* | 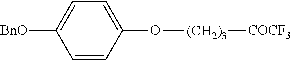 | 65% (100 μM) | 103 | 28 |
| 23.2* | 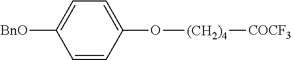 | 64% (100 μM) | 86.2 | 23.7 |
| 23.3* | 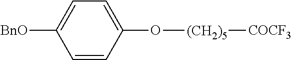 | 61% (100 μM) | 109 | 30 |
| 23.4* | 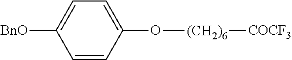 | 34% (100 μM) | | |
| 23.5* | 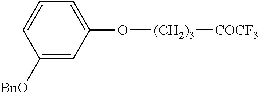 | 69% (100 μM) | 51.6 | 14.2 |
| 23.6* | 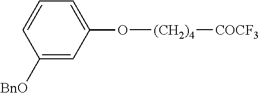 | 66% (100 μM) | 83.4 | 23.8 |
| 23.7* | 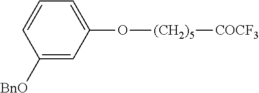 | 38% (100 μM) | 329 | 66.7 |
| 23.8* | 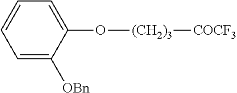 | 20% (100 μM) | | |
| 23.9* | 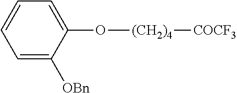 | 47% (100 μM) | | |
| 23.10* | 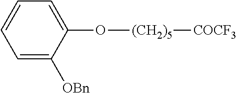 | 66% (100 μM) | 32.7 | 9.0 |
| 23.11* | 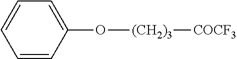 | 13% | | |
| 23.12* | 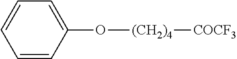 | 3% (100 μM) | | |
| 24.1* | 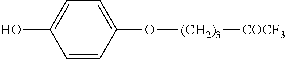 | 6% (100 μM) | | |
| 24.2* | 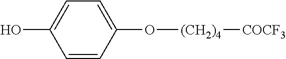 | 21% (100 μM) | | |
| 24.3* | 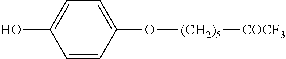 | 17% (100 μM) | | |
| 24.4* | 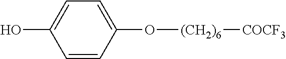 | 42% (100 μM) | 130 | 26.4 |
| 24.5* | 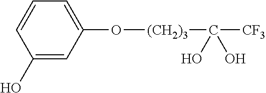 | 3% (100 μM) | | |
| 24.6* | 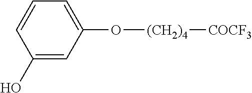 | 9% (100 μM) | | |
| 24.8* | 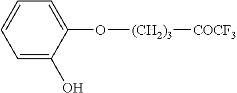 | 4% (100 μM) | | |
| 24.10* | 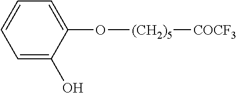 | 19% (100 μM) | | |
| 27.1* | 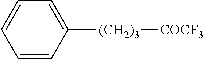 | 5% (100 μM) | | |
| 27.2* | 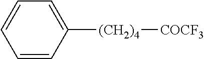 | 21% (100 μM) | | |
| 27.3* | 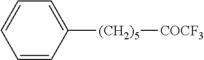 | 36% (100 μM) | | |
| 27.4* | 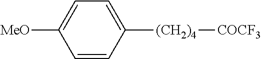 | 13% (100 μM) | | |
| 30* | 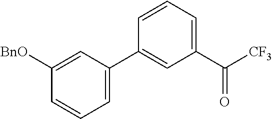 | 36% (100 μM) | 433 | 109 |
| 35* | 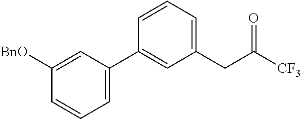 | 21% (100 μM) | | |
| 40.1* | 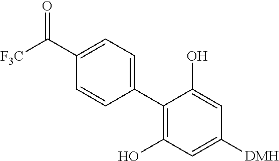 | 85% (100 μM) | 36 | 9 |
| 40.3* | 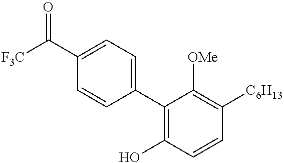 | 81% (100 μM) | | |
| 46.3* | 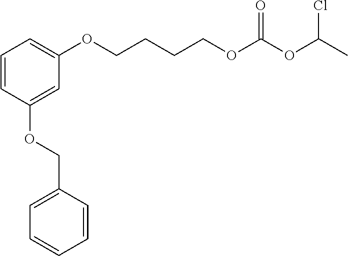 | 35% (100 μM) | | |
| 46.4* | 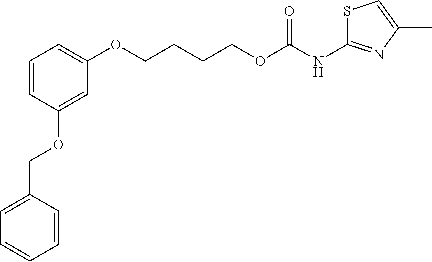 | 24% (100 μM) | | |
| 48.7* | 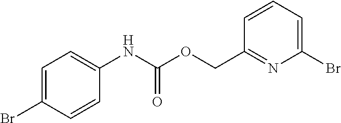 | 55% (100 μM) | | |
| 48.8* | 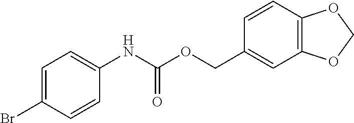 | 32% (100 μM) | | |
| 53.3* | 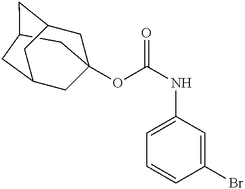 | 44% (100 μM) | | |
| 53.7* | 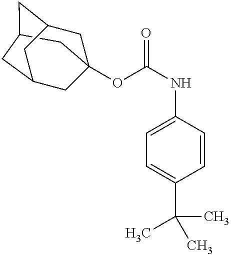 | 34% (100 μM) | | |
| 53.8* | 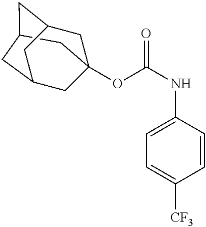 | 54% (100 μM) | | |
| 53.11* | 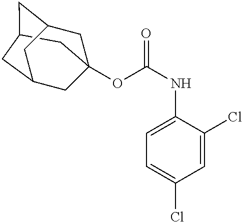 | 89% (100 μM) | | |
| 53.12* | 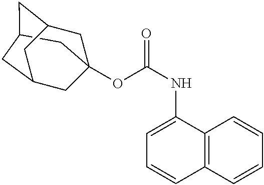 | 17% (100 μM) | | |
| 53.13* | 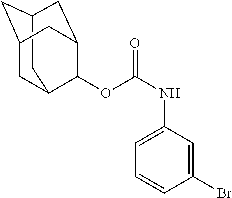 | 76% (100 μM) | | |
| 53.19* | 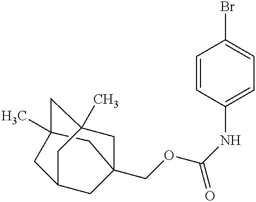 | 78% (100 μM) | | |
| 53.23* | 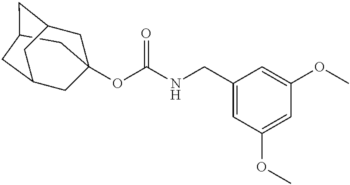 | 22% (100 μM) | | |
| 53.27* | 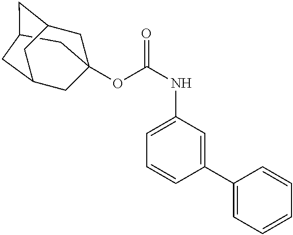 | 88% (100 μM) | | |
| 53.29* | 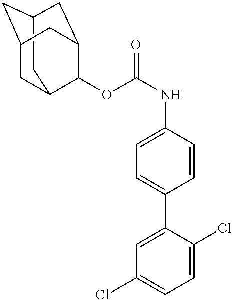 | 84% (100 μM) | | |
| 53.30* | 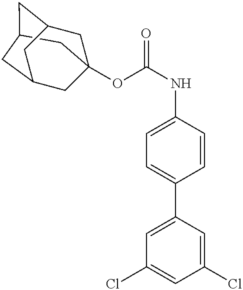 | 72% (100 μM) | | |
| 53.31* | 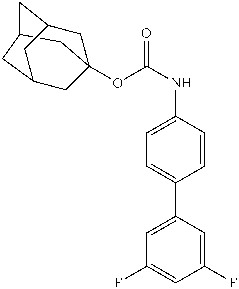 | 54% (100 μM) | | |
| 53.32* | 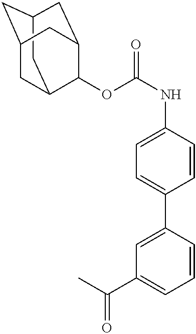 | 18% (100 μM) | | |
| 53.33* | 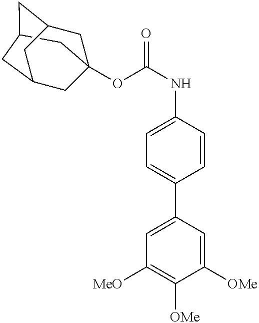 | 21% (100 μM) | | |
| 53.35* | 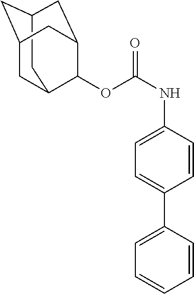 | 78% (100 μM) | | |
| 53.36* | 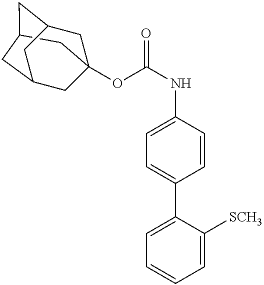 | 12% (100 μM) | | |
| 53.37* | 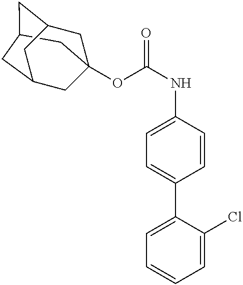 | 61% (100 μM) | | |
| 53.45* | 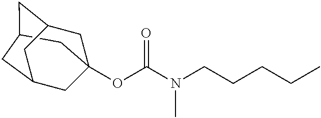 | 13% (100 μM) | | |
| 53.46* | 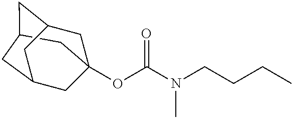 | 14% (100 μM) | | |
| 53.47* | 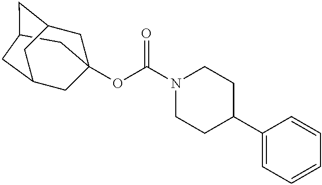 | 22% (100 μM) | | |
| 65.1* | 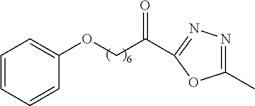 | 6% (100 μM) | | |
| 74.2* | 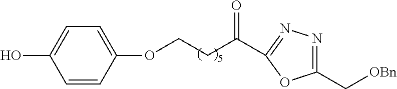 | 29% (100 μM) | | |
| 83.2* | 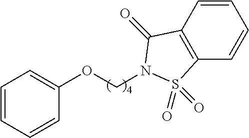 | 2% (100 μM) | | |
| 83.3* | 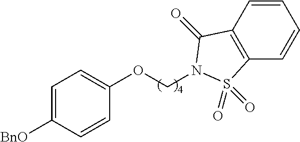 | 1% (100 μM) | | |

^{a}The group BnO— is Ph—CH₂—O— where Ph is Phenyl.
^{b}The group DMH is as shown on Scheme 8.
*Novel compounds.

TABLE 2
MGL Inhibitors of Formula (I) (R—X—Y)

| Compound | | % Inhibition | | | |
|---|---|---|---|---|---|
| number | Structure | (Concentration) | IC₅₀ | | Kᵢ |
| 4.1* | 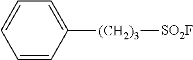 | | | | 1 nM-100 μM |
| 4.2* | 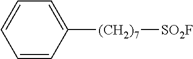 | | | | 1 nM-100 μM |
| 4.3* | 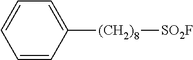 | | | | 1 nM-100 μM |
| 18* | 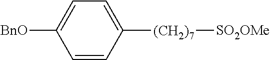 | | | | 1 nM-100 μM |
| 24.7* | 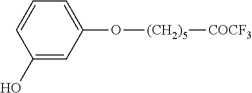 | | | | 1 nM-100 μM |
| 24.9* | 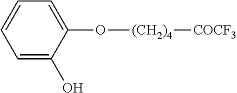 | | | | 1 nM-100 μM |
| 39.1b | 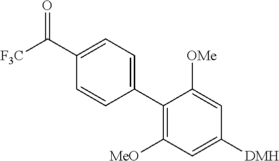 | | | | 1 nM-100 μM |
| 39.2 | 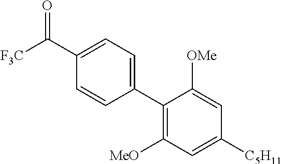 | | | | 1 nM-100 μM |
| 39.3* | 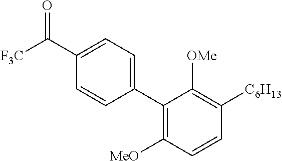 | | | | 1 nM-100 μM |
| 39.4 | 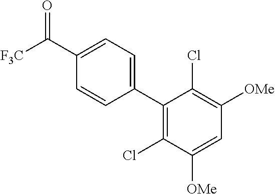 | | | | 1 nM-100 μM |
| 46.1* | 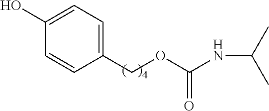 | | | | 1 nM-100 μM |
| 46.2* | 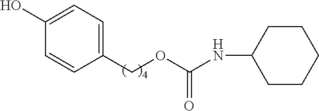 | | | | 1 nM-100 μM |
| 46.5* | 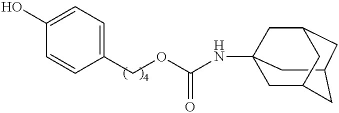 | | | | 1 nM-100 μM |
| 48.1 | 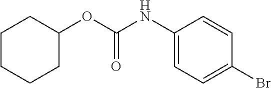 | 98% (100 μM) | | | |
| 48.2 | 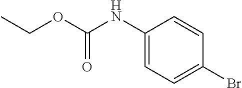 | 44% (100 μM) | | | |
| 48.3 | 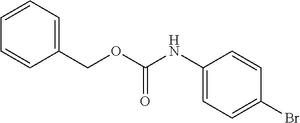 | 92% (100 μM) | | | |
| 48.4 | 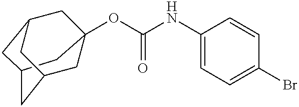 | 99% (100 μM) | 15.4 | μM | 3.9 μM |
| 48.5* | 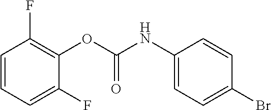 | | | | 1 nM-100 μM |
| 48.6 | 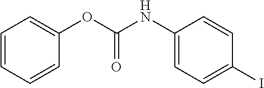 | 52% (100 μM) | | | |
| 48.9 | 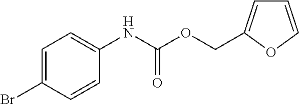 | 29% (100 μM) | | | |
| 52.1 | 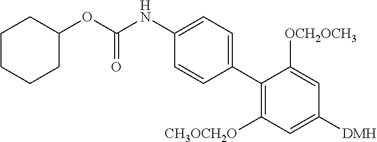 | | | | 1 nM-100 μM |
| 52.2 | 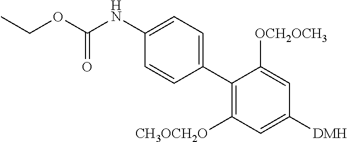 | 92% (100 μM) | | | |
| 52.3 | 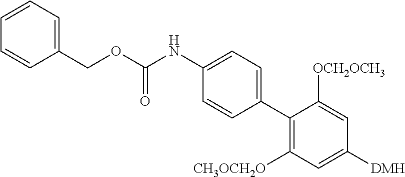 | 96% (100 μM) | | | |
| 52.4 | 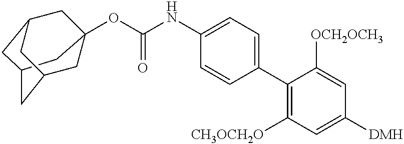 | | | | 1 nM-100 μM |
| 53.1 | 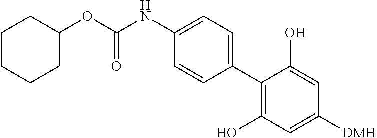 | 41% (100 μM) | | | |
| 53.2 | 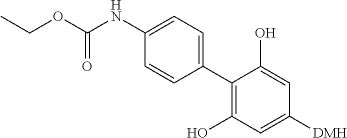 | 63% (100 μM) | | | |
| 53.4* | 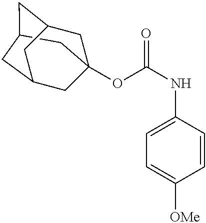 | | | | 1 nM-100 μM |
| 53.5 | 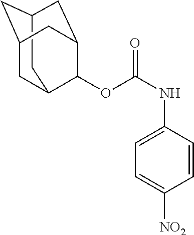 | 88% (100 μM) | | | |
| 53.6* | 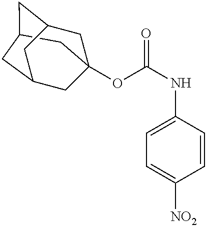 | | | | 1 nM-100 μM |
| 53.10* | 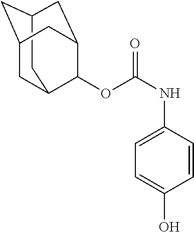 | | | | 1 nM-100 μM |
| 53.14* | 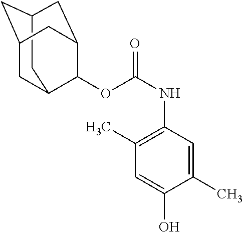 | | | | 1 nM-100 μM |
| 53.15* | 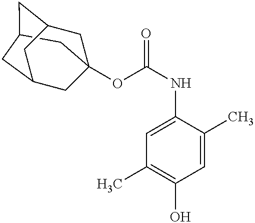 | | | | 1 nM-100 μM |
| 53.16* | 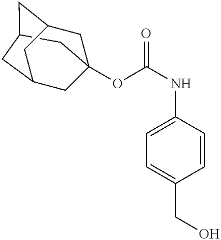 | | | | 1 nM-100 μM |
| 53.17* | 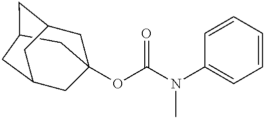 | | | | 1 nM-100 μM |
| 53.18* | 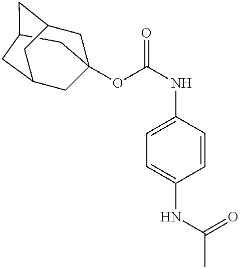 | | | | 1 nM-100 μM |
| 53.15* | 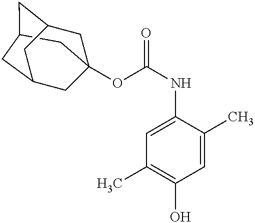 | | | | 1 nM-100 μM |
| 53.20* | 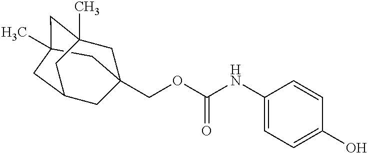 | | | | 1 nM-100 μM |
| 53.21* | 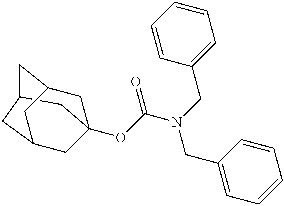 | | | | 1 nM-100 μM |
| 53.22* | 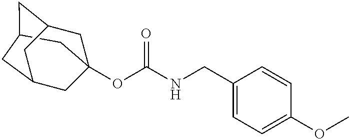 | | | | 1 nM-100 μM |
| 53.9* | 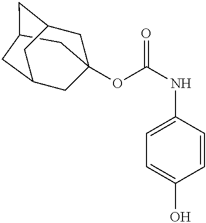 | | | | 1 nM-100 μM |
| 53.24* | 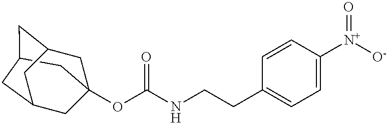 | | | | 1 nM-100 μM |
| 53.25* | 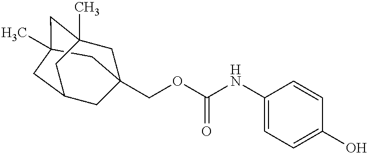 | | | | 1 nM-100 μM |
| 53.26* | 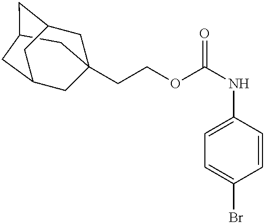 | | | | 1 nM-100 μM |
| 53.28* | 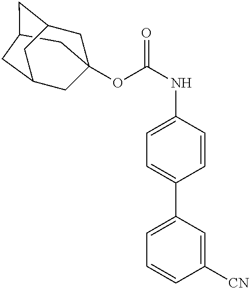 | | | | 1 nM-100 μM |
| 53.34* | 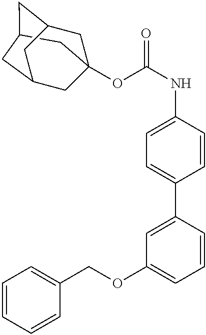 | | | | 1 nM-100 μM |
| 53.38* | 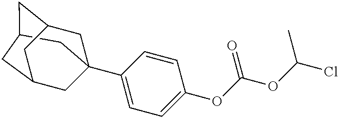 | | | | 1 nM-100 μM |
| 53.39* | 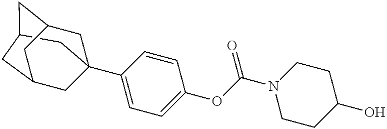 | | | | 1 nM-100 μM |
| 53.40* | 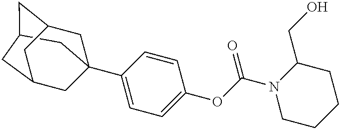 | | | | 1 nM-100 μM |
| 53.41* | 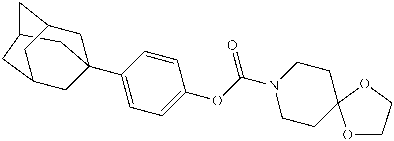 | | | | 1 nM-100 μM |
| 53.42* | 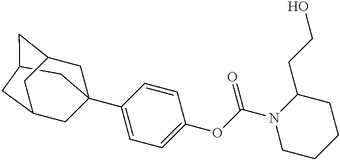 | | | | 1 nM-100 μM |
| 53.43* | 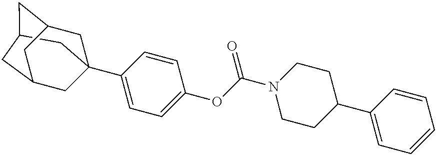 | | | | 1 nM-100 μM |
| 53.44 | 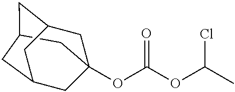 | | | | 1 nM-100 μM |
| 53.48 | 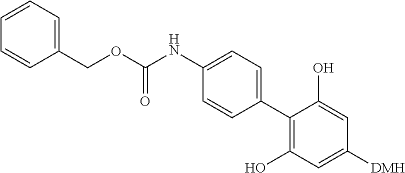 | 70% (100 μM) | | | |
| 53.49 | 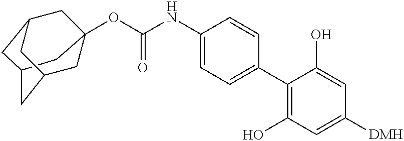 | 50% (100 μM) | | | |
| 57.1 | 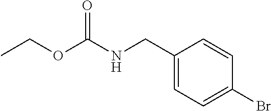 | 44% (100 μM) | | | |
| 57.2 | 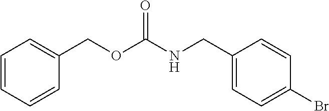 | 92% (100 μM) | | | |
| 57.3* | 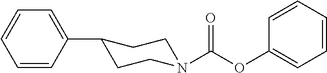 | | | | 1 nM-100 μM |
| 57.4* | 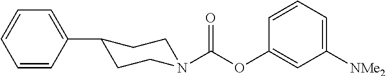 | | | | 1 nM-100 μM |
| 57.5* | 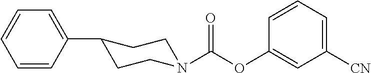 | | | | 1 nM-100 μM |
| 59.1* | 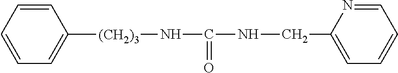 | | | | 1 nM-100 μM |
| 59.2* | 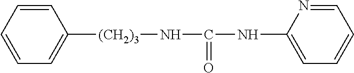 | | | | 1 nM-100 μM |
| 59.3* | 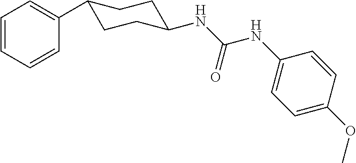 | | | | 1 nM-100 μM |
| 59.4* | 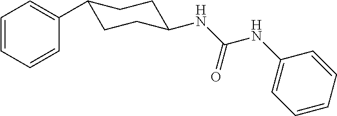 | | | | 1 nM-100 μM |
| 59.5* | 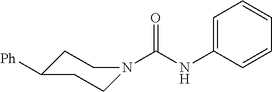 | | | | 1 nM-100 μM |
| 59.6 | 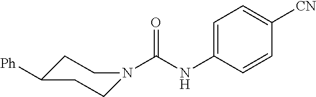 | | | | 1 nM-100 μM |
| 59.7 | 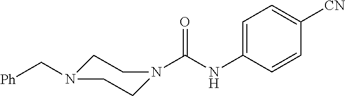 | | | | 1 nM-100 μM |
| 65.2* | 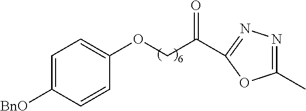 | | | | 1 nM-100 μM |
| 66* | 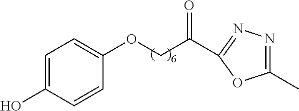 | | | | 1 nM-100 μM |
| 73.*1 | 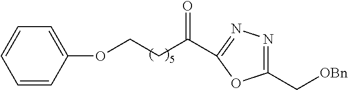 | | | | 1 nM-100 μM |
| 73.2* | 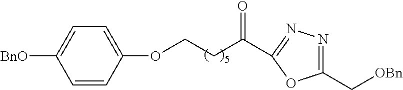 | | | | 1 nM-100 μM |
| 74.1* | 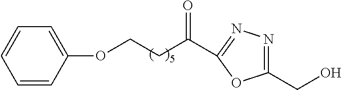 | | | | 1 nM-100 μM |
| 78* | 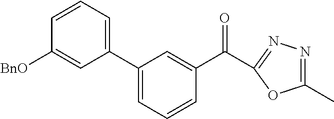 | | | | 1 nM-100 μM |
| 81* | 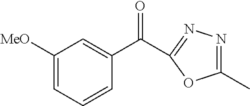 | | | | 1 nM-100 μM |
| 81.1* | 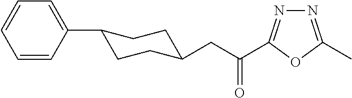 | | | | 1 nM-100 μM |
| 83.1* | 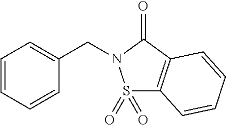 | | | | 1 nM-100 μM |
| 83.4* | 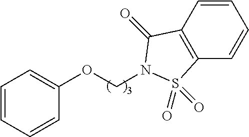 | | | | 1 nM-100 μM |
| 83.5* | 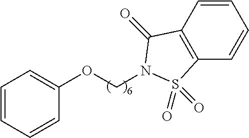 | | | | 1 nM-100 μM |
| 83.6* | 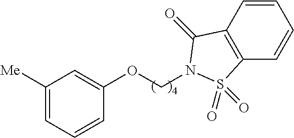 | | | | 1 nM-100 μM |
| 83.7* | 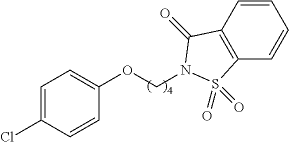 | | | | 1 nM-100 μM |
| 83.8* | 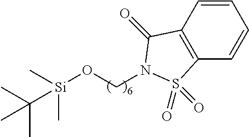 | | | | 1 nM-100 μM |
| 83.9* | 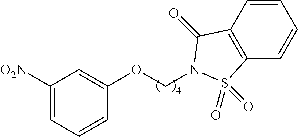 | | | | 1 nM-100 μM |
| 84* | 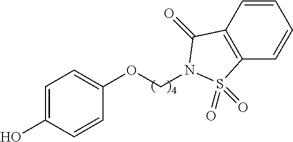 | | | | 1 nM-100 μM |
| 84.1* | 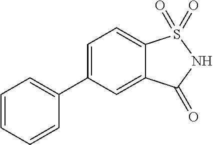 | | | | 1 nM-100 μM |
| 84.2* | 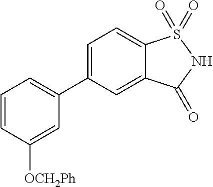 | | | | 1 nM-100 μM |
| 84.3* | 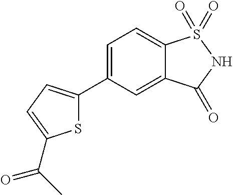 | | | | 1 nM-100 μM |
| 84.4* | 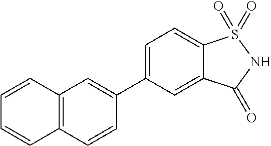 | | | | 1 nM-100 μM |
| 84.5* | 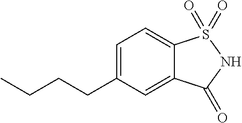 | | | | 1 nM-100 μM |
| 84.6* | 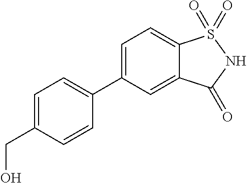 | | | | 1 nM-100 μM |
| 84.7* | 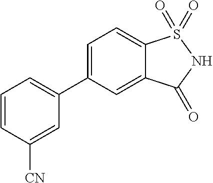 | | | | 1 nM-100 μM |
| 84.8* | 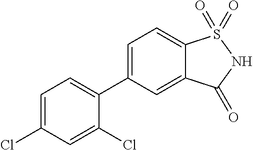 | | | | 1 nM-100 μM |
| 84.9* | 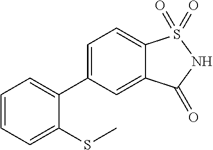 | | | | 1 nM-100 μM |
| 84.10* | 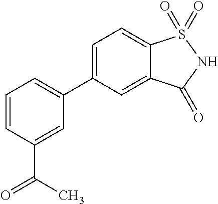 | | | | 1 nM-100 μM |
| 84.11* | 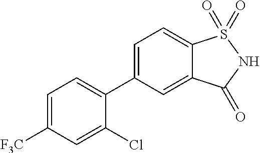 | | | | 1 nM-100 μM |
| 84.12* | 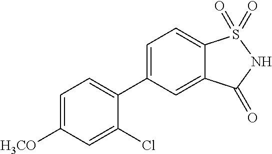 | | | | 1 nM-100 μM |
| 84.13* | 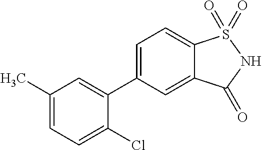 | | | | 1 nM-100 μM |
| 84.14* | 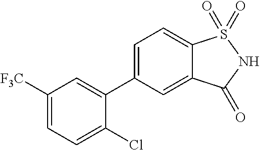 | | | | 1 nM-100 μM |
| 84.15* | 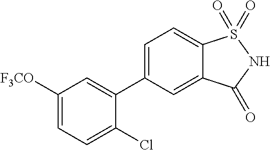 | | | | 1 nM-100 μM |
| 84.16* | 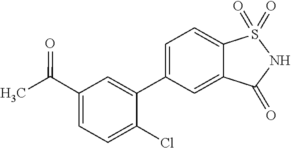 | | | | 1 nM-100 μM |
| 84.17* | 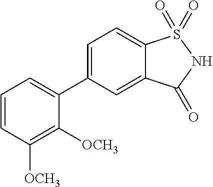 | | | | 1 nM-100 μM |
| 84.18* | 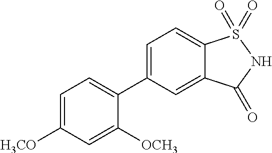 | | | | 1 nM-100 μM |
| 84.19* | 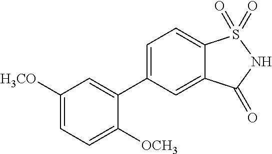 | | | | 1 nM-100 μM |
| 84.20* | 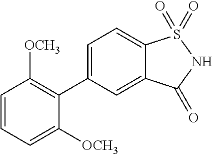 | | | | 1 nM-100 μM |
| 87.1 | 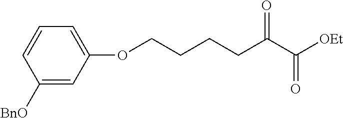 | 82% (100 μM) | | | |
| 87.2 | 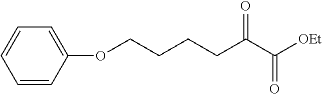 | | | | 1 nM-100 μM |
| 87.3 | 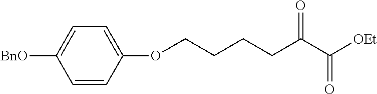 | | | | 1 nM-100 μM |
| 87.4 | 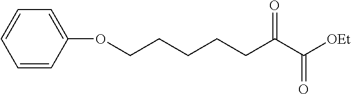 | 66% (100 μM) | | | |
| 87.7 | 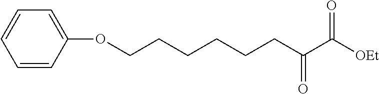 | | | | 1 nM-100 μM |
| 89.1 | 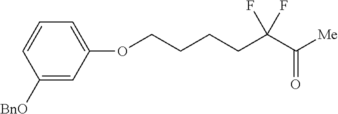 | | 17.9 | μM | 4.5 μM |
| 89.2 | 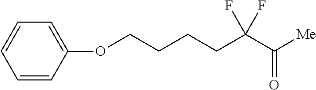 | | | | 1 nM-100 μM |
| 89.4 | 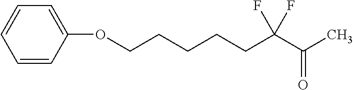 | 72% (100 μM) | | | |
| 89.7 | 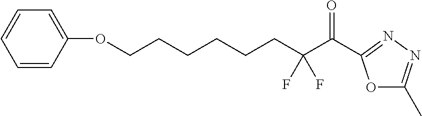 | | 0.071 | μM | 0.017 μM |
| 89.8 | 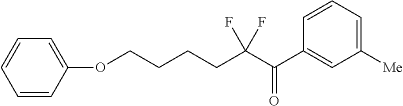 | 55% (100 μM) | | | |
| 89.9 | 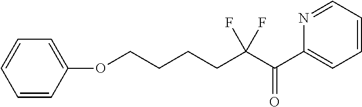 | 59% (100 μM) | 0.99 | μM | 0.25 μM |
| 89.9 Hydrate form | 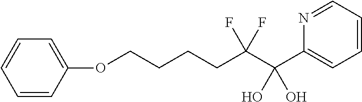 | 69% (100 μM) | | | |
| 89.10 | 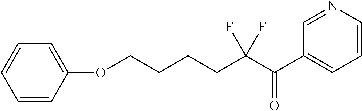 | 25% (100 μM) | | | |
| 89.11 | 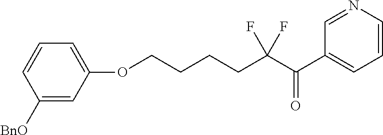 | 65% (100 μM) | | | |
| 89.12 | 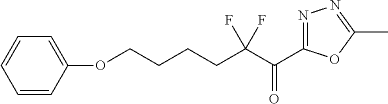 | 73% (100 μM) | 0.215 | μM | 0.054 μM |
| 89.13 | 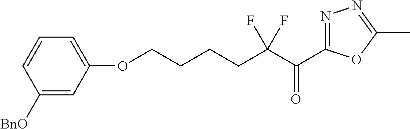 | | 0.072 | μm | |
| 89.14 | 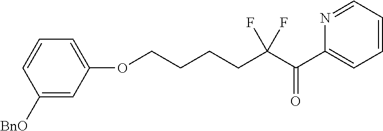 | | | | 1 nM-100 μM |
| 89.15* | 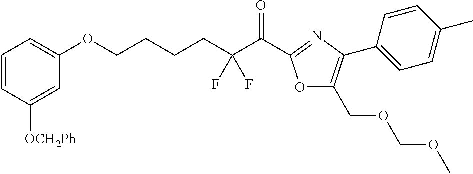 | | | | 1 nM-100 μM |
| 89.16* | 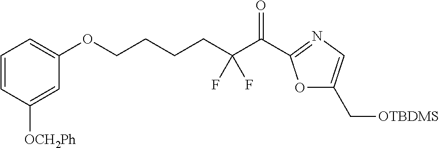 | | | | 1 nM-100 μM |
| 91.1 | 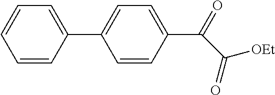 | | 51.6 | μM | 12.9 μM |
| 91.2 | 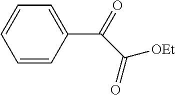 | 23% (100μM) | | | |
| 91.3 | 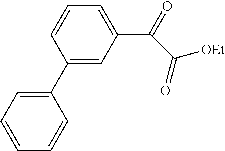 | | | | 1 nM-100 μM |
| 91.4 | 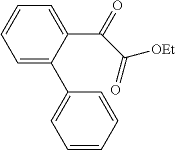 | | | | 1 nM-100 μM |
| 91.5 | 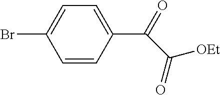 | 79% (100 μM) | | | |
| 91.6 | 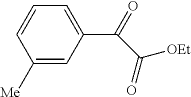 | | | | 1 nM-100 μM |
| 93.1 | 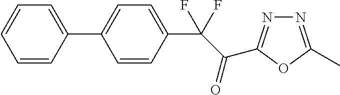 | 50% (100 μM) | 0.34 | μM | 0.086 μM |
| 93.2 | 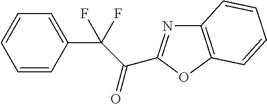 | | | | 1 nM-100 μM |
| 93.5 | 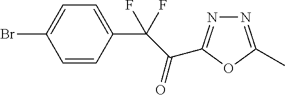 | 97% (100 μM) | 1.69 | μM | 0.42 μM |
| 93.7 | 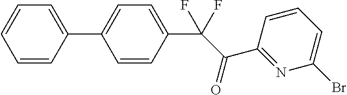 | 91% (100 μM) | | | |
| 93.8 | 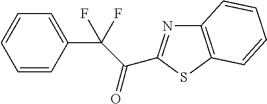 | 32% (100 μM) | | | |
| 93.9 | 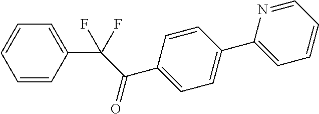 | | | | 1 nM-100 μM |
| 96.1 | 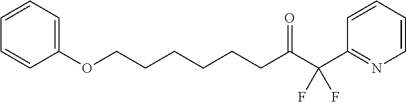 | 76% (100μM) | | | |
| 96.2 | 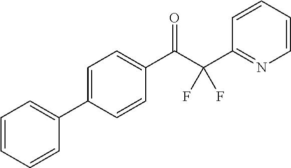 | 86% (100 μM) | | | |

^{a}The group BnO— is Ph—CH₂—O— where Ph is Phenyl.
^{b}The group DMH is as shown on Scheme 8.
*Novel compounds.

The inhibitory compounds can be synthesized by chemical means as described in the Examples below. Some inhibitory compounds may be commercially available. Novel compounds may be synthesized from commercially available starting material. The inhibitory compounds need not be made exclusively from the illustrative syntheses. A person of skill in the art understands that additional methods of making the inhibitory compounds exist. A person of skill in the art also understands that general synthetic schemes for the compounds disclosed herein can be understood from the illustrative schemes below.

### 3. Methods of Inhibition and Modulation

This disclosure is also directed to a method of modulating cannabinoid receptors in a biological sample by using the compounds of Formula (I), and pharmaceutically acceptable salts thereof. The method comprises (a) measuring the level of a cannabinergic ligand in the biological sample, (b) contacting the sample with a compound of Formula (I), thereby inhibiting an enzyme that hydrolyzes the cannabinergic ligand, and (c) measuring the level of the cannabinergic ligand in the contacted sample, the cannabinoid receptors being modulated if the level of the cannabinergic ligand in the contacted sample is the same or greater than the level of the cannabinergic ligand in the uncontacted sample.

In some instances, the enzyme inhibited is MGL. Testing of some compounds of Formula (I) shows inhibition of MGL in both in vitro and in vivo systems. Inhibition of MGL has the effect of preventing the degradation of endocannabinoid ligands and increasing or maintaining the level of endocannabinoid ligands in a system. Thus, the disclosed compounds, when administered in a therapeutically effective amount, increase or maintain the in vivo concentration of endogenous cannabinergic ligands in a subject, thereby enhancing or maintaining activation of cannabinoid receptors. In other instances, the inhibitor also inhibits FAAH in addition to MGL. The joint inactivation of both enzymes leads to enhanced therapeutic benefits because cannabinoid receptors can be modulated by additional cannabinergic ligands.

### 4. Methods of Treatment Using MGL Inhibitory Compounds

### Disorders

Some of the physiological effects provided by modulation of the cannabinoid receptors by cannabinergic ligands are useful to treat a disorder in a subject. Such treatable physiological effects include, but are not limited to, neuroprotection; reduction of inflammation; reduction of pain; reduction of central pain; reduction of peripheral pain; modulation of memory; sleep inducement; modulation of the immune system; hypotension; reduction of emesis; effects on gastrointestinal motility; effects on motor function; effects on intestinal transit and colonic propulsion; modulation of appetite; and modulation of fertility. Inhibition of MGL activity increases or maintains the concentration of existing levels of endogenous cannabinergic ligands and thereby increases or maintains the magnitude and duration of the physiological effect provided by those cannabinergic ligands. Therefore, the disclosed compounds, and therapeutic formulations containing such compounds, enhance or maintain the magnitude and duration of the physiological effects produced by a cannabinergic ligand in a subject when administered in therapeutically effective amounts.

Disorders that can be treated by inhibition of MGL and/or MGL and FAAH and indirect stimulation of the cannabinoid receptors include, for example: appetite disorders, metabolic disorders, movement disorders, inflammation, pain, neuropathic pain (e.g., neuropathic low back pain, complex regional pain syndrome, post trigeminal neuralgia, causalgia, toxic neuropathy, reflex sympathetic dystrophy, diabetic neuropathy, chronic neuropathy caused by chemotherapeutic agents), central pain, peripheral pain, neuropathy (e.g., diabetic neuropathy, pellagric neuropathy, alcoholic neuropathy, Beriberi neuropathy, burning feet syndrome), neurodegenerative diseases including multiple sclerosis, Parkinson's disease, Huntington's chorea, Alzheimer's disease, amyotrophic lateral sclerosis; memory disorders, mood disorders, sleep disorders, gastrointestinal motility disorders such as irritable bowel syndrome and diarrhea; cardiovascular disease, hypertension, osteoporosis, osteoarthritis, emesis, epilepsy, mental disorders such as schizophrenia and depression; glaucoma, cachexia, insomnia, traumatic brain injury, spinal cord injury, seizures, excitotoxin exposure, ischemia, AIDS wasting syndrome, psychological disorders including anxiety disorders (e.g., panic disorder, acute stress disorder, post-traumatic stress disorder, substance-induced anxiety disorders, obsessive-compulsive disorder, agoraphobia, specific phobia, social phobia), to modulate the immune system; to regulate fertility; to prevent or reduce diseases associated with motor function such as Tourette's syndrome; to provide neuroprotection, to produce peripheral vasodilation; to slow down intestinal transit and colonic propulsion; to treat several types of cancer, as well as other ailments in which a growing family of bioactive lipid mediators is implicated.

The disclosed inhibitory compounds and pharmaceutical formulations can also be used in combination with one or more agents treating and/or targeting the disorder or the endogenous cannabinergic system. Such agents include, but are not limited to, CB1 cannabinoid receptor agonists, CB2 cannabinoid receptor agonists, analgesics, FAAH inhibitors, anandamide transport inhibitors, COX-2 enzyme inhibitors, anxiolytics, antidepressants, and opioids. For example, these compounds and pharmaceutical formulations can be used in conjunction with other cannabinergic ligands that act directly on the CB1 and CB2 receptors.

In certain instances, the cannabinergic ligand is 2-arachidonoylglycerol. The disclosed compounds have high potential to be used as research tools to probe MGL and related lipase mechanisms of catalysis, and to uncover the biological roles of lipid mediators such as 2-arachidonoylglycerol. For example, the disclosed compounds can be used as in vivo imaging agents; to maintain the level of 2-arachidonoylglycerol in vitro to study the effect of 2-arachidonoylglycerol in cells and to enhance the levels of 2-arachidonoylglycerol in vivo in order to study the effect of 2-arachidonoylglycerol on humans and animals. The disclosed compounds can be used to characterize cells, for example, to determine if a cell type has cannabimimetic or lipase activity. For example, the disclosed compounds can be used to determine if a cell population expresses MGL by contacting the cells with a disclosed compound and then determining if there is an increase in the concentration of 2-arachidonoylglycerol. The inhibitors disclosed in this application can also be used as an aid in drug design, for example as a control in assays for testing other compounds for their ability to inhibit MGL and to determine the structure activity requirements of MGL inhibitors.

The disclosed compounds can also be used to prepare prodrugs. Prodrugs are known to those skilled in the art of pharmaceutical chemistry, and provide benefits such as increased adsorption and half-life. Those skilled in the art of drug delivery will readily appreciate that the pharmacokinetic properties of Formula (I) can be controlled by an appropriate choice of moieties to produce prodrug derivatives.

Formulation

This disclosure is also directed to a pharmaceutical formulation comprising at least one compound of Formula (I), and a pharmaceutically-acceptable carrier. Such formulations are suitable for administration to a subject. The pharmaceutical formulation can be used for treating a disorder described above.

Any suitable pharmaceutically acceptable carrier known in the art can be used as long as it does not affect the inhibitory activity of a compound of Formula (I). Carriers may be used that efficiently solubilize the agents. Carriers include, but are not limited to, a solid, liquid, or a mixture of a solid and a liquid. The carriers can take the form of capsules, tablets, pills, powders, lozenges, suspensions, emulsions, or syrups. The carriers can include substances that act as flavoring agents, lubricants, solubilizers, suspending agents, binders, stabilizers, tablet disintegrating agents, and encapsulating materials. Other examples of suitable physiologically acceptable carriers are described in Remington's Pharmaceutical Sciences (21st ed. 2005), incorporated into this disclosure by reference.

Non-limiting examples of materials which can serve as pharmaceutically-acceptable carriers include: (1) sugars, such as lactose, glucose, and sucrose; (2) starches, such as corn starch and potato starch; (3) cellulose and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline, (18) Ringer's solution, (19) ethyl alcohol; (20) phosphate buffer solutions; and (21) other non-toxic compatible substances employed in pharmaceutical formulations.

The formulations can conveniently be presented in unit dosage form and can be prepared by any methods known in the art of pharmacy. The amount of compound of Formula (I) which can be combined with a carrier material to produce a single-dosage form will vary depending upon the subject being treated, the particular mode of administration, the particular condition being treated, among others. The amount of active ingredient that can be combined with a carrier material to produce a single-dosage form will generally be that amount of the compound that produces a therapeutic effect. Generally, out of one hundred percent, this amount will range from about 1 percent to about ninety-nine percent of active ingredient, in some instances from about 5 percent to about 70 percent, in other instances from about 10 percent to about 30 percent.

Methods of preparing these formulations or compositions include the step of bringing into association a compound disclosed in this application with a carrier and, optionally, one or more accessory ingredients. In general, the formulations are prepared by uniformly and intimately bringing into association a compound of Formula (I) with liquid carriers, or timely divided solid carriers, or both, and then, if necessary, shaping the product.

In solid dosage forms of the disclosed compounds for oral administration (e.g., capsules, tablets, pills, dragees, powders, granules, and the like), the active ingredient is mixed with one or more additional ingredients, such as sodium citrate or dicalcium phosphate, and/or any of the following: (1) fillers or extenders, such as, but not limited to, starches, lactose, sucrose, glucose, mannitol, and/or silicic acid; (2) binders, such as, but not limited to, carboxymethylcellulose, alginates, gelatin, polyvinyl pyrrolidone, sucrose, and/or acacia; (3) humectants, such as, but not limited to, glycerol; (4) disintegrating agents, such as, but not limited to, agar, calcium carbonate, potato or tapioca starch, alginic acid, certain silicates, and sodium carbonate; (5) solution retarding agents, such as, but not limited to, paraffin; (6) absorption accelerators, such as, but not limited to, quaternary ammonium compounds; (7) wetting agents, such as, but not limited to, cetyl alcohol and glycerol monostearate; (8) absorbents, such as, but not limited to, kaolin and bentonite clay; (9) lubricants, such as, but not limited to, talc, calcium stearate, magnesium stearate, solid polyethylene glycols, sodium lauryl sulfate, and mixtures thereof; and (10) coloring agents. In the case of capsules, tablets, and pills, the pharmaceutical compositions can also comprise buffering agents. Solid compositions of a similar type can also be employed as fillers in soft and hard-filled gelatin capsules using such excipients as lactose or milk sugars, as well as high molecular weight polyethylene glycols, and the like.

In powders, the carrier is a finely-divided solid, which is mixed with an effective amount of a finely-divided agent. Powders and sprays can contain, in addition to a compound of Formula (I), excipients, such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Tablets for systemic oral administration can include one or more excipients as known in the art, such as, for example, calcium carbonate, sodium carbonate, sugars (e.g., lactose, sucrose, mannitol, sorbitol), celluloses (e.g., methyl cellulose, sodium carboxymethyl cellulose), gums (e.g., arabic, tragacanth), together with one or more disintegrating agents (e.g., maize, starch, or alginic acid, binding agents, such as, for example, gelatin, collagen, or acacia), lubricating agents (e.g., magnesium stearate, stearic acid, or talc), inert diluents, preservatives, disintegrants (e.g., sodium starch glycolate), surface-active and/or dispersing agent. A tablet can be made by compression or molding, optionally with one or more accessory ingredients.

In solutions, suspensions, emulsions or syrups, an effective amount of a disclosed compound is dissolved or suspended in a carrier, such as sterile water or an organic solvent, such as aqueous propylene glycol. Other compositions can be made by dispersing the agent in an aqueous starch or sodium carboxymethyl cellulose solution or a suitable oil known to the art. The liquid dosage forms can contain inert diluents commonly used in the art, such as, for example, water or other solvents, solubilizing agents and emulsifiers, such as, but not limited to, ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan, and mixtures thereof.

Besides inert diluents, the oral compositions can also include adjuvants, such as wetting agents, emulsifying and suspending agents, sweetening, flavoring, coloring, perfuming, and preservative agents.

Suspensions can contain, in addition to the active compound, suspending agents as, for example, ethoxylated isostearyl alcohols, polyoxyethylene sorbitol and sorbitan esters, microcrystalline cellulose, aluminum metahydroxide, bentonite, agar and tragacanth, and mixtures thereof.

Formulations of the pharmaceutical compositions for rectal or vaginal administration can be presented as a suppository, which can be prepared by mixing one or more compounds of this disclosure with one or more suitable non-irritating excipients or carriers comprising, for example, cocoa butter, polyethylene glycol, a suppository wax or a salicylate, and which is solid at RT but liquid at body temperature and, thus, will melt in the rectum or vaginal cavity and release the agents. Formulations suitable for vaginal administration also include, but are not limited to, pessaries, tampons, creams, gels, pastes, foams, or spray formulations containing such carriers as are known in the art to be appropriate.

Dosage forms for the topical or transdermal administration of a compound of this disclosure include, but are not limited to, powders, sprays, ointments, pastes, creams, lotions, gels, solutions, patches, and inhalants. The active compound can be mixed under sterile conditions with a pharmaceutically-acceptable carrier, and with any preservatives, buffers, or propellants.

Ointments, pastes, creams, and gels can contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of a compound of Formula (I) to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the compound in a polymer matrix or gel.

The compounds of Formula (I) are administered in a therapeutically effective amount to a patient in need of such treatment. Such an amount is effective in treating a disorder of the patient. This amount can vary, depending on the activity of the agent utilized, the nature of the disorder, and the health of the patient. A skilled practitioner will appreciate that the therapeutically-effective amount of a compound of Formula (I) can be lowered or increased by fine-tuning and/or by administering more than one compound of Formula (I), or by administering a compound of Formula (I) together with a second agent (e.g., antibiotics, antifungals, antivirals, NSAIDS, DMARDS, steroids, etc.). Therapeutically-effective amounts can be easily determined, for example, empirically by starting at relatively low amounts and by step-wise increments with concurrent evaluation of beneficial effect (e.g., reduction in symptoms). The actual effective amount will be established by dose/response assays using methods standard in the art (Johnson et al., Diabetes., (1993) 42:1179). As is known to those in the art, the effective amount will depend on bioavailability, bioactivity, and biodegradability of the compound of Formula (I).

A therapeutically-effective amount is an amount that is capable of reducing a symptom of a disorder in a subject. Accordingly, the amount will vary with the subject being treated. Administration of the compound of Formula (I) can be hourly, daily, weekly, monthly, yearly, or a single event. For example, the effective amount of the compound can comprise from about 1 μg/kg body weight to about 100 mg/kg body weight. In one embodiment, the effective amount of the compound comprises from about 1 μg/kg body weight to about 50 mg/kg body weight. In a further embodiment, the effective amount of the compound comprises from about 10 μg/kg body weight to about 10 mg/kg body weight. When one or more compounds of Formula (I) or agents are combined with a carrier, they can be present in an amount of about 1 weight percent to about 99 weight percent, the remainder being composed of the pharmaceutically-acceptable carrier.

Administration

Methods of administration of the therapeutic formulations comprising the compounds of Formula (I) can be by any of a number of methods known in the art. These methods include, but are not limited to, local or systemic administration. Exemplary routes of administration include, but are not limited to, oral, parenteral, transdermal, intradermal, intramuscular, intraperitoneal, intravenous, subcutaneous, intranasal (e.g., nebulizer, inhaler, aerosol dispenser), colorectal, rectal, intravaginal, and any combinations thereof. In addition, it may be desirable to introduce pharmaceutical compositions of the disclosed compounds into the central nervous system by any suitable route, including intraventricular and intrathecal injection. Intraventricular injection can be facilitated by an intraventricular catheter, for example, attached to a reservoir, such as an Ommaya reservoir. Methods of introduction can be provided by rechargeable or biodegradable devices, e.g., depots. Furthermore, administration can occur by coating a device, implant, stent, or prosthetic. The compounds of Formula (I) can also be used to coat catheters in any situation where catheters are inserted in the body.

The therapeutic formulations containing a compound of Formula (I) can also be administered as part of a combinatorial therapy with other agents. Combination therapy refers to any form of administration combining two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either simultaneously or sequentially. Thus, an individual who receives such treatment can have a combined (conjoint) effect of different therapeutic compounds.

In other instances, for example, in the case of inflammatory conditions, a therapeutic formulation containing a compound of Formula (I) can be administered in combination with one or more other agents useful in the treatment of inflammatory diseases or conditions. Agents useful in the treatment of inflammatory diseases or conditions include, but are not limited to, anti-inflammatory agents, or antiphlogistics. Exemplary antiphlogistics include, but are not limited to, glucocorticoids, such as cortisone, hydrocortisone, prednisone, prednisolone, fluorcortolone, triamcinolone, methylprednisolone, prednylidene, paramethasone, dexamethasone, betamethasone, beclomethasone, fluprednylidene, desoxymethasone, fluocinolone, flunethasone, diflucortolone, clocortolone, clobetasol and fluocortin butyl ester; immunosuppressive agents such as anti-TNF agents (e.g., etanercept, infliximab) and IL-1 inhibitors; penicillamine; non-steroidal anti-inflammatory drugs (NSAIDs) which encompass anti-inflammatory, analgesic, and antipyretic drugs such as salicyclic acid, celecoxib, difunisal and from substituted phenylacetic acid salts or 2-phenylpropionic acid salts, such as alclofenac, ibutenac, ibuprofen, clindanac, fenclorac, ketoprofen, fenoprofen, indoprofen, fenclofenac, diclofenac, flurbiprofen, piprofen, naproxen, benoxaprofen, carprofen and cicloprofen; oxican derivatives, such as piroxican; anthranilic acid derivatives, such as mefenamic acid, flufenamic acid, tolfenamic acid and meclofenamic acid, anilino-substituted nicotinic acid derivatives, such as the fenamates miflumic acid, clonixin and flunixin; heteroarylacetic acids wherein heteroaryl is a 2-indol-3-yl or pyrrol-2-yl group, such as indomethacin, oxmetacin, intrazol, acemetazin, cinmetacin, zomepirac, tolmetin, colpirac and tiaprofenic acid; idenylacetic acid of the sulindac type; analgesically active heteroaryloxyacetic acids, such as benzadac; phenylbutazone; etodolac; nabunetone; and disease modifying antirheumatic drugs (DMARDs) such as methotrexate, gold salts, hydroxychloroquine, sulfasalazine, ciclosporin, azathioprine, and leflunomide. Other therapeutics useful in the treatment of inflammatory diseases or conditions include antioxidants. Antioxidants can be natural or synthetic. Antioxidants are, for example, superoxide dismutase (SOD), 21-aminosteroids/aminochromans, vitamin C or E, etc. Many other antioxidants are known to those of skill in the art. The compounds of Formula (I) can serve as part of a treatment regimen for an inflammatory condition, which may combine many different anti-inflammatory agents. For example, the subject compounds can be administered in combination with one or more of an NSAID, DMARD, or immunosuppressant. The subject compounds can also be administered in combination with methotrexate. The subject antibodies can also be administered in combination with a TNF-α inhibitor.

In the case of cardiovascular disease conditions, and particularly those arising from atherosclerotic plaques, which are thought to have a substantial inflammatory component, the therapeutic formulation including a compound of Formula (I) can be administered in combination with one or more other agents useful in the treatment of cardiovascular diseases. Agents useful in the treatment of cardiovascular diseases include, but are not limited to, β-blockers such as carvedilol, metoprolol, bucindolol, bisoprolol, atenolol, propranolol, nadolol, timolol, pindolol, and labetalol; antiplatelet agents such as aspirin and ticlopidine; inhibitors of angiotensin-converting enzyme (ACE) such as captopril, enalapril, lisinopril, benazopril, fosinopril, quinapril, ramipril, spirapril, and moexipril; and lipid-lowering agents such as mevastatin, lovastatin, simvastatin, pravastatin, fluvastatin, atorvastatin, and rosuvastatin.

In the case of cancer, the subject compounds can be administered in combination with one or more anti-angiogenic factors, chemotherapeutics, or as an adjuvant to radiotherapy. It is further envisioned that the administration of the subject compounds will serve as part of a cancer treatment regimen, which may combine many different cancer therapeutic agents.

The disclosure is further illustrated by the following examples, which are not to be construed as limiting this disclosure in scope or spirit to the specific procedures described in this disclosure. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the sprint of the present disclosure and/or scope of the appended claims.

### EXAMPLES

### Testing of Inhibitory Compounds

Certain compounds were tested for their MGL inhibitory activity, which is expressed as % of inhibition or IC₅₀/Ki values in Tables 1 and 2. The percentage of inhibition describes the percentage by which the inhibitor reduces the velocity/rate of 2-AG hydrolysis by MGL. The IC₅₀ is the concentration of the inhibitor, which results in 50% inhibition of the velocity/rate of 2-AG hydrolysis by MGL. The Kᵢ value is the affinity constant and describes the affinity of the inhibitor for the MGL. The lower the IC₅₀/Kᵢ values, the higher the affinity of the inhibitor for the enzyme and the higher its inhibitory activity. A detailed description of the methods used to test inhibitory activity of compounds is given below. The compounds in Table 2 are also assayed for their inhibitory activity as described below, and their activity or expected activity ranges are provided.

### 1. Partial Purification of MGL

Monoacylglycerol lipase enzyme was partially purified from adult Sprague-Dawley rat brains purchased from Pel-Free-ze Biologicals according to a procedure disclosed in Lang et al., Anal. Biochem. (1996) 238:40-45. These rat brains are homogenized in 5 volumes of ice-cold buffer (0.32 M sucrose, 10 mM Tris base, 5 mM EDTA, pH 7.4) then centrifuged at 17400 g for 30 min. The supernatant was further centrifuged at 124,000 g for 90 min. The supernatant from the last certifugation step (cytosol fraction) is resuspended in TME buffer (25 mM Tris base, 5 mM MgCl₂, 1 mM EDTA, pH 7.4) for the MGL preparation. Aliquots (1 ml) from the preparation are flash frozen in liquid nitrogen and stored at −80° C. until used. Protein concentration of the enzyme suspension is determined using the BioRad protein assay kit.

### 2. MGL Enzyme Assay

All compound solutions are made to a concentration of 10 mM in DMSO. To test the stability of the potentially therapeutic compounds in enzyme assay conditions, 25 nmoles of the compound are incubated in TME buffer (final volume of 250 μL) for 20 min at 37° C. Samples (100 μL) are taken at the start of the assay and after 20 min, diluted 1:5 with acetonitrile and centrifuged (20,000 RCF, five min, RT) to precipitate the proteins. The resulting supernatant is injected onto the HPLC. Calculations for determining the percent compound remaining are described in the following equation:

% R=Peak Area(T20)/Peak Area(T0)

To determine whether or not the compounds are good substrates for MGL, 25 nmoles of the compound were incubated with 30 μg enzyme preparation in TME buffer (final volume 250 μL). The reaction mixture is treated in the same manner as described above. Concentrations of 2-arachidonoylglycerol (2-AG) and arachidonic acid (AA) are calculated using external standards. The rate of AA formation is calculated using the following equation:

Rate=(T20−T0)/20 min/30 μg

The inhibition of 2-AG metabolism is measured by mixing 25 nmoles of the compound with 25 nmoles 2-AG, and 30 μg enzyme preparation in TME buffer (final volume of 250 μL) as disclosed in Lang et al., Anal. Biochem. (1996) 238:40-45, Qin et al., Anal. Biochem. (1998) 261:8-15 and Lang et al., J. Med. Chem. (1999) 42:896-902, for the FAAH enzyme assay. Again the reaction mixture is treated in the same manner as described above and the concentrations of 2-AG and AA are calculated using external standards. Percent inhibition is calculated using the following equation:

% Inhib.=(AA20−AA0)c/(AA20−AA0)s

where (AA20−AA0)c is the amount of arachidonic acid formed over 20 min from 2-AG with the inhibitor present and (AA20−AA0)s is the amount of arachidonic acid formed over 20 min from 2-AG when the inhibitor is not present. In the IC₅₀ studies of the disclosed analogs various concentrations of compound are incubated with 25 nmoles 2-AG, and 30 μg enzyme preparation in TME buffer (final volume of 250 μL). The reaction mixtures are treated as described above and the amount of AA formed was calculated. Prizm software (GraphPad Software, Inc.) is utilized to calculate IC₅₀ and Kᵢ values.

### 3. HPLC Conditions for Enzyme Assay

Chromatographic separation was achieved using an Ultrasphere ODS Pre-column (4.6×45 mm) from Beckman. Hardware consisted of a Waters Millennium HPLC system with a 20 μL injection loop. The mobile phase consisted of 8.5% o-phosphoric acid:acetonitrile (3:7), run isocratically at a rate of 1 mL/min and detection at 204 nm. The total run time was 8 min with 2-AG eluting at 3.0 min, and AA at 6.0 min.

### 4. Synthesized Compounds

Some representative MGL inhibitors of Formula (I) that have been synthesized are depicted in Tables 1 and 2.

### Synthesis of Sulfonyl Fluorides

Phenylalkylsulfonyl fluorides 4.1, 4.2, and 4.3 (Table 2) were synthesized by the method depicted in Scheme 1 starting from commercially available phenylalkyl alcohols 1.1, 1.2, and 1.3.

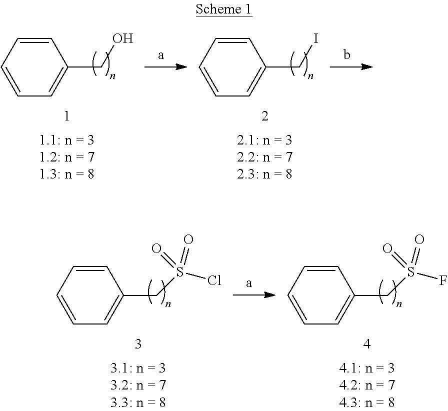

Reagents and conditions for the steps in Scheme 1 were as follows: Step a: PPh₃, imidazole, I₂, MeCN/Et₂O, 0° C. to RT, 72-85%; Step b (i) t-BuLi, Et₂O/pentane, −78° C., (ii) SO₂Cl₂, −78° C., 19-23%; Step c: NH₄F, acetone, reflux, 91-93%.

### A. Phenylalkyl Iodides (2)

A round bottom flask was charged with phenylalkyl alcohol (1) (1 equiv.), acetonitrile/diethyl ether mixture (1:2), triphenyl phosphine (1.3 equiv.), imidazole (1.3 equiv.), and iodine (1.3 equiv.). The solution was blanketed with argon and capped, and the reaction stirred for 4-5 hours at RT. The resulting mixture was diluted with diethyl ether, washed with water, aqueous sodium thiosulfate, and brine, dried (MgSO₄), and evaporated. Purification by flash column chromatography on silica gel (10% diethyl ether-hexane) gave phenylalkyl iodide 2 in 72-85% yield.

### B. Phenylalkylsulfonyl Chlorides (3)

A solution of phenylalkyl iodide (2) (1 equiv.) in a mixture of dry n-pentane/diethyl ether (3:2) was cooled to −78° C. under argon, and t-BuLi (2.2 equiv., using a 1.7 M solution of t-BuLi in hexane) was added dropwise over a 2-min period. The mixture was stirred for 10 min at −78° C. and then was transferred by cannula to a cooled (−78° C.) and dry solution of SO₂Cl₂ in n-pentane over a 20-min period. Following the addition, the reaction mixture was stirred for 1 hour at −78° C. and then allowed to warm to RT over a 3 hour period. The reaction mixture was quenched with dropwise addition of water, then diluted with diethyl ether and the organic phase was separated. The aqueous phase was extracted with diethyl ether, the combined organic layer was dried (MgSO₄), and the solvent was evaporated. Purification by flash column chromatography on silica gel gave phenylalkylsulfonyl chloride 3 in 19-23% yield.

### C. Phenylalkylsulfonyl Fluorides (4)

To a stirred solution of phenylalkylsulfonyl chloride (3) (1 equiv.) in dry acetone, was added anhydrous NH₄F (2 equiv.) and the mixture refluxed for 2 hours. The reaction mixture was cooled to RT, the solvent was evaporated, and the residue obtained was dissolved in diethyl ether. The ethereal solution was successively washed with water and brine, dried (MgSO₄), and concentrated under reduced pressure. Purification by flash column chromatography on silica gel gave phenylalkylsulfonyl fluoride 4 in 91-93% yield.

### D. Selected data of synthesized phenylalkylsulfonyl fluorides (4)

### 3-Phenyl-propanesulfonyl fluoride (4.1)

4.1 was confirmed as follows: ¹H NMR (200 MHz, CDCl₃) δ 7.46-7.15 (m, 5H), 3.40-3.27 (m, 2H), 2.82 (t, J=7.3 Hz, 2H), 2.40-2.21 (m, 2H); mass spectrum m/z (relative intensity) 202 (M⁺, 27), 91 (100).

### 7-Phenyl-heptanesulfonyl fluoride (4.2)

4.2 was confirmed as follows: Mass spectrum m/z (relative intensity) 258 (M⁺, 10), 105 (9), 91 (100).

### 8-Phenyl-octanesulfonyl fluoride (4.6)

4.6 was confirmed as follows: ¹H NMR (200 MHz, CDCl₃) δ 7.45-7.05 (m, 5H), 3.40-3.25 (m, 2H), 2.60 (t, J=7.1 Hz, 2H), 2.10-1.20 (m, 12H).

### Synthesis of Sulfonyl Chlorides 12.1-12.4 and Sulfonyl fluorides (13.1-13.4) and (14.1-14.4)

Sulfonyl fluorides (13.1), (13.2), (13.3), (13.4), (14.1), (14.2), (14.3), (14.4) were synthesized by a method depicted in Scheme 2 starting from commercially available 2- or 3- or 4-anisaldehyde and the appropriate phenoxyalkyl bromide.

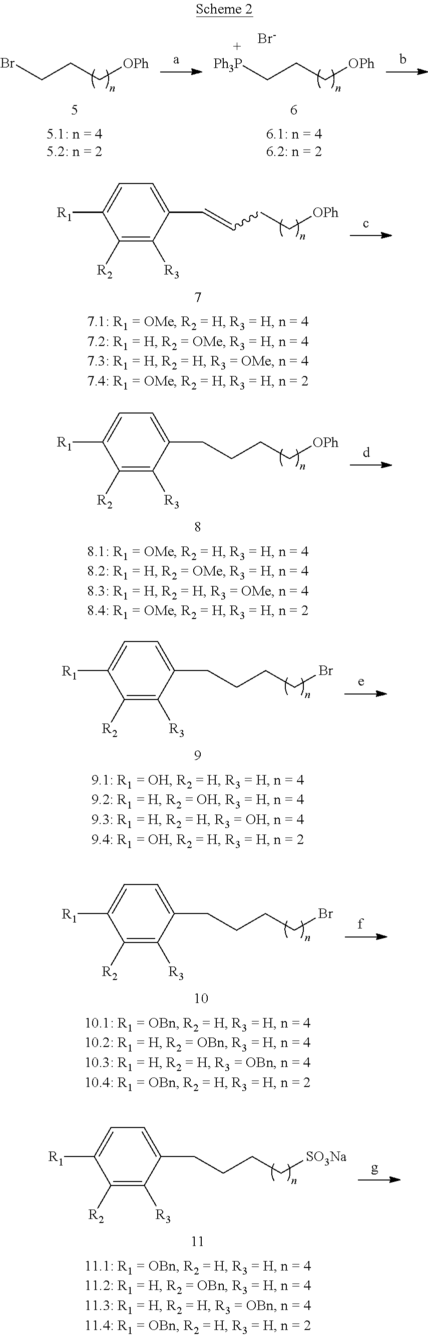

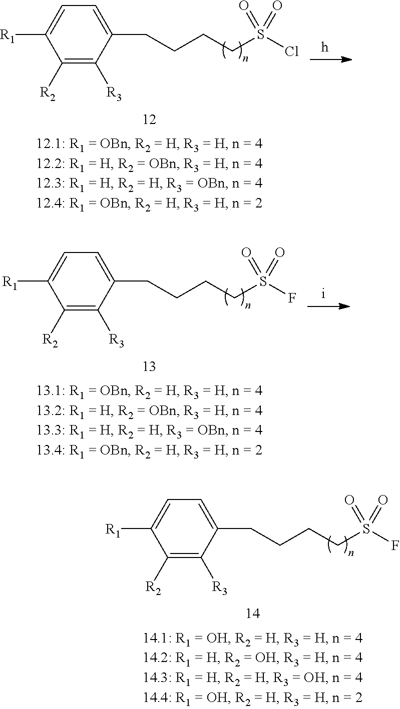

Reagents and conditions for the steps in Scheme 2 were as follows: Step a: Ph₃P, PhH, reflux, 85-87%; Step b: (Me₃Si)₂N⁻K⁺, THF, 0° C., then 2- or 3- or 4-anisaldehyde 91-93%; Step c: H₂, Pd/C, AcOEt, 30 psi, RT, 6 hours, 95-96%; Step d: BBr₃, CH₂Cl₂, −30° C. to RT, 2 hours, 90-93%; Step e: K₂CO₃, acetone, BnBr, reflux, 6 hours, 76-78%; Step f: Na₂SO₃, EtOH/H₂O, reflux, 6 hours or microwave; Step g: SOCl₂, PhH/DMF, N₂, 50° C., 3 hours, 37-40% from 10; Step h: NH₄F, acetone, N₂, reflux, 2 hours, 91-93%; Step i: BF₃.OEt₂, HS(CH₂)₂SH, N₂, RT, 1 hour, 68-70%.

### 6-Phenoxyhexyltriphenylphosphonium bromide (6.1)

A mixture of 6-phenoxyhexyl bromide (5.1) (2.8 g, 10.9 mmol) and triphenylphosphine (314 g, 12 mmol) in anhydrous benzene (100 mL), under an argon atmosphere, was refluxed for two days. The reaction mixture was allowed to cool to RT, and the precipitating product (6.1) was isolated by filtration under reduced pressure and washed with anhydrous diethyl ether (4.75 g, white solid, melting point 143-145° C., 84% yield).

6.1 was confirmed as follows: ¹H NMR (500 MHz, CDCl₃) δ 7.89-7.85 (m as dd, 6H), 7.81-7.75 (m as td, 3H), 7.71-7.67 (m as td, 6H), 7.25 (t, J=7.7 Hz, 2H), 6.91 (t, J=7.7 Hz, 1H), 6.84 (d, J=7.7 Hz, 2H) 3.95-3.85 (m and t overlapping, especially 3.90, t, J=6.3 Hz, 4H), 1.79-1.65 (m, 6H), 1.49 (quintet, J=7.7 Hz, 2H).

### 4-Phenoxybutyltriphenylphosphonium bromide (6.2)

The title compound was synthesized as in 6.1 using 4-phenoxybutyl bromide (5.2) (22.0 g, 95.9 mmol) and triphenylphosphine (27.6 g, 105.5 mmol) in anhydrous benzene (50 mL), to give 6.1 (40.0 g, white solid, melting point 185-186° C., 85% yield).

6.2 was confirmed as follows: ¹H NMR (500 MHz, CDCl₃) δ 7.88-7.84 (m as dd, 6H), 7.78-7.76 (m as td, 3H), 7.68-7.65 (m, 6H), 7.25 (t, J=7.7 Hz, 2H), 6.92 (t, J=7.7 Hz, 1H), 6.82 (d, J=7.7 Hz, 2H), 4.09 (t, J=4.5 Hz, 2H), 4.04-3.98 (m, 2H), 2.25 (quintet, J=6.4 Hz, 2H), 1.92-1.86 (m, 2H).

### 1-(4-Methoxyphenyl)-7-phenoxy-1-heptene (7.1)

To a suspension of 6-phenoxyhexyltriphenylphosphonium bromide (6.1) (4.60 g, 8.86 mmol) in dry THF (80 mL) at 0° C., under an argon atmosphere was added potassium bis(trimethylsilyl)amide (1.76 g, 8.86 mmol). The resulting slurry was stirred for 5 min at the same temperature, and then a solution of 4-methoxybenzaldehyde (0.61 g, 4.46 mmol) in dry THF (10 mL) was added. The reaction mixture was stirred for an additional 10 min and quenched with saturated aqueous NH₄Cl (20 mL). The resulting mixture was warmed to RT, diluted with Et₂O (100 mL), and the organic phase was separated and the aqueous phase extracted with Et₂O. The combined organic layer was washed with brine, dried over MgSO₄, and the solvent evaporated under reduced pressure. The residue obtained was purified through a short column of silica gel, eluting with 5% Et₂O-hexane, to give the product (7.1) (1.21 g, 92% yield, predominantly cis, cis:trans=96:4) as a colorless liquid.

7.1 was confirmed as follows: ¹H NMR (500 MHz, CDCl₃) δ 7.27 (t, J=7.5 Hz, 2H), 7.21 (d, J=8.7 Hz, 2H), 6.92 (t, J=7.5 Hz, 1H), 6.91-6.86 (m, overlapping signals, 4H), 6.35 (d, J=11.5 Hz, 1H), 5.57 (dt, J=11.5 Hz, J=7.5 Hz, 1H), 3.94 (t, J=6.0 Hz, 2H), 3.81 (s, 3H), 2.41-2.20 (m, 2H), 1.78 (quintet, J=6.7 Hz, 2H), 1.58-1.48 (m, 4H).

### 1-(3-Methoxyphenyl)-7-phenoxy-1-heptene (7.2)

1-(3-Methoxyphenyl)-7-phenoxy-1-heptene (7.2) was synthesized as described above in 7.1 using 6.1 (3.20 g 6.16 mmol), dry THF (30 mL), potassium bis(trimethylsilyl)amide (1.23 g, 6.16 mmol), and 3-methoxybenzaldehyde (0.28 g, 2.05 mmol). The title compound (7.2) was isolated as a colorless liquid after purification by flash column chromatography (0.564 g, 93% yield, predominantly cis, cis:trans=95:5).

7.2 was confirmed as follows: ¹H NMR (500 MHz, CDCl₃) δ 7.27-7.21 (m, 3H), 6.92 (t, J=7.0 Hz, 1H), 6.90-6.86 (m, 3H), 6.81 (t, J=1.5 Hz, 1H), 6.78 (dd, J=8.5 Hz, J=1.5 Hz, 1H), 6.39 (d, J=11.7 Hz, 1H), 5.67 (dt, J=11.7 Hz, J=7.5 Hz, 1H), 3.94 (t, J=6.5 Hz, 2H), 3.80 (s, 3H), 2.37 (q, J=6.5, 2H), 1.78 (quintet, J=6.5 Hz, 2H), 1.56-1.48 (m, 4H).

### 1-(2-Methoxyphenyl)-7-phenoxy-1-heptene (7.3)

1-(2-Methoxyphenyl)-7-phenoxy-1-heptene (7.3) was synthesized as described above for 7.1 using 6.1 (2.0 g, 3.85 mmol), dry THF (30 mL), potassium bis(trimethylsilyl)amide (0.77 g, 3.85 mmol), and 2-methoxybenzaldehyde (0.20 g, 1.47 mmol). The title compound (7.3) was isolated as a colorless liquid after purification by flash column chromatography (0.396 g, 91% yield, predominantly cis, cis:trans=93:7).

7.3 was confirmed as follows: ¹H NMR (500 MHz, CDCl₃) δ 7.29-7.21 (m, 4H), 6.94-6.87 (m, 5H), 6.52 (d, J=11.2 Hz, 1H), 5.73 (dt, J=11.2 Hz, J=7.5 Hz, 1H), 3.93 (t, J=6.7 Hz, 2H), 3.83 (s, 3H), 2.28 (m as q, J=7.2 Hz, 2H), 1.76 (quintet, J=7.2 Hz, 2H), 1.53-1.46 (m, 4H).

### 1-(4-Methoxyphenyl)-7-phenoxy-1-pentene (7.4)

1-(4-Methoxyphenyl)-7-phenoxy-1-pentene (7.4) was synthesized as described in 7.1 using 6.2 (29.0 g, 58.8 mmol), dry THF (200 mL), potassium bis(trimethylsilyl)amide (11.7 g, 58.8 mmol) and 4-methoxybenzaldehyde (2.9 g, 14.7 mmol). The title compound (7.4) was isolated as a colorless liquid after purification by flash column chromatography (3.69 g, 93% yield, predominantly cis, cis:trans=96:4).

7.4 was confirmed as follows: ¹H NMR (500 MHz, CDCl₃) δ 7.26 (t, J=7.5 Hz, 2H), 7.22 (d, J=8.7 Hz, 2H), 6.92 (t, J=7.5 Hz, 1H), 6.87 (d, J=7.5 Hz, 2H), 6.85 (d, J=8.7 Hz, 2H), 6.39 (d, J=11.5 Hz, 1H), 5.60 (dt, J=11.5 Hz, J=7.0 Hz, 1H), 3.98 (t, J=6.0 Hz, 2H), 3.80 (s, 3H), 2.51 (m as qd, J=7.5 Hz, J=2.1 Hz, 2H), 1.94 (quintet, J=6.7 Hz 2H).

### 1-(4-Methoxyphenyl)-7-phenoxy-heptane (8.1)

To a stirred solution of 7.1 (1.19 g, 4.03 mmol) in AcOEt (40 mL) at RT was added 10% Pd/C (0.18 g, 15% w/w), and the resulting suspension was hydrogenated (30 psi, 6 hrs). The catalyst was removed by filtration through celite, and the filtrate was evaporated under reduced pressure to give the title compound (8.1) as a white solid (1.14 g, 95% yield, melting point 32-34° C.).

8.1 was confirmed as follows: ¹H NMR (500 MHz, CDCl₃) δ 7.30 (t, J=8.5 Hz, 2H), 7.11 (d, J=8.2 Hz, 2H), 6.95 (t, J=8.5 Hz, 1H), 6.92 (d, J=8.5 Hz 2H), 6.84 (d, J=8.2 Hz, 2H), 3.97 (t, J=6.7 Hz, 2H), 3.81, (s, 3H) 2.57 (t, J=7.5 Hz, 2H), 1.78 (quintet, J=6.7 Hz, 2H), 1.62 (quintet, J=7.5 Hz, 2H), 1.48 (quintet, J=7.5 Hz, 2H), 1.44-1.34 (m, 4H).

### 1-(3-Methoxyphenyl)-7-phenoxy-heptane (8.2)

1-(3-Methoxyphenyl)-7-phenoxy-heptane (8.2) was synthesized as described in 8.1 using 7.2 (0.55 g, 1.86 mmol), AcOEt (20 mL), and 10% Pd/C (0.080 g, 15% w/w). The title compound (8.2) was isolated as a colorless viscous liquid (0.53 g, 96% yield).

8.2 was confirmed as follows: ¹H NMR (500 MHz, CDCl₃) δ 7.27 (t, J=7.0 Hz, 2H), 7.19 (t, J=7.4 Hz, 1H), 6.92 (t, J=7.0 Hz, 1H), 6.89 (d, J=7.0 Hz, 2H), 6.77 (d, J=7.4 Hz, 1H), 6.73-6.71 (m, 2H), 3.94 (t, J=6.5 Hz, 2H), 3.79 (s, 3H), 2.58 (t, J=7.5 Hz, 2H), 1.77 (quintet, J=6.7 Hz, 2H), 1.62 (quintet, J=7.2 Hz, 2H), 1.50-1.42 (m, 2H), 1.42-1.34 (m, 4H).

### 1-(2-Methoxyphenyl)-7-phenoxy-heptane (8.3)

1-(2-Methoxyphenyl)-7-phenoxy-heptane (8.3) was synthesized as described in 8.1 using 7.3 (0.35 g, 1.18 mmol), AcOEt (20 mL), and 10% Pd/C (0.050 g, 14% w/w). The title compound (8.3) was isolated as a colorless viscous liquid (0.33 g, 95% yield).

8.3 was confirmed as follows: ¹H NMR (500 MHz, CDCl₃) δ 7.27 (t, J=7.5 Hz, 2H), 7.16 (t, J=7.5 Hz, 1H), 7.12 (d, J=7.5 Hz, 1H), 6.94-6.83 (m, 5H), 3.95 (t, J=6.5 Hz, 2H), 3.81 (s, 3H), 2.60 (t, J=7.7, 2H), 1.78 (quintet, J=7.0 Hz, 2H), 1.59 (quintet, J=7.0 Hz, 2H), 1.48-1.43 (m, 2H), 1.42-1.38 (m, 4H).

### 1-(4-Methoxyphenyl)-5-phenoxy-pentane (8.4)

1-(4-Methoxyphenyl)-5-phenoxy-pentane (8.4) was synthesized as described in 8.1 using 7.4 (3.67 g, 13.69 mmol), AcOEt (100 mL), and 10% Pd/C (0.550 g, 15% w/w). The title compound (8.3) was isolated as a white solid (m p 32-34° C.) in 95% yield (3.52 g).

8.4 was confirmed as follows: ¹H NMR (500 MHz, CDCl₃) δ 7.27 (t, J=7.5 Hz, 2H), 7.09 (d, J=8.5 Hz, 2H), 6.92 (t, J=7.5 Hz, 1H), 6.88 (d, J=7.5 Hz, 2H), 6.82 (d, J=8.5 Hz, 2H), 3.94 (t, J=6.5 Hz, 2H), 3.78 (s, 3H), 2.58 (t, J=7.7 Hz, 2H), 1.80 (quintet, J=6.7 Hz, 2H), 1.66 (quintet, J=7.0 Hz, 2H), 1.49 (quintet, J=7.5 Hz, 2H).

### 7-Bromo-1-(4-hydroxy-phenyl)-heptane (9.1)

To a stirred solution of 8.1 (1.1 g, 3.69 mmol) in anhydrous CH₂Cl₂, (40 mL), at −30° C., under an argon atmosphere was added BBr₃ (8 mL, 8 mmol, using a 1 M solution in CH₂Cl₂) and the mixture gradually warmed to RT (2 hours). Unreacted boron tribromide was destroyed by addition of aqueous saturated NaHCO₃ solution (10 mL) to the reaction mixture at 0° C. The resulting mixture was warmed to RT and diluted with Et₂O (40 mL). The organic layer was separated and the aqueous phase extracted with Et₂O. The combined organic layer was washed with brine, dried over MgSO₄, and the solvent evaporated under reduced pressure. The residue obtained was chromatographed through a short column of silica gel, eluting with 20% Et₂O-hexane to give 8.1 (0.930 g, 93% yield) as a viscous liquid.

9.1 was confirmed as follows: ¹H NMR (500 MHz, CDCl₃) δ 7.03 (d, J=8.5 Hz, 2H), 6.74 (d, J=8.5 Hz, 2H), 4.59 (br s, 1H), 3.34 (t, J=6.7 Hz, 2H), 2.53 (t, J=7.7 Hz, 2H), 1.84 (quintet, J=7.0 Hz, 2H), 1.57 (quintet, J=7.5 Hz, 2H), 1.46-1.38 (m, 2H), 1.36-1.31 (m, 4H).

### 7-Bromo-1-(3-hydroxy-phenyl)-heptane (9.2)

7-Bromo-1-(3-hydroxy-phenyl)-heptane (9.2) was synthesized as in 9.1 using 8.2 (0.50 g, 1.68 mmol), in anhydrous CH₂Cl₂ (16 mL), and BBr₃ (1 M solution in CH₂Cl₂, 3.7 mL, 3.7 mmol). The title compound (9.2) was isolated as a viscous liquid after purification by flash column chromatography (0.420 g, 92% yield).

9.2 was confirmed as follows: ¹H NMR (500 MHz, CDCl₃) δ 7.14 (t, J=8.0 Hz, 1H), 6.75 (d, J=8.0 Hz, 1H), 6.66-6.63 (d and dd overlapping, 2H), 4.67 (br s, 1H), 3.40 (t, J=6.7 Hz, 2H), 2.56 (t, J=7.7 Hz, 2H), 1.85 (quintet, J=7.0 Hz, 2H), 1.62 (quintet, J=7.5 Hz, 2H), 1.46-1.38 (m, 2H), 1.36-1.32 (m, 4H).

### 7-Bromo-1-(2-hydroxy-phenyl)-heptane (9.3)

7-Bromo-1-(2-hydroxy-phenyl)-heptane (9.3) was synthesized as in 9.1 using 8.3 (0.30 g, 1.01 mmol) in anhydrous CH₂Cl₂(10 mL), and BBr₃ (1 M solution in CH₂Cl₂,2.2 mL, 2.2 mmol). The title compound (9.3) was isolated as a viscous liquid after purification by flash column chromatography (0.247 g, 90% yield).

9.3 was confirmed as follows: ¹H NMR (500 MHz, CDCl₃) δ 7.11 (dd, J=7.5 Hz, J=1.5 Hz, 1H), 7.07 (td, J=7.5 Hz, J=1.5 Hz, 1H), 6.87 (td, J=7.5 Hz, J=1.5 Hz, 1H), 6.75 (dd, J=7.5 Hz, J=1.5 Hz, 1H), 4.62 (br s, 1H), 3.40 (t, J=7.0 Hz, 2H), 2.60 (t, J=8.0 Hz, 2H), 1.85 (quintet, J=6.7 Hz, 2H), 1.62 (quintet, J=7.2 Hz, 2H), 1.4 (quintet, J=7.5 Hz, 2H), 1.40-1.35 (m, 4H).

### 5-Bromo-1-(4-hydroxy-phenyl)-pentane (9.4)

5-Bromo-1-(4-hydroxy-phenyl)-pentane (9.4) was synthesized as in 9.1 using 8.4 (3.43 g, 12.7 mmol) in anhydrous CH₂Cl₂ (120 mL), and BBr₃ (1 M solution in CH₂Cl₂, 32 mL, 32 mmol). The title compound (9.4) was isolated as a viscous liquid after purification by flash column chromatography (2.84 g, 92% yield).

9.4 was confirmed as follows: ¹H NMR (500 MHz, CDCl₃) δ 7.04 (d, J=8.7 Hz, 2H), 6.75 (d, J=8.7 Hz, 2H), 4.68 (br s, 1H), 3.34 (t, J=6.7 Hz, 2H), 2.55 (t, J=7.7 Hz, 2H), 1.88 (quintet, J=7.7 Hz, 2H), 1.60 (quintet, J=7.7 Hz, 2H), 1.46 (quintet, J=7.5 Hz, 2H).

### 7-Bromo-1-(4-benzyloxy-phenyl)-heptane (10.1)

To a stirred solution of 9.1 (0.9 g, 3.32 mmol) in anhydrous acetone (40 mL), was added anhydrous K₂CO₃ (1.38 g, 10 mmol) and benzyl bromide (0.624 g, 3.65 mmol) and the mixture was refluxed for 6 hours. The reaction mixture was cooled to RT, diluted with acetone, and solid materials were filtered off. The filtrate was evaporated under reduced pressure, and the residue obtained was dissolved in diethyl ether (50 mL). The ethereal solution was washed with water and brine, dried (MgSO₄), and evaporated. Purification by flash column chromatography on silica gel (5% Et₂O-hexane) afforded 10.1 (0.938 g, 78% yield) as a white solid (melting point 32-34° C.).

10.1 was confirmed as follows: ¹H NMR (500 MHz, CDCl₃) δ 7.43 (d, J=7.0 Hz, 2H), 7.38 (t, J=7.0 Hz, 2H), 7.32 (t, J=7.0 Hz 1H), 7.08 (d, J=8.7 Hz, 2H) 6.90 (d, J=8.7 Hz 2H), 5.04 (s, 2H), 3.34 (t, J=7.0 Hz, 2H), 2.54 (t, J=7.7 Hz, 2H), 1.85 (quintet, H=7.5 Hz, 2H), 1.58 (quintet, J=7.5 Hz, 2H), 1.46-1.38 (m, 2H), 1.37-1.30 (m, 4H).

### 7-Bromo-1-(3-benzyloxy-phenyl)-heptane (10.2)

7-Bromo-1-(3-benzyloxy-phenyl)-heptane (10.2) was prepared as in 10.1 using 9.2 (0.4 g, 1.48 mmol), K₂CO₃ (0.612 g, 4.44 mmol) and benzyl bromide (0.278 g, 1.63 mmol). The title compound (10.2) was isolated as a viscous liquid after purification by flash column chromatography (0.411 g, 77% yield).

10.2 was confirmed as follows: ¹H NMR (500 MHz, CDCl₃) δ 7.44 (d, J=7.5 Hz, 2H), 7.39 (t, J=7.5 Hz, 2H), 7.32 (t, J=7.5 Hz 1H), 7.19 (t, J=7.2 Hz, 1H) 6.83-6.77 (m, 3H), 5.05 (s, 2H), 3.40 (t, J=6.77 Hz, 2H), 2.56 (t, J=7.7 Hz, 2H), 1.84 (quintet, J=7.0 Hz, 2H), 1.60 (quintet, J=7.7 Hz, 2H), 1.42 (quintet, J=7.0 Hz, 2H), 1.35-1.32 (m, 4H).

### 7-Bromo-1-(2-benzyloxy-phenyl)-heptane (10.3)

7-Bromo-1-(2-benzyloxy-phenyl)-heptane (10.3) was prepared as in 10.1 using 9.3 (0.23 g, 0.85 mmol), K₂CO₃ (0.352 g, 2.55 mmol) and benzyl bromide (0.16 g, 0.935 mmol). The title compound (10.3) was isolated as a viscous liquid after purification by flash column chromatography (0.24 g, 78% yield).

10.3 was confirmed as follows: ¹H NMR (500 MHz, CDCl₃) δ 7.44 (d, J=7.5 Hz, 2H), 7.39 (t, J=7.5 Hz, 2H), 7.32 (t, J=7.5 Hz 1H), 7.18-7.13 (m, 2H), 6.92-6.88 (m, 2H), 5.08 (s, 2H), 3.37 (t, J=7.0 Hz, 2H), 2.67 (t, J=7.7 Hz, 2H), 1.82 (quintet, J=7.2 Hz, 2H), 1.62 (quintet, J=7.5 Hz, 2H), 1.39 (quintet, J=7.7 Hz, 2H), 1.36-1.32 (m, 4H).

### 5-Bromo-1-(4-benzyloxy-phenyl)-pentane (10.4)

5-Bromo-1-(4-benzyloxy-phenyl)-pentane (10.4) was prepared as in 10.1 using 9.4 (2.99 g, 12.3 mmol), K₂CO₃ (4.24 g, 30.75 mmol) and benzyl bromide (2.31 g, 13.53 mmol). The title compound (10.4) was isolated as a white semi-solid after purification by flash column chromatography (3.11 g, 76% yield).

10.4 was confirmed as follows: ¹H NMR (500 MHz, CDCl₃) δ 7.42 (d, J=7.5 Hz, 2H), 7.37 (t, J=7.5 Hz, 2H), 7.31 (t, J=7.5 Hz 1H), 7.08 (d, J=8.5 Hz, 2H), 6.90 (d, J=8.5 Hz, 2H), 5.03 (s, 2H), 3.39 (t, J=6.7 Hz, 2H), 2.56 (t, J=7.7 Hz, 2H), 1.87 (quintet, J=6.7 Hz, 2H), 1.61 (quintet J=7.7 Hz, 2H), 1.46 (quintet J=6.7 Hz, 2H).

### 7-(4-Benzyloxy-phenyl)-heptanesulfonic acid sodium salt (11.1)

A stirred mixture of 10.1 (0.9 g, 2.50 mmol) and anhydrous Na₂SO₃ (0.423 g, 3.36 mmol) in EtOH (20 mL)/H₂O (10 ml) was heated under reflux (6 hours) or microwaved using a CEM-discover system (ram time: 2 min, hold time: 5 min, temperature: 150° C., pressure: 250 psi, power: 250 W). The reaction mixture was cooled to RT, and the solvent evaporated under reduced pressure. The residue obtained was scrupulously dried under high vacuum, and the crude product (11.1, pale yellow solid) was used in the next step without further purification.

### 7-(3-Benzyloxy-phenyl)-heptanesulfonic acid sodium salt (11.2)

Following the procedure described for 11.1 using the 10.2 (0.4 g, 1.1 mmol), Na₂SO₃ (0.19 g, 1.5 mmol) and EtOH (8 mL)/H₂O (4 ml) mixture, the crude 11.2 was obtained and used in the next step without further purification.

### 7-(2-Benzyloxy-phenyl)-heptanesulfonic acid Sodium salt (11.3)

Following the procedure described for 11.1 using 10.3 (0.231 g, 0.64 mmol), Na₂SO₃ (0.11 g, 0.89 mmol) and EtOH (8 mL)/H₂O (4 ml) mixture, the crude 11.3 was obtained and used in the next step without further purification.

5-(4-Benzyloxy-phenyl)-pentanesulfonic acid Sodium salt (11.4)

Following the procedure described for 11.1 using 10.4 (0.95 g, 2.85 mmol), Na₂SO₃ (0.50 g, 4.0 mmol) and EtOH (25 mL)/H₂O (7 ml) mixture, the crude 11.4 was obtained and used in the next step without further purification.

### 7-(4-Benzyloxy-phenyl)-heptanesulfonyl chloride (12.1)

To a stirred suspension of 11.1 (0.96 g, 2.50 mmol) in anhydrous benzene (20 mL)/DMF (2 ml), was added thionyl chloride (0.89 g, 7.5 mmol) and the resulting mixture was heated at 50° C. for 3 hours under argon. The reaction mixture was quenched by dropwise addition of water (10 mL) at RT and extracted with diethyl ether. The organic layer was washed with brine, dried (MgSO₄), and the solvent evaporated under reduced pressure. Purification by flash column chromatography on silica gel (20% diethyl ether-hexane) afforded 12.1 in 40% yield from 10.1 (0.38 g, white solid, melting point 33-35° C.).

12.1 was confirmed as follows: ¹H NMR (500 MHz, CDCl₃) δ 7.44 (d, J=7.5 Hz, 2H), 7.38 (t, J=7.5 Hz, 2H), 7.32 (t, J=7.5 Hz 1H), 7.08 (d, J=8.5 Hz, 2H), 6.90 (d, J=8.5 Hz, 2H), 5.04 (s, 2H), 3.64 (m as t, half of an AA′XX′ system, 2H), 2.55 (t, J=7.5 Hz, 2H), 2.03 (quintet, J=7.7 Hz, 2H), 1.62-1.54 (m, 2H), 1.52-1.46 (m, 2H), 1.40-1.30 (m, 4H).

### 7-(3-Benzyloxy-phenyl)-heptanesulfonyl chloride (12.2)

7-(3-Benzyloxy-phenyl)-heptanesulfonyl chloride (12.2) was synthesized as described in 12.1 using 11.2 (0.42 g, 1.1 mmol) and thionyl chloride (0.36 g, 3 mmol) in benzene (9 mL)/DMF (1 mL). Purification by flash column chromatography on silica gel gave the title compound (0.163 g, 39% yield from 10.2) as a viscous liquid.

12.2 was confirmed as follows: ¹H NMR (500 MHz, CDCl₃) δ 7.44 (d, J=7.5 Hz, 2H), 7.39 (t, J=7.5 Hz, 2H), 7.32 (t, J=7.5 Hz, 1H), 7.19 (t, J=7.2 Hz, 1H), 6.82-6.77 (m, 3H), 5.05 (s, 2H), 3.64 (m as t, half of an AA′XX′ system, 2H), 2.58 (t, J=7.5 Hz, 2H), 2.02 (quintet, J=7.5 Hz, 2H), 1.62 (quintet, J=7.5 Hz, 2H), 1.48 (quintet, J=7.5 Hz, 2H), 1.42-1.32 (m, 4H).

### 7-(2-Benzyloxy-phenyl)-heptanesulfonyl chloride (12.3)

7-(2-Benzyloxy-phenyl)-heptanesulfonyl chloride (12.3) was synthesized as described in 12.1 using 11.3 (0.46 g, 0.64 mmol) and thionyl chloride (0.228 g, 1.92 mmol) in benzene (9 mL)/DMF (1 mL). Purification by flash column chromatography on silica gel gave the title compound (0.092 g, 38% yield from 10.3) as a viscous liquid.

12.3 was confirmed as follows: ¹H NMR (500 MHz, CDCl₃) δ 7.44 (d, J=7.5 Hz, 2H), 7.39 (t, J=7.5 Hz, 2H), 7.33 (t, J=7.5 Hz 1H), 7.18-7.33 (m, 2H), 6.92-6.88 (m, 2H), 5.08 (s, 2H), 3.58 (m as t, half of an AA′XX′ system, 2H), 2.67 (t, J=7.7 Hz, 2H), 1.99 (quintet, J=7.5 Hz, 2H), 1.62 (quintet, J=7.5 Hz, 2H), 1.46-1.4 (m, 2H), 1.36-1.32 (m, 4H).

### 5-(4-Benzyloxy-phenyl)-pentanesulfonyl chloride (12.4)

5-(4-Benzyloxy-phenyl)-pentanesulfonyl chloride (12.4) was synthesized as described in 12.1 using 11.4 (0.96 g, 2.85 mmol) and thionyl chloride (1.00 g, 8.55 mmol) in benzene (27 mL)/DMF (3 mL). Purification by flash column chromatography on silica gel gave the title compound (0.36 g, 37% yield from 10.4) as a white solid (melting point 58-60° C.).

12.4 was confirmed as follows: ¹H NMR (500 MHz, CDCl₃) δ 7.43 (d, J=7.5 Hz, 2H), 7.38 (t, J=7.5 Hz, 2H), 7.32 (t, J=7.5 Hz 1H), 7.07 (d, J=8.7 Hz, 2H), 6.90 (d, J=8.7 Hz, 2H), 5.04 (s, 2H), 3.64 (m as t, half of an AA′XX′ system, 2H), 2.56 (t, J=7.2 Hz, 2H), 2.06 (quintet, J=7.7 Hz, 2H), 1.66 (quintet, J=7.5 Hz, 2H), 1.46 (quintet, J=7.7 Hz, 2H).

### 7-(4-Benzyloxy-phenyl)-heptanesulfonyl fluoride (13.1)

To a stirred solution of 12.1 (0.345 g, 0.9 mmol) in dry acetone (20 mL), was added anhydrous NH₄F (0.066 g, 1.8 mmol) and the mixture refluxed for 2 hours. The reaction mixture was cooled to RT, the solvent was evaporated, and the residue obtained was dissolved in diethyl ether (20 mL). The ethereal solution was successively washed with water and brine, dried (MgSO₄), and concentrated under reduced pressure. Purification by flash column chromatography on silica gel (20% diethyl ether-hexane) afforded 13.1 (0.306 g, 93% yield) as a white solid (melting point 35-38° C.).

13.1 was confirmed as follows: ¹H NMR (500 MHz, CDCl₃) δ 7.43 (d, J=7.5 Hz, 2H), 7.38 (t, J=7.5 Hz, 2H), 7.32 (t, J=7.5 Hz 1H), 7.08 (d, J=8.7 Hz, 2H), 6.90 (d, J=8.7 Hz, 2H), 5.04 (s, 2H), 3.36-3.32 (m, 2H), 2.54 (t, J=7.5 Hz, 2H), 1.94 (quintet, J=7.5 Hz, 2H), 1.62-1.54 (m, 2H), 1.52-1.44 (m, 2H), 1.40-1.30 (m, 4H).

### 7-(3-Benzyloxy-phenyl)-heptanesulfonyl fluoride (13.2)

7-(3-Benzyloxy-phenyl)-heptanesulfonyl fluoride (13.2) was prepared as in 13.1 using 12.2 (0.149 g, 0.39 mmol) and NH₄F (0.029 g, 0.78 mmol) in dry acetone (10 mL). Purification by flash column chromatography on silica gel gave the title compound (0.128 g, 91% yield) as a viscous liquid.

13.2 was confirmed as follows: ¹H NMR (500 MHz, CDCl₃) δ 7.43 (d, J=7.5 Hz, 2H), 7.39 (t, J=7.5 Hz, 2H), 7.32 (t, J=7.5 Hz, 1H), 7.19 (t, J=7.2 Hz, 1H), 6.82-6.77 (m, 3H), 5.05 (s, 2H), 3.36-3.32 (m, 2H), 2.58 (t, J=7.5 Hz, 2H), 1.93 (quintet, J=7.7 Hz, 2H), 1.61 (quintet, J=7.5 Hz, 2H), 1.48 (quintet, J=7.2 Hz, 2H), 1.42-1.32 (m, 4H).

### 7-(2-Benzyloxy-phenyl)-heptanesulfonyl fluoride (13.3)

7-(2-Benzyloxy-phenyl)-heptanesulfonyl fluoride (13.3) was prepared as in 13.1 using 12.3 (0.09 g, 0.236 mmol) and NH₄F (0.018 g, 0.486 mmol) in dry acetone (10 mL). Purification by flash column chromatography gave the title compound (0.079 g, 92% yield) as a viscous liquid.

13.3 was confirmed as follows: ¹H NMR (500 MHz, CDCl₃) δ 7.43 (d, J=7.2 Hz, 2H), 7.39 (t, J=7.2 Hz, 2H), 7.33 (t, J=7.2 Hz 1H), 7.17-7.14 (m, 2H), 6.92-6.89 (m, 2H), 5.08 (s, 2H), 3.35-3.32 (m, 2H), 2.67 (t, J=7.5 Hz, 2H), 1.89 (quintet, J=7.7 Hz, 2H), 1.62 (quintet, J=7.5 Hz, 2H), 1.46-1.4 (m, 2H), 1.36-1.32 (m, 4H).

### 5-(4-Benzyloxy-phenyl)-pentanesulfonyl fluoride (13.4)

5-(4-Benzyloxy-phenyl)-pentanesulfonyl fluoride (13.4) was synthesized as described in 13.1 using 12.4 (0.3 g, 0.87 mmol) and NH₄F (0.06 g, 1.64 mmol) in dry acetone (40 mL). Purification by flash column chromatography on silica gel gave the title compound (0.266 g, 91% yield) as a white solid (m p 66-68° C.).

13.4 was confirmed as follows: ¹H NMR (500 MHz, CDCl₃) δ 7.43 (d, J=7.5 Hz, 2H), 7.38 (t, J=7.5 Hz, 2H), 7.32 (t, J=7.5 Hz 1H), 7.08 (d, J=8.0 Hz, 2H), 6.90 (d, J=8.0 Hz, 2H), 5.04 (s, 2H), 3.35-3.32 (m, 2H), 2.58 (t, J=7.5 Hz, 2H), 1.96 (quintet, J=7.7 Hz, 2H), 1.65 (quintet J=7.5 Hz, 2H), 1.50 (quintet, J=7.5 Hz, 2H).

### 7-(4-Hydroxy-phenyl)-heptanesulfonyl fluoride (14.1)

To a solution of 13.1 (0.182 g, 0.5 mmol) in ethanedithiol (10 mL), at RT, under an argon atmosphere was added BF₃.Et₂O (0.282 g, 2.0 mmol). The reaction mixture was stirred at RT for 1 hour and then diluted with diethyl ether (20 mL) and water (10 mL). The organic layer was separated and the aqueous phase extracted with diethyl ether. The combined organic layer was washed with brine, dried over MgSO₄, and concentrated under reduced pressure. The residue obtained was chromatographed through a column of silica gel eluting with 50% diethyl ether-hexane to give 14.1 (0.096 g, 70% yield) as a white solid (melting point 47-51° C.).

14.1 was confirmed as follows: ¹H NMR (500 MHz, CDCl₃) δ 7.08 (d, J=9.0 Hz, 2H), 6.90 (d, J=9.0 Hz, 2H), 4.08 (br s, 2H), 3.36-3.32 (m, 2H), 2.55 (t, J=8.0 Hz, 2H), 1.98-1.90 (m, 2H), 1.62-1.54 (m, 2H), 1.52-1.44 (m, 2H) 1.38-1.34 (m, 4H).

### 7-(3-Hydroxy-phenyl)-heptanesulfonyl fluoride (14.2)

7-(3-Hydroxy-phenyl)-heptanesulfonyl fluoride (14.2) was synthesized as described in 14.1 using 13.2 (0.1 g, 0.26 mmol) in ethanedithiol (5 mL) and BF₃.Et₂O (0.14 g, 1.0 mmol). Purification by flash column chromatography on silica gel gave 14.2 (0.049 g, 69% yield) as a viscous liquid.

14.2 was confirmed as follows: ¹H NMR (500 MHz, CDCl₃) δ 7.14 (t, J=7.5 Hz, 1H), 6.74 (d, J=7.5 Hz, 1H), 6.66-6.64 (m, 2H), 4.70 (br s 1H), 3.36-3.32 (m, 2H), 2.56 (t, J=7.7 Hz, 2H), 1.94 (quintet, J=7.7 Hz, 2H), 1.61 (quintet, J=7.5 Hz, 2H), 1.49 (quintet, J=7.2 Hz, 2H), 1.42-1.32 (m, 4H).

### 7-(2-Hydroxy-phenyl)-heptanesulfonyl fluoride (14.3)

7-(2-Hydroxy-phenyl)-heptanesulfonyl fluoride (14.3) was synthesized as described in 14.1 using 13.3 (0.065 g, 0.17 mmol) in ethanedithiol (5 mL) and BF₃.Et₂O (0.092 g, 0.65 mmol). Purification by flash column chromatography gave 14.3 (0.033 g, 70% yield) as a viscous liquid.

14.3 was confirmed as follows: ¹H NMR (500 MHz, CDCl₃) δ 7.11-7.06 (m, 2H), 6.87 (dt, J=7.7 Hz, J=1.0 Hz, 1H), 6.75 (dd, J=7.7 Hz, J=1.0 Hz, 1H), 4.70 (br s, 1H), 3.35-3.32 (m, 2H), 2.61 (t, J=7.2 Hz, 2H), 1.94 (quintet, J=7.7 Hz, 2H), 1.66-1.58 (m, 2H), 1.52-1.46 (m, 2H), 1.42-1.34 (m, 4H).

### 5-(4-Hydroxy-phenyl)-pentanesulfonyl fluoride (14.4)

5-(4-Hydroxy-phenyl)-pentanesulfonyl fluoride (14.4) was synthesized as described in 14.1 using 13.4 (0.28 g, 0.83 mmol) in ethanedithiol (10 mL) and BF₃.Et₂O (0.47 g, 3.32 mmol). Purification by flash column chromatography on silica gel gave 14.4 (0.139 g, 68% yield) as a white solid (m p 32-35° C.).

14.4 was confirmed as follows: ¹H NMR (500 MHz, CDCl₃) δ 7.02 (d, J=8.2 Hz, 2H), 6.76 (d, J=8.2 Hz, 2H), 4.65 (br s, 1H), 3.36-3.32 (m, 2H), 2.58 (t, J=7.2 Hz, 2H), 1.96 (quintet, J=7.7 Hz, 2H), 1.64 (quintet, J=7.5 Hz, 2H), 1.50 (quintet, J=7.5 Hz, 2H).

### Synthesis of Sulfonyl fluoride 17

Sulfonyl fluoride (17) (shown in Scheme 3) was synthesized by a method depicted in Scheme 3 starting from commercially available 4-phenoxybutyl bromide (5.2).

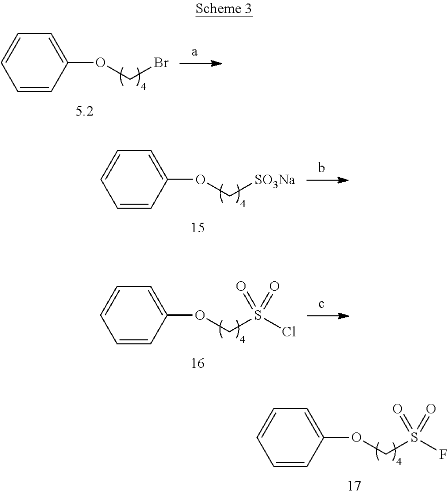

Reagents and conditions for the steps in Scheme 3 were as follows: Step a: Na₂SO₃, EtOH/H₂O, reflux, 6 hours or microwave; Step b: SOCl₂, PhH/DMF, N₂, 50° C., 3 hours, 40%; Step c: NH₄F, acetone, N₂, reflux, 2 hours, 91%.

### 4-Phenoxybutyl Sulfonic Acid Sodium Salt (15)

Following the procedure described for 11.1 using 5.2 (1.0 g, 4.37 mmol), Na₂SO₃ (0.77 g, 6.11 mmol), and EtOH (30 mL)/H₂O (10 mL) mixture, the crude (15) was obtained and used in the next step without further purification.

### 4-Phenoxybutyl sulfonyl chloride (16)

4-Phenoxybutyl sulfonyl chloride (16) was synthesized as described in 12.1 using 15 (1.0 g, 4.37 mmol) and thionyl chloride (1.55 g, 13.0 mmol) in benzene (40 mL)/DMF (4 mL). Purification by flash column chromatography on silica gel afforded 15 (0.434 g, 40% yield) as a white solid (melting point 65-67° C.).

16 was confirmed as follows: ¹H NMR (500 MHz, CDCl₃) δ 7.29 (t, J=8.2 Hz, 2H), 6.97 (t, J=8.2 Hz, 1H), 6.89 (d, J=8.2 Hz, 2H), 4.04 (t, J=5.7 Hz, 2H), 3.80 (m as t, half of an AA′XX′ system, 2H), 2.29 (quintet, J=7.7 Hz, 2H), 2.01 (quintet, J=7.7 Hz, 2H).

### 4-Phenoxybutylsulfonyl fluoride (17)

4-Phenoxybutylsulfonyl fluoride (17) was synthesized as in 13.1 using 16 (0.4 g, 1.6 mmol) and NH₄F (0.118 g, 3.2 mmol) in dry acetone (20 mL). Purification by flash column chromatography on silica gel gave 17 (0.338 g, 91% yield) as a white solid (m p 74-76° C.).

17 was confirmed as follows: ¹H NMR (500 MHz, CDCl₃) δ 7.29 (t, J=7.5 Hz, 2H), 6.97 (t, J=7.5 Hz, 1H), 6.89 (d, J=7.5 Hz, 2H), 4.03 (t, J=5.5 Hz, 2H), 3.52-3.48 (m, 2H), 2.20 (quintet, J=7.7 Hz, 2H), 2.00 (quintet, J=8.0 Hz, 2H).

### Synthesis of sulfonyl esters (18)

Sulfonyl ester 18 (shown in Scheme 4) was synthesized by a method depicted in Scheme 4 starting from 12.1.

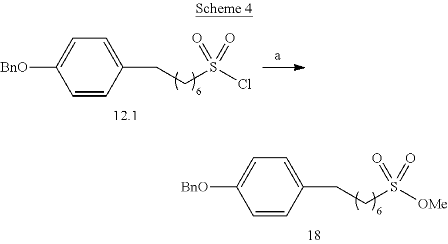

Reagents and conditions for Scheme 4 were as follows: Step a: MeOH, RT, overnight, 82%.

### 7-(4-Benzyloxy-phenyl)-heptane-1-sulfonic acid methyl ester (18)

A solution of 12.1 (0.050 g, 0.13 mmol) in MeOH (5 mL) was stirred at RT overnight. The solvent was evaporated under reduced pressure and the residue obtained was dissolved in diethyl ether (20 mL). The ethereal solution was washed with water and brine, dried (MgSO₄), and evaporated under reduced pressure. Purification by flash column chromatography on silica gel (20% diethyl ether-hexane) gave the pure compound 18 (0.046 g, 82% yield), as a white solid (m p 57-59° C.).

18 was confirmed as follows: ¹H NMR. (500 MHz, CDCl₃) δ 7.43 (d, J=7.5 Hz, 2H), 7.38 (t, J=7.5 Hz, 2H), 7.32 (t, J=7.5 Hz 1H), 7.08 (d, J=8.7 Hz, 2H), 6.90 (d, J=8.7 Hz, 2H), 5.04 (s, 2H), 3.88 (s, 3H), 3.08 (m as t, half of an AA′XX′ system, J=7.7 Hz, 2H), 2.54 (t, J=7.7 Hz, 2H), 1.85 (quintet, J=7.7 Hz, 2H), 1.56 (quintet, J=7.0 Hz, 2H), 1.46-1.39 (m, 2H), 1.38-1.30 (m, 4H).

### Synthesis of trifluoromethyl ketones (23.1-12 and 24.1-10)

Trifluoromethyl ketones 23.1-12 and 24.1-10 were synthesized by a method depicted in Scheme 5 starting from commercially available 2- or 3- or 4-(benzyloxy)phenol (19) and the appropriate ω-bromo-n-alkyl acid ethyl ester. 4-Phenoxy-butanoic acid (21.11) and 5-phenoxy-pentanoic acid (21.12) were also commercially available materials. Compound 24.5 was isolated in its hydrate form.

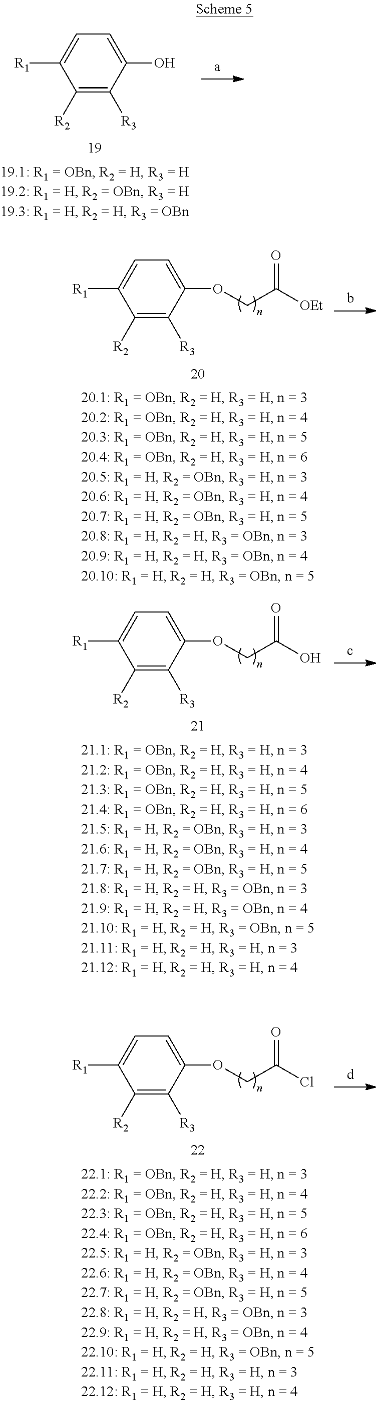

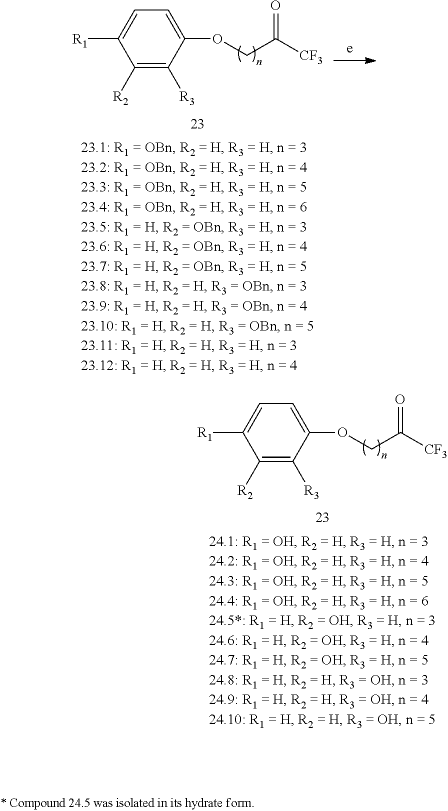

Reagents and conditions for the steps in Scheme 5 were as follows: Step a: K₂CO₃, 18-crown-6, Br-(CH₂)ₙ—COOEt, RT; Strep b: KOH, EtOH/H₂O, RT, 80-93% from 19; Step c: (COCl)₂, CH₂Cl₂, RT; Step d: (i) pyridine, CF₃COOCOCF₃, CH₂Cl₂, −78° C. to 0° C., (ii) H₂O, 0° C. to RT, 57-63% from 21; Step e: H₂, Pd/C, EtOH, RT, 70-97%.

### Esters (20)

A mixture of benzyloxyphenol (19) (1 equiv.), ω-bromo-n-alkyl acid ethyl ester (1.2 equiv.), potassium carbonate (1.2 equiv.), and 18-crown-6 (1 equiv.) in anhydrous acetonitrile was stirred overnight at RT under an argon atmosphere. The reaction mixture was evaporated, and the residue was partitioned between water and diethyl ether. The organic phase was separated, washed with brine, dried (MgSO₄), and the solvent was removed under reduced pressure to leave the crude product (20). This product contains small amounts of unreacted ω-bromo-n-alkyl acid ethyl ester. It was used in the next step without purification. For analytical purposes 20.7 and 20.4 were further purified by flash column chromatography (20% diethyl ether-hexane) on silica gel. For a ¹H NMR spectrum and an alternative method for the preparation of 20.4 see description for the synthesis of α-keto-heterocycles.

### 6-[3-(Benzyloxy)phenoxy]hexanoic acid ethyl ester (20.7)

Following the procedure described for esters, 6-[3-(Benzyloxy)phenoxy]hexanoic acid ethyl ester (20.7) was as a colorless oil.

20.7 was confirmed as follows: ¹H NMR (500 MHz, CDCl₃) δ 7.41 (d, J=7.3 Hz, 2H), 7.36 (t, J=7.3 Hz, 2H), 7.30 (t, J=7.3 Hz, 1H), 7.14 (t, J=8.2 Hz, 1H), 6.57-6.52 (m, 2H), 6.49 (dd, J=8.2 Hz, J=1.8 Hz, 1H), 5.01 (s, 2H), 4.11 (q, J=7.2 Hz, 2H), 3.91 (t, J=6.5 Hz, 2H), 2.31 (t, J=7.5 Hz, 2H), 1.80-1.73 (m, 2H), 1.72-1.64 (m, 2H), 1.51-1.43 (m, 2H), 1.24 (t, J=7.2 Hz, 3H).

### Acids (21)

A mixture of the crude ester (20) and KOH (1.3 equiv.) in EtOH/H₂O (10:1 mixture) was heated under reflux for 3-4 hours. The reaction mixture was cooled to RT, and the solvent was removed under reduced pressure. The residue obtained was dissolved in water, and the pH was adjusted to 1 using concentrated HCl solution. The precipitated crude acid was isolated by filtration and dissolved in ethyl acetate. The resulting solution was washed with brine, dried (MgSO₄), and the solvent was evaporated to give the product 21 in 80-93% yield (from 19).

### Selected data of synthesized acids (21)

### 4-[4-(Benzyloxy)phenoxy]butanoic acid (21.1)

According to the procedure described above for acids, 4-[4-(Benzyloxy)phenoxy]butanoic acid (21.1) was obtained as a white solid with a melting point of 125-126° C.

21.1 was confirmed as follows: ¹H NMR (500 MHz, CDCl₃) δ 10.95 (br s, 1H), 7.41 (d, J=7.3 Hz, 2H), 7.37 (t, J=7.3 Hz, 2H), 7.31 (t, J=7.3 Hz, 1H), 6.90 (m as d, J=9.0 Hz, 2H), 6.81 (m as d, J=9.0 Hz, 2H), 5.01 (s, 2H), 3.97 (t, J=6.3 Hz, 2H), 2.58 (t, J=7.5 Hz, 2H), 2.09 (quintet, J=6.7 Hz, 2H); IR (neat) 2904, 2865, 1704, 1509 cm⁻¹.

### 5-[4-(Benzyloxy)phenoxy]pentanoic acid (21.2)

According to the procedure described above for acids, 5-[4-(Benzyloxy)phenoxy]pentanoic acid (21.2) was obtained as a white solid with a melting point of 127-128° C.

21.2 was confirmed as follows: ¹H NMR (500 MHz, CDCl₃) δ 11.04 (br s, 1H), 7.42 (d, J=7.3 Hz, 2H), 7.37 (t, J=7.3 Hz, 2H), 7.31 (t, J=7.3 Hz, 1H), 6.89 (d, J=8.9 Hz, 2H), 6.81 (d, J=8.9 Hz, 2H), 5.01 (s, 2H), 3.92 (t, J=6.4 Hz, 2H), 2.44 (t, J=7.1 Hz, 2H), 1.85-1.79 (m, 4H); IR (neat) 2954, 2864, 1694, 1509 cm⁻¹.

### 6-[4-(Benzyloxy)phenoxy]hexanoic acid (21.3)

According to the procedure described above for acids, 6-[4-(Benzyloxy)phenoxy]hexanoic acid (21.3) was obtained as a white solid with a melting point of 100-101° C.

21.3 was confirmed as follows: ¹H NMR (500 MHz, CDCl₃) δ 11.00 (br s, 1H), 7.42 (d, J=7.3 Hz, 2H), 7.37 (t, J=7.3 Hz, 2H), 7.31 (t, J=7.3 Hz, 1H), 6.89 (d, J=9.0 Hz, 2H), 6.81 (d, J=9.0 Hz, 2H), 5.01 (s, 2H), 3.90 (t, J=6.4 Hz, 2H), 2.39 (t, J=7.4 Hz, 2H), 1.78 (quintet, J=6.8 Hz, 2H), 1.71 (quintet, J=7.5 Hz, 2H), 1.60-1.45 (m, 2H); IR (neat) 2945, 2863, 1693, 1508 cm⁻¹.

### 7-[4-(Benzyloxy)phenoxy]heptanoic acid (21.4)

According to the procedure described above for acids, 7-[4-(Benzyloxy)phenoxy]heptanoic acid (21.4) was obtained as a white solid with a melting point of 118-119° C.

21.4 was confirmed as follows: ¹H NMR (500 MHz, CDCl₃) δ 11.20 (br s, 1H), 7.42 (d, J=7.3 Hz, 2H), 7.37 (t, J=7.3 Hz, 2H), 7.31 (t, J=7.3 Hz, 1H), 6.89 (d, J=9.0 Hz, 2H), 6.81 (d, J=9.0 Hz, 2H), 5.01 (s, 2H), 3.89 (t, J=6.4 Hz, 2H), 2.36 (t, J=7.4 Hz, 2H), 1.79-1.72 (m, 2H), 1.70-1.63 (m, 2H), 1.51-1.37 (m, 4H).

### 4-[3-(Benzyloxy)phenoxy]butanoic acid (21.5)

According to the procedure described above for acids, 4-[3-(Benzyloxy)phenoxy]butanoic acid (21.5) was obtained as a white solid with a melting point of 76-77° C.

### 5-[3-(Benzyloxy)phenoxy]pentanoic acid (21.6)

According to the procedure described above for acids, 5-[3-(Benzyloxy)phenoxy]pentanoic acid (21.6) was obtained as a white solid with a melting. m p 71-72° C.

21.6 was confirmed as follows: ¹H NMR (500 MHz, CDCl₃) δ 10.82 (br s, 1H), 7.45 (d, J=7.3 Hz, 2H), 7.38 (t, J=7.3 Hz, 2H), 7.32 (t, J=7.3 Hz, 1H), 7.17 (t, J=8.2 Hz, 1H), 6.57 (dd, J=8.2 Hz, J=2.0 Hz, 1H), 6.54 (t, J=2.0 Hz, 1H), 6.50 (dd, J=8.2 Hz, J=2.0 Hz, 1H), 5.04 (s, 2H), 3.95 (t, J=5.7 Hz, 2H), 2.44 (t, J=6.7 Hz, 2H), 1.87-1.80 (m, 4H).

### 6-[3-(Benzyloxy)phenoxy]hexanoic acid (21.7)

According to the procedure described above for acids, 6-[3-(Benzyloxy)phenoxy]hexanoic acid (21.7) was obtained as a white solid with a melting point of 72-73° C.

21.7 was confirmed as follows: ¹H NMR (500 MHz, CDCl₃) δ 11.31 (br s, 1H), 7.42 (d, J=7.3 Hz, 2H), 7.38 (t, J=7.3 Hz, 2H), 7.32 (t, J=7.3 Hz, 1H), 7.16 (t, J=8.2 Hz, 1H), 6.56 (dd, J=8.2 Hz, J=1.8 Hz, 1H), 6.54 (t, J=1.8 Hz, 1H), 6.50 (dd, J=8.2 Hz, J=1.8 Hz, 1H), 5.04 (s, 2H), 3.93 (t, J=6.5 Hz, 2H), 2.39 (t, J=7.5 Hz, 2H), 1.83-1.75 (m, 2H), 1.74-1.67 (m, 2H), 1.56-1.48 (m, 2H).

### 4-[2-(Benzyloxy)phenoxy]butanoic acid (21.8)

According to the procedure described above for acids, 4-[2-(Benzyloxy)phenoxy]butanoic acid (21.8) was obtained as a white solid with a melting point of 75-76° C.

21.8 was confirmed as follows: ¹H NMR (500 MHz, CDCl₃) δ 9.50 (br s, 1H), 7.44 (d, J=7.4 Hz, 2H), 7.37 (t, J=7.4 Hz, 2H), 7.30 (t, J=7.4 Hz, 1H), 6.95-6.86 (m, 4H), 5.12 (s, 2H), 4.09 (t, J=5.9 Hz, 2H), 2.61 (t, J=7.1 Hz, 2H), 2.15 (quintet, J=6.5 Hz, 2H); IR (neat) 1693, 1590 cm⁻¹.

### 5-[2-(Benzyloxy)phenoxy]pentanoic acid (21.9)

According to the procedure described above for acids, 5-[2-(Benzyloxy)phenoxy]pentanoic acid (21.9) was obtained as a white solid with a melting point of 74-75° C.

21.9 was confirmed as follows: ¹H NMR (500 MHz, CDCl₃) δ 11.02 (br s, 1H), 7.44 (d, J=7.4 Hz, 2H), 7.36 (t, J=7.4 Hz, 2H), 7.29 (t, J=7.4 Hz, 1H), 6.95-6.85 (m, 4H), 5.12 (s, 2H), 4.05 (t, J=5.9 Hz, 2H), 2.45 (t, J=7.1 Hz, 2H), 1.92-1.82 (m, 4H).

### 6-[2-(Benzyloxy)phenoxy]hexanoic acid (21.10)

According to the procedure described above for acids, 6-[2-(Benzyloxy)phenoxy]hexanoic acid (21.10) was obtained as a white solid with a melting point of 77-78° C.

21.10 was confirmed as follows: ¹H NMR (500 MHz, CDCl₃) δ 10.92 (br s, 1H), 7.44 (d, J=7.3 Hz, 2H), 7.36 (t, J=7.3 Hz, 2H), 7.29 (t, J=7.3 Hz, 1H), 6.95-6.84 (m, 4H), 5.12 (s, 2H), 4.03 (t, J=6.4 Hz, 2H), 2.36 (t, J=7.2 Hz, 2H), 1.85 (quintet, J=6.7 Hz, 2H), 1.71 (quintet, J=7.3 Hz, 2H), 1.59-1.51 (m, 2H).

### Carboxylic acid chlorides (22)

To a solution of acid (21) (1 equiv.) in anhydrous CH₂Cl₂ at RT, under an argon atmosphere was added oxalyl chloride (2 equiv.) over a 2-min period. The mixture was stirred for 2 hours, solvent and excess oxalyl chloride were removed under reduced pressure, and the crude carboxylic acid chloride (22) was used in the next step without further purification.

### Trifluoromethyl ketones (23)

To a solution of carboxylic acid chloride (22) in anhydrous CH₂Cl₂ at −78° C. under an argon atmosphere were added successively trifluoroacetic anhydride (6 equiv.) and dry pyridine (8 equiv.). The reaction mixture was stirred at −78° C. for 2 hours, and then it was allowed to warm to 0° C. and stirred for an additional 2 hours. Water was added dropwise, the resulting mixture was warmed to RT, and extracted with CH₂Cl₂. The organic layer was washed with brine, dried (MgSO₄), and the solvent was evaporated. Following the workup, the crude mixture was chromatographed on a silica gel column (eluting with 30% diethyl ether-hexane), and the fraction that contains the product was concentrated and dried in high vacuum (in the presence of P₂O₅) to give compound 23 in 57-63% yield (from 21).

### Selected data of synthesized trifluoromethyl ketones (23)

### 1,1,1-Trifluoro-5-[4-(benzyloxy)phenoxy]-2-pentanone (23.1)

According to the procedure described above for trifluoromethyl ketones, 1,1,1-Trifluoro-5-[4-(benzyloxy)phenoxy]-2-pentanone (23.1) was obtained as a white solid with a melting point of 59-61° C.

23.1 was confirmed as follows: ¹H NMR (500 MHz, CDCl₃) δ 7.41 (d, J=7.3 Hz, 2H), 7.37 (t, J=7.3 Hz, 2H), 7.30 (t, J=7.3 Hz, 1H), 6.89 (m as d, J=9.0 Hz, 2H), 6.79 (m as d, J=9.0 Hz, 2H), 5.01 (s, 2H), 3.96 (t, J=5.7 Hz, 2H), 2.93 (t, J=7.0 Hz, 2H), 2.14 (quintet, J=6.5 Hz, 2H); IR (neat) 1765, 1509 cm⁻¹.

### 1,1,1-Trifluoro-6-[4-(Benzyloxy)phenoxy]-2-hexanone (23.2)

According to the procedure described above for trifluoromethyl ketones, 1,1,1-Trifluoro-6-[4-(Benzyloxy)phenoxy]-2-hexanone (23.2) was obtained as a white solid with a melting point of 95.5-96° C.

23.2 was confirmed as follows: ¹H NMR (500 MHz, CDCl₃) δ 7.42 (d, J=7.3 Hz, 2H), 7.38 (t, J=7.3 Hz, 2H), 7.31 (t, J=7.3 Hz, 1H), 6.90 (d, J=8.9 Hz, 2H), 6.81 (d, J=8.9 Hz, 2H), 5.01 (s, 2H), 3.93 (t, J=6.4 Hz, 2H), 2.82 (t, J=7.1 Hz, 2H), 1.88 (quintet, J=7.1 Hz, 2H), 1.81 (quintet, J=6.6 Hz, 2H); IR (neat) 1759, 1509 cm⁻¹.

### 1,1,1-Trifluoro-7-[4-(Benzyloxy)phenoxy]-2-heptanone (23.3)

According to the procedure described above for trifluoromethyl ketones, 1,1,1-Trifluoro-7-[4-(Benzyloxy)phenoxy]-2-heptanone (23.3) was obtained as a white solid with a melting point of 59-60° C.

23.3 was confirmed as follows: IR (neat) 1761, 1509 cm⁻¹.

### 1,1,1-Trifluoro-8-[4-(Benzyloxy)phenoxy]-2-octanone (23.4)

According to the procedure described above for trifluoromethyl ketones, 1,1,1-Trifluoro-8-[4-(Benzyloxy)phenoxy]-2-octanone (23.4) was obtained as a white solid with a melting point of 82-83° C.

23.4 was confirmed as follows: ¹H NMR (500 MHz, CDCl₃) δ 7.42 (d, J=7.3 Hz, 2H), 7.38 (t, J=7.3 Hz, 2H), 7.31 (t, J=7.3 Hz, 1H), 6.89 (d, J=8.9 Hz, 2H), 6.81 (d, J=8.9 Hz, 2H), 5.01 (s, 2H), 3.90 (t, J=6.4 Hz, 2H), 2.73 (t, J=7.1 Hz, 2H), 1.80-1.67 (m, 4H), 1.52-1.45 (m, 2H), 1.44-1.36 (m, 2H).

### 1,1,1-Trifluoro-5-[3-(Benzyloxy)phenoxy]-2-pentanone (23.5)

According to the procedure described above for trifluoromethyl ketones, 1,1,1-Trifluoro-5-[3-(Benzyloxy)phenoxy]-2-pentanone (23.5) was obtained as a colorless viscous oil.

23.5 was confirmed as follows: ¹H NMR (500 MHz, CDCl₃) δ 7.42 (d, J=7.3 Hz, 2H), 7.38 (t, J=7.3 Hz, 2H), 7.31 (t, J=7.3 Hz, 1H), 7.16 (t, J=8.2 Hz, 1H), 6.58 (dd, J=8.2 Hz, J=2.0 Hz, 1H), 6.52 (t, J=2.0 Hz, 1H), 6.48 (dd, J=8.2 Hz, J=2.0 Hz, 1H), 5.03 (s, 2H), 3.96 (t, J=5.9 Hz, 2H), 2.92 (t, J=6.9 Hz, 2H), 2.14 (quintet, J=6.5 Hz, 2H); IR (neat) 1763, 1591 cm⁻¹.

### 1,1,1-Trifluoro-6-[3-(Benzyloxy)phenoxy]-2-hexanone (23.6)

According to the procedure described above for trifluoromethyl ketones, 1,1,1-Trifluoro-6-[3-(Benzyloxy)phenoxy]-2-hexanone (23.6) was obtained as a colorless viscous oil.

23.6 was confirmed as follows: ¹H NMR (500 MHz, CDCl₃) δ 7.42 (d, J=7.3 Hz, 2H), 7.38 (t, J=7.3 Hz, 2H), 7.32 (t, J=7.3 Hz, 1H), 7.17 (t, J=8.2 Hz, 1H), 6.58 (dd, J=8.2 Hz, J=2.0 Hz, 1H), 6.53 (t, J=2.0 Hz, 1H), 6.49 (dd, J=8.2 Hz, J=2.0 Hz, 1H), 5.04 (s, 2H), 3.96 (t, J=5.9 Hz, 2H), 2.81 (t, J=6.8 Hz, 2H), 1.91-1.78 (m, 4H).

### 1,1,1-Trifluoro-7-[3-(Benzyloxy)phenoxy]-2-heptanone (23.7)

According to the procedure described above for trifluoromethyl ketones, 1,1,1-Trifluoro-7-[3-(Benzyloxy)phenoxy]-2-heptanone (23.7) was obtained as a colorless viscous oil.

### 1,1,1-Trifluoro-5-[2-(Benzyloxy)phenoxy]-2-pentanone (23.8)

According to the procedure described above for trifluoromethyl ketones, 1,1,1-Trifluoro-5-[2-(Benzyloxy)phenoxy]-2-pentanone (23.8) was obtained as a white solid with a melting point of 50-51° C.

23.8 was confirmed as follows: ¹H NMR (500 MHz, CDCl₃) δ 7.42 (d, J=7.4 Hz, 2H), 7.36 (t, J=7.4 Hz, 2H), 7.30 (t, J=7.4 Hz, 1H), 6.96-6.89 (m, 4H), 5.09 (s, 2H), 4.06 (t, J=5.9 Hz, 2H), 2.98 (t, J=7.0 Hz, 2H), 2.16 (quintet, J=6.5 Hz, 2H); IR (neat) 1763, 1593 cm⁻¹.

### 1,1,1-Trifluoro-6-[2-(Benzyloxy)phenoxy]-2-hexanone (23.9)

According to the procedure described above for trifluoromethyl ketones, 1,1,1-Trifluoro-6-[2-(Benzyloxy)phenoxy]-2-hexanone (23.9) was obtained as a colorless viscous oil.

23.9 was confirmed as follows: ¹H NMR (500 MHz, CDCl₃) δ 7.42 (d, J=7.4 Hz, 2H), 7.35 (t, J=7.4 Hz, 2H), 7.29 (t, J=7.4 Hz, 1H), 6.93 (d, J=7.4 Hz, 1H), 6.91-6.86 (m and t overlapping, especially 6.90, t, J=3.9 Hz, 3H), 5.10 (s, 2H), 4.04 (t, J=5.9 Hz, 2H), 2.80 (t, J=6.9 Hz, 2H), 1.93-1.82 (m, 4H).

### 1,1,1-Trifluoro-7-[2-(Benzyloxy)phenoxy]-2-heptanone (23.10)

According to the procedure described above for trifluoromethyl ketones, 1,1,1-Trifluoro-7-[2-(Benzyloxy)phenoxy]-2-heptanone (23.10) was obtained as a white solid with a melting point 31-32° C.

23.10 was confirmed as follows: ¹H NMR (500 MHz, CDCl₃) δ 7.43 (d, J=7.3 Hz, 2H), 7.36 (t, J=7.3 Hz, 2H), 7.30 (t, J=7.3 Hz, 1H), 6.95-6.85 (m, 4H), 5.11 (s, 2H), 4.03 (t, J=6.4 Hz, 2H), 2.70 (t, J=7.1 Hz, 2H), 1.86 (quintet, J=6.7 Hz, 2H), 1.75 (quintet, J=7.3 Hz, 2H), 1.59-1.50 (m, 2H).

### 1,1,1-Trifluoro-5-phenoxy-2-pentanone (23.11)

According to the procedure described above for trifluoromethyl ketones, 1,1,1-Trifluoro-5-phenoxy-2-pentanone (23.11) was obtained as a colorless viscous oil.

23.11 was confirmed as follows: ¹H NMR (500 MHz, CDCl₃) δ 7.28 (t, J=7.4 Hz, 2H), 6.95 (t, J=7.4 Hz, 1H), 6.87 (d, J=7.4 Hz, 2H), 4.00 (t, J=5.8 Hz, 2H), 2.95 (t, J=7.0 Hz, 2H), 2.17 (quintet, J=6.4 Hz, 2H); ¹³C NMR (126 MHz, CDCl₃) δ 191.6 (q, J=35 Hz, C═O), 158.9, 129.9, 121.4, 116.0 (q, J=292 Hz, CF₃), 114.8, 66.1, 33.5, 22.8; IR (neat) 1763, 1601, 1588, 1498 cm⁻¹; mass spectrum m/z (relative intensity) 232 (M⁺, 25), 139 (24), 94 (100), 77 (16), 69 (27). Exact mass calculated for C₁₁H₁₁O₂F₃; 232.0711; found, 232.0714.

### 1,1,1-Trifluoro-6-phenoxy-2-hexanone (23.12)

According to the procedure described above for trifluoromethyl ketones, 1,1,1-Trifluoro-6-phenoxy-2-hexanone (23.12) was obtained as a white solid with a melting point of 50-51° C.

23.12 was confirmed as follows: ¹H NMR (500 MHz, CDCl₃) δ 7.28 (t, J=7.4 Hz, 2H), 6.94 (t, J=7.4 Hz, 1H), 6.88 (d, J=7.4 Hz, 2H), 3.98 (t, J=5.9 Hz, 2H), 2.83 (t, J=6.7 Hz, 2H), 1.95-1.80 (m, 4H); ¹³C NMR (126 MHz, CDCl₃) δ 191.7 (q, J=35 Hz, C═O), 159.2, 129.9, 121.2, 116.0 (q, J=291 Hz, CF₃), 114.8, 67.5, 36.4, 28.6, 19.8; IR (neat) 1759, 1601, 1585, 1500 cm⁻¹.

### Trifluoromethyl ketones (24)

To a solution of trifluoromethyl ketone (23) (1 equiv.) in EtOH was added 10% Pd/C (7% w/w), and the resulting suspension was stirred vigorously under hydrogen atmosphere, overnight at RT. The catalyst was removed by filtration through Celite, and the filtrate was evaporated under reduced pressure. The residue obtained was chromatographed on a silica gel column (eluting with 60% diethyl ether-hexane), and the fraction that contains the product was concentrated and dried in high vacuum (in the presence of P₂O₅) to give compound 24 in 70-97% yield. Especially in case of compound 24.5 the hydrate was isolated in 80% yield.

### Selected data of synthesized trifluoromethyl ketones (24)

### 1,1,1-Trifluoro-5-[4-(hydroxy)phenoxy]-2-pentanone (24.1)

According to the procedure described above for trifluoromethyl ketones (24), 1,1,1-Trifluoro-5-[4-(hydroxy)phenoxy]-2-pentanone (24.1) was obtained as a colorless viscous oil.

24.1 was confirmed as follows: ¹H NMR (500 MHz, CDCl₃) δ 6.76 (m as br s, 4H), 4.51 (br s, 1H), 3.95 (t, J=5.8 Hz, 2H), 2.95 (t, J=7.0 Hz, 2H), 2.15 (quintet, J=6.5 Hz, 2H); ¹³C NMR (126 MHz, CDCl₃) δ 191.5 (q, J=35 Hz, C═O), 152.7, 149.8, 116.2, 115.7, 115.6 (q, J=292 Hz, CF₃), 66.7, 33.2, 22.5; IR (neat) 3379 br, 1763, 1509 cm⁻¹.

### 1,1,1-Trifluoro-6-[4-(hydroxy)phenoxy]-2-hexanone (24.2)

According to the procedure described above for trifluoromethyl ketones (24), 1,1,1-Trifluoro-6-[4-(hydroxy)phenoxy]-2-hexanone (24.2) was obtained as a white solid with a melting point of 63-64° C.

24.2 was confirmed as follows: ¹H NMR (500 MHz, CDCl₃) δ 6.76 (m as br s, 4H), 4.57 (br s, 1H), 3.92 (t, J=6.4 Hz, 2H), 2.82 (t, J=7.1 Hz, 2H), 1.88 (quintet, J=7.1 Hz, 2H), 1.81 (quintet, J=6.6 Hz, 2H); IR (neat) 3398 br, 1754, 1509 cm⁻¹.

### 1,1,1-Trifluoro-7-[4-(hydroxy)phenoxy]-2-heptanone (24.3)

According to the procedure described above for trifluoromethyl ketones (24), 1,1,1-Trifluoro-7-[4-(hydroxy)phenoxy]-2-heptanone (24.3) was obtained as a colorless viscous oil.

24.3 was confirmed as follows: IR (neat) 3386 br, 1762, 1509 cm⁻¹.

### 1,1,1-Trifluoro-8-[4-(hydroxy)phenoxy]-2-octanone (24.4)

According to the procedure described above for trifluoromethyl ketones (24), 1,1,1-Trifluoro-8-[4-(hydroxy)phenoxy]-2-octanone (24.4) was obtained as a white solid with a melting point of 61-62° C.

24.4 was confirmed as follows: ¹H NMR (500 MHz, CDCl₃) δ 6.77 (m as d, J=9.1 Hz, 2H), 6.75 (m as d, J=9.1 Hz, 2H), 4.40 (br s, 1H), 3.89 (t, J=6.4 Hz, 2H), 2.73 (t, J=7.1 Hz, 2H), 1.80-1.67 (m, 4H), 1.52-1.45 (m, 2H), 1.44-1.36 (m, 2H).

### 1,1,1-Trifluoro-2,2-dihydroxy-5-[3-(hydroxy)phenoxy]pentane (24.5)

According to the procedure described above for trifluoromethyl ketones (24), 1,1,1-Trifluoro-2,2-dihydroxy-5-[3-(hydroxy)phenoxy]pentane (24.5) was obtained as a white solid with a melting point of 76-77° C.

24.5 was confirmed as follows: ¹H NMR (500 MHz, CDCl₃/DMSO-d₆) δ 8.53 (br s, exchange with D₂O, 1H), 7.06 (t, J=8.2 Hz, 1H), 6.47-6.42 (m, 2H), 6.39 (dd, J=8.2 Hz, J=1.9 Hz, 1H), 5.49 (br s, exchange with D₂O, 2H), 3.99 (t, J=6.1 Hz, 2H), 2.05 (m, 2H), 1.95 (t, J=7.1 Hz, 2H); IR (neat) 3300 br, 1605 cm⁻¹.

### 1,1,1-Trifluoro-6-[3-(hydroxy)phenoxy]-2-hexanone (24.6)

According to the procedure described above for trifluoromethyl ketones (24), 1,1,1-Trifluoro-6-[3-(hydroxy)phenoxy]-2-hexanone (24.6) was obtained as a colorless viscous oil.

24.6 was confirmed as follows: ¹H NMR (500 MHz, CDCl₃) δ 7.11 (t, J=8.2 Hz, 1H), 6.46 (dd, J=8.2 Hz, J=2.2 Hz, 1H), 6.42 (dd, J=8.2 Hz, J=2.2 Hz, 1H), 6.39 (t, J=2.2 Hz, 1H), 5.19 (br s, 1H), 3.94 (t, J=5.9 Hz, 2H), 2.81 (t, J=6.8 Hz, 2H), 1.90-1.77 (m, 4H).

### 1,1,1-Trifluoro-7-[3-(hydroxy)phenoxy]-2-heptanone (24.7)

According to the procedure described above for trifluoromethyl ketones (24), 1,1,1-Trifluoro-7-[3-(hydroxy)phenoxy]-2-heptanone (24.7) was obtained as an orange viscous oil.

### 1,1,1-Trifluoro-5-[2-(hydroxy)phenoxy]-2-pentanone (24.8)

According to the procedure described above for trifluoromethyl ketones (24), 1,1,1-Trifluoro-5-[2-(hydroxy)phenoxy]-2-pentanone (24.8) was obtained as a colorless viscous oil.

24.8 was confirmed as follows: ¹H NMR (500 MHz, CDCl₃) δ 6.95 (d, J=7.7 Hz, 1H), 6.89 (m as quintet, J=3.9 Hz, 1H), 6.83 (d, J=4.2 Hz, 2H), 5.52 (br s, 1H), 4.11 (t, J=6.0 Hz, 2H), 2.96 (t, J=6.9 Hz, 2H), 2.23 (quintet, J=6.5 Hz, 2H).

### 1,1,1-Trifluoro-6-[2-(hydroxy)phenoxy]-2-hexanone (24.9)

According to the procedure described above for trifluoromethyl ketones (24), 1,1,1-Trifluoro-6-[2-(hydroxy)phenoxy]-2-hexanone (24.9) was obtained as a white solid with a melting point of 51-52° C.

24.9 was confirmed as follows: ¹H NMR (500 MHz, CDCl₃) δ 6.94 (d, J=7.7 Hz, 1H), 6.90-6.86 (m, 1H), 6.85-6.82 (m, 2H), 5.60 (br s, 1H), 4.07 (t, J=5.7 Hz, 2H), 2.83 (t, J=6.3 Hz, 2H), 1.94-1.84 (m, 4H).

### 1,1,1-Trifluoro-7-[2-(hydroxy)phenoxy]-2-heptanone (24.10)

According to the procedure described above for trifluoromethyl ketones (24), 1,1,1-Trifluoro-7-[2-(hydroxy)phenoxy]-2-heptanone (24.10) was obtained as a white semi-solid.

24.10 was confirmed as follows: ¹H NMR (500 MHz, CDCl₃) δ 6.84 (d, J=7.3 Hz, 1H), 6.80-6.72 (m, 3H), 5.58 (br s, 1H), 3.95 (t, J=6.4 Hz, 2H), 2.66 (t, J=7.1 Hz, 2H), 1.75 (quintet, J=6.7 Hz, 2H), 1.66 (quintet, J=7.3 Hz, 2H), 1.46-1.38 (m, 2H).

### Synthesis of Trifluoromethyl Ketones (27)

Trifluoromethyl ketones (27.1-4) were synthesized by a method depicted in Scheme 6. 4-Phenyl-butyric acid (25.1), 5-phenyl-pentanoic acid (25.2), 6-phenyl-hexanoic acid (25.3) and 5-(4-methoxy-phenyl)-pentanoic acid (25.4) were commercially available starting materials.

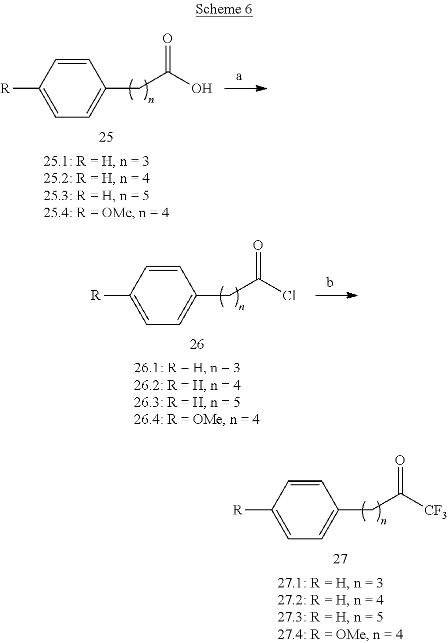

Reagents and conditions for the steps in Scheme 6 were as follows: Step a: (COCl)₂, CH₂Cl₂, RT; Step b: (i) pyridine, CF₃COOCOCF₃, CH₂Cl₂, −78° C. to 0° C., (ii) H₂O, 0° C. to RT, 61-63% from 25.

The synthesis of compounds 27 was carried out analogous to the preparation of compounds 23.

### Selected data of synthesized Trifluoromethyl Ketones 27

### 1,1,1-Trifluoro-5-phenyl-2-pentanone (27.1)

27.1 was synthesized as a colorless viscous oil.

27.1 was confirmed as follows: IR (neat) 1762, 1604, 1498, 1454, 1403 cm⁻¹.

### 1,1,1-Trifluoro-6-phenyl-2-hexanone (27.2)

27.2 was synthesized as a colorless viscous oil.

27.2 was confirmed as follows: ¹H NMR (500 MHz, CDCl₃) δ 7.27 (t, J=7.5 Hz, 2H), 7.17 (t, J=7.5 Hz, 1H), 7.15 (d, J=7.5 Hz, 2H), 2.70 (t, J=7.2 Hz, 2H), 2.63 (t, J=7.7 Hz, 2H), 1.76-1.62 (m, 4H); ¹³C NMR (126 MHz, CDCl₃) δ 191.7 (q, J=35 Hz, C═O), 142.0, 128.8, 128.7, 126.3, 116.0 (q, J=292 Hz, CF₃), 36.6, 35.8, 30.8, 22.4; IR (neat) 1763, 1604, 1497, 1454, 1404 cm⁻¹.

### 1,1,1-Trifluoro-7-phenyl-2-heptanone (27.3)

27.3 was synthesized as a colorless viscous oil.

27.3 was confirmed as follows: ¹H NMR (500 MHz, CDCl₃) δ 7.27 (t, J=7.5 Hz, 2H), 7.18 (t, J=7.5 Hz, 1H), 7.16 (d, J=7.5 Hz, 2H), 2.69 (t, J=7.2 Hz, 2H), 2.61 (t, J=7.7 Hz, 2H), 1.70 (quintet, J=7.6 Hz, 2H), 1.64 (quintet, J=7.6 Hz, 2H), 1.37 (quintet, J=7.7 Hz, 2H); IR (neat) 1763, 1604, 1497, 1454, 1402 cm⁻¹; mass spectrum m/z (relative intensity) 244 (M⁺, 21), 175 (8), 117 (20), 91 (100), 77 (6). Exact mass calculated for C₁₃H₁₅OF₃; 244.1075; found, 244.1073.

### 1,1,1-Trifluoro-6-(4-methoxy-phenyl)-2-hexanone (27.4)

27.4 was synthesized as a colorless viscous oil.

27.4 was confirmed as follows: ¹H NMR (500 MHz, CDCl₃) δ 7.07 (d, J=8.4 Hz, 2H), 6.82 (d, J=8.4 Hz, 2H), 3.77 (s, 3H), 2.71 (t, J=6.9 Hz, 2H), 2.58 (t, J=7.4 Hz, 2H), 1.70 (quintet, J=7.1 Hz, 2H), 1.62 (quintet, J=6.8 Hz, 2H); IR (neat) 1763, 1612, 1584, 1512 cm⁻¹.

### Synthesis of Trifluoromethyl Ketones 30 and 35

Trifluoromethyl ketones (30 and 35) were synthesized by a method depicted in Scheme 7. 3-(Methoxycarbonyl)phenylboronic acid, 3-benzyloxyphenylboronic acid and 3-benzyloxybromobenzene (28) were commercially available starting materials while (3-bromophenyl)acetic acid methyl ester (31) was synthesized from commercially available 3-bromophenylacetic acid following a method disclosed in Luning et al., Eur. J. Org. Chem. (2002) 3294-3303.

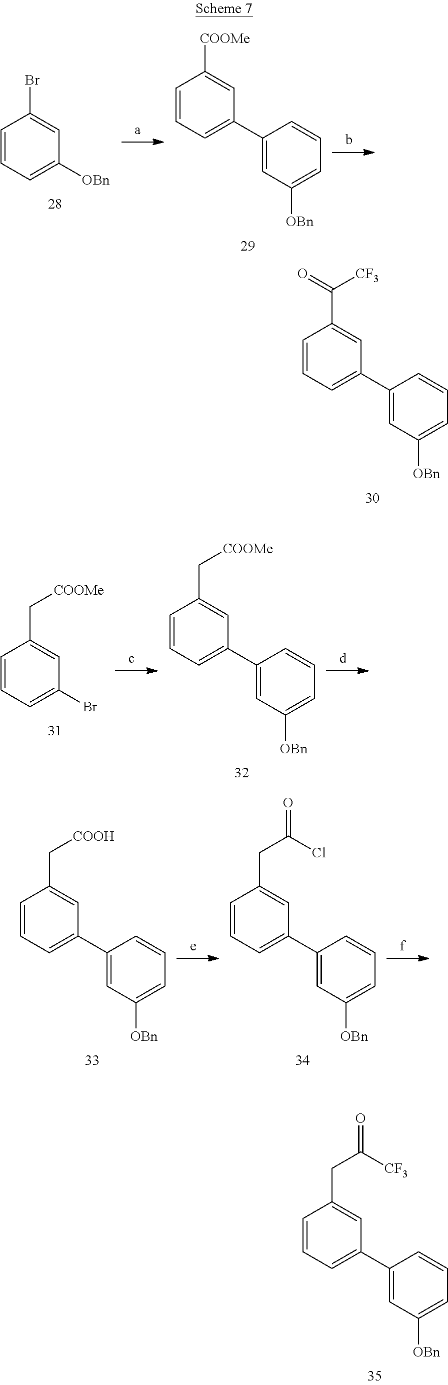

Reagents and conditions for the steps in Scheme 7 were as follows: Step a: 3-(methoxycarbonyl)phenylboronic acid, Ba(OH₂), Pd(PPh₃)₄, DME/H₂O, microwave, see Luning text, 50%; Step b: TMS-CF₃, TBAF, PhCH₃, N₂, −78° C. to RT, 18 hours, 65%; Step c: 3-benzyloxyphenylboronic acid, Ba(OH₂), Pd(PPh₃)₄, DME/H₂O, microwave, see Luning text, 48%; Step d: KOH, EtOH/H₂O, 50° C., 2 hours; Step e: (COCl)₂, CH₂Cl₂, RT, 2 hours; Step f: (i) CF₃COOCOCF₃, pyridine, CH₂Cl₂, 0° C. to RT, (ii) H₂O, 0° C. to RT, 37% from 32.

### 3′-Benzyloxy-biphenyl-3-carboxylic acid methyl ester (29)

A degassed mixture of 3-benzyloxy-phenyl bromide (28) (0.176 g, 0.67 mmol), 3-methoxycarbonylphenylboronic acid (0.18 g, 1 mmol), barium hydroxide (0.25 g, 1.47 mmol), Pd(PPh₃)₄ (0.077 g, 0.067 mmol), DME (5 mL) and H₂O (3 mL) was microwaved with vigorous stirring using a CEM-discover system (ram time: 2 min, hold time: 5 min, temperature: 120° C., pressure: 200 psi, power: 250 W). The crude reaction mixture filtered through a plug of celite and concentrated in vacuo. The residue obtained was purified by flash column chromatography (25% diethyl ether-hexane) to give the title compound (29) (0.118 g, 60% yield) as a viscous liquid.

29 was confirmed as follows: ¹H NMR (500 MHz, CDCl₃) δ 8.27 (t, J=1.5 Hz, 1H), 8.20 (dd, J=8.0 Hz, J=1.5 Hz, 1H), 7.76 (dd, J=8.0 Hz, J=2.0 Hz, 1H), 7.50 (t, J=8.0 Hz, 1H), 7.47 (d, J=7.5 Hz, 2H), 7.42-7.32 (m, 4H), 7.25-7.22 (m, 2H), 7.00 (dd, J=8.2 Hz, J=2.0 Hz, 1H), 5.13 (s, 2H), 3.95 (s, 3H).

### 1,1,1-Trifluoro-2-(3-benzyloxy-biphenyl-3-yl)-2-ethanone (30)

A solution of 29 (0.1 g, 0.314 mmol) in anhydrous toluene (5 mL) was cooled to −78° C., under nitrogen, and trifluoromethylrimethylsilane (62.5 mg, 0.44 mmol) was added. The mixture was stirred for 15 min at −78° C., a 1 M anhydrous solution of tetrabutylammonium fluoride in THF (0.026 ml, 0.026 mmol) was added and the resultant mixture was gradually warmed to RT. After stirring for 12 hours at RT, the reaction mixture was diluted with 4 N HCl solution (2 mL) and stirred for an additional 2 hour period. The organic layer was separated and the aqueous layer was extracted with diethyl ether (20 mL). The combined organic layer was washed with aqueous saturated NaHCO₃ solution (5 mL) and brine, dried (MgSO₄), and concentrated under reduced pressure. The residue was purified by flash column chromatography on silica gel (25% diethyl ether-hexane) and the fraction that contains the product (30) and its hydrate form (2:1 ratio by ¹H NMR) was concentrated and dried in high vacuum (in the presence of P₂O₅) to give pure compound (30) (0.0876 g, 76% yield) as a viscous liquid.

30 was confirmed as follows: ¹H NMR (500 MHz, CDCl₃) δ 8.26 (s, 1H), 8.04 (d, J=7.5 Hz, 1H), 7.90 (d, J=8.0 Hz, 1H), 7.61 (t, J=7.5 Hz, 1H), 7.47 (d, J=8.0 Hz, 2H), 7.44-7.38 (m, 3H), 7.3 (t, J=7.2 Hz, 1H), 7.22-7.20 (m, 2H), 7.03 (dd, J=8.0 Hz, J=2.5 Hz, 1H), 5.08 (s, 2H).

### 2-(3-Benzyloxy-biphenyl-3-yl)-acetic acid methyl ester (32)

2-(3-Benzyloxy-biphenyl-3-yl)-acetic acid methyl ester (32) was synthesized following the procedure described for the preparation of 29 using 3-bromo-phenyl acetic acid methyl ester (31) (0.31 g, 1.35 mmol), 3-benzyloxy-phenyl boronic acid (0.45 g, 2 mmol), barium hydroxide (0.5 g, 3 mmol) and Pd(PPh₃)₄ (0.15 g, 0.13 mmol), in DME (10 mL)/water (4 mL). Purification by flash column chromatography on silica gel gave pure compound (32) (0.22 g, 49% yield) as a white solid (melting point 50-52° C.).

32 was confirmed as follows: ¹H NMR (500 MHz, CDCl₃) δ 7.49-7.45 (m, 4H), 7.42-7.32 (m, 5H), 7.27 (d, J=7.0 Hz, 1H), 7.21 (t, J=2.5 Hz, 1H) 7.19 ((dd, J=7.5 Hz, J=1.0 Hz, 1H), 6.97 (dd, J=8.0 Hz, J=2.5 Hz 1H), 5.1 (s, 2H), 3.71 (s, 3H), 3.69 (s, 2H).

### 2-(3-Benzyloxy-biphenyl-3-yl)-acetic acid (33)

A mixture of 26 (0.1 g, 0.3 mmol) and KOH (0.08 g, 1.2 mmol) in wet EtOH (5 mL) was heated at 50° C., under nitrogen for 2 hours. The reaction mixture was cooled to RT, and the solvent evaporated under reduced pressure. The residue obtained was dissolved in water (5 mL) and the pH was adjusted to 1 using 5% aqueous HCl solution (2 mL). The precipitated crude acid was isolated by filtration and dissolved in ethyl acetate. The resulting solution was washed with brine, dried (MgSO₄), and concentrated under reduced pressure to give 33 as a white solid (0.087 g, 91%), which was used in the next step without further purification.

### 1,1,1-Trifluoro-3-(3-benzyloxy-biphenyl-3-yl)-2-propanone (35)

To a solution of acid (33) (0.08 g, 0.25 mmol) in anhydrous CH₂Cl₂ at RT, under nitrogen, was added oxalyl chloride (0.25 mL, 0.5 mmol) over a 2-min period. The mixture was stirred for 2 hours, solvent and excess oxalyl chloride were removed under reduced pressure, and the crude carboxylic acid chloride (34) was used in the next step without further purification.

To a solution of 34 in anhydrous CH₂Cl₂ at 0° C. under a nitrogen atmosphere were added successively trifluoroacetic anhydride (1 mL, 1.5 mmol) and dry pyridine (0.16 mmol, 0.16 mL). The reaction mixture was stirred at 0° C. for 10 min, and then it was allowed to warm to RT and stirred for an additional 2 hour period. Water was added dropwise at 0° C., the resulting mixture was warmed to RT, and extracted with CH₂Cl₂. The organic layer was washed with dilute aqueous HCl solution, and saturated aqueous NaHCO₃ solution, dried (MgSO₄) and the solvent was evaporated. Following the workup, the crude mixture was chromatographed on a silica gel column (eluting with 30% diethyl ether-hexane) to give compound (35) (0.033 g, 36% yield) as a viscous liquid.

35 was confirmed as follows: ¹H NMR (500 MHz, CDCl₃) δ 7.56 (d, J=8.0 Hz, 1H), 7.49 (d, J=7.0 Hz, 2H), 7.47-7.41 (m, 4H), 7.40-7.35 (m, 3H), 7.23-7.19 (m, 3H), 7.03 (dd, J=8.0 Hz, J=2.5 Hz, 1H), 5.15 (s, 2H), 4.01 (s, 2H).

### Synthesis of Trifluoromethyl ketones 39.1-4 and 40.1, 40.3

Trifluoromethyl ketones (39.1-4 and 40.1, 40.3) (shown in Scheme 8) were synthesized by a method depicted in Scheme 8. Resorcinol dimethyl ether 36.1 and 4′-bromo-2,2,2-trifluoroacetophenone were commercially available starting materials while olivetol dimethyl ether (36.2) was synthesized following a method disclosed in Nikas, et al. (2002) Synth. Commun., 32:1751 and Nikas, et al. (2002) J. Labelled Compd. Radiopharm., 45:1065. The resorcinol dimethyl ethers (36.3 and 36.4) were synthesized by methylation of commercially available 4-hexylresorcinol and 4,6-dichlororesorcinol respectively.

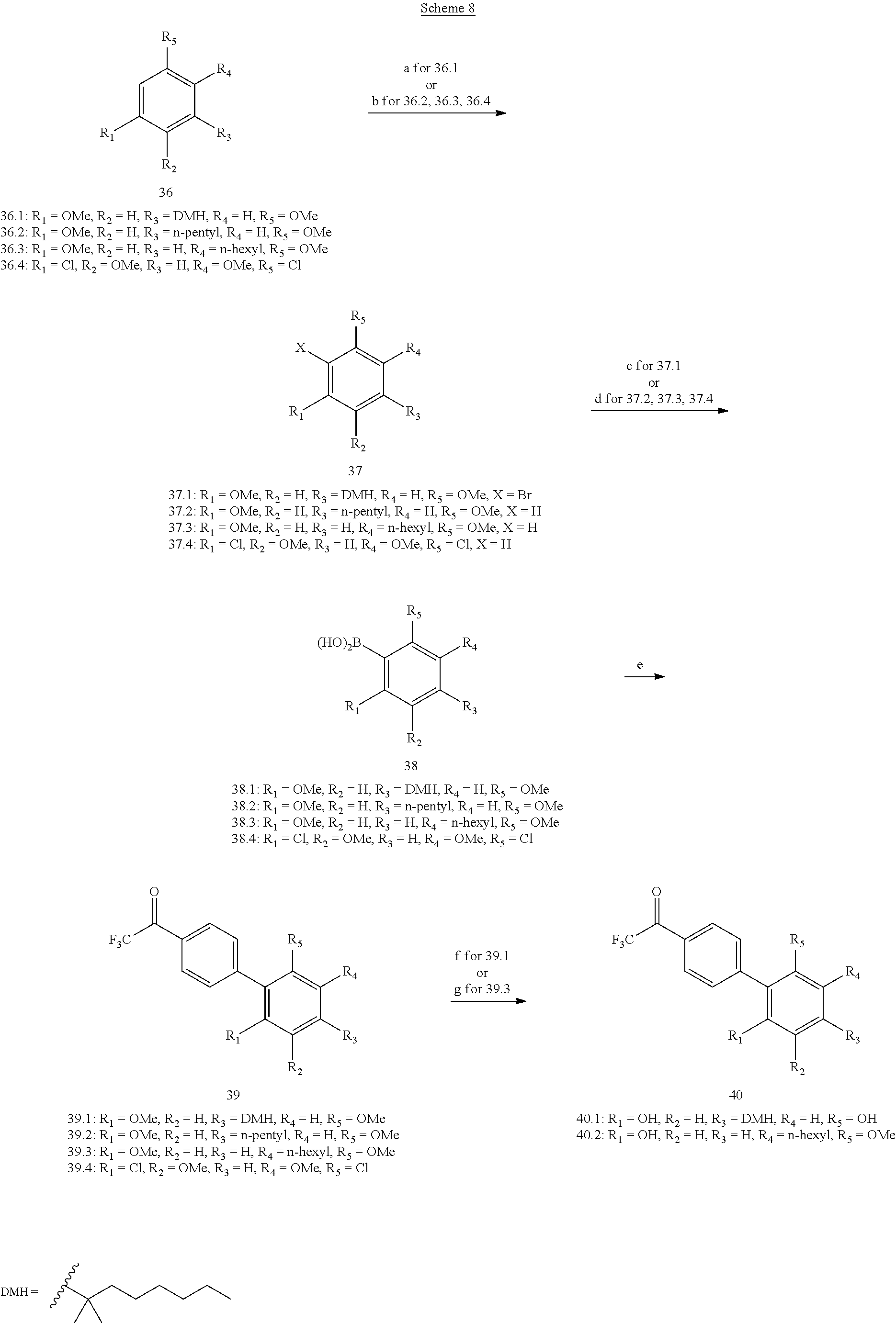

Reagents and conditions for the steps in Scheme 8 were as follows: Step a: Br₂, 18-crown-6, CH₂Cl₂, RT, 20 min, 97%; Step b: MeI, K₂CO₃, DMF, RT, 3-5 hours, 83-95%; Step c (i) n-BuLi, THF, −78° C., 15 min, (ii) B(OMe)₃, -78° C. to RT, 12 hours then aqueous HCl, 83%; Step d: (i) n-BuLi, THF, −78° C. to −10° C., 2.5-7.5 hours, (ii) B(OMe)₃, −78° C. to RT, overnight then aqueous HCl, 75-85%; Step e: 4′-bromo-2,2,2-trifluoroacetophenone, Pd(PPh₃)₄, Ba(OH)₂.8H₂O, DME/H₂O, microwave, 115° C., 300 W, 4-6 min, 63-78%; Step f: BBr₃, CH₂Cl₂, −78° C. to RT, 4 hours, 68%; Step g: n-Bu₄NI, BCl₃, CH₂Cl₂, −78° C. to 0° C., 2 hours, 68%.

### 2-Bromo-5-(1,1-dimethylheptyl)-1,3-dimethoxybenzene (37.1)

To a vigorously stirred solution of 36.1 (2.09 g, 7.93 mmol) and 18-crown-6 in methylene chloride (70 mL) at RT was added bromine dropwise (0.43 mL, 8.30 mmol). Stirring was continued for 20 min, and the reaction mixture was successively washed with 10% sodium thiosulphate, a saturated sodium bicarbonate solution, and brine. The organic layer was dried over MgSO₄, and evaporated, and the crude oil was purified by flash column chromatography (3% diethyl ether in hexane) to afford the title compound in 97% yield (2.66 g) as a colorless oil.

37.1 was confirmed as follows: ¹H NMR (500 MHz, CDCl₃) δ 6.54 (s, 2H), 3.90 (s, 6H), 1.58 (m, 2H), 1.29 (s, 6H), 1.25-1.19 (m, 6H), 1.05 (m, 2H), 0.85 (t, J=6.9 Hz, 3H).

### Intermediates 37.2, 37.3 and 37.4

A mixture of resorcinol (36.2 or 36.3 or 36.4) (1 equiv.), methyl iodide (2.2 equiv.) and potassium carbonate (2.5 equiv.) in anhydrous dimethylformamide was stirred for 3-5 hours at RT under an argon atmosphere. The reaction mixture was diluted with water and extracted with ethyl acetate. The organic phase was washed with water, brine, dried (MgSO₄), and the solvent was removed under reduced pressure. The residue was purified by flash column chromatography on silica gel (diethyl ether-hexane) to give the product in 83-95% yields.

### Selected data of synthesized intermediates 37.2 and, 37.3

### 1,3-Dimethoxy-5-pentylbenzene (37.2)

37.2 was confirmed as follows: ¹H NMR (500 MHz, CDCl₃) δ 6.34 (d, J=2.0 Hz, 2H), 6.29 (t, J=2.0 Hz, 1H), 3.77 (s, 6H), 2.54 (t, J=7.2 Hz, 2H), 1.64-1.57 (m, 2H), 1.38-1.27 (m, 4H), 0.89 (t, J=7.3 Hz, 3H).

### 1,3-Dimethoxy-4-hexylbenzene (37.3)

37.3 was confirmed as follows: ¹H NMR (500 MHz, CDCl₃) δ 7.02 (d, J=8.5 Hz, 1H), 6.43 (d, J=2.5 Hz, 1H), 6.40 (dd, J=8.5 Hz, J=2.5 Hz, 1H), 3.78 (s, 3H), 3.77 (s, 3H), 2.52 (t, J=7.5 Hz, 2H), 1.56-1.50 (m, 2H), 1.36-1.25 (m, 6H), 0.88 (t, J=7.0, 3H).

### 2,6-Dimethoxy-4-(2-methyloctan-2-yl)phenylboronic acid (38.1)

To a stirred solution of 37.1 (2.78 g, 8.0 mmol) in anhydrous THF (20 ml) under an argon atmosphere at −78° C. was added n-BuLi (5.5 ml, 8.8 mmol using 1.6 M solution in hexane) over a 30 min period. Stirring was continued at −78° C. for 15 min, and then trimethyl borate (2.7 ml, 24 mmol) was added. Following addition, the reaction mixture was allowed to warm to RT over a 12 hour period. The pH was adjusted to 6.5 by addition of 5% aqueous HCl solution at 0° C., and the mixture was extracted with dichloromethane. The organic layer was washed with brine, dried (MgSO₄), and the solvent was evaporated under reduced pressure. The residue was purified by flash column chromatography on silica gel (12% acetone in hexane) to give 38.1 as colorless oil, in 83% yield (2.1 g).

38.1 was confirmed as follows: ¹H NMR (500 MHz, CDCl₃) δ 6.54 (s, 2H), 3.89 (s, 6H), 1.55 (m, 2H), 1.26 (s, 6H), 1.19-1.23 (m, 6H), 1.05 (m, 2H), 0.85 (t, J=6.8 Hz, 3H).

### Boronic acids 38.2, 38.3, and 38.4

To a solution of the resorcinol dimethyl ether (37.2 or 37.3 or 37.4, 1 equiv.) in dry THF, under an argon atmosphere at −78° C. was added n-BuLi dropwise (1.1 equiv. using a 1.6 solution in hexanes). The mixture was stirred for 1-6 hours at −78° C., and then it was warmed to −10° C. and stirred for an additional 1.5 hour. The reaction mixture was cooled to −78° C. and (MeO)₃B (5 equiv.) was added. Following the addition, the mixture was warmed to RT and stirred overnight. The reaction was quenched by the dropwise addition of water, the pH was adjusted to 4 using a 5% aqueous HCl solution, and the mixture was extracted with AcOEt. The organic layer was washed with brine, dried (MgSO₄), and the solvent was evaporated under reduced pressure. The residue was purified by flash column chromatography on silica gel (acetone in hexane) to give boronic acid derivative (38.2 or 38.3 or 38.4) in 75-85% yields.

### Selected data of synthesized boronic acids 38.2, 38.3, and 38.4

### 4-Pentyl-2,6-dimethoxyphenyl boronic acid (38.2)

38.2 was confirmed as follows: ¹H NMR (500 MHz, CDCl₃) δ 7.18 (s, 2H), 6.45 (s, 2H), 3.90 (s, 6H), 2.61 (t, J=8.3 Hz, 2H), 1.63 (qt, J=6.9 Hz, 2H), 1.41-1.29 (m, 4H), 0.91 (t, J=7.2 Hz, 3H).

### 3-Hexyl-2,6-dimethoxyphenyl-boronic acid (38.3)

38.3 was confirmed as follows: ¹H NMR (500 MHz, CDCl₃) δ 7.28 (d, J=8.1 Hz, 1H), 6.72 (d, J=8.1 Hz, 1H), 3.89 (s, 3H), 3.78 (s, 3H), 2.57 (m as t, J=8.5 Hz, 2H), 1.62-1.56 (m, 2H), 1.37-1.29 (m, 6H), 0.89 (t, J=7.5 Hz, 3H)

### Trifluoromethyl ketones (39)

A degassed mixture of boronic acid (38) (1.1 equiv.) 4′-bromo-2,2,2-trifluoroacetophenone (1.0 equiv.), Ba(OH)₂′8H₂O (1.5 equiv.) Pd(PPh₃)₄ (0.03 equiv.), 1,2-dimethoxyethane and H₂O was heated for 4-6 min at 115° C. under microwave irradiation (300 W) using a CEM Discover system. The reaction mixture was cooled to RT, diluted with ethyl acetate, and filtered through a short pad of silica gel. The filtrate diluted with brine and extracted with ethyl acetate. The organic layer was dried over MgSO₄, the solvent was evaporated, and the residue was purified by flash column chromatography on silica gel (acetone-hexane) to give 39 in 63-78% yields.

### Selected data of synthesized trifluoromethyl ketones (39)

### 1-(2′,6′-Dimethoxy-4′-pentylbiphenyl-4-yl)-2,2,2-trifluoroethanone (39.2)

39.2 was confirmed as follows: ¹H NMR (500 MHz, CDCl₃) δ 8.13 (d, J=8.1 Hz, 2H), 7.47 (d, J=8.1 Hz, 2H), 6.49 (s, 2H), 3.74 (s, 6H), 2.64 (t, J=7.8 Hz, 2H), 1.72-1.64 (m, 2H), 1.43-1.35 (m, 4H), 0.93 (t, J=7.5 Hz, 3H).

### 1-(2′,6′-Dimethoxy-3′-hexylbiphenyl-4-yl) 2,2,2-trifluoroethanone (39.3)

39.3 was confirmed as follows: ¹H NMR (500 MHz, CDCl₃) δ 8.16 (d, J=8.5 Hz, 2H), 7.56 (d, 8.5 Hz, 2H), 7.17 (d, J=8.3 Hz, 1H), 6.73 (d, J=8.3 Hz, 1H), 3.73 (s, 3H), 3.27 (s, 3H), 2.61 (t, J=7.3 Hz, 2H), 1.61 (qt, J=6.8 Hz, 2H), 1.42-1.29 (m, 6H), 0.89 (t, J=7.1 Hz, 3H).

### 1-(2′,6′-dihydroxy-4′-(2-methyloctan-2-yl)biphenyl-4-yl)-2,2,2-trifluoroethanone (40.1)

To a solution of 39.1 (500 mg, 1.145 mmol) in dry dichloromethane at 0° C. under an argon atmosphere was added boron tribromide (2.8 mL, using 1 M solution in CH₂Cl₂). Following the addition, the mixture was stirred until the reaction was completed (4 hours). Unreacted boron tribromide was destroyed by dropwise addition of water at 0° C. The resulting mixture was warmed to RT and diluted with dichloromethane. The organic layer was washed with saturated sodium bicarbonate solution, brine, dried over MgSO₄, and evaporated. Purification by flash column chromatography (18% acetone in hexane) gave the title compound in 68% yield (0.318 mg).

40.1 was confirmed as follows: ¹H NMR (500 MHz, CDCl₃) δ 8.21 (d, J=8.3 Hz, 2H), 7.68 (d, J=8.3 Hz, 2H), 6.54 (s, 2H), 4.77 (s, 2H, OH), 1.60-1.55 (m, 2H), 1.28 (s, 6H), 1.27-1.19 (m, 6H), 1.16-1.08 (m, 2H), 0.86 (t, J=6.5 Hz, 3H).

### 2,2,2-Trifluoro-1-(3′-hexyl-6′-hydroxy-2′-methoxybiphenyl-4-yl)ethanone (40.3)

Compound 39.3 (1 equiv.) and n-Bu₄NI (3 equiv.) were stirred in dry CH₂Cl₂ at −78° C. under nitrogen. A solution of BCl₃ (3.2 mL, using 1 M solution in CH₂Cl₂) was added over a 2 min period. After 5 min, the solution was warmed to 0° C., and stirring was continued for 2 hours. The reaction was quenched with ice-water, the resulting mixture was stirred for 30 min, and partially concentrated to remove CH₂Cl₂. Water was added, and the mixture was extracted with diethyl ether. The combined organic layer was washed with saturated aqueous NaCl solution, dried over MgSO₄, and evaporated. Purification by flash column chromatography on silica gel (18% acetone in hexane) gave the product (40.3) in 68% yield (270 mg).

40.3 was confirmed as follows: ¹H NMR (500 MHz, CDCl₃) δ 8.21 (d, J=8.0 Hz, 2H), 7.52 (d, J=8.0 Hz, 2H), 7.02 (d, J=8.3 Hz, 1H), 6.51 (d, J=8.3 Hz, 1H), 4.76 (s, 1H), 3.96 (s, 3H), 2.57 (t, J=7.8 Hz, 2H), 1.60 (qt, J=7.9 Hz, 2H), 1.42-1.28 (m, 6H), 0.89 (t, J=7.2 Hz, 3H).

### Synthesis of carbamates (46.1-46.3)

The carbamates 46.1, 46.2 or 46.3 were synthesized by a method depicted in Scheme 9 starting from commercially available 4-(4-methoxyphenyl)butanol (41).

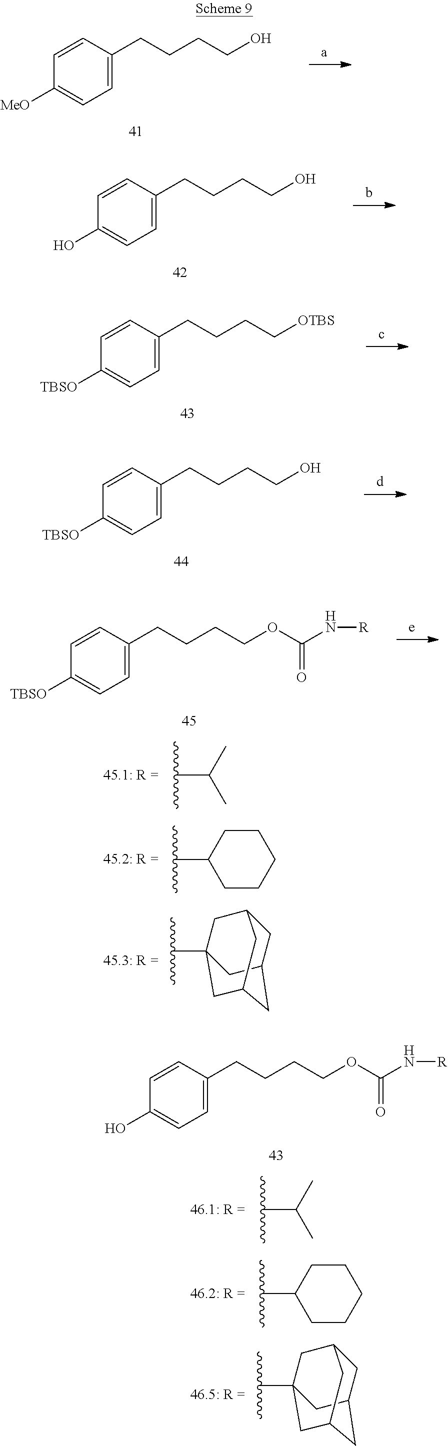

Reagents and conditions for the steps in Scheme 9 were as follows: Step a: BBr₃, CH₂Cl₂, −10° C. to RT, 42%; Step b: TBSCl, DMF, RT, 80%; Step c: Sc(OTf)₃, MeCN/H₂O, RT, 73%; Step d: (i) carbonyldiimidazole, CH₂Cl₂, 0° C., (ii) RNH₂, RT, 46-53%; Step e: TBAF, THF, −10° C. to RT, 75-82%.

### 4-(4-Hydroxyphenyl)butanol (42)

To a stirred solution of 4-(4-methoxyphenyl)butanol (1 equiv.) in dry dichloromethane at −10° C. under an argon atmosphere was added boron tribromide (2.7 equiv., using a 1 M solution of boron tribromide in CH₂Cl₂). Stirring was continued at that temperature until completion of the reaction (4 hours). Unreacted boron tribromide was destroyed by addition of aqueous saturated NaHCO₃ solution at 0° C. The resulting mixture diluted with CH₂Cl₂ and water, the organic phase was separated, washed with brine, dried (MgSO₄), and evaporated. Purification by flash column chromatography on silica gel (30% diethyl ether-hexane) afforded the title compound in 42% yield.

### 1-(tert-Butyldimethylsilyloxy)-4-(tert-butyldimethylsilyloxybutyl)-benzene (43)

To a solution of imidazole (4 equiv.) in DMF was added 4-(4-hydroxyphenyl)butanol (1 equiv.) in DMF followed by tert-butyldimethylsilyl chloride (3 equiv.) in DMF. The reaction was allowed to stir at RT for 15 hours and then quenched by addition of saturated aqueous NaHCO₃ solution. The resulting mixture was extracted with diethyl ether, the ethereal extract was washed with water and brine, and dried over MgSO₄. Solvent evaporation and purification by flash column chromatography on silica gel (3% diethyl ether-hexane) afforded the title compound in 80% yield.

### 4-(4-tert-Butyldimethylsilyloxy)butanol (44)

To a solution of 1-(tert-butyldimethylsilyloxy)-4-(tert-butyldimethylsilyloxybutyl)-benzene (1 equiv.) in a mixture of acetonitrile/water (1:2.5) at RT was added scandium triflate (0.05 equiv.). The reaction mixture was stirred for 1 hour, diluted by addition of CH₂Cl₂ and the organic phase was separated. The aqueous phase was extracted with CH₂Cl₂ and the combined organic layer washed with brine, dried (MgSO₄), and evaporated. Purification by flash column chromatography on silica gel (20% diethyl ether-hexane) gave the title compound in 73% yield.

### Intermediate carbamates (45)

To a suspension of carbonyldiimidazole (1.5 equiv.) in anhydrous dichloromethane at 0° C. was added 4-(4-tert-butyldimethylsilyloxy)butanol (1 equiv.) in dichloromethane. The reaction mixture was stirred at RT for 1 hour, and then the appropriate amine (1.1 equiv.) was added. Stirring was continued until completion of the reaction (8-10 hours). The reaction mixture was diluted with diethyl ether and 10% aqueous HCl solution. The organic phase was separated, washed with brine, dried (MgSO₄), and evaporated. Purification by flash column chromatography on silica gel (10% diethyl ether-hexane) gave intermediate carbamate (45) in 46-53% yield.

### Carbamates (46)

To a stirred solution of intermediate carbamate (45) (1 equiv.) in THF at −10° C. was added dropwise tetra-n-butylammonium fluoride hydrate (1.3 equiv.) in THF. The reaction mixture was allowed to warm to RT, stirred for 1 hour and diluted with diethyl ether. The organic phase was separated, washed with water and brine, dried (MgSO₄), and evaporated. Purification by flash column chromatography on silica gel gave carbamate (46) in 75-82% yield.

### Selected data of synthesized carbamates (46)

### 4-(4-Hydroxyphenyl)butanol isopropylcarbamate (46.1)

46.1 was confirmed as follows: ¹H NMR (400 MHz, CDCl₃) δ 7.01 (d, J=8.4 Hz, 2H), 6.76 (d, J=8.4 Hz, 2H), 4.55 (br s, 1H), 4.06 (t as br s, 2H), 3.81 (m, 1H), 2.54 (t, J=5.8 Hz, 2H), 1.71-1.59 (m, 4H), 1.14 (d, J=6.5 Hz, 6H).

### 4-(4-Hydroxyphenyl)butanol cyclohexylcarbamate (46.2)

46.2 was confirmed as follows: ¹H NMR (400 MHz, CDCl₃) δ 6.99 (d, J=8.3 Hz, 2H), 6.75 (d, J=8.3 Hz, 2H), 6.23 (br s, 1H), 4.51 (br s, 1H), 4.05 (t as br s, 2H), 3.48 (m, 1H), 2.55 (t, J=5.8 Hz, 2H), 1.97-1.85 (m, 2H), 1.75-1.05 (m, 12H).

### Synthesis of Carbamates 48.1-6, 52.1-4 and 53.1-4

The carbamates (48.1-6, 52.1-4 and 53.1-4) were synthesized by a method depicted in Scheme 10 using commercially available 4-bromoaniline (47.1), 4-iodoaniline (47.2), cyclohexanole, 1-adamantanol, 2,6-difluorophenol, phenol, benzyl chloroformate, ethyl chloroformate, triphosgene, and the resorcinol derivative (49).

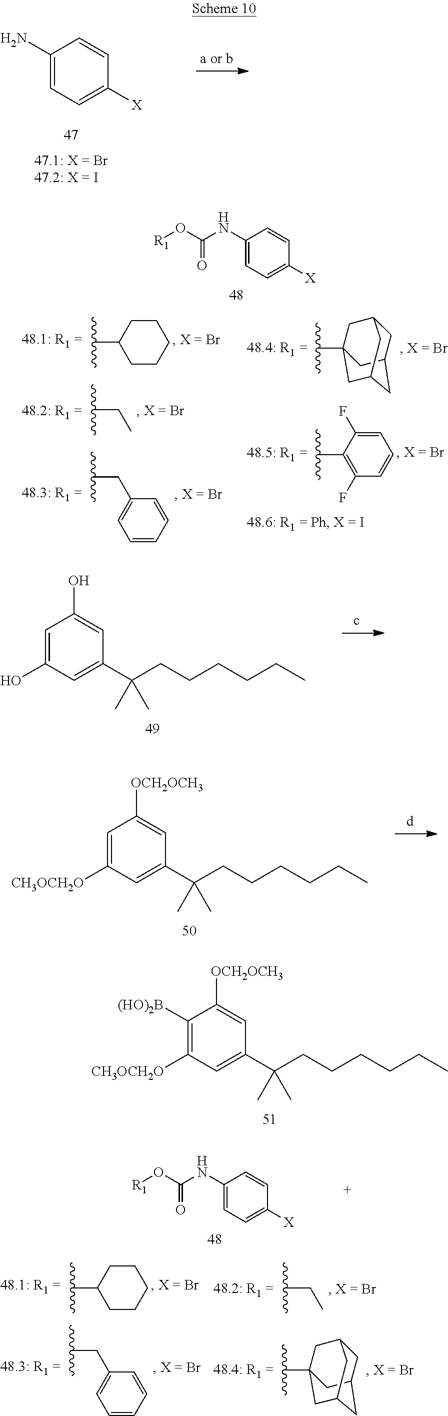

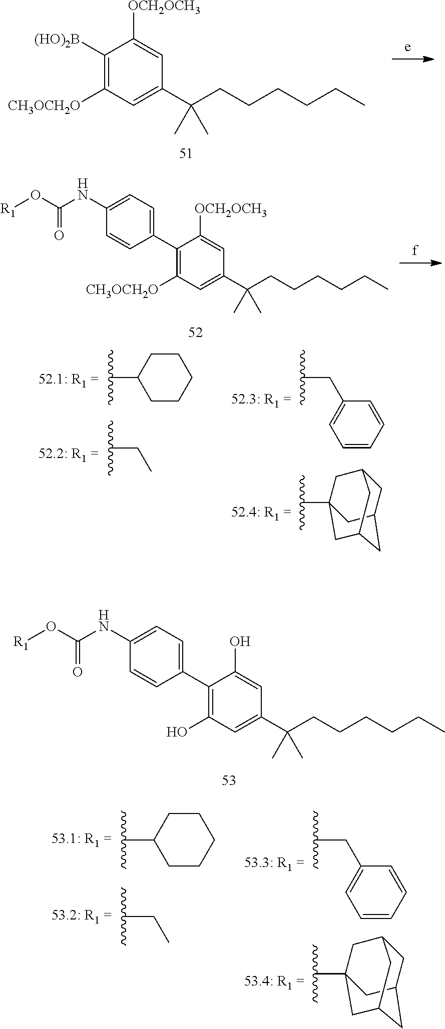

Reagents and conditions for the steps in Scheme 10 were as follows: Step a: BnOCOCl or EtOCOCl, Na₂CO₃, toluene, RT, 4-6 hours, 88-92%; Step b: (i) (Cl₃CO)₂CO, toluene, reflux, 4-6 hours, (ii) R₁OH, RT, 5 hours, 70-78%; Step c: CH₃OCH₂Cl, DIPEA, CH₂Cl₂, 0° C. to RT, 4 hours, 75%; Step d: (i) n-BuLi, −10° C., 1.5 hours, (ii) B(OMe)₃, −78° C. to RT, overnight then aqueous HCl, 81%; Step e: Pd(PPh₃)₄, Ba(OH)₂.8H₂O, DME/H₂O, microwave, 110° C., 4-6 min, 58-77%; Step f: 5N HCl, THF/i-PrOH, RT, 12-18 hours, 60-72%.

Intermediate carbamates 48.2, and 48.3

To a stirred suspension of 4-bromoaniline (47.1) (1 equiv.) and sodium carbonate (1.5 equiv.) in anhydrous toluene at RT was added ethyl or benzyl chloroformate. Stirring was continued for 4-6 hours at the same temperature. Insoluble materials were filtered off, and the filtrate was washed, with water and dried over MgSO₄. Solvent evaporation under reduced pressure and purification by flash column chromatography on silica gel (diethyl ether-hexane) gave pure products (48.2 or 48.3 respectively) in 88-92% yields.

### Intermediate carbamates 48.1, 48.4, 48.5, and 48.6

To a stirred suspension of aryl amine (47.1 or 47.2) (1 equiv.) and sodium carbonate (1.5 equiv.) in anhydrous toluene, at RT under argon atmosphere was added triphosgene (1.2 equiv.). The reaction mixture was heated under reflux until TLC analysis indicated the total consumption of starting material (4-6 hours). The reaction mixture was cooled to RT, filtered, and the appropriate alcohol (1.1 equiv.) was added to the filtrate. The resulting mixture was stirred at RT for 5 hours and the solvent was evaporated under reduced pressure. Purification by flash column chromatography on silica gel gave the pure product in 70-78% yield.

### General Procedure for carbamates 48.7-48.9, 53.3-53.8, 53.11, 53.12-53.15, 53.18 and 53.26

To a stirred solution of substituted aryl amine in 1,2-dimethoxy ethane (1 equiv.), was added triphosgene (0.33 equiv.), and the reaction was irradiated with (Biotage) microwave, for 8 min at 110° C. The reaction mixture was cooled, an appropriate alcohol (1 equivalent) was added and again irradiated with microwave for another 10 min at 120° C. The reaction mixture was cooled, and ethyl acetate was added. The combined reaction mixture was washed with a 5% aqueous sodium bicarbonate solution, and then dried (MgSO₄). Solvent was evaporated under reduced pressure. Purification by flash column chromatography on silica gel gave the pure product in 65-85% yield.

### General Procedure for 1-chloroethyl carbonates 46.3, 53.38, and 53.44

1-chloroethyl carbonochloridate (0.55 mol) and the indicated alcohol (0.5 mol) were dissolved in dichloromethane (60 ml) and cooled to O′C Pyridine (0.055 mol) is then added dropwise while maintaining the temperature below 15′C. The mixture is then stirred at RT until no alcohol remains in solution as indicated by TLC analysis (generally 4-5 hours). The resulting mixture is then washed with 1N hydrochloric acid (10 ml), then with a saturated solution of potassium carbonate (10 ml), and then twice with water (2×10 ml). The organic phase is dried with MgSO₄ and solvent is evaporated under reduced pressure. The resulting carbonate is purified by flash column chromatography to give pure carbonate in 72-85% yields.

### General procedure for Carbamates 53.21, 53.23, 53.24, 53.39-53.43 and 53.45-53.47:

To a solution of the indicated carbonate (1 equiv.) in tetrahydrofuran (10 ml) is added to a solution of the amine (1 equiv.) in tetrahydrofuran (30 ml) missed with a 5 M solution of potassium carbonate (20 ml) while maintaining temperature at 5-10° C. The mixture is then stirred at RT until TLC indicates complete consumption of amine (1-5 hrs). The organic phase is separated, washed with a saturated solution of NaCl (20 ml), dried with MgSO₄, and concentrated under reduced pressure. The resulting carbamate is purified by column chromatography, to give pure carbamate in 65-82% yield.

### Selected data of synthesized intermediate carbamates (48)

### (4-Bromophenyl)carbamic acid cyclohexyl ester (48.1)

48.1 was confirmed as follows: ¹H NMR (500 MHz, CDCl₃) δ 7.40 (d, J=8.7 Hz, 2H), 7.28 (br d, J=8.7 Hz, 2H), 6.58 (br s, 1H, NH), 4.75 (m, 1H), 1.96-1.89 (m, 2H), 1.78-1.70 (m, 2H), 1.59-1.52 (m, 1H), 1.50-1.34 (m, 4H), 1.31-1.22 (m, 1H).

### (4-Bromophenyl)carbamic acid benzyl ester (48.3)

48.3 was confirmed as follows: ¹H NMR (500 MHz, CDCl₃) δ 7.44-7.22 (m, 9H), 6.65 (br s, 1H, NH), 5.21 (s, 2H)

### (4-Iodophenyl)carbamic acid phenyl ester (48.6)

48.6 was confirmed as follows: ¹H NMR (500 MHz, CDCl₃) δ 7.63 (d, J=8.5 Hz, 2H), 7.40 (t, J=8.5 Hz, 2H), 7.28-7.22 (m, 3H), 7.18 (d, J=8.0 Hz, 2H), 6.93 (br s, 1H, NH)

### 1,3-Bis(methoxymethoxy)-5-(1,1-dimethylheptyl)-benzene (50)

To a stirred solution of resorcinol (49) (1.00 g, 4.23 mmol) and N-ethyldiisopropylamine (3.04 mL, 16.92 mmol) in CH₂Cl₂ at 0° C. was added chloromethyl methyl ether (0.82 mL, 10.15 mmol) over 15 min period. The solution was warmed to RT, stirred for 4 hours and volatiles were removed in vacuo. The residue was purified by flash column chromatography on silica gel (diethyl ether-hexane) to give the title compound in 75% yield.

### 2,6-Bis(methoxymethoxy)-4-(1,1-dimethylheptyl)-phenyl boronic acid (51)

1,3-Bis(methoxymethoxy)-5-(1,1-dimethylheptyl)-benzene (50) (1 equiv.) was dissolved in dry THF (10 mL). The solution was cooled to −10° C., and n-BuLi (1.1 equiv. using 1.6 solution in hexanes) was added dropwise. The mixture was stirred for an additional 1.5 hours, and then cooled to −78° C. (MeO)₃B (5 equiv.) was then added. The reaction mixture was allowed to warm to RT and stirred overnight. The mixture was diluted with water, stirred for 30 min and the pH was adjusted to 4 with dilute aqueous HCl. The mixture was extracted with EtOAc, the organic layer was dried (MgSO₄), and the solvent was evaporated. Purification by flash column chromatography (hexane-acetone) gave the title compound in 81% yield.

51 was confirmed as follows: ¹H NMR (500 MHz, CDCl₃) δ 7.21 (s, 2H), 6.86 (s, 2H), 5.31 (s, 4H), 3.53 (s, 6H), 1.62-1.56 (m, 2H), 1.31-1.18 (m, 12H, especially 1.29, s, 6H), 1.12-1.04 (m, 2H), 0.87 (t, J=6.5 Hz, 3H).

### Carbamates (52)

A degassed mixture of boronic acid (51) (1.1 equiv.), 4-bromo-2,2,2-trifluoroacetophenone (1.0 equiv.), Ba(OH)₂.8H₂O (1.5 equiv.), Pd(PPh₃)₄ (0.03 equiv.), 1,2-dimethoxy ethane and water was heated for 4-6 min at 110° C. under microwave irradiation using a CEM discover system. The reaction mixture was cooled to RT, diluted with ethyl acetate, and filtered through a short pad of silica gel. The filtrate diluted with brine and extracted with ethyl acetate. The organic layer was dried over MgSO₄, the solvent was evaporated, and the residue was purified by flash column chromatography on silica gel (acetone-hexane) to give product 52 in 58-77% yields.

### Selected data of synthesized carbamates (52)

### 2′,6′-Bis(methoxymethoxy)-4′-(1,1-dimethylheptyl)[1,1′-biphenyl]-4-yl carbamic acid ethyl ester (52.2)

52.2 was confirmed as follows: ¹H NMR (500 MHz, CDCl₃) δ 7.40 (br d, J=8.9 Hz, 2H), 7.34 (d, J=8.9 Hz, 2H), 6.86 (s, 2H), 6.58 (br s, 1H, NH), 5.00 (s, 4H), 4.24 (q, J=7.5 Hz, 2H), 3.31 (s, 6H), 1.61-1.57 (m, 2H), 1.32 (t, J=7.5 Hz, 3H), 1.29 (s, 6H), 1.27-1.20 (m, 6H), 1.17-1.10 (m, 2H), 0.86 (t, J=7.6 Hz, 3H) 2′,6′-Bis(methoxymethoxy)-4′-(1,1-dimethylheptyl)[1,1′-biphenyl]-4-yl carbamic acid benzyl ester (52.3)

52.3 was confirmed as follows: ¹H NMR (500 MHz, CDCl₃) δ 7.43-7.33 (m, 9H), 6.86 (s, 2H), 6.68 (br s NH), 5.22 (s, 2H), 5.00 (s, 2H), 3.30 (s, 6H), 1.60-1.57 (m, 2H), 1.29 (s, 2H), 1.28-1.22 (m, 6H), 1.17-1.10 (m, 2H), 0.86 (t, J=7.0 Hz, 3H)

### 2. Carbamates (53)

To a stirred solution of 52 (1.0 equiv.) in isopropyl alcohol/THF mixture (1:1) were added few drops of 5N HCl solution. This mixture was stirred overnight at RT and evaporated to dryness. The residue was purified by flash column chromatography on silica gel (acetone-hexane) to give the product 53 in 60-72% yields.

### 3. Selected data of synthesized carbamates (53)

### 2′,6′-Dihydroxy-4′-(2-methyloctan-2-yl)biphenyl-4-yl carbamic acid cyclohexyl ester (53.1)

53.1 was confirmed as follows: ¹H NMR (500 MHz, CDCl₃) δ 7.59 (br d, J=8.3 Hz, 2H), 7.38 (d, J=8.3 Hz, 2H), 6.66 (br s, 1H, NH), 6.56 (s, 2H), 4.82-4.74 (m and s, overlapping, 3H, especially 4.76, s, 2H, OH), 1.99-1.93 (m, 2H), 1.79-1.74 (m, 2H), 1.61-1.54 (m, 2H), 1.52-1.37 (m, 6H), 1.33-1.18 (m and s, overlapping, 12H, especially 1.27, s, 6H, —C(CH₃)₂), 1.17-1.08 (m, 2H), 0.86 (t, J=7.0 Hz, 3H)

### 2′,6′-Dihydroxy-4′-(1,1-dimethylheptyl)[1,1′-biphenyl]-4-yl carbamic acid ethyl ester (53.2)

53.2 was confirmed as follows: ¹H NMR (500 MHz, CDCl₃) δ 7.58 (br d, J=8.3 Hz, 2H), 7.38 (d, J=8.3 Hz, 2H), 6.68 (br s, 1H, NH), 6.56 (s, 2H), 4.74 (s, 2H, OH), 4.27 (q, J=7.5 Hz, 2H), 1.59-1.54 (m, 2H), 1.34 (t, J=7.5 Hz, 3H), 1.27 (s, 6H), 1.24-1.19 (m, 6H), 1.15-1.08 (m, 2H), 0.86 (t, J=7.2 Hz, 3H).

### 2′,6′-Dihydroxy-4′-(2-methyloctan-2-yl)biphenyl-4-yl carbamic acid benzyl ester (53.3)

53.3 was confirmed as follows: ¹H NMR (500 MHz, CDCl₃) δ 7.59 (d, J=8.0 Hz, 2H), 7.44-7.36 (m, 7H), 6.77 (br s, 1H, NH), 6.56 (s, 2H), 5.23 (s, 2H), 4.73 (s, 2H), 1.58-1.55 (m, 2H), 1.28-1.18 (m and s, overlapping, 12H, especially 1.26, s, 6H, —C(CH₃)₂), 1.15-1.09 (m, 2H), 0.86 (t, J=7.0 Hz, 3H).

### Synthesis of Carbamates 57.1 and 57.2

The carbamates 57.1 and 57.2 were synthesized by a method depicted in Scheme 11 starting from commercially available 4-bromobenzyl bromide (54).

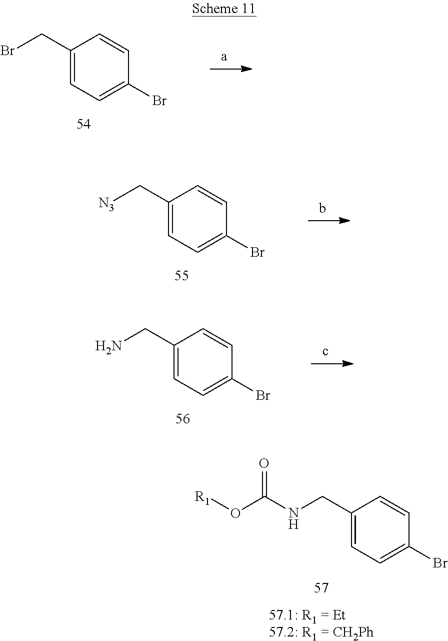

Reagents and conditions for the steps in Scheme 11 were as follows: Step a: NaN₃, DMF, 50° C., 3 hours, 92%; Step b: PPh₃, THF/CH₃OH, reflux, 1.5 hours, 63%; Step c: R₁OCOCl, Na₂CO₃, toluene, RT, 4-6 hours, 82-90%.

### 2. 4-Bromobenzyl azide (55)

A mixture of 4-bromobenzyl bromide (54) and sodium azide (2.0 equiv.) in DMF was stirred at 50° C. for 3 hours. The reaction mixture was diluted with water and extracted with CH₂Cl₂. The combined organic extract was dried over MgSO₄, and concentrated in vacuo. The residue was purified by flash chromatography to yield 55 as colorless oil in 92% yield.

55 was confirmed as follows: ¹H NMR (500 MHz, CDCl₃) δ 7.51 (d, J=8.5 Hz, 2H), 7.19 (d, J=8.5 Hz, 2H), 4.31 (s, 2H). IR (Neat): 2091, 1592, 1488 cm⁻¹.

### 3. 4-Bromobenzyl amine (56)

To a stirred solution of azide 55 (0.75 g, 3.54 mmol) in anhydrous methanol (10 mL) was added triphenylphosphine (1.39 g, 5.31 mmol) and the mixture was heated under reflux for 1.5 hours. The reaction mixture was cooled to RT, and the solvent was removed under reduced pressure. The residue was purified by flash column chromatography on silica gel (acetone-hexane) to yield product 56 in 63% yield (0.41 g).

### 4. Carbamates (57)

To a stirred suspension of 4-bromobenzyl amine 56 (1 equiv.) and sodium carbonate (1.5 equiv.) in anhydrous toluene at RT was added ethyl or benzyl chloroformate. Stirring was continued for 4-6 hours at the same temperature, insoluble materials were filtered off, and the filtrate was washed with water and dried over MgSO₄. Solvent evaporation under reduced pressure and purification by flash column chromatography on silica gel (diethyl ether-hexane) gave pure product (57.1 or 57.2 respectively) in 82-90% yields.

### 5. Selected data of synthesized carbamates (57)

### (4-Bromobenzyl)carbamic acid benzyl ester (57.2)

57.2 was confirmed as follows: ¹H NMR (500 MHz, CDCl₃) δ 7.44 (d, J=7.7 Hz, 2H), 7.38-7.30 (m, 5H), 7.16 (d, J=7.7 Hz, 2H), 5.13 (s, 2H), 5.09 (br s, 1H, NH), 4.32 (d, J=5.5 Hz, 2H).

### Carbamates 57.3, 57.4, and 57.5

The carbamates 57.3, 57.4 and 57.5 were synthesized by a method depicted in Scheme 12.

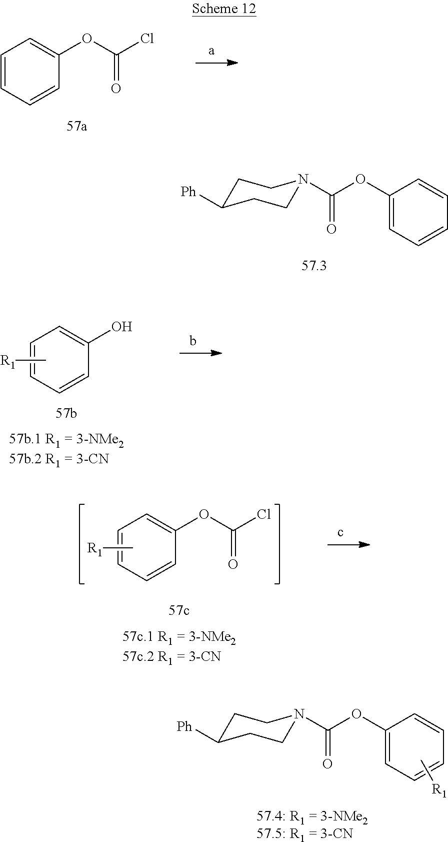

Reagents and conditions for the steps in Scheme 12 were as follows: Step a: 4-phenylpiperidine, CH2Cl2, RT, 12 hours; Step b: (i) NaH, THF, 0° C.—RT, 1 hour (ii) triphosgene, RT, 1 hour; Step c: 4-phenylpiperazine, RT, 12 hours.

### 1. Carbamates (57.3)

To a stirred solution of 4-phenyl piperazine (2.2 equiv.) in anhydrous CH₂Cl₂ at RT was added phenylchoroformate 57a (1.0 equiv.) and the resulting mixture stirred at the same temperature (12 hours). The mixture was washed with 5% HCl (aq.), the organic layer was washed with brine and dried over MgSO₄. Solvent evaporation under reduced pressure gave pure product (57.3 in 96% yield).

57.3 was confirmed as follows: ¹H NMR (500 MHz, CDCl₃) δ 7.41-7.35 (m, 4H), 7.27-7.25 (m, 3H), 7.23 (t, J=7.5 Hz, 1H), 7.15 (d, J=7.7 Hz, 2H), 4.47 (t, J=15 Hz, 2H), 3.12 (t, J=11.5 Hz, 1H), 2.97 (t, J=12.0 Hz, 1H), 2.77 (tt, J=12.5 Hz, J=3.5 Hz, J=3.5 Hz), 1.96 (d, J=13 Hz, 2H), 1.79 (dq, J=12.0 Hz, J=4.5 Hz).

### 2. Carbamates (57.4 and 57.5)

To a stirred solution of phenols 57b.2 or 57b.2 (1.0 equiv.) in THF at 0° C. was added NaH (60% dispersion in mineral oil, 1.05 equiv.) and the resulting mixture gradually warmed to RT. Triphosgene (0.33 equiv.) was added, and the mixture stirred for additional 2 hours at the same temperature. This mixture (1.0 equiv) was added to a solution of 4-phenylpiperazine (2.2 equiv.) in CH₂Cl₂, and the mixture stirred for 12 hours. The mixture was washed with 1N NaOH (aq.), the organic layer was washed with brine, and then dried over MgSO₄. Solvent evaporation under reduced pressure gave a crude product, which was purified by column chromatography on silica ge (25% EtOAc:Hexane) to give (57.4 and 57.5 in 45-48% yield (from 57b.1 or 57b.2).

### 3. Compound 57.4

57.4 was confirmed as follows: ¹H NMR (500 MHz, CDCl₃) δ 7.38-7.34 (m, 2H), 7.26 (t, J=7.0 Hz, 2H), 7.22 (t, J=8.5 Hz, 1H), 7.10 (t, J=8.5 Hz, 1H), 6.58 (dd, J=7.7 Hz, J=2.5 Hz, 1H), 6.52-6.48 (m, 2H), 4.47 (t, J=15.0 Hz, 2H), 3.10 (m, 1H), 2.97 (m overlapping with singlet 4H), 2.95 (s, 3H), 2.77 (tt, J=12.5 Hz, J=3.5 Hz, J=3.5 Hz), 1.96 (d, J=13 Hz, 2H), 1.79 (dq, J=12.0 Hz, J=4.5 Hz).

### 4. Compound 57.5

57.5 was confirmed as follows: ¹H NMR (500 MHz, CDCl₃) δ 7.51-7.46 (m, 3H), 7.44-7.40 (m, 1H), 7.36-7.32 (m, 2H), 7.26-7.22 (m, 3H), 4.41 (t, J=14.0 Hz, 2H), 3.12 (t, J=12.2 Hz, 1H), 2.98 (t, J=13.0 Hz, 1H), 2.76 (tt, J=12.5 Hz, J=4.0 Hz, J=4.0 Hz), 1.96 (d, J=14.0 Hz, 2H), 1.79 (dd, J=13.0 Hz, J=4.0 Hz).

### Synthesis of Ureas 59.1 and 59.2

Ureas 59.1 and 59.2 were synthesized by a method depicted in Scheme 13 starting from commercially available 3-phenyl-propyl isocyanate (58) and 2-aminomethyl-pyridine or 2-aminopyridine.

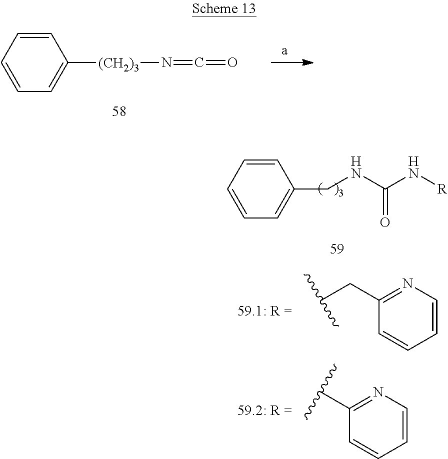

Reagents and conditions for Scheme 13 were as follows: Step a: R—NH₂, THF or benzene, 0° C. to reflux 85-93%.

### 2. N-(3-phenylpropyl)-N′-(2-pyridinylmethyl)-urea (59.1)

To a solution of 3-phenylpropyl isocyanate (1.8 mmol) in anhydrous THF (10 mL) at 0° C. under an argon atmosphere was added 2-aminomethyl-pyridine (1.8 mmol). The reaction mixture was stirred at 0° C. for 10 min, the solvent was evaporated under reduced pressure, and the resultant solid was recrystallized from CH₂Cl₂/Et₂O to give pure 59.1 in 92% yield (white solid, melting point 89-90° C.). When anhydrous benzene was used as solvent the product was directly crystallized out and isolated by filtration (93% yield).

59.1 was confirmed as follows: ¹H NMR (500 MHz, CDCl₃) δ 8.47 (d, J=4.4 Hz, 1H), 7.61 (td, J=7.6 Hz, J=1.1 Hz, 1H), 7.28-7.22 (m, 3H), 7.19-7.10 (m, 4H), 5.97 (t, J=4.9 Hz, 1H, NH), 5.30 (br s, 1H, NH), 4.45 (d, J=5.4 Hz, 2H), 3.20 (td as q, J=6.4 Hz, 2H), 2.61 (t, J=7.6 Hz, 2H), 1.79 (quintet, J=7.3 Hz, 2H); IR (neat), 3320, 3028, 2941, 2860, 1620, 1594, 1568, cm⁻¹.

### 3. N-(3-phenylpropyl)-N′-(2-pyridinyl)-urea (59.2)

To a stirred solution of 3-phenylpropyl isocyanate (2 mmol) in anhydrous THF (15 mL) at 0° C. under an argon atmosphere was added 2-amino-pyridine (2 mmol). Following the addition, the reaction mixture was heated under reflux for 2 hours, the solvent was evaporated under reduced pressure, and the resultant solid was recrystallized from CH₂Cl₂/Et₂O to give pure 59.2 in 85% yield (white solid, melting point 127-128° C.).

59.2 was confirmed as follows: ¹H NMR (500 MHz, CDCl₃) δ 9.72 and 9.66 (s and br s, overlapping, 2H, NH), 8.16 (d, J=4.3 Hz, 1H), 7.58 (t, J=7.1 Hz, 1H), 7.30 (t, J=7.4 Hz, 2H), 7.24 (d, J=7.4 Hz, 2H), 7.21 (t, J=7.4 Hz, 1H), 6.94 (d, J=7.1 Hz, 1H), 6.87 (m as t, J=6.4 Hz, 1H), 3.44 (td as q, J=6.4 Hz, 2H), 2.75 (t, J=7.6 Hz, 2H), 1.97 (quintet, J=7.2 Hz, 2H); IR (neat) 3221, 3054, 2980, 2918, 1682, 1602, 1583, 1549, 1480 cm⁻¹.

### Synthesis of Ureas 59.3-59.7

Ureas 59.3 though 59.7 were synthesized by a method depicted in Scheme 14.

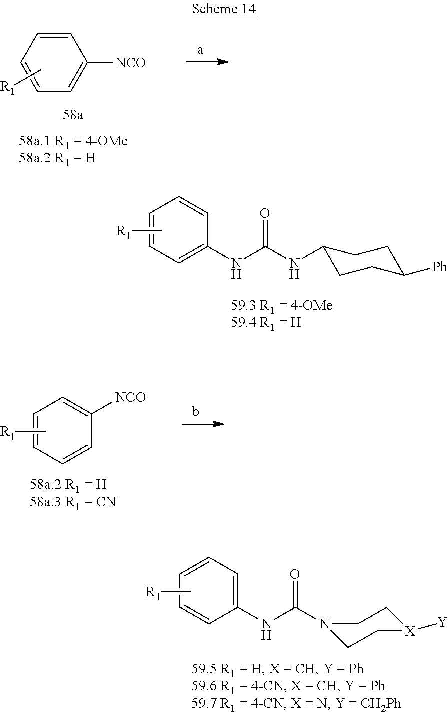

Reagents and conditions for the steps in Scheme 14 were as follows:

Step a: 4-phenyl-cyclohexylamine, CH₂Cl₂, RT, 2 hours;
Step b: 4-phenylpiperidine or 4-benzylpiperazine, CH₂Cl₂, RT, 2 hours.

### 2. N-(4-methoxyphenyl)-N′-(4-phenylcyclohexyl)-urea (59.3)

To a stirred solution of 4-methoxyphenyl isocyanate 58a.1 (1 equiv)) in CH₂Cl₂ at RT under an argon atmosphere was added 4-phenyl-cyclohexyl amine (2.2 equiv.) and the resulting mixture stirred for 2 hours. The mixture was washed with 5% HCl (aq.), the organic layer was dried over MgSO₄, and the solvent removed under reduced pressure. The crude material was purified by flash column chromatography on silica gel to give 59.3 in 77% yield.

59.3 was confirmed as follows: ¹H NMR (500 MHz, CD3OD) δ 7.36 (dd, J=8.5 Hz, J=1.0 Hz, 2H), 7.30-7.23 (m, 6H), 7.16 (t, J=7.0 Hz, 1H), 6.98 (t, J=7.01 Hz, 1H), 3.64 (tt, J=12.0 Hz, J=4.5 Hz, J=4.0 Hz, 1H), 2.54 (tt, J=11.8 Hz, J=3.5 Hz, J=3.5 Hz), 2.10 (d, J=13.5 Hz, 2H), 1.94 (d, J=13.5 Hz, 2H), 1.65 (dq, J=12.5 Hz, J=3.5 Hz, 2H), 1.39 (dd, J=12.5 Hz, J=3.5 Hz, 2H).

### 3. N-(phenyl)-N′-(4-phenylcyclohexy)-urea (59.4)

59.4 was confirmed as follows: ¹H NMR (500 MHz, CD3OD) δ 7.29-7.22 (m, 7H), 7.16 (t, J=7.0 Hz, 1H), 6.85 (d, J=8.5 Hz, 2H), 3.64 (tt, J=12.0 Hz, J=4.0 Hz, J=4.0 Hz, 1H), 2.53 (tt, J=12.0 Hz, J=3.5 Hz, J=3.5 Hz), 2.11 (d, J=8.5 Hz, 2H), 1.93 (d, J=9.0 Hz, 2H), 1.64 (dq, J=13.0 Hz, J=3.0 Hz, 2H), 1.38 (dq, J=12.5 Hz, J=3.5 Hz, 2H).

### 4. N-(phenyl)-(4-phenylpiperidine)-1-carboxamide (59.5)

To a stirred solution of phenyl isocyanate 58a.2 (1 equiv)) in CH₂Cl₂ at RT under an argon atmosphere was added 4-phenyl-piperidine (2.2 equiv.) and the resulting mixture stirred for 2 hours. The mixture was washed with 5% HCl (aq.), the organic layer was dried over MgSO₄, and the solvent removed under reduced pressure. The crude material was purified by flash column chromatography on silica gel to give 59.5 in 73% yield.

59.5 was confirmed as follows: ¹H NMR (500 MHz, CDCl₃) ′δ 7.41-7.30 (m, 7H), 7.27-7.24 (m, 3H), 6.42 (brs, 1H), 4.25 (d, J=13.5 Hz, 2H), 3.04 (dt, J=12.5 Hz, J=2.0 Hz, 2H), 2.76 (tt, J=12 Hz, J=3.8 Hz, J=3.8 Hz, 1H), 1.96 (d, J=12.5 Hz, 2H), 1.77 (dq, J=13.0 Hz, J=4.5 Hz, 2H).

### 5. N-(4-cyanophenyl)-(4-phenylpiperidine)-1-carboxamide (59.6)

N-(4-cyanophenyl)-(4-phenylpiperidine)-1-carboxamide (59.6) was prepared as described for 59.5 using 4-cyanophenyl isocyanate 58.a.3 (1 equiv.) and 4-phenyl piperidine (2.2 equivalent) in CH₂Cl₂ to give 59.6 in 75% yield.

59.6 was confirmed as follows: ¹H NMR (500 MHz, CDCl₃) δ 7.56 (d, J=8.5 Hz, 2H), 7.52 (d, J=9.0 Hz, 2H), 7.33 (t, J=7.2 Hz, 2H), 7.25-7.20 (m, 3H), 6.76 (brs, 1H), 4.23 (d, J=13.5 Hz, 2H), 3.05 (dt, J=13.0 Hz, J=2.0 Hz, 2H), 2.75 (tt, J=12 Hz, J=3.5 Hz, J=3.5 Hz, 1H), 1.95 (d, J=12.5 Hz, 2H), 1.77 (dq, J=12.5 Hz, J=4.0 Hz, 2H).

### 6. N-(4-cyanophenyl)-(4-benzylpiperidine)-carboxamide (59.7)

N-(4-cyanophenyl)-(4-benzylpiperidine)-carboxamide (59.7) was prepared as described for 59.5 using 4-cyanophenyl isocyanate 58a.3 (1 equiv)) and 4-benzyl-piperidine (2.2 equiv.) in CH₂Cl₂ to give 59.7 in 76% yield.

59.7 was confirmed as follows: ¹H NMR (500 MHz, CDCl₃) δ 7.58-7.55 (m, 2H), 7.52-7.50 (m, 2H), 7.38-7.29 (m, 5H), 6.70 (brs, 1H), 3.60 (s, 2H), 3.56-3.54 (m, 4H), 2.54-5.51 (m, 4H).

### Synthesis of α-Keto-oxadiazoles 65.1, 65.2, and 66

α-Keto-oxadiazoles 65.1, 65.2 and 66 were synthesized by a method depicted in Scheme 15 starting from 60.1 or 60.2 and 2-methyl-oxadiazole (63). Phenol (60.1) and 4-benzyloxy-phenol (60.2) were commercially available while 2-methyl-oxadiazole (63) was prepared by a method disclosed in Ainsworth et al., J. Org. Chem. Soc., (1966) 31:3442-3444, and in Ohmoto et al., J. Med. Chem., (2001) 44:1268-1285.

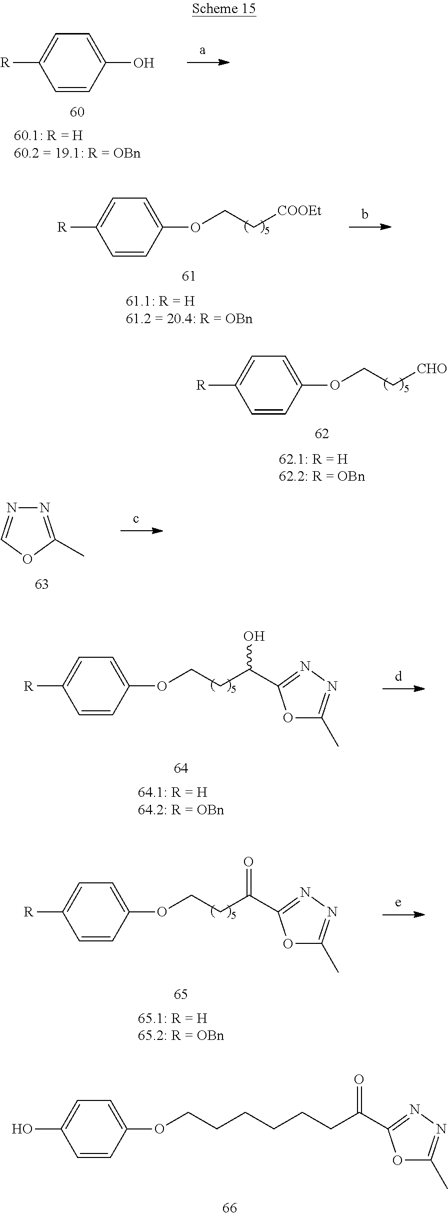

Reagents and conditions for the steps in Scheme 15 were as follows: Step a: Br(CH₂)₆COOEt, K₂CO₃, 18-crown-6, acetone, 50° C., 12 hours, 90-92%; Step b: DIBAL-H, THF, −78° C., 1 hour, 63-65%; Step c: n-BuLi, MgBr₂.Et₂O, THF −78° C. to −50° C., then addition to 62.1 or 62.2, CeCl₃, −78° C., 52-55%; Step d: Dess-Martin periodinane, CH₂Cl₂, RT, 80-82%; Step e: Pd/C, H₂, AcOEt, RT, 71%.

### 2. 7-(Phenoxy)heptanoic acid ethyl ester (61.1)

To a solution of 60.1 (0.7 g, 7.5 mmol) in dry acetone (50 mL), under a nitrogen atmosphere, was added 18-crown-6 (1.584 g, 6 mmol), anhydrous potassium carbonate (2.07 g, 15 mmol), and ethyl 7-bromoheptanoate (1.18 g, 5 mmol) successively. The mixture was stirred at 50° C. overnight, cooled to RT, and the solvent removed in vacuo. The residue obtained was partitioned between diethyl ether (50 mL), and water (10 mL). The organic phase was separated and the aqueous layer extracted with diethyl ether. The combined organic layer was washed with brine, dried (MgSO₄), and the solvent was removed under reduced pressure. Purification by flash column chromatography (20% diethyl ether-hexane) afforded 61.1 (1.72 g, 92% yield) as a colorless liquid.

61.1 was confirmed as follows: ¹H NMR (500 MHz, CDCl₃) δ 7.27 (dt, J=7.7 Hz, J=1.5 Hz, 2H), 6.92 (dt, J=7.7 Hz, J=1.5 Hz 1H), 6.89 (d, J=7.7 Hz, 2H), 4.12 (q, J=7.0 Hz, 2H), 3.95 (t, J=6.2 Hz, 2H), 2.31 (t, J=7.7 Hz, 2H), 1.78 (quintet, J=6.5 Hz, 2H), 1.66 (quintet, J=7.5 Hz, 2H), 1.49 (quintet, J=7.2 Hz, 2H), 1.40 (quintet, J=8.2, 2H), 1.25 (t, J=7.0 Hz, 2H).

### 3. 7-[4-(Benzyloxy)phenoxy]heptanoic acid ethyl ester (61.2/20.4)

An alternative method for the synthesis of the title compound was carried out analogous to the preparation of 61.1 using 60.2 (0.45 g, 2.255 mmol), 18-crown-6 (1.056 g, 4 mmol), potassium carbonate (1.38 g, 10 mmol), and Br(CH₂)₆COOEt, (0.8 g, 3.37 mmol) in dry acetone (40 mL). Purification by flash column chromatography on silica gel (20% diethyl ether-hexane) gave 61.2/20.4 (1.08 g, 90% yield) as a white solid (melting point 57-61° C.).

61.2/20.4 was confirmed as follows: ¹H NMR (500 MHz, CDCl₃) δ 7.42 (d, J=7.5 Hz, 2H), 7.37 (t, J=7.5 Hz, 2H), 7.31 (t, J=7.5 Hz, 1H), 6.89 (d, J=8.7 Hz, 2H), 6.82 (d, J=8.7 Hz, 2H), 5.01 (s, 2H), 4.12 (q, J=7.0 Hz, 2H), 3.89 (t, J=6.5 Hz, 2H), 2.30 (t, J=7.5 Hz, 2H), 1.76 (quintet, J=6.7 Hz, 2H), 1.66 (quintet, J=7.5 Hz, 2H), 1.47 (quintet, J=7.2 Hz, 2H), 1.38 (quintet, J=6.7 Hz, 2H), 1.25 (t, J=7.0 Hz, 2H).

### 4. 7-(Phenoxy)heptanal (62.1)

To a stirred solution of 61.1 (0.56 g, 2.24 mmol) in dry THF (20 mL), at −78° C., under a nitrogen atmosphere was added diisobutylaluminum hydride (5 mL, 5 mmol, using a 1 M solution in hexanes) dropwise. The reaction mixture was stirred at the same temperature for 30 min and then quenched by dropwise addition of potassium sodium tartrate (10% solution in water). The resulting mixture was warmed to RT and stirred vigorously for 1 hour. The organic layer was separated and the aqueous phase extracted with diethyl ether. The combined organic layer was washed with brine, dried (MgSO₄), and concentrated in vacuo. The residue was purified by column chromatography on silica gel, eluting with 25% diethyl ether-hexane to give 62.1 (0.26 g, 65% yield) as a colorless viscous liquid.

62.1 was confirmed as follows: ¹H NMR (500 MHz, CDCl₃) δ 9.80 (s, 1H), 7.27 (t, J=7.5 Hz, 2H), 6.93 (d, J=7.5 Hz, 2H), 6.89 (d, J=7.5 Hz, 2H), 3.95 (t, J=6.2 Hz, 2H), 2.45 (t, J=7.2 Hz, 2H), 1.79 (quintet, J=6.7 Hz, 2H), 1.67 (quintet, J=7.0 Hz, 2H), 1.50 (quintet, J=6.7 Hz, 2H), 1.41 (quintet, J=7.7 Hz, 2H).

### 5. 7-[4-(Benzyloxy)phenoxy]heptanal 62.2

7-[4-(Benzyloxy)phenoxy]heptanal 62.2 was synthesized analogous to the preparation of 62.1 using 61.2/20.4 (0.624 g, 2 mmol) and diisobutylaluminum hydride (4.5 mL, 4.5 mmol, using a 1 M solution in hexanes) in THF (20 mL). Purification by flash column chromatography on silica gel gave 62.2 (0.39 g, 63% yield) as a white solid (melting point 65-67° C.).

62.2 was confirmed as follows: ¹H NMR (500 MHz, CDCl₃) δ 9.80 (s, 1H), 7.42 (d, J=7.2 Hz, 2H), 7.38 (t, J=7.2 Hz, 2H), 7.31 (t, J=7.2 Hz, 1H), 6.90 (d, J=8.7 Hz, 2H), 6.82 (d, J=8.7 Hz, 2H), 5.01 (s, 2H), 3.90 (t, J=6.2 Hz, 2H), 2.44 (dt, J=7.2 Hz, J=2.0 Hz, 2H), 1.76 (quintet, J=7.5 Hz, 2H), 1.67 (quintet, J=7.5 Hz, 2H), 1.48 (quintet, J=7.2 Hz, 2H), 1.40 (quintet, J=7.7 Hz, 2H).

### 6. 7-Phenoxy-1-(5-methyl-1,3,4-oxadiazol-2-yl)-heptan-1-ol (64.1)

To a stirred solution of 63 (0.252 g, 3 mmol) in anhydrous THF (5 mL), at −78° C., under a nitrogen atmosphere, was added n-BuLi (1.2 mL, 3 mmol, using a 2.5 M solution in hexanes) dropwise. Stirring was continued for 15 min at −78° C., and then MgBr₂.Et₂O (0.774 g, 3 mmol) was added. The resulting mixture was warmed to −50° C. over a 2 hour period, and then it was transferred by cannula to a cooled (−78° C.) slurry of 62.1 (0.125 g, 0.6 mmol) and CeCl₃, (0.738 g, 3 mmol) in anhydrous THF (6 mL), which was previously stirred at RT for 2 hours under nitrogen. Following the addition, the resultant mixture was allowed to warm to RT over a 4 hour period. The reaction mixture was quenched with dropwise addition of 5% aqueous AcOH solution (10 mL), diluted with AcOEt (20 mL), and the organic phase was separated. The aqueous layer extracted with AcOEt, the combined organic layer was washed with an aqueous saturated NaHCO₃ solution and brine, dried (MgSO₄), and the solvent was evaporated under reduced pressure. The residue obtained was purified by flash column chromatography on silica gel (75% ethyl acetate-hexane) to give 64.1 (92.5 mg, 53% yield) as a white solid (melting point 50-52° C.).

64.1 was confirmed as follows: ¹H NMR (500 MHz, CDCl₃) δ 7.27 (t, J=7.7 Hz, 2H), 6.93 (t, J=7.7 Hz, 1H), 6.89 (d, J=7.7 Hz, 2H), 4.91 (t, J=6.2 Hz, 1H), 3.95 (t, J=6.7 Hz, 2H), 2.80 (br s, 1H), 2.54 (s, 3H), 1.98-1.90 (m, 2H), 1.78 (quintet, J=6.7 Hz, 2H), 1.54-1.40 (m, 6H).

### 7. 7-(4-Benzyloxy-phenoxy)-1-(5-methyl-1,3,4-oxadiazol-2-yl)-heptan-1-ol (64.2)

The synthesis was carried out analogous to the preparation of 64.1 using 62.2 (0.1 g, 0.32 mmol), cerium chloride (0.44 g, 1.6 mmol) and 63 (0.42 g, 1.6 mmol). Purification by flash column chromatography on silica gel gave pure 64.2 (0.077 mg, 55% yield) as a white solid (melting point 98-100° C.).

64.2 was confirmed as follows: ¹H NMR (500 MHz, CDCl₃) δ 7.42 (d, J=7.5 Hz, 2H), 7.38 (t, J=7.5 Hz, 2H), 7.31 (t, J=7.5 Hz, 1H), 6.90 (d, J=8.7 Hz, 2H), 6.82 (d, J=8.7 Hz, 2H), 5.01 (s, 2H), 4.90 (q, J=6.0 Hz, 1H), 3.89 (t, J=6.5 Hz, 2H), 2.54 (s, 3H), 2.51 (d, J=6.0 Hz, 1H), 2.00-1.92 (m, 2H), 1.75 (quintet, J=7.5 Hz, 2H), 1.56-1.40 (m, 6H).

### 8. 7-Phenoxy-1-(5-methyl-1,3,4-oxadiazol-2-yl)-heptan-1-one (65.1)

To a solution of 64.1 (64 mg, 0.22 mmol) in wet methylene chloride (5 mL) at RT, under nitrogen was added Dess-Martin periodinane (140 mg, 0.33 mmol) and the resulting suspension stirred for 2 hours. The reaction mixture was diluted with Na₂S₂O₃ (10% in H₂O) and saturated aqueous NaHCO₃ solution, and the organic phase was separated. The aqueous layer was extracted with AcOEt, and the combined organic layer was washed with brine, dried (MgSO₄), and evaporated under reduced pressure. The residue obtained was purified by flash column chromatography on silica gel (50% ethyl acetate-hexane) to give 65.1 (52 mg, 82% yield) as a white solid (melting point 75-77° C.).

65.1 was confirmed as follows: ¹H NMR (500 MHz, CDCl₃) δ 7.27 (t, J=7.5 Hz, 2H), 6.93 (t, J=7.5 Hz, 1H), 6.89 (d, J=7.5 Hz, 2H), 3.95 (t, J=6.2 Hz, 2H), 3.15 (t, J=7.2 Hz, 2H), 2.64 (s, 3H), 1.84-1.77 (m, 4H), 1.52-1.44 (m, 4H).

### 9. 7-(4-Benzyloxy-phenoxy)-1-(5-methyl-1,3,4-oxadiazol-2-yl)-heptan-1-one (65.2)

The synthesis was carried out analogous to the preparation of 65.1 using 64.2 (60 mg, 0.15 mmol) and Dess-Martin periodinane (0.127 g, 0.3 mmol) in wet CH₂Cl₂ (5 mL). Purification by flash column chromatography on silica gel gave pure compound 65.2 (47.5 mg, 80% yield) as a white solid (melting point 118-120° C.).

65.2 was confirmed as follows: ¹H NMR (500 MHz, CDCl₃) δ 7.42 (d, J=7.5 Hz, 2H), 7.38 (t, J=7.5 Hz, 2H), 7.31 (t, J=7.5 Hz, 1H), 6.90 (d, J=8.7 Hz, 2H), 6.82 (d, J=8.7 Hz, 2H), 5.01 (s, 2H), 3.90 (t, J=6.2 Hz, 2H), 3.14 (t, J=7.5 Hz, 2H), 2.64 (s, 3H), 1.84-1.74 (m, 4H), 1.54-1.44 (m, 4H).

### 10. 7-(4-Hydroxy-phenoxy)-1-(5-methyl-1,3,4-oxadiazol-2-yl)-heptan-1-one (66)

To a solution of 65.2 (30 mg, 0.076 mmol) in AcOEt (5 mL) was added 10% Pd/C (6 mg, 20% w/w) and the resulting suspension was stirred vigorously under hydrogen atmosphere, overnight at RT. The catalyst was removed by filtration through Celite, and the filtrate was evaporated under reduced pressure. The residue obtained was purified by flash column chromatography on silica gel (60% ethyl acetate-hexane) to give pure compound 66 (0.016 g, 71% yield) as a white solid (melting point 134-135° C.).

66 was confirmed as follows: ¹H NMR (500 MHz, CDCl₃) δ 6.80-6.74 (m, 4H), 4.56 (br s, 1H), 3.89 (t, J=6.5 Hz, 2H), 3.14 (t, J=7.2 Hz, 2H), 2.64 (s, 3H), 1.84-1.74 (m, 4H), 1.54-1.44 (m, 4H).

### Synthesis of α-Keto-oxadiazoles 73.1, 73.2, 74.1, and 74.2

α-Keto-oxadiazoles 73.1, 73.2 74.1 and 74.2 were synthesized by a method depicted in Scheme 16 starting from 7-(phenoxy)heptanoic acid ethyl ester (61.1), 7-[4-(benzyloxy)phenoxy]heptanoic acid ethyl ester (61.2), and commercially available methyl glycolate (69).

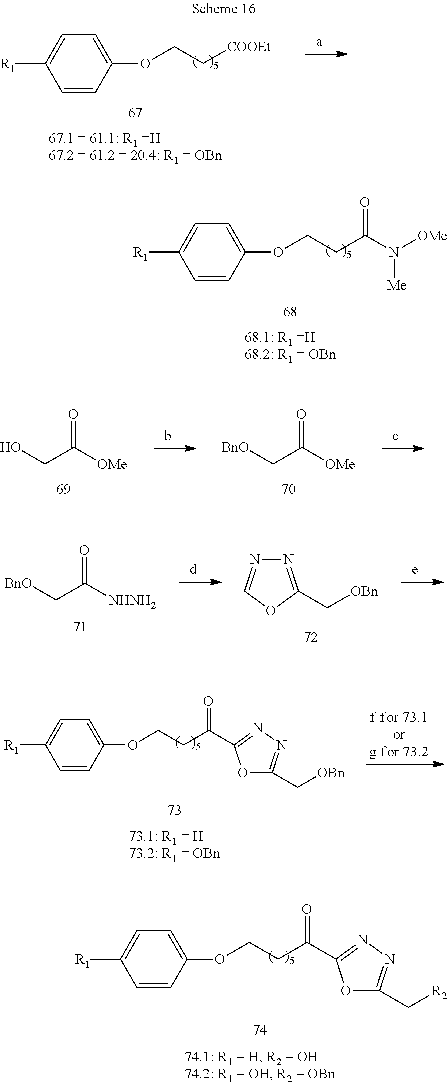

Reagents and conditions for the steps in Scheme 16 were as follows:

Step a: (MeO)MeNH₂⁺Cl⁻, n-BuLi, THF, −78° C., 15 min, then addition of 67, −78° C., 40 min, 85-87%; Step b: BnBr, Ag₂O, Et₂O, RT, 24 hours, 70%; Step c: H₂NNH₂.H₂O, MeOH, reflux, 3 hours; Step d: CH(OMe)₃, p-TSA, reflux, 3 hours, 49% from 70; Step e: n-BuLi, MgBr₂.Et₂O, THF −78° C. to −30° C., 2 hours, then addition of 68.1 or 68.2, −30° C. to 0° C., 4 hours, 53-55%; Step f: 1,4-cyclohexadiene, 10% Pd/C, AcOH/MeOH, 45° C., 2 hours, 25%; Step g: H₂, 10% Pd/C, AcOEt, RT, overnight, 75%.

### 1. 7-Phenoxy-(N-methoxy-N-methyl)-heptane-carboxamide (68.1)

The title compound was synthesized analogously to 68.2 (see description below) using dry N,O-dimethylhydroxylamine hydrochloride (488 mg, 5 mmol) in anhydrous THF (40 mL), n-BuLi (2.5 M solution in hexanes, 4 mL, 10 mmol) and 67.2 (250 mg, 1 mmol). The crude obtained after workup was chromatographed over a column of silica gel, eluting with 50% ethyl acetate-petroleum ether to afford 68.1 as a colorless liquid in 87% yield (230 mg).

68.1 was confirmed as follows: ¹H NMR (500 MHz, CDCl₃) δ 7.27 (t, J=8.0 Hz, 2H), 6.92 (t, J=8.0 Hz, 1H), 6.89 (d, J=8.0 Hz, 2H), 3.95 (t, J=6.5 Hz, 2H), 3.68 (s, 3H), 3.18 (s, 3H), 2.43 (t, J=7.5 Hz, 2H), 1.79 (quintet, J=6.5 Hz, 2H), 1.67 (quintet, J=7.5 Hz, 2H), 1.50 (quintet, J=7.0 Hz, 2H), 1.42 (quintet, J=7.0 Hz, 2H).

### 2. 7-[(4-Benzyloxy-phenoxy)-N-methoxy-N-methyl]-heptane-carboxamide (68.2)

To a stirred suspension of N,O-dimethylhydroxylamine hydrochloride (dry, 680 mg, 7 mmol) in anhydrous THF (40 mL) at −78° C., under an argon atmosphere, was added n-BuLi (2.5 M solution in hexanes, 5.6 mL, 14 mmol) dropwise. The mixture was stirred for 15 min after removing the dry ice/acetone bath (to ensure complete dissolution of the salt), cooled again to −78° C., and a solution of 67.1 (500 mg, 1.4 mmol) in anhydrous THF (10 mL) was added dropwise. The reaction mixture was stirred for an additional 40 min at the same temperature, diluted with aqueous NH₄Cl, and the resulting mixture warmed to RT. The organic layer was separated, and the aqueous layer extracted with ethyl acetate (2×20 mL). The combined organic layer was washed with brine (20 mL), dried over MgSO₄, and the solvent evaporated under reduced pressure. The crude product was chromatographed over a column of silica gel, eluting with 50% ethyl acetate-petroleum ether to afford 68.2 as a white solid (melting point 54-55° C.) in 85% yield (440 mg).

68.2 was confirmed as follows: ¹H NMR (500 MHz, CDCl₃) δ 7.42 (d, J=7.0 Hz, 2H), 7.37 (t, J=7.0 Hz, 2H), 7.32 (t, J=7.0 Hz, 1H), 6.90 (d, J=9.0 Hz, 2H), 6.81 (d, J=9.0 Hz, 2H), 5.01 (s, 2H), 3.90 (t, J=6.2 Hz, 2H), 3.68 (s, 3H), 3.18 (s, 3H), 2.43 (t, J=7.5 Hz, 2H), 1.77 (quintet, J=6.7 Hz, 2H), 1.67 (quintet, J=7.7 Hz, 2H), 1.48 (quintet, J=7.7 Hz, 2H), 1.40 (quintet, J=7.5 Hz, 2H).

### 3. Methyl-2-benzyloxy-acetate (70)

To a stirred solution of methyl glycolate (2 g, 22.2 mmol) in anhydrous diethyl ether (100 mL), at RT, under a nitrogen atmosphere, was added silver(I) oxide (10.3 g, 44.4 mmol). The suspension was stirred for 15 min and benzyl bromide (4.5 g, 26.3 mmol) was added. The mixture was stirred at the same temperature for 24 hours, and the insoluble materials were removed by filtration through a short pad of celite. The filtrate was concentrated under reduced pressure, and the crude product chromatographed over a column of silica gel, eluting with 20% diethyl ether-petroleum ether to give 70, as a colorless liquid in 70% yield (2.8 g).

70 was confirmed as follows: ¹H NMR (500 MHz, CDCl₃) δ 7.39-7.29 (m, 5H), 4.62 (s, 2H), 4.16 (s, 2H), 3.78 (s, 3H).

### 4. 2-Benzyloxy-acetic hydrazide (71)

A mixture of 70 (2.75 g, 15.3 mmol) in methanol (50 mL) and hydrazine hydrate (65% in water, 2.3 g, 30 mmol) was heated under reflux for 3 hours. The reaction mixture was concentrated under reduced pressure and the residue was diluted with benzene. The solvent was evaporated and the crude product was further dried under high vacuum (6 hours) to give 71 (2.75 g), as a light yellow waxy material, which was used in the next step without further purification.

71 was confirmed as follows: ¹H NMR (500 MHz, CDCl₃) δ 7.72 (br s, 1H, NH), 7.39-7.29 (m, 5H), 4.56 (s, 2H), 4.07 (s, 2H), 3.82 (br s, 2H, NH₂).

### 5. 2-Benzyloxymethyl-1,3,4-oxadiazole (72)

To a mixture of 71, (2.7 g, 15 mmol) and trimethyl orthoformate (5 mL) was added p-TSA, (anhydrous, 255 mg, 1.5 mmol). The mixture was refluxed for 3 hours, and the excess trimethyl orthoformate evaporated under reduced pressure. The crude product was purified over a column of silica gel, eluting with 30% acetone-petroleum ether to give 72 as a colorless liquid (1.4 g), in 49% yield (two steps).

72 was confirmed as follows: ¹H NMR (500 MHz, CDCl₃) δ 8.43 (s, 1H), 7.39-7.31 (m, 5H), 4.77 (s, 2H), 4.64 (s, 2H).

### 6. 7-(Phenoxy)-1-(5-benzyloxymethyl-1,3,4-oxadiazol-2-yl)-heptan-1-one (73.1)

The title compound was synthesized analogously to 73.2 (see description below) using 72 (190 mg, 1 mmol), n-BuLi (2.5 M solution in hexane, 0.4 mL, 1 mmol), MgBr₂Et₂O (284 mg, 1.1 mmol) and 68.1 (132 mg, 0.5 mmol). The crude obtained after workup was chromatographed over a column of silica gel, eluting with 30% ethyl acetate-petroleum ether to give 73.1 as a white solid (melting point 61-63° C.) in 53% yield (104 mg).

73.1 was confirmed as follows: ¹H NMR (500 MHz, CDCl₃) δ 7.38-7.32 (m, 5H), 7.27 (t, J=7.0 Hz, 2H), 6.92 (t, J=7.0 Hz, 1H), 6.90 (d, J=7.0 Hz, 2H), 4.77 (s, 2H), 4.68 (s, 2H), 3.95 (t, J=6.2 Hz, 2H), 3.16 (t, J=7.2 Hz, 2H), 1.86-1.76 (m, 4H), 1.56-1.43 (m, 4H).

### 7. 7-(4-Benzyloxy-phenoxy)-1-(5-benzyloxymethyl-1,3,4-oxadiazol-2-yl)-heptan-1-one (73.2)

To a stirred solution of 72 (380 mg, 2 mmol) in anhydrous THF (40 mL), at −78° C., under an argon atmosphere, was added n-BuLi (2.5 M solution in hexane, 0.8 mL, 2 mmol) dropwise. Stirring was continued for 15 min at the same temperature, and then MgBr₂Et₂O (568 mg, 2.2 mmol) was added. The mixture was warmed to −30° C. over a 2 hour period, and then a solution of 68.2 (370 mg, 1 mmol) in THF (10 mL) was added. The mixture was gradually warmed to 0° C. and maintained at the same temperature for 4 hours. The reaction mixture was diluted with aqueous NH₄Cl solution (20 mL) and ethyl acetate (50 ml) and gradually warmed to RT. The organic layer was separated and the aqueous layer was extracted with ethyl acetate (2×20 mL). The combined organic layer was washed with brine (30 mL), dried over MgSO₄, and the solvent evaporated under reduced pressure. The crude product was chromatographed over a column of silica gel, eluting with 30% ethyl acetate-petroleum ether to give 73.2 as a white solid (melting point 95-97° C.) in 55% yield (275 mg).

73.2 was confirmed as follows: ¹H NMR (500 MHz, CDCl₃) δ 7.42 (d, J=8.0 Hz, 2H), 7.40-7.34 (m, 6H), 7.28-7.33 (m, 2H), 6.90 (d, J=8.5 Hz, 2H), 6.81 (d, J=8.5 Hz, 2H), 5.01 (s, 2H), 4.77 (s, 2H), 4.68 (s, 2H), 3.90 (t, J=6.2 Hz, 2H), 3.16 (t, J=7.2 Hz, 2H), 1.86-1.74 (m, 4H), 1.54-1.44 (m, 4H).

### 8. 1-(5-hydroxymethyl-1,3,4-oxadiazol-2-yl)-7-phenoxy-heptan-1-one (74.1)

To a stirred suspension of 73.1 (80 mg, 0.2 mmol) and Pd/C (160 mg) in AcOH/MeOH (1:10 mixture, 5 mL) at 45° C. was added 1,4-cyclohexadiene (304 mg, 4 mmol) over a period of 30 min. The mixture was stirred for an additional 2 hours at the same temperature. The catalyst was removed by filtration through celite and the filtrate was evaporated under reduced pressure. The residue was purified through a column of silica, eluting with 45% ethyl acetate in petroleum ether to give 74.1 as a white solid (melting point 83-85° C.) in 25% yield (15 mg).

74.1 was confirmed as follows: ¹H NMR (400 MHz, CDCl₃) δ 7.27 (t, J=7.0 Hz, 2H), 6.92 (t, J=7.0 Hz, 1H), 6.90 (d, J=7.0 Hz, 2H), 4.88 (s, 2H), 3.95 (t, J=6.5 Hz, 2H), 3.16 (t, J=7.2 Hz, 2H), 2.56 (br s, 1H, OH), 1.88-1.74 (m, 4H), 1.59-1.44 (m, 4H).

### 9. 1-(5-benzyloxymethyl-1,3,4-oxadiazol-2-yl)-7-(4-hydroxy-phenoxy)-heptan-1-one (74.2)

A mixture of 73.2 (50 mg, 0.1 mmol) and Pd/C (10 mg) in AcOEt (5 mL) was stirred vigorously under hydrogen overnight at RT. The catalyst was removed by filtration through celite and the filtrate was evaporated under reduced pressure. The crude material was purified through a column of silica gel, eluting with 45% ethyl acetate-petroleum ether to give 74.2 as a white solid in 75% yield (31 mg).

74.2 was confirmed as follows: ¹H NMR (500 MHz, CDCl₃) δ 7.38-7.31 (m, 5H), 6.79-6.73 (m, 4H), 4.77 (s, 2H), 4.68 (s, 2H), 4.50 (br s, 1H, OH), 3.90 (t, J=6.2 Hz, 2H), 3.15 (t, J=7.2 Hz, 2H), 1.84-1.74 (m, 4H), 1.56-1.44 (m, 4H).

### Synthesis of α-Keto-oxadiazoles 78 and 81

α-Keto-oxadiazoles 78 and 81 were synthesized by a method depicted in Scheme 17 starting from commercially available 3-benzyloxybromobenzene (28) and 3-anisaldehyde (79).

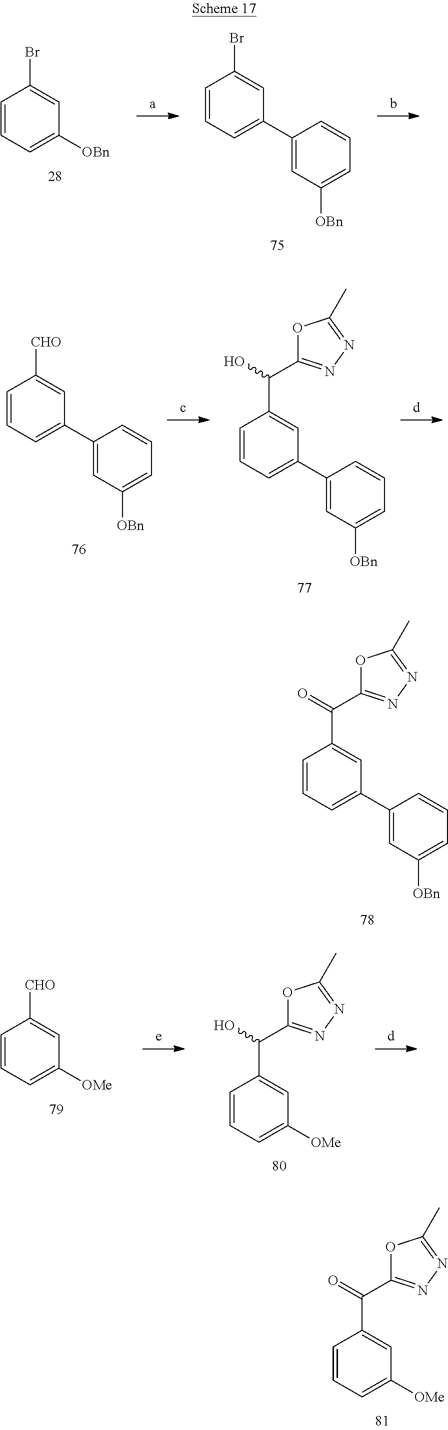

Reagents and conditions for the steps in Scheme 17 were as follows:

Step a: 3-cyanophenylboronic acid, Ba(OH₂), Pd(PPh₃)₄, DME/H₂O, reflux, 6 hours, 52%; Step b: DIBAL-H, THF, −78° C., 1 hour, 65%; Step c: 63, n-BuLi, MgBr₂.Et₂O, THF −78° C. to −45° C., 2 hours, then addition of 76, −78° C. to −45° C., 2 hours, 59%; Step d: Dess-Martin periodinane, CH₂Cl₂, 50° C., 2 hours, 80-82%; Step e: 63, n-BuLi, MgBr₂.Et₂O, THF, −78° C. to −50° C., 2 hours, then addition of 79, −78° C., 2 hours, 57%.

### 1. 3-(3-Benzyloxy-phenyl)benzonitrile (75)

A degassed mixture of 3-benzyloxy-phenyl bromide (28) (0.2 g, 0.76 mmol), 3-cyanophenylboronic acid (0.223 g, 1.52 mmol), barium hydroxide (0.285 g, 1.67 mmol), Pd(PPh₃)₄ (0.088 g, 0.076 mmol), DME (5 mL) and H₂O (3 mL) was heated (80° C.) for 6 hours with vigorous stirring under an argon atmosphere. The reaction mixture was cooled to RT, diluted with ethyl acetate, and filtered through a plug of celite. The filtrate was diluted with brine; the organic phase was separated, dried (MgSO₄), and concentrated in vacuo. The residue obtained was purified by flash column chromatography (20% diethyl ether-hexane) to give 75 (0.130 g, 60% yield) as a viscous liquid.

75 was confirmed as follows: ¹H NMR (500 MHz, CDCl₃) δ 7.85 (t, J=2.5 Hz, 1H), 7.78 (dt, J=7.5 Hz, J=1.5 Hz, 1H), 7.63 (dt, J=8.0 Hz, J=1.5 Hz, 1H), 7.53 (t, J=8.0 Hz, 1H), 7.46 (d, J=7.5 Hz, 2H), 7.44-7.37 (m, 3H), 7.35 (t, J=7.0 Hz, 1H), 7.18-7.14 (m, 2H), 7.02 (dd, J=8.5 Hz, J=2.5 Hz, 1H), 5.17 (s, 2H).

### 2. 3-(3-Benzyloxy-phenyl)benzaldehyde (76)

To a stirred solution of 75 (0.12 g, 0.42 mmol) in anhydrous THF (10 mL) at −78° C., under a nitrogen atmosphere was added diisobutylaluminum hydride (0.5 mL, 0.5 mmol, using a 1 M solution in hexane) dropwise. The reaction mixture was stirred at the same temperature for 1 hour, and then quenched by dropwise addition of potassium sodium tartrate (10% solution in water). The resulting mixture was warmed to RT, diluted with diethyl ether (20 mL), and stirred vigorously for 1 hour. The organic phase was separated and the aqueous phase was extracted with diethyl ether. The combined organic layer was washed with brine, dried (MgSO₄), and evaporated under reduced pressure. The residue obtained was purified by flash column chromatography on silica gel (20% diethyl ether-hexane) to give 76 (0.091 g, 75% yield) as a viscous liquid.

76 was confirmed as follows: ¹H NMR (500 MHz, CDCl₃) δ 10.08 (s, 1H), 8.09 (t, J=1.5 Hz, 1H), 7.85 (dt, J=7.5 Hz, J=1.5 Hz, 2H), 7.60 (t, J=7.7 Hz, 1H), 7.47 (d, J=7.5 Hz, 2H), 7.44-7.32 (m, 3H), 7.35 (t, J=7.5 Hz, 1H), 7.27-7.21 (m, 2H), 7.02 (dd, J=7.7 Hz, J=2.0 Hz, 1H), 5.14 (s, 2H).

### 3. 1-(3′-Benzyloxy-1,1′-biphenyl-3-yl)-1-(5-methyl-1,3,4-oxadiazol-2-yl)-methanol (77)

To a stirred solution of 63 (0.118 g, 1.5 mmol), in dry THF (5 mL), at −78° C., under a nitrogen atmosphere, was added n-BuLi (0.6 mL, 1.5 mmol, using a 2.5M solution in hexane) dropwise. Stirring continued for 10 min at −78° C., and then MgBr₂.Et₂O (0.4 g, 1.5 mmol) was added. The resulting mixture was warmed to −45° C. over a 2 hour period, cooled back to −78° C., and a solution of 76 (0.081 g, 0.28 mmol) in dry THF (5 mL) was added dropwise. Following the addition, the reaction mixture was warmed to −45° C. over a 2 hour period and then diluted with aqueous NH₄Cl solution (5 mL) and AcOEt (20 mL). The resulting mixture was gradually warmed to RT, the organic phase was separated, and the aqueous phase extracted with AcOEt. The combined organic layer was washed with brine, dried (MgSO₄), and evaporated under reduced pressure. The residue obtained was purified by flash column chromatography on silica gel (75% ethyl acetate-hexane) to give 77 (0.052 g, 50% yield) as a colorless viscous liquid.

77 was confirmed as follows: ¹H NMR (500 MHz, CDCl₃) δ 7.69 (m as br s, 1H), 7.59-7.56 (m, 1H), 7.47-7.45 (m, 4H), 7.40 (t, J=7.0 Hz, 2H), 7.37-7.32 (m, 2H), 7.20 (t, J=2.0 Hz, 1H), 7.18 (d J=8.0 Hz, 1H), 6.98 (dd, J=8.5 Hz, J=2.0 Hz, 1H), 6.08 (s, 1H), 5.12 (s, 2H), 3.22 (br s, 1H), 2.45, (s, 3H).

### 4. 1-(3′-Benzyloxy-1,1′-biphenyl-3-yl)-1-(5-methyl-1,3,4-oxadiazol-2-yl)-ketone (78)

To a solution of 77 (45 mg, 0.12 mmol) in wet CH₂Cl₂ (5 mL) at RT, under nitrogen, was added Dess-Martin periodinane (102 mg, 0.24 mmol) and the resulting suspension stirred for 2 hours at 50° C. The reaction mixture was cooled to RT, diluted with Na₂S₂O₃ (10% in H₂O) and saturated aqueous NaHCO₃ solution, and the organic phase was separated. The aqueous layer was extracted with AcOEt and the combined organic layer was washed with brine, dried MgSO₄, and evaporated under reduced pressure. The residue obtained was purified by flash column chromatography on silica gel (60% ethyl acetate-hexane) to give 78 (35.52 mg, 80% yield) as a white solid (melting point 97-99° C.).

78 was confirmed as follows: ¹H NMR (500 MHz, CDCl₃) δ 8.70 (t, J=2.0 Hz, 1H), 8.54 (d, J=8.0 Hz, 1H), 7.90 (d, J=8.0 Hz, 1H), 7.62 (t, J=8.0 Hz, 1H), 7.48 (d, J=7.5 Hz, 2H), 7.42-7.40 (m, 3H), 7.34 (t, J=7.5 Hz, 1H), 7.27-7.25 (m, 2H), 7.01 (dd, J=7.0 Hz, J=2.0 Hz, 1H), 5.15 (s, 2H), 2.71 (s, 3H).

### 5. 1-(3-Methoxy-phenyl)-1-(5-methyl-1,3,4-oxadiazol-2-yl)-methanol (80)

The synthesis was carried out analogous to the preparation of 77 using 79 (0.14 g, 1.03 mmol) and 63 (0.29 g, 3.45 mmol). Purification by flash column chromatography on silica gel gave compound 80 (0.12 g, 53.4% yield) as a viscous liquid.

80 was confirmed as follows: ¹H NMR (500 MHz, CDCl₃) δ 7.30 (t, J=7.5 Hz, 1H), 7.06-7.02 (m, 2H), 6.90 (dd, J=7.5 Hz, J=2.5 Hz, 1H), 6.05 (d, J=5.0 Hz, 1H), 3.83 (s, 3H), 3.67 (d, J=5.0 Hz, 1H), 2.49 (s, 3H).

### 6. 1-(3-Methoxy-phenyl)-1-(5-methyl-1,3,4-oxadiazol-2-yl)-ketone (81)

1-(3-Methoxy-phenyl)-1-(5-methyl-1,3,4-oxadiazol-2-yl)-ketone (81) was synthesized as in 78 using 80 (0.1 g, 0.454 mmol) and Dess-Martin periodinane (0.38 g, 0.9 mmol) in wet CH₂Cl₂ (10 mL). Purification by flash column chromatography on silica gel gave compound 81 (0.080 g, 82% yield) as a viscous liquid.

81 was confirmed as follows: ¹H NMR (500 MHz, CDCl₃) δ 8.10 (dt, J=7.5 Hz, J=1.5 Hz, 1H), 7.99 (t, J=1.5 Hz, 1H), 7.47 (t, J=7.5 Hz, 1H), 7.24 (dd, J=7.5 Hz, J=1.5 Hz, 1H), 3.90 (s, 3H), 2.70 (s, 3H).

### Synthesis of α-Keto-oxadiazole 81.1

α-Keto-oxadiazole 81.1 was synthesized by a method depicted in Scheme 18.

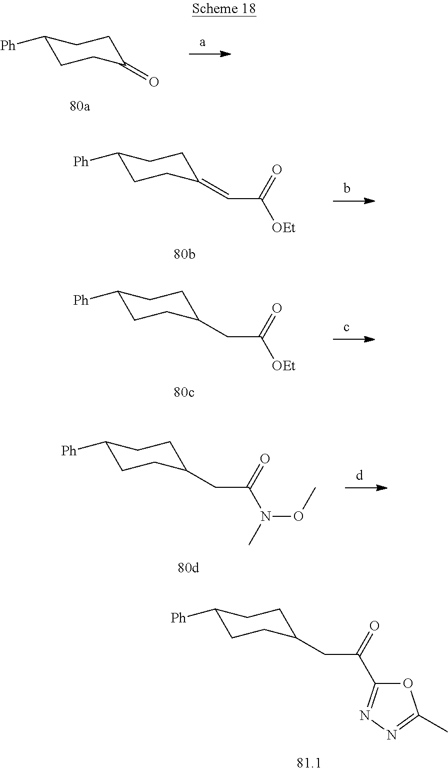

Reagents and conditions for the steps in Scheme 18 were as follows: Step a: NaH, (EtO)₂POCH₂CO₂Et, 0° C.—RT, 2 hours, 91%; Step b: H2, Pd/C, 50 p.s.i., RT, 6 hours, 95%; Step c: Me(OMe)NH.HCl, i-Pr-MgCl, THF, −20° C. to 0° C. 87%; Step d: 63, n-BuLi, MgBr₂ Et₂O, THF, −78° C. to −50° C., 2 hours, then addition of 80d −30 to 0° C., 4 hours, 56%.

### 1. Ethyl-2-(4-phenylcyclohexylidene) acetate 80b

To a solution of triethyl phosphonoacetate (3.5 equivl) in anhydrous THF, at 0° C. under an argon atmosphere, was added sodium hydride (3.5 equiv 60% dispersion in mineral oil). The suspension was stirred for 15 min at the same temperature, and a solution of 80a (1 equiv) in anhydrous THF was added dropwise. The reaction was gradually warmed to RT and stirred for additional 2 hours and upon completion was quenched by the addition of saturated aqueous NH₄Cl. The mixture diluted with diethyl ether (100 mL) and the aqueous phase extracted with diethyl ether. The combined organic layer was washed with brine, dried over MgSO₄, and the solvent was evaporated under reduced pressure. The residue was purified by flash column chromatography on silica gel using (20% EtOAc:Heaxne) to 80b as a colorless liquid in 91% yield.

80b was confirmed as follows: ¹H NMR (500 MHz, CDCl₃) δ 7.33-7.30 (m, 2H), 7.40-7.20 (m, 3H), 5.79 (s, 1H), 4.19 (q, J=7.0 Hz, 2H), 3.99 (d, J=15.0 Hz, 1H), 2.81 (dt, J=15.0 Hz, J=3.5 Hz, 1H), 2.39 (dq, J=13.2 Hz, J=3.5 Hz, 2H), 2.07 (dq, J=13.2 Hz, J=3.5 Hz, 2H), 1.66 (dq, J=13.2 Hz, J=4.0 Hz, 2H), 1.31 (t, J=7.0 Hz, 3H).

### 2. Ethyl-2-(4-phenylcyclohexyl)acetate (80c)

A mixture of 80b (1 equiv.) and 10% Pd/C (0.16 equiv) in EtOH was placed in a Parr apparatus (Parr Instrument Co, Moline, Ill.) and treated with hydrogen at 50 psi for 6 hours. The catalyst was removed by filtration through a pad of celite and the filtrate was evaporated under reduced pressure to give 80c as a colorless liquid in 95% yield, used in the next step without further purification.

80c was confirmed as follows: ¹H NMR (500 MHz, CDCl₃) δ 7.34-7.30 (m, 2H), 7.28-7.20 (m, 3H), 4.18 (q, J=7.2 Hz, 2H), 2.53-2.46 (m, 2H), 2.70 (d, J=6.5 Hz, 2H), 1.95-1.86 (m, 5H), 1.55 (dq, J=12.5 Hz, J=3.5 Hz, 2H), 1.30 (t, J=7.2 Hz, 3H), 1.21 (dq, J=12.5, J=2 Hz, 2H).

### 3. N-Methoxy-N′-methyl-2-(4-phenylcyclohexyl)acetamide (80d)

N-Methoxy-N′-methyl-2-(4-phenylcyclohexyl)acetamide (80d) was prepared as described for 68 (scheme 16).

80d was confirmed as follows: ¹H NMR (500 MHz, CDCl₃) δ 7.33-7.29 (m, 2H), 7.23-7.18 (m, 3H), 3.72 (s, 3H), 3.22 (s, 3H), 2.49 (dt, J=14.5 Hz, J=4.5 Hz, 1H), 2.39 (d, J=6.5 Hz, 2H), 1.96-1.90 (m, 5H), 1.55 (dq, J=13.2 Hz, J=3.0 Hz, 2H), 1.22-1.16 (m, 2H).

### 4. 1-(5-Methyl-1,3,4-oxadiazol-2-yl)-2-(4-phenylcyclohexyl)ethanone (81.1)

1-(5-Methyl-1,3,4-oxadiazol-2-yl)-2-(4-phenylcyclohexyl)ethanone 81.1 was synthe-sized as described for 73 (scheme 16).

81.1 was confirmed as follows: ¹H NMR (500 MHz, CDCl₃) δ 7.32-7.28 (m, 2H), 7.23-7.17 (m, 3H), 3.07 (d, J=7.0 Hz, 2H), 2.65 (s, 3H), 2.49 (tt, J=13.5 Hz, J=4.5 Hz, 1H), 2.18-2.08 (m, 1H), 1.92 (d, J=11.5 Hz, 4H), 1.55-1.48 (m, 2H) 1.31-1.22 (m, 2H).

### Synthesis of saccharin analogs (83.1-83.9 and 84)

Saccharin analogs 83.1, 83.2, 83.3, 83.4, 83.5, 83.6, 83.7, 83.8, 83.9 and 84 (shown in Scheme 19) were synthesized by a method depicted in Scheme 19 starting from commercially available saccharin (82) and the appropriate bromide.

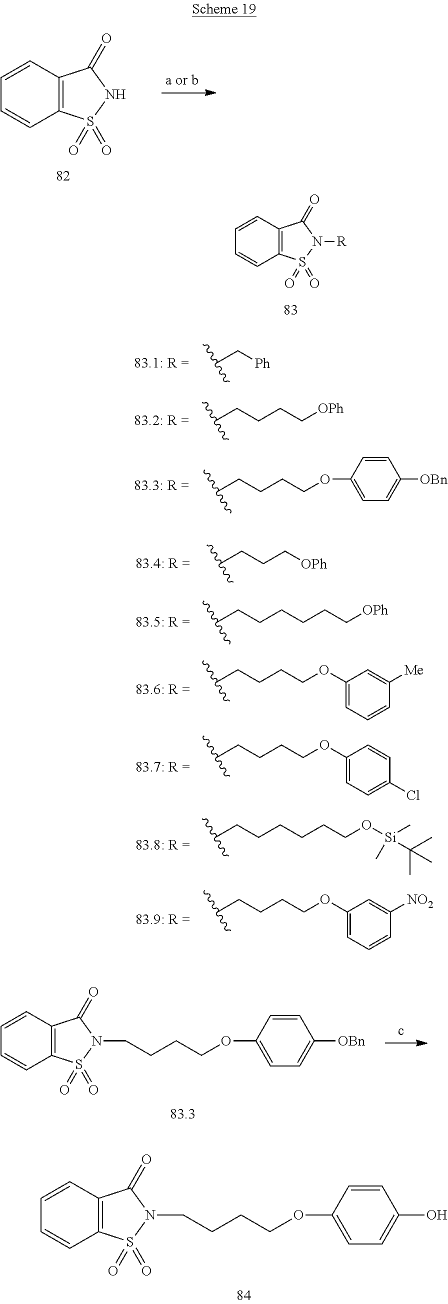

Reagents and conditions for the steps in Scheme 19 were as follows:

Step a: (i) NaH, THF, 0° C. to RT, 1 hour, (ii) RBr, DMF, 80° C., 4 hours, 66-67%; Step b: (i) NaH, DMF, RT, 15 min, (ii) RBr, DMF, microwave, 150° C., 10 min, 45-65%; Step c: 1,4-cyclohexadiene, Pd/C, EtOH, 50° C., 2 hours, 56%.

### 1. N-(Phenylmethyl)saccharin (83.1)

To a stirred solution of saccharin 82 (0.154 g, 0.75 mmol) in anhydrous THF (10 mL) at 0° C., under nitrogen atmosphere was added NaH (0.019 g, 0.8 mmol, using a 60% dispersion in mineral oil) and the resulting slurry was gradually warmed to RT over 1 hour period. Solvent was removed under reduced pressure, and the saccharin sodium salt was dissolved in anhydrous DMF (5 mL). To this solution, was added a solution of benzyl bromide (0.051 g, 0.3 mmol) in DMF (5 mL), under nitrogen, at RT, and the mixture warmed to 80° C. and stirred for 4 hours. The reaction mixture was cooled to RT and diluted with dropwise addition of water (5 mL) and AcOEt (20 mL). The organic layer was separated and the aqueous layer extracted with AcOEt. The combined organic layer was washed with brine, dried (MgSO₄), and the solvent removed in vacuo. The residue was purified by flash column chromatography on silica gel (50% diethyl ether-hexane) to give 83.1 (0.054 g, 66% yield), as a white solid (melting point 106-108° C.).

83.1 was confirmed as follows: ¹H NMR (500 MHz, CDCl₃) δ 8.06 (d, J=7.0 Hz, 1H), 7.93 (d, J=7.0 Hz, 1H), 7.87 (td, J=7.0 Hz, J=1.2 Hz, 1H), 7.83 (td, J=7.0 Hz, J=1.2 Hz, 1H), 7.51 (d, J=7.5 Hz, 2H), 7.36 (t, J=7.5 Hz, 2H), 7.31 (t, J=7.0 Hz, 1H), 4.91 (s, 2H).

### 2. N-(4-Phenoxy-butyl)saccharin (83.2)

The synthesis was carried out analogous to the preparation of 83.1 using 82 (0.23 g, 1.25 mmol), NaH (0.030 g, 1.25 mmol) and 4-phenoxy-butyl bromide (0.115 g, 0.5 mmol) in DMF (5 mL). Purification by flash column chromatography on silica gel gave 83.2 (0.1 g, 67% yield) as a white solid (melting point 92-94° C.).

83.2 was confirmed as follows: ¹H NMR (500 MHz, CDCl₃) δ 8.07 (d, J=7.0 Hz, 1H), 7.93 (d, J=7.0 Hz, 1H), 7.87 (td, J=7.0 Hz, J=1.2 Hz, 1H), 7.83 (td, J=7.0 Hz, J=1.2 Hz, 1H), 7.27 (t, J=7.5 Hz, 2H), 6.93 (t, J=7.5 Hz, 1H), 6.90 (d, J=7.5 Hz, 2H), 4.02 (t, J=6.5 Hz, 2H), 3.88 (t, J=7.2 Hz, 2H), 2.07 (quintet, J=6.9 Hz, 2H), 1.92 (quintet, J=6.9 Hz, 2H).

### 3. N-[4-(4-Benzyloxy-phenoxy)-butyl]saccharin (83.3)

The synthesis was carried out analogous to the preparation of 83.1 using 82 (0.307 g, 1.5 mmol), NaH (0.036 g, 1.5 mmol) and 4-(4-benzyloxy-phenoxy)-butyl bromide (0.2 g, 0.6 mmol) in DMF (5 mL). Purification by flash column chromatography on silica gel gave 83.3 (0.150 g, 66% yield) as a white solid (melting point 82-84° C.).

83.3 was confirmed as follows: ¹H NMR (500 MHz, CDCl₃) δ 8.06 (d, J=7.0 Hz, 1H), 7.92 (d, J=7.0 Hz, 1H), 7.87 (td, J=7.0 Hz, J=1.2 Hz, 1H), 7.83 (td, J=7.0 Hz, J=1.2 Hz, 1H), 7.42 (d, J=7.5 Hz, 2H), 7.37 (t, J=7.5 Hz, 2H), 7.31 (t, J=7.5 Hz, 1H), 6.89 (d, J=9.0 Hz, 2H), 6.82 (d, J=9.0 Hz, 2H), 5.00 (s, 2H), 3.97 (t, J=6.4 Hz, 2H), 3.86 (t, J=7.2 Hz, 2H), 2.05 (quintet, J=6.9 Hz, 2H), 1.89 (quintet, J=6.9 Hz, 2H).

### 4. N-(3-Phenoxypropyl)saccharin (83.4)

The title compound was synthesized analogously to 83.8 (see experimental below), using a solution of saccharin (92 mg, 0.5 mmol) in DMF (anhydrous, 4 mL), NaH (60% dispersion in mineral oil, 21 mg, 0.52 mmol) and a solution of 3-phenoxypropyl bromide (130 mg, 0.6 mmol) in anhydrous DMF (1 mL). The crude obtained after workup was purified by flash column chromatography on silica gel (25% ethyl acetate-petroleum ether) to give 83.4 as a white solid (melting point 83-86° C.) in 65% yield (130 mg).

83.4 was confirmed as follows: ¹H NMR (500 MHz, CDCl₃) δ 8.08 (dd, J=7.5 Hz, J=1.5 Hz, 1H), 7.95 (dd, J=7.5 Hz, J=1.5 Hz 1H), 7.89 (td, J=7.5 Hz, J=1.5 Hz, 1H), 7.85 (td, J=7.5 Hz, J=1.5 Hz, 1H), 7.30 (t, J=8.0 Hz, 2H), 6.97 (t, J=8.0 Hz, 1H), 6.93 (d, J=8.0 Hz, 2H), 4.11 (t, J=6.2 Hz, 2H), 4.04 (t, J=7.1 Hz, 2H), 2.36 (quintet, J=7.5 Hz, 2H).

### 5. N-(6-Phenoxyhexyl)saccharin (83.5)

The title compound was synthesized analogously to 83.8 (see description below) using a solution of saccharin (92 mg, 0.5 mmol) in anhydrous DMF (4 mL), NaH (60% dispersion in mineral oil, 21 mg, 0.52 mmol), and a solution of 6-phenoxybutyl bromide (154 mg, 0.6 mmol) in DMF (anhydrous, 1 mL). The crude product obtained after workup was purified by flash column chromatography on silica gel (20% ethyl acetate-petroleum ether) to give 83.5 as a white solid (melting point 64-66° C.) in 50% yield (90 mg).

83.5 was confirmed as follows: ¹H NMR (500 MHz, CDCl₃) δ 8.07 (dd, J=7.5 Hz, J=1.5 Hz, 1H), 7.93 (dd, J=7.5 Hz, J=1.5 Hz 1H), 7.88 (td, J=7.5 Hz, J=1.5 Hz, 1H), 7.84 (td, J=7.5 Hz, J=1.5 Hz, 1H), 7.28 (t, J=7.5 Hz, 2H), 6.94 (t, J=7.5 Hz, 1H), 6.90 (d, J=7.5 Hz, 2H), 3.98 (t, J=6.5 Hz, 2H), 3.81 (t, J=7.5 Hz, 2H), 1.91 (quintet, J=7.2 Hz, 2H), 1.83 (quintet, J=7.2 Hz, 2H), 1.61-1.48 (m, 4H).

### 6. N-[4-(3-Methyl-phenoxy)-butyl]saccharin (83.6)

The title compound was synthesized analogously to 83.8 (see description below) using a solution of saccharin (92 mg, 0.5 mmol) in anhydrous DMF (4 mL), NaH (60% dispersion in mineral oil, 21 mg, 0.52 mmol) and a solution of 1-(4-bromobutoxy)-3-methylbenzene (146 mg, 0.6 mmol) in DMF (anhydrous, 1 mL). The crude product obtained after workup was purified by flash column chromatography on silica gel (25% ethyl acetate-petroleum ether) to give 83.6 as a viscous liquid in 55% yield (95 mg).

83.6 was confirmed as follows: ¹H NMR (500 MHz, CDCl₃) δ 8.08 (dd, J=7.5 Hz, J=1.5 Hz, 1H), 7.94 (dd, J=7.5 Hz, J=1.5 Hz 1H), 7.88 (td, J=7.5 Hz, J=1.5 Hz, 1H), 7.85 (td, J=7.5 Hz, J=1.5 Hz, 1H), 7.17 (t, J=7.2 Hz, 1H), 6.77 (dd, J=7.2 Hz, 1.5 Hz, 1H), 6.74 (t, J=1.5 Hz, 1H), 6.72 (dd, J=7.2 Hz, J=1.5 Hz, 1H), 4.03 (t, J=6.5 Hz, 2H), 3.89 (t, J=7.5 Hz, 2H), 2.34 (s, 3H), 2.08 (quintet, J=7.5 Hz, 2H), 1.92 (quintet, J=7.0 Hz, 2H).

### 7. N-[4-(4-Chloro-phenoxy)-butyl]saccharin (83.7)

The title compound was synthesized analogously to 83.8 (see description below) using a solution of saccharin (92 mg, 0.5 mmol) in anhydrous DMF (4 mL), NaH (60% dispersion in mineral oil, 21 mg, 0.52 mmol) and a solution of 1-(4-bromobutoxy)-4-chlorobenzene (158 mg, 0.6 mmol) in anhydrous DMF (1 mL). The crude product obtained after workup was purified by flash column chromatography on silica gel (25% ethyl acetate-petroleum ether) to give 83.7 as a white solid (m p 85-88° C.) in 57% yield (104 mg).

83.7 was confirmed as follows: ¹H NMR (500 MHz, CDCl₃) δ 8.08 (d, J=7.2 Hz, 1H), 7.94 (d, J=7.2 Hz, 1H), 7.89 (td, J=7.2 Hz, J=1.2 Hz, 1H), 7.85 (td, J=7.2 Hz, J=1.2 Hz, 1H), 7.23 (d, J=8.7 Hz, 2H), 6.84 (d, J=8.7 Hz, 2H), 4.01 (t, J=6.2 Hz, 2H), 3.89 (t, J=7.5 Hz, 2H), 2.07 (quintet, J=7.5 Hz, 2H), 1.92 (quintet, J=8.2 Hz, 2H).

### 8. N-(6-tert-Butyldimethylsilyloxy-hexyl)saccharin (83.8)

To a solution of saccharin (92 mg, 0.5 mmol) in anhydrous DMF (4 mL), at RT, under a nitrogen atmosphere, was added NaH (60% dispersion in mineral oil, 21 mg, 0.52 mmol). The mixture was stirred at the same temperature for additional 15 min, a solution of (6-bromohexyloxy)-tert-butyldimethylsilane (177 mg, 0.6 mmol) in DMF (1 mL) was added, and the resulting mixture microwaved at 150° C. for 10 min. The reaction mixture was cooled to RT and diluted with water (5 mL) and AcOEt (10 mL). The organic layer was separated, and the aqueous layer extracted with AcOEt (2×10 mL). The combined organic layer was washed with brine, dried (MgSO₄), and the solvent removed in vacuum. The residue was purified by flash column chromatography on silica gel (20% ethyl acetate-petroleum ether) to give 83.8 (89 mg, 45% yield), as a viscous liquid.

83.8 was confirmed as follows: ¹H NMR (400 MHz, CDCl₃) δ 8.07 (dd, J=7.4 Hz, J=1.5 Hz, 1H), 7.93 (dd, J=7.4 Hz, J=1.5 Hz 1H), 7.88 (td, J=7.4 Hz, J=1.5 Hz, 1H), 7.84 (td, J=7.4 Hz, J=1.5 Hz, 1H), 3.79 (t, J=7.5 Hz, 2H), 3.62 (t, J=6.4 Hz, 2H), 1.88 (quintet, J=7.3 Hz, 2H), 1.55 (quintet, J=6.7 Hz, 2H), 1.50-1.34 (m, 4H), 0.91 (s, 9H), 0.07 (s, 6H).

### 9. N-[4-(3-Nitro-phenoxy)-butyl]saccharin (83.9)

The title compound was synthesized analogously to 83.8, using a solution of saccharin (92 mg, 0.5 mmol) in anhydrous DMF (4 mL), NaH (60% dispersion in mineral oil, 21 mg, 0.52 mmol) and a solution of 1-(4-bromobutoxy)-3-nitrobenzene (164 mg, 0.6 mmol) in anhydrous DMF (1 mL). The crude product obtained after workup was purified by flash column chromatography on silica gel (25% ethyl acetate-petroleum ether) to give 83.9 as a white solid (m p 87-89° C.) in 55% yield (95 mg).

83.9 was confirmed as follows: ¹H NMR (500 MHz, CDCl₃) δ 8.09 (dd, J=7.2 Hz, J=1.5 Hz, 1H), 7.95 (dd, J=7.2 Hz, J=1.5 Hz 1H), 7.90 (td, J=7.2 Hz, J=1.5 Hz, 1H), 7.86 (td, J=7.2 Hz, J=1.5 Hz, 1H), 7.83 (dd, J=8.0 Hz, J=1.8 Hz, 1H), 7.74 (t, J=1.8 Hz, 1H), 7.43 (t, J=8.0 Hz, 1H), 7.24 (dd, J=8.0 Hz, J=1.8 Hz, 1H), 4.12 (t, J=6.2 Hz, 2H), 3.91 (t, J=7.0 Hz, 2H), 2.10 (quintet, J=7.5 Hz, 2H), 1.98 (quintet, J=7.2 Hz, 2H).

### 10. N-[4-(4-Hydroxy-phenoxy)-butyl]saccharin (84)

To a stirred solution of 83.3 (0.1 g, 0.23 mmol) in EtOH (5 mL) was added 10% Pd/C (0.1 g, 100% w/w) and 1,4-cyclohexadiene (92 mg, 1.15 mmol) and the resulting suspension was stirred vigorously at 50° C. for 2 hours. The reaction mixture was cooled to RT, the catalyst was removed by filtration through Celite, and the filtrate was evaporated under reduced pressure. The residue was purified by flash column chromatography on silica gel (60% diethyl ether-hexane) to give 84 (0.044 g, 56% yield) as a white solid (melting point 107-109° C.).

84 was confirmed as follows: ¹H NMR (500 MHz, CDCl₃) δ 8.06 (d, J=7.0 Hz, 1H), 7.92 (d, J=7.0 Hz, 1H), 7.87 (td, J=7.0 Hz, J=1.2 Hz, 1H), 7.83 (td, J=7.0 Hz, J=1.2 Hz, 1H), 6.79 (d, J=9.0 Hz, 2H), 6.74 (d, J=9.0 Hz, 2H), 4.38 (br s, 1H), 3.96 (t, J=6.4 Hz, 2H), 3.89 (t, J=7.2 Hz, 2H), 2.05 (quintet, J=6.9 Hz, 2H), 1.89 (quintet, J=6.9 Hz, 2H).

### Synthesis of α-keto-esters and α,α-difluoromethylene-ketones

α-Keto-esters 87.1-4 and 87.7 as well as α,α-difluoromethylene-ketones 89.1, 89.2, 89.4, and 89.7-14 were synthesized by the methods depicted in Scheme 20. 3-Benzyloxyphenol (85.1), 4-benzyloxyphenol (85.5), 2-benzyloxyphenol (85.6), 4-phenoxybutyl bromide (86.2), 5-phenoxypentyl bromide (86.4), 6-phenoxyhexyl bromide (86.7), 3-methyl-phenyl magnesium bromide, 2-bromopyridine, and 3-bromopyridine were commercially available materials. The 2-methyl-oxadiazole (63), 2-bromopyridine, and 3-bromopyridine were served as precursors for the preparation of the respective organolithium reagent using commercially available n-BuLi.

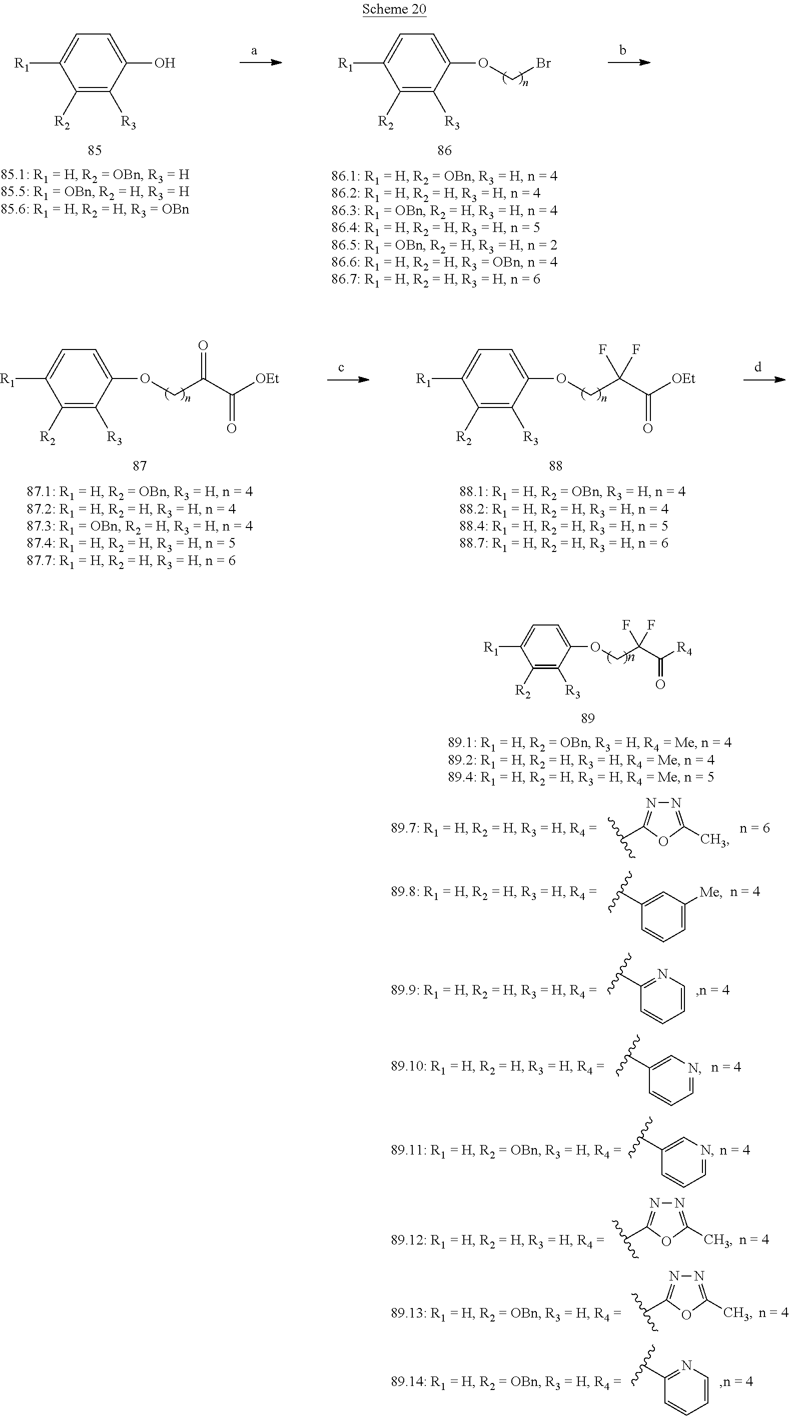

Reagents and conditions for the steps in Scheme 20 were as follows: Step a: Br-(CH₂)ₙ-Br, K₂CO₃, acetone or MeCN, reflux, 10-12 hours, 68-74% for n=4, 5, 6 or NaH, DMF, RT to 80° C., 6 hours, 64% for n=2; Step b: Mg, THF, RT to gentle reflux, then addition to (COOEt)₂, THF, −78° C. to 10° C., 1 hour, 45-65%; Step c: DAST, CHCl₃, reflux, 3 hours, 72-88% or DAST CHCl₃, microwave, 100° C., 300 W, 3-5 min, 74-86%; Step d: R₄Li, THF or Et₂O, −78° C. to RT, 1-2 hours, 65-78%.

### 1. Bromides (86)

A mixture of phenol derivative 85 (1 equiv.), α,ω-dibromoalkane (1.5 equiv.) and anhydrous potassium carbonate was stirred under refluxed in dry acetone or acetonitrile for 10-12 hours, cooled to RT, and solid materials were filtered off The filtrate was evaporated, water was added to the residue, and the mixture was extracted with diethyl ether. The ethereal layer was washed with 10% sodium hydroxide solution, water, brine, dried (MgSO₄), and concentrated under reduced pressure. Purification by flash column chromatography on silica gel (diethyl ether-hexane) gave compound 86 as colorless viscous oil in 68-74% yields.

### 2. Bromide 86.5

A mixture of 4-benzyloxy-phenol 85.5 (1 equiv.) and NaH in anhydrous dimethylformamide was stirred at RT for 15 min under argon. To this mixture was added 1,2-dibromoethane (1.5 equiv.) and stirring was continued at 80° C. for 6 hours. The reaction mixture was cooled to RT, diluted with water and extracted with diethyl ether. The ethereal layer was washed with water and brine, dried (MgSO₄), and concentrated under reduced pressure. Purification by flash column chromatography on silica gel (diethyl ether-hexane) gave product 86.5 in 64% yield.

### 3. Selected data of synthesized bromides (86)

### 1-Bromo-4-[3-(benzyloxy)phenoxy]butane (86.1)

86.1 was confirmed as follows: ¹H NMR (500 MHz, CDCl₃) δ 7.43 (d, J=7.6 Hz, 2H), 7.39 (t, J=7.6 Hz, 2H), 7.32 (t, J=7.6 Hz, 1H), 7.17 (t, J=8.5 Hz, 1H), 6.58 (dd, J=8.5 Hz, J=2.5 Hz, 1H), 6.54 (t, J=2.5 Hz, 1H), 6.50 (dd, J=8.5 Hz, J=2.5 Hz, 1H), 5.04 (s, 2H), 3.97 (t, J=6.3 Hz, 2H), 3.48 (t, J=6.5 Hz, 2H), 2.06 (m, 2H), 1.93 (m, 2H).

### 1-Bromo-2-[4-(benzyloxy)phenoxy]ethane (86.5)

86.5 was confirmed as follows: ¹H NMR (500 MHz, CDCl₃) δ 7.43 (d, J=7.3 Hz, 2H), 7.38 (t, J=7.3 Hz, 2H), 7.32 (t, J=7.3 Hz, 1H), 6.92 (d, J=8.5 Hz, 2H), 6.85 (d, J=8.5 Hz, 2H), 5.07 (s, 2H), 4.24 (t, J=6.5 Hz, 2H), 3.61 (t, J=6.5 Hz, 2H).

### 4. α-Keto-esters (87)

To a three-neck round bottom flask containing Mg turnings (1.2 equiv.) equipped with a magnetic stirrer and dimroth condenser was added a solution of alkyl bromide 86 (1 equiv.) in anhydrous THF via syringe and external heating under argon atmosphere. The reaction mixture was refluxed gently for 30-40 min and then it was cooled to RT, before conveying it to a dropping funnel. The Grignard reagent was added dropwise to a solution of diethyl oxalate (1.5 equiv.) in THF at −78° C. The reaction mixture was warmed to 10° C. within 1 hour and then was quenched by the addition of saturated ammonium chloride solution. The organic layer was separated, the aqueous layer was extracted with diethyl ether and the combined organic layer was washed with brine, dried over MgSO₄, and evaporated. The residue was purified by flash column chromatography on silica gel (diethyl ether-hexane) to give pure compound 87 in 45-65% yields.

### 5. Selected data of synthesized α-Keto-esters (87)

### 2-Oxo-6-[4-(benzyloxy)phenoxy]hexanoic acid ethyl ester (87.3)

87.3 was confirmed as follows: ¹H NMR (500 MHz, CDCl₃) δ 7.44 (d, J=7.5 Hz, 2H), 7.40 (t, J=7.5 Hz, 2H), 7.34 (t, J=7.5 Hz, 1H), 6.91 (d, J=9.0 Hz, 2H), 6.83 (d, J=9.0 Hz, 2H), 5.03 (s, 2H), 4.33 (q, J=7.5 Hz, 2H), 3.94 (t, J=5.8 Hz, 2H), 2.95 (t, J=7.0 Hz, 2H), 1.90-1.79 (m, 4H), 1.38 (t, J=7.5 Hz, 3H).

2-Oxo-7-phenoxy-heptanoic acid ethyl ester (87.4)

87.4 was confirmed as follows: ¹H NMR (500 MHz, CDCl₃) δ 7.27 (t, J=7.6 Hz, 2H), 6.93 (t, J=7.6 Hz, 1H), 6.88 (d, J=7.6 Hz, 2H), 4.32 (q, J=7.5 Hz, 2H), 3.96 (t, J=6.0 Hz, 2H), 2.88 (t, J=7.5 Hz, 2H), 1.81 (qt, J=6.5 Hz, 2H), 1.72 (qt, J=7.5 Hz, 2H), 1.56-1.49 (m, 2H), 1.37 (t, J=7.5 Hz, 3H).

### 6. α,α-Difluoro-esters (88)

To a stirred solution of α-keto-ester 87 (1 equiv.) in anhydrous chloroform at RT under an argon atmosphere was added diethylaminosulfur trifluoride (1.1 equiv.). The reaction mixture was heated under gentle reflux for 3 hours then it was cooled to RT and poured into ice-water. The organic layer was separated, washed with sat. NaHCO₃ solution and dried over MgSO₄. Volatiles were removed under reduced pressure and the crude product was purified by flash chromatography on silica gel (diethyl ether-hexane) to give pure compound 88 in 72-88% yields.

Alternatively, the reaction mixture was heated using microwave irradiation (300 W, 100° C., 3-5 min) This was followed by work up and purification as described above to give compound 88 in 74-86% yields.

### 7. Selected data of synthesized α,α-difluoro-esters (88)

### 2,2-Difluoro-6-[3-(benzyloxy)phenoxy]hexanoic acid ethyl ester (88.1)

88.1 was confirmed as follows: ¹H NMR (500 MHz, CDCl₃) δ 7.43 (d, J=7.5 Hz, 2H), 7.38 (t, J=7.5 Hz, 2H), 7.32 (t, J=7.5 Hz, 1H), 7.17 (t, J=8.5 Hz, 1H), 6.57 (dd, J=8.5 Hz, J=2.5 Hz, 1H), 6.53 (t, J=2.5 Hz, 1H), 6.50 (dd, J=8.5 Hz, J=2.5 Hz, 1H), 5.04 (s, 2H), 4.32 (q, J=7.4 Hz, 2H), 3.94 (t, J=6.5 Hz, 2H), 2.19-2.08 (m, 2H), 1.82 (qt, J=7.9 Hz, 2H), 1.71-1.63 (m, 2H), 1.34 (t, J=7.4 Hz, 3H).

### 8. α,α-difluoromethylene-ketones (89)

To a stirred solution of α,α-difluoro-ester 88 (1 equiv.) in anhydrous THF or diethyl ether at −78° C. under an argon atmosphere was added the appropriate organolithium or organomagnesium reagent (1.1-1.5 equiv.) dropwise. The reaction mixture was allowed to warm to RT over 1-2 hours period, and then was quenched by the addition of saturated ammonium chloride solution. The organic phase was separated, the aqueous layer was extracted with diethyl ether or methylene chloride, and the combined organic layer was washed with water and brine, dried (MgSO₄), and evaporated under reduced pressure. Purification by flash column chromatography on silica gel (diethyl ether-hexane or acetone-hexane) gave compound 89 in 65-78% yields.

### 9. Selected data of synthesized α,α-difluoromethylene-ketones (89)

### 3,3-Difluoro-8-phenoxy-2-octanone (89.4)

89.4 was confirmed as follows: ¹H NMR (500 MHz, CDCl₃) δ 7.28 (m as t, J=7.5 Hz, 2H), 6.93 (m as t, J=7.5 Hz, 1H), 6.89 (m as d, J=7.5 Hz, 2H), 3.96 (t, J=6.3 Hz, 2H), 2.33 (t, J=1.5 Hz, 3H), 2.06-1.95 (m, 2H), 1.83-1.77 (m, 2H), 1.56-1.51 (m, 4H).

### 2,2-Difluoro-8-phenoxy-1-(5-methyl-1,3,4-oxadiazol-2-yl)-octan-1-one (89.7, mixture of keto and hydrate form in a 2.2:1 ratio)

89.7 was confirmed as follows: ¹H NMR (500 MHz, CDCl₃) δ 7.28 (t, J=7.2 Hz, 2H, keto form), 7.27 (t, J=7.2 Hz, 2H, hydrate form), 6.93 (t, J=7.2 Hz, 1H from keto form and 1H from hydrate form, overlapping), 6.89 (d, J=7.2 Hz, 2H, hydrate form), 6.88 (d, J=7.2 Hz, 2H, keto form), 4.46 (br s, 2H, OH, hydrate form), 3.95 (t, J=6.5 Hz, 2H, hydrate form), 3.94 (t, J=6.5 Hz, 2H, keto form), 2.70 (s, 3H, keto form), 2.60 (s, 3H, hydrate form), 2.44-2.32 (m, 2H, keto form), 2.16-2.00 (m, 2H, hydrate form), 1.82-1.74 (m, 2H from keto form and 2H from hydrate form, overlapping), 1.64-1.40 (m, 6H from keto form and 6H from hydrate form, overlapping); IR (neat) 3237 (br), 2942, 2866, 1734, 1600 cm⁻¹.

### 2,2-Difluoro-6-phenoxy-1-(pyridin-2-yl)-hexan-1-one (89.9)

89.9 was confirmed as follows: ¹H NMR (500 MHz, CDCl₃) δ 8.65 (m as dt, J=6.1 Hz, J=0.5 Hz, 1H), 8.03 (m as dt, J=8.0 Hz, J=1.0 Hz, 1H), 7.81 (ddd, J=8.0 Hz, J=8.0 Hz, J=1.7 Hz, 1H), 7.44 (ddd, J=8.0 Hz, J=6.1 Hz, J=1.7 Hz, 1H), 7.19 (t, J=7.8 Hz, 2H), 6.85 (t, J=7.8 Hz, 1H), 6.78 (d, J=7.8 Hz, 2H), 3.95 (t, J=6.5 Hz, 2H), 2.60-2.43 (m, 2H), 1.73 (qt, J=7.3 Hz, 2H), 1.67-1.62 (m, 2H).

### 2,2-Difluoro-6-phenoxy-1-(pyridin-3-yl)-hexan-1-one (89.10, hydrate form)

89.10 was confirmed as follows: ¹H NMR (500 MHz, DMSO-d₆) δ 8.70 (d, J=1.5 Hz, 1H), 8.53 (dd, J=5.2 Hz, J=1.5 Hz, 1H), 7.88 (dt, J=7.6 Hz, J=1.4 Hz, 1H), 7.39 (dd, J=7.6 Hz, J=5.2 Hz, 1H), 7.27 (t, J=7.4 Hz, 2H), 7.03 (s, 2H), 6.94-6.88 (m, 3H), 3.95 (t, J=6.2 Hz, 2H), 2.12-1.99 (m, 2H), 1.74 (qt, J=7.1 Hz, 2H), 1.61-1.53 (m, 2H).

### 2,2-Difluoro-6-[3-(benzyloxy)phenoxy]-1-(pyridin-3-yl)-hexan-1-one (89.11, hydrate form)

89.11 was confirmed as follows: ¹H NMR (500 MHz, CD₃OD) δ 8.68 (d, J=1.7 Hz, 1H), 8.52 (dd, J=5.1 Hz, J=1.7 Hz, 1H), 7.98 (dt, J=8.0 Hz, J=1.4 Hz, 1H), 7.46 (dd, J=8.0 Hz, J=5.1 Hz, 1H), 7.42 (d, J=7.4 Hz, 2H), 7.36 (t, J=7.4 Hz, 2H), 7.29 (t, J=7.4 Hz, 1H), 7.13 (t, J=8.2 Hz, 1H), 6.55 (dd, J=8.2 Hz, J=2.5 Hz, 1H), 6.51 (t, J=2.5 Hz, 1H), 6.47 (dd, J=8.2 Hz, J=2.5 Hz, 1H), 5.04 (s, 2H), 3.92 (t, J=6.5 Hz, 2H), 2.02 (m, 2H), 1.76 (qt, J=7.2 Hz, 2H), 1.64 (qt, J=7.3 Hz, 2H).

### Synthesis of α-Keto-esters (91.1-6) and α,α-difluoromethylene-ketones (93.1, 93.2, 93.5, and 93.7-9)

α-Keto-esters 91.1-6 as well as α,α-difluoromethylene-ketones 93.1, 93.2, 93.5, and 93.7-9 were synthesized by the methods depicted in Scheme 21. 4-Bromobiphenyl (90.1), bromobenzene (90.2), 3-bromobiphenyl (90.3), 2-bromobiphenyl (90.4), benzoxazole, benzothiazole, 2,6-dibromopyridine, and 2-(4-bromophenyl)pyridine were commercially available materials. The 2-methyl-oxadiazole (63), benzoxazole, benzothiazole, 2,6-dibromopyridine, and 2-(4-bromophenyl)pyridine were served as precursors for the preparation of the respective organolithium agents using commercially available n-BuLi.

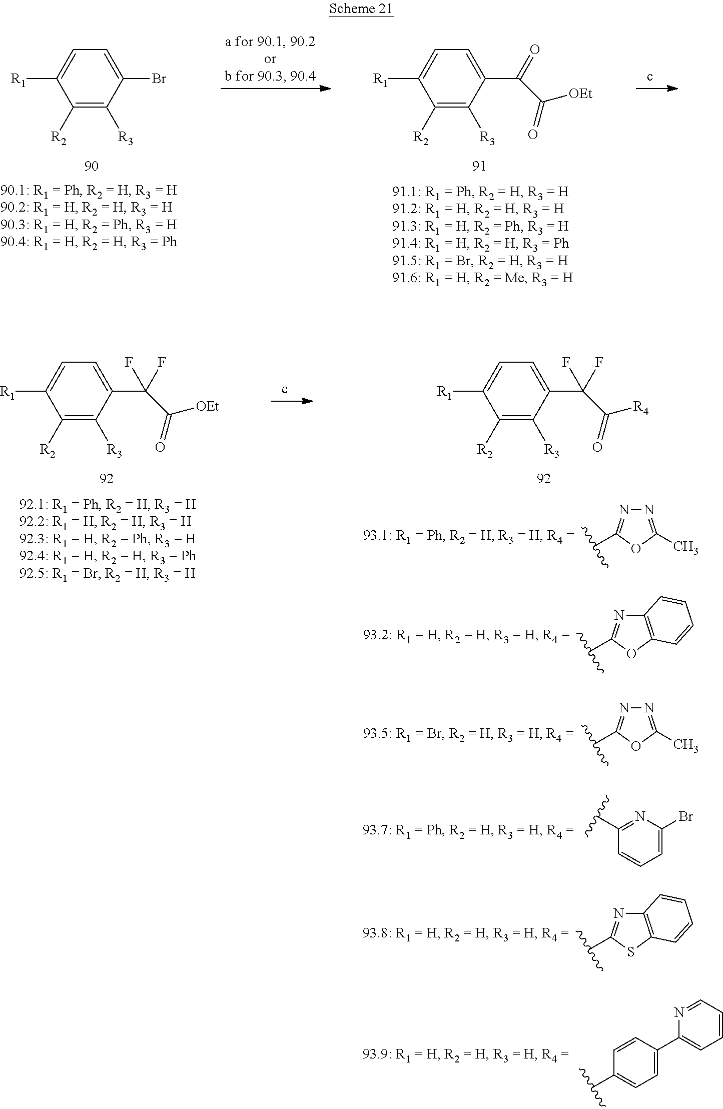

Reagents and conditions for the steps in Scheme 21 were as follows: Step a: Mg, THF, reflux, then addition to (COOEt)₂, THF, −78° C. to 10° C., 1 hour, 55-65%; Step b: n-BuLi, THF, −78° C., 15 min, then addition to (COOEt)₂, THF, −78° C. to 0° C., 1 hours, 58-68%; Step c: DAST CHCl₃, microwave, 100° C., 300 W, 3-5 min, 78-86%; Step d: R₄Li, THF or Et₂O, −78° C. to RT, 1-2 hours, 68-80%.

### 1. α-Keto-esters (91)

To a three-neck round bottom flask containing Mg turnings (1.2 equiv.) equipped with a magnetic stirrer and dimroth condenser was added a solution of alkyl bromide 90 (1 equiv.) in anhydrous THF via syringe and external heating under argon atmosphere. The reaction mixture was refluxed gently for 30-40 min, and then cooled to RT, before conveying it to a dropping funnel. The Grignard reagent was added dropwise to a solution of diethyl oxalate (1.5 equiv.) in THF at −78° C. The reaction mixture was warmed to 10° C. within 1 hour, and then quenched by the addition of saturated ammonium chloride solution. The organic layer was separated, the aqueous layer was extracted with diethyl ether, and the combined organic layer was washed with brine, dried over MgSO₄, and evaporated. The residue was purified by flash column chromatography on silica gel (diethyl ether-hexane) to give pure compound 91 in 55-65% yields.

### 2. Selected data of synthesized α-Keto-esters (91)

### Ethyl 2-(biphenyl-4-yl)-2-oxoacetate (91.1)

91.1 was confirmed as follows: ¹H NMR (500 MHz, CDCl₃) δ 8.09 (d, J=8.5 Hz, 2H), 7.74 (d, J=8.5 Hz, 2H), 7.65 (d, J=7.2 Hz, 2H), 7.49 (t, J=7.2 Hz, 2H), 7.43 (t, J=7.2 Hz, 1H), 4.47 (q, J=7.5 Hz, 2H), 1.45 (t, J=7.5 Hz, 3H).

### Ethyl 2-(4-bromophenyl)-2-oxoacetate (91.5)

91.5 was confirmed as follows: ¹H NMR (500 MHz, CDCl₃) δ 7.92 (d, J=8.5 Hz, 2H), 7.68 (d, J=8.5 Hz, 2H), 3.98 (s, 3H).

### 3. α,α-Difluoro-esters (92)

To a solution of α-keto-ester 91 (1 equiv.) in anhydrous chloroform at RT was added diethylaminosulfur trifluoride (1.1 equiv.) and the reaction mixture was heated using microwave irradiation (300 W, 100° C.) for 3-5 min. The reaction mixture was cooled to RT and poured into ice-water. The organic layer was separated, washed with sat. NaHCO₃ solution, and then dried over MgSO₄. Volatiles were removed under reduced pressure and the crude product was purified by flash chromatography on silica gel (diethyl ether-hexane) to give pure compound 92 in 78-86% yields.

### 4. Selected data of synthesized α,α-difluoro-esters (92)

### Ethyl 2-(biphenyl-4-yl)-2,2-difluoroacetate (92.1)

92.1 was confirmed as follows: ¹H NMR (500 MHz, CDCl₃) δ 7.68 (d, J=9.0 Hz, half of AA′BB′ system, 2H), 7.66 (d, J=9.0 Hz, half of AA′BB′ system, 2H), 7.58 (d, J=7.5 Hz, 2H), 7.46 (t, J=7.5 Hz, 2H), 7.39 (t, J=7.5 Hz, 1H), 4.32 (q, J=7.6 Hz, 2H), 1.33 (t, J=7.6 Hz, 3H).

### Ethyl 2,2-difluoro-2-phenylacetate (92.2)

92.2 was confirmed as follows: ¹H NMR (500 MHz, CDCl₃) δ 7.62 (d, J=7.0 Hz, 2H), 7.50-7.43 (m, 3H), 4.29 (q, J=7.0 Hz, 2H), 1.29 (t, J=7.0 Hz, 3H)

### Ethyl 2-(biphenyl-3-yl)-2,2-difluoroacetate (92.3)

92.3 was confirmed as follows: ¹H NMR (500 MHz, CDCl₃) δ 7.83 (br s, 1H), 7.72 (d, J=7.0 Hz, 1H), 7.58-7.61 (m, 3H), 7.53 (t, J=7.5 Hz, 1H), 7.47 (t, J=7.5 Hz, 2H), 7.39 (t, J=7.5 Hz, 1H), 4.32 (q, J=7.5 Hz, 2H), 1.32 (t, J=7.5 Hz, 3H).

### Ethyl 2-(4-bromophenyl)-2,2-difluoroacetate (92.5)

92.5 was confirmed as follows: ¹H NMR (500 MHz, CDCl₃) δ 7.61 (d, J=8.0 Hz, 2H), 7.49 (d, J=8.0 Hz, 2H), 3.85 (s, 3H).

### 5. α,α-difluoromethylene-ketones (93)

To a stirred solution of α,α-difluoro-ester 92 (1 equiv.) in anhydrous THF or diethyl ether at −78° C. under an argon atmosphere was added the appropriate organolithium reagent (1.1 equiv.) dropwise. The reaction mixture was allowed to warm to RT over 1-2 hours period, and then quenched by the addition of saturated ammonium chloride solution. The organic phase was separated, the aqueous layer was extracted with diethyl ether or methylene chloride, and the combined organic layer was washed with water and brine, dried (MgSO₄), and evaporated under reduced pressure. Purification by flash column chromatography on silica gel (diethyl ether-hexane or acetone-hexane) gave compound (89) in 68-80% yields.

### 6. Selected data of synthesized α,α-difluoromethylene-ketones (93)

### 2-(Biphenyl-4-yl)-2,2-difluoro-1-(5-methyl-1,3,4-oxadiazol-2-yl)ethanone (93.1)

93.1 was confirmed as follows: ¹H NMR (500 MHz, acetone-d₆) δ 7.73 (d, J=8.5 Hz, 1H), 7.71 (d, J=8.0 Hz, 1H), 7.68 (d, J=8.5 Hz, 1H), 7.50 (t, J=8.0 Hz, 2H), 7.41 (t, J=8.0 Hz, 1H), 2.54 (s, 3H).

### 2-(4-Bromophenyl)-2,2-difluoro-1-(5-methyl-1,3,4-oxadiazol-2-yl)ethanone (93.5)

93.5 was confirmed as follows: ¹H NMR (500 MHz, acetone-d₆) δ 7.65 (d, J=8.0 Hz, 2H), 7.55 (d, J=8.0 Hz, 2H), 2.54 (s, 3H).

### 2-(Biphenyl-4-yl)-1-(6-bromopyridin-2-yl)-2,2-difluoroethanone (93.7)

93.7 was confirmed as follows: ¹H NMR (500 MHz, CDCl₃) δ 8.03 (dd, J=7.7 Hz, J=1.2 Hz, 1H), 7.87 (d, J=8.2 Hz, 2H), 7.69 (t, J=7.7 Hz, 1H), 7.67 (d, J=8.2 Hz, 2H), 7.63 (dd, J=7.7 Hz, J=1.2 Hz, 1H), 7.59 (d, J=7.5 Hz, 2H), 7.45 (t, J=7.5 Hz, 2H), 7.37 (t, J=7.5 Hz, 1H).

### Synthesis of α,α-difluoromethylene-ketones 96.1 and 96.2

α,α-difluoromethylene-ketones 96.1 and 96.2 were synthesized by the method depicted in Scheme 22 using commercially available ethyl bromodifluoroacetate, 6-phenoxyhexyl bromide, and 4-bromobiphenyl.

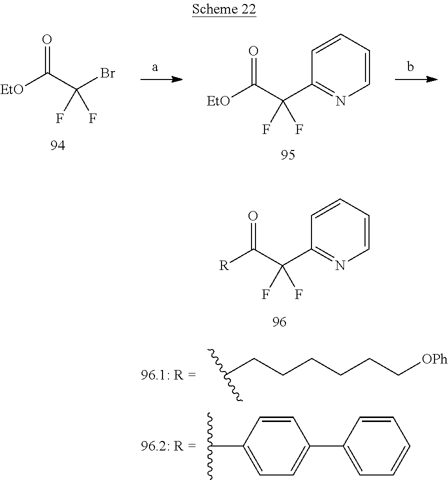

Reagents and conditions for the steps in Scheme 22 were as follows:

Step a: 2-bromopyridine, Cu, DMSO, 50° C., 2 hours, 82%; Step b: RMgBr, THF, −78° C. to 10° C., 1 hour, 50-67%.

### 1. Ethyl 2-(2-pyridyl)-2,2-difluoroacetate (95)

To a solution of ethyl bromodifluoroacetate (1.1 equiv.) and 2-bromopyridine (1 equiv.) in DMSO was added copper bronze (2.2 equiv.) and the mixture was heated to 50° C. with stirring for 2 hours. The reaction mixture was cooled to RT and diluted with ethyl acetate. A solution of potassium dihydrogen phosphate was added, and the mixture stirred for 30 min before filtering. The copper salts were washed with ethyl acetate. and the organic layer was washed with water. Solvent evaporation and purification by flash column chromatography on silica gel (diethyl ether-hexane) gave the title compound as a colorless oil in 82% yield.

### 2. α,α-difluoromethylene-ketones (96)

To a three-neck round bottom flask containing Mg turnings (1.2 equiv.) equipped with a magnetic stirrer and condenser was added a solution of the appropriate bromide (1 equiv.) in dry THF via syringe. The reaction mixture was refluxed gently for 30-40 min, cooled to RT, and transferred to the addition funnel. The Grignard reagent was added dropwise to a solution of ethyl 2-(2-pyridyl)-2,2-difluoroacetate (1 equiv.) in THF at −78° C. The reaction mixture was warmed to 10° C. within 1 hour and then quenched by the addition of saturated ammonium chloride solution. The organic layer was separated, the aqueous layer was extracted with diethyl ether, and the combined organic layer was washed with brine, dried over MgSO₄, and evaporated. The residue was purified by flash column chromatography on silica gel (diethyl ether-hexane) to give pure compound 96 in 50-67% yield.

### 3. Selected data of synthesized α,α-difluoromethylene-ketones (96)

### 1,1-Difluoro-8-phenoxy-1-(pyridin-2-yl)octan-2-one (96.1)

96.1 was confirmed as follows: ¹H NMR (500 MHz, CDCl₃) δ 8.62 (d, J=5.0 Hz, 1H), 7.84 (t, J=7.5 Hz, 1H), 7.71 (d, J=5.0 Hz, 1H), 7.40 (dd, J=7.5 Hz, J=5.0 Hz, 1H), 7.27 (t, J=8.3 Hz, 2H), 6.93 (t, J=8.3 Hz, 1H), 6.87 (d, J=8.3 Hz, 2H), 3.94 (t, J=6.5 Hz, 2H), 2.86 (t, J=7.5 Hz, 2H), 1.77 (qt, J=7.5 Hz, 2H), 1.70 (qt, J=7.0 Hz, 2H), 1.48 (qt, J=7.0 Hz, 2H), 1.39 (qt, J=7.4 Hz, 2H).

### 1-(Biphenyl-4-yl)-2,2-difluoro-2-(pyridin-2-yl)ethanone (96.2)

96.2 was confirmed as follows: ¹H NMR (500 MHz, CDCl₃) δ 8.63 (d, J=4.5 Hz, 1H), 8.13 (d, J=8.5 Hz, 2H), 7.91 (td, J=7.5 Hz, J=1.5 Hz, 1H), 7.86 (d, J=8.5 Hz, 1H), 7.65 (d, J=8.5 Hz, 2H), 7.60 (d, J=7.5 Hz, 2H), 7.46 (t, J=7.5 Hz, 2H), 7.38-7.43 (m, 2H).

IR (neat)=3060, 1707, 1273, 1146, 906.

Some of the compounds included in this disclosure were isolated in their hydrate form or as mixtures of the keto and the hydrate form. A method for converting the hydrate to the keto form is given below.

A solution of the hydrate form or mixture of hydrate/keto forms in an anhydrous solvent (for example benzene) was stirred at RT in the presence of a drying agent (for example molecular sieves) for approximately 0.5-4 hours under an argon atmosphere. The drying agent was removed by filtration and the filtrate was evaporated to give the keto form quantitatively. Alternatively, the hydrate form or the mixture of hydrate/keto forms was dried under high vacuum in the presence of a drying agent (for example P₂O₅) to give the keto form.

### EQUIVALENTS

Those skilled in the art will recognize, or be able to ascertain, using no more than routine experimentation, numerous equivalents to the specific embodiments described specifically in this disclosure. Such equivalents are intended to be encompassed in the scope of the following claims.

1. A method of modulating a cannabinoid receptor in a biological sample, comprising:
(a) measuring the level of a cannabinergic ligand in the biological sample;
(b) contacting the sample with a compound having formula R—X—Y, the compound inhibiting an enzyme that hydrolyzes the cannabinergic ligand:
wherein:
Y is selected from the group consisting of: 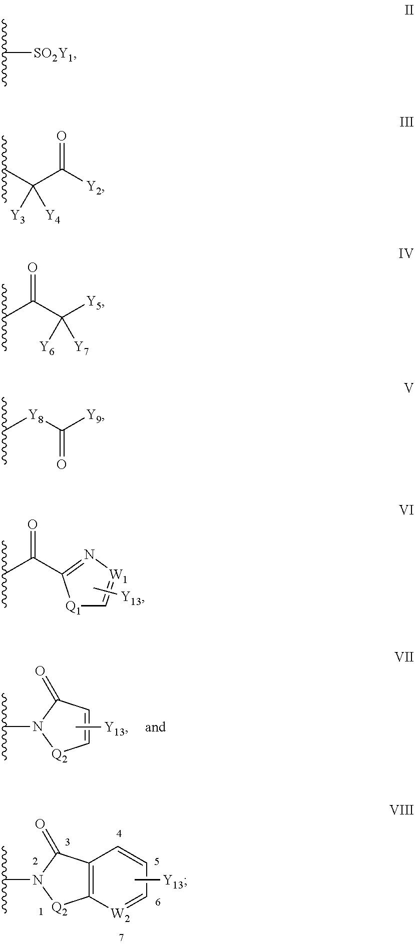
wherein:
Y₁ is —F, —Cl, —O-alkyl, —O-cycloalkyl, —O-heterocyclic, —O-aryl, —O-heteroaryl, or —O-adamantyl;
Y₂ is —H, —OH, —NH₂, —OMe, —OEt, —CF₃, —C≡CH, —CH₂—C≡CH, —CH═CH₂, fluoroalkyl, —C₁₋₅-alkyl, -aryl, -alkyl-aryl, -aryl-alkyl, -aryl-alkyl-Y₁₄, -aryl-heteroaryl, -aryl-aryl, -heteroaryl, -heteroaryl-alkyl, -heteroaryl-alkyl-Y₁₄, -heteroaryl-aryl, -heteroaryl-heteroaryl, -cycloalkyl, -cycloalkyl-alkyl, -cycloalkyl-alkyl-Y₁₄, -heterocyclic, -heterocyclic-alkyl, -heterocyclic-alkyl-Y₁₄, -adamantyl, —C₁₋₅-alkyl-Y₁₄, -aryl-Y₁₄, -heteroaryl-Y₁₄, -cycloalkyl-Y₁₄, -heterocyclic-Y₁₄, or -adamantyl-Y₁₄;
Y₃ and Y₄ are each independently —F, —Cl, or —OH; or Y3 and Y4 taken together form a ketone;
Y₅ is —F, —CONH₂, —SO₂NH₂, —COOH, —COOMe, —COOEt, —CF₃, —C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₆ and Y₇ are each independently —F, —Cl, or —OH;
Y₈ is NH, O, or heterocycle;
Y₉ is —OY₁₀, —N(Y₁₁)Y₁₂, or heterocycle;
Y₁₀ is alkyl, aryl, benzyl, difluorophenyl, fluorophenyl, heteroaryl, cycloalkyl, adamantyl, heterocyclic, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₁₁ is —H, -alkyl, -aryl, or -alkyl-aryl;
Y₁₂ is alkyl, aryl, heteroaryl, cycloalkyl, adamantyl, heterocyclic, —C₁₋₅-alkyl-Y₁₄, —C₁₋₅-alkyl-aryl, —C₁₋₅-alkyl-heteroaryl, -aryl-(Y₁₄)₁₋₄, -heteroaryl-Y₁₄, -cycloalkyl-Y₁₄, -adamantyl-Y₁₄, or -heterocyclic-Y₁₄; or Y₁₁ and Y₁₂ when taken together along with the N to which they are bonded form a 5- or 6-membered saturated heterocylic ring, the ring containing up to one additional heteroatom selected from the group consisting of N, O, and S;
Y₁₃ is —H, —OH, —SH, —NH₂, —CN, —N₃, —NCS, —NCO, —CONH₂, —SO₂NH₂, —COOH, —COOMe, —COOEt, —NO₂, —CF₃, —SO₃H, —P(O)(OH)₂, —C≡CH, —CH₂—C≡CH, —CH═CH₂, fluoroalkyl, —C₁₋₆-alkyl, aryl, heteroaryl, cycloalkyl, adamantyl, heterocyclic, —C₁₋₆-alkyl-Y₁₄; aryl Y₁₄, -heteroaryl-Y₁₄, -cycloalkyl-Y₁₄, -adamantyl-Y₁₄, or -heterocyclic-Y₁₀;
Y₁₄ is —H, —F, —Cl, Br, —I, —OH, —OMe, —OEt, —OPh, —OBn, —SH, —NH₂, —CN, —N₃, —NCS, —NCO, —CONH₂, —SO₂NH₂, —COOH, —COOMe, —COOEt, —NO₂, -alkyl, —CF₃, —SO₃H, —P(O)(OH)₂, —CH₂—C≡CH, —CH═CH₂, or —NHCOCH₃, or —CH₂OH;
W₁ is CH or N if Y₁₃ is not bonded to W₁, or W₁ is C if Y₁₃ is bonded to W₁;
W₂ is CH or N if W₂ is not bonded to Y₁₃, or W₂ is C if W₂ is bonded to Y₁₃; if W₂ is N then it can occupy position 4, 5, 6, or 7 in VIII;
Q₁ is —CH₂, —O, —S, or —NH if Q₁ is not bonded to Y₁₃; Q₁ is —CH or —N if Q₁ is bonded to Y₁₃;
Q₂ is —SO₂, —C(O), or —S(O);
wherein:
X is —(CH₂)ₙ—, —(CH₂)ⱼ-A-(CH₂)ₖ—, cycloalkyl, or heterocycle, wherein:
A is —CH═CH—, C═O, O, S, or NH;
n is an integer from 0 to 15;
j is an integer from 0 to 10;
k is an integer from 0 to 10;
wherein:
R is selected from the group consisting of: 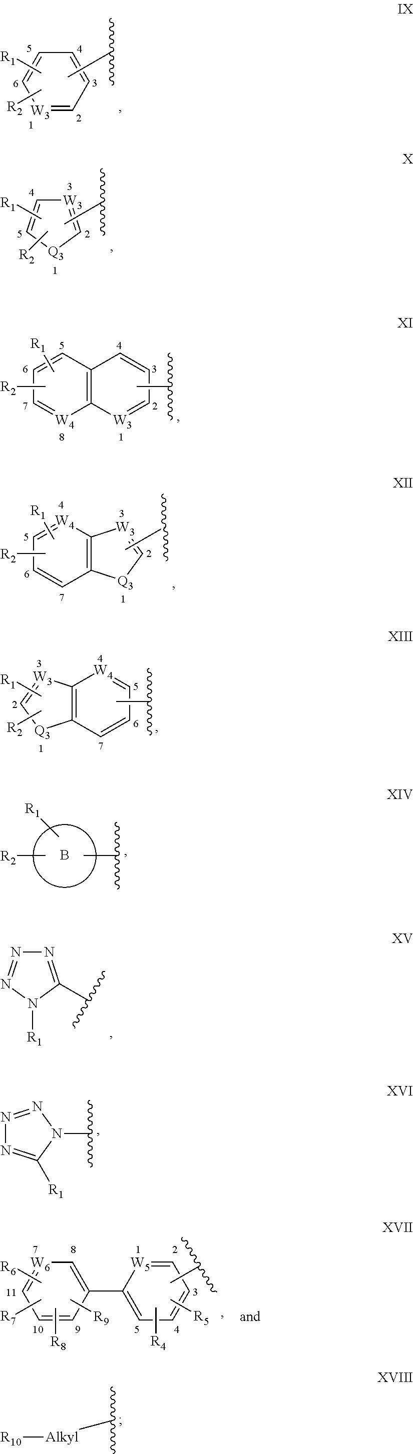
wherein:
W₃ is CH, O, or N if W₃ is not bonded to X or R₁ or R₂; W₃ is C if W₃ is bonded to X or R₁ or R₂; if W₃ is N then it can occupy position 1, 2, 3, 4, 5 or 6 in IX, position 2, 3, 4 or 5 in X, position 1, 2, 3 or 4 in XI, position 2 or 3 in XII, and position 2 or 3 in XIII;
W₄ is CH or N if W₄ is not bonded to X or R₁ or R₂; W₄ is C if W₄ is bonded to X or R₁ or R₂; if W₄ is N then it can occupy position 5, 6, 7 or 8 in XI, position 4, 5, 6 or 7 in XII and position 4, 5, 6 or 7 in XIII;
W₅ is CH or N if W₅ is not bonded to X or R₄ or R₅; W₅ is C if W₅ is bonded to X or R₄ or R₅; if W₅ is N then it can occupy position 1, 2, 3, 4 or 5 in XVII;
W₆ is CH or N if W₆ is not bonded to R₆ or R₇ or R₈ or R₉; W₆ is C if W₆ is bonded to R₆ or R₇ or R₈ or R₉; if W₆ is N then it can occupy position 7, 8, 9, 10 or 11 in XVII;
Q₃ is CH₂, O, S or NH if Q₃ is not bonded to X or R₁ or R₂; Q₃ is CH or N if Q₃ is bonded to X or R₁ or R₂;
B is adamantyl or heteroadamantyl;
R₁ and R₂ are each independently —H, —F, —Cl, —Br, —I, —OH, —SH, —NH₂, —CN, —N3, —NCS, —NCO, —CONH₂, —SO₂NH₂, —COOH, —NO₂, —CHO, —CF₃, —SO₃H, —SO₂Cl, —SO₂F, —O—P(O)(OH)₂, —O—P(O)(O-alkyl)₂, —O—P(O)(OH)(O-alkyl), —P(O)(O-alkyl)₂, —P(O)(OH)(O-alkyl), —Sn(alkyl)₃, —Si(alkyl)₃, —C≡CH, —CH₂—C≡CH, —CH═CH₂, -alkyl-R₃, -cycloalkyl-R₃, -heterocyclic-R₃, -aryl-R₃, -heteroaryl-R₃, -alkyl-cycloalkyl-R₃, -alkyl-heterocyclic-R₃, -alkyl-aryl-R₃, -alkyl-heteroaryl-R₃, —Z-alkyl-R₃, —Z-cycloalkyl-R₃, —Z-heterocyclic-R₃, —Z-aryl-R₃, —Z-heteroaryl-R₃, —Z-alkyl-cycloalkyl-R₃, —Z-alkyl-heterocyclic-R₃, —Z-alkyl-aryl-R₃, —Z-alkyl-heteroaryl-R₃, -aryl-Z-alkyl-R₃, -aryl-Z-cycloalkyl-R₃, -aryl-Z-heterocyclic-R₃, -aryl-Z-aryl-R₃, -aryl-Z-heteroaryl-R₃, -aryl-Z-alkyl-cycloalkyl-R₃, -aryl-Z-alkyl-heterocyclic-R₃, -aryl-Z-alkyl-aryl-R₃, -aryl-Z-alkyl-heteroaryl-R₃, —CH(alkyl-R₃)₂, —C(alkyl-R₃)₃, —N(alkyl-R₃)₂, —C(O)N(alkyl-R₃)₂, —SO₂N(alkyl-R₃)₂, or adamantyl;
Z is —O, —S, —NH, —C(O), —C(O)O, —OC(O), —C(O)NH, —NHC(O), —SO, —SO₂, —SO₂NH, —NHSO₂, —SO₂O, or —OSO₂;
R₃ is —H, —F, —Cl, —Br, —I, -Me, -Et, —OH, —OAc, —SH, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, —C≡CH, —CH₂—C≡CH, or —CH═CH₂;
R₄, R₅, R₆, R₇, R₈, and R₉ are each independently —H, —F, —Cl, —Br, —I, —OH, —OMe, —OEt, —OCH₂OCH₃, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, -alkyl, or -alkyl-R₃; and
R₁₀ is —H, —F, —Cl, —Br, —I, —OH, —OMe, —OEt, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —O Si(alkyl)₃, —C≡CH, —CH₂—C≡CH, or —CH═CH₂;
wherein:
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0, then R can not be IX, X, XI, XII, XIII, or XVIII when one of R₁ or R₂ is H;
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0-3, and R is XVII, then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂;
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0; then R can not be IX, X, XI, XII, XIII, or XVIII when one of R₁ or R₂ is H; and
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0-3, and R is XVII; then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂; and
(c) measuring the level of the cannabinergic ligand in the contacted sample,
the cannabinoid receptor being modulated if the level of the cannabinergic ligand in the contacted sample is the same or greater than the level of the cannabinergic ligand in the uncontacted sample.

2. The method of claim 1 wherein the enzyme is monoacylglycerol lipase.

3. The method of claim 2 wherein the cannabinergic ligand is 2-arachidonoylglycerol.

4. The method of claim 1, wherein the enzyme is fatty acid amide hydrolase.

5. The method of claim 4, wherein the cannabinergic ligand is anandamide.

6. The method of claim 1, wherein the cannabinoid receptor is CB1.

7. The method of claim 1, wherein the cannabinoid receptor is CB2.

8. The method of claim 1, wherein the compound having formula R—X—Y is a compound listed in Table 2.

9. A method of treating a neuropathy in a subject, comprising:
(a) administering to the subject a therapeutically effective amount of a compound having formula R—X—Y,
wherein:
Y is selected from the group consisting of: 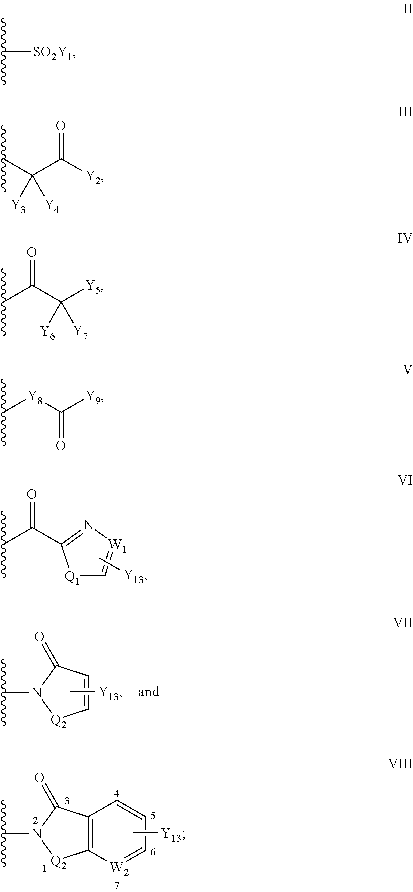
wherein:
Y₁ is —F, —Cl, —O-alkyl, —O-cycloalkyl, —O-heterocyclic, —O-aryl, —O-heteroaryl, or —O-adamantyl;
Y₂ is —H, —OH, —NH₂, —OMe, —OEt, —CF₃, —C≡CH, —CH₂—C≡CH, —CH═CH₂, fluoroalkyl, —C₁₋₅-alkyl, -aryl, -alkyl-aryl, -aryl-alkyl, -aryl-alkyl-Y₁₄, -aryl-heteroaryl, -aryl-aryl, -heteroaryl, -heteroaryl-alkyl, -heteroaryl-alkyl-Y₁₄, -heteroaryl-aryl, -heteroaryl-heteroaryl, -cycloalkyl, -cycloalkyl-alkyl, -cycloalkyl-alkyl-Y₁₄, -heterocyclic, -heterocyclic-alkyl, -heterocyclic-alkyl-Y₁₄, -adamantyl, —C₁₋₅-alkyl-Y₁₄, -aryl-Y₁₄, -heteroaryl-Y₁₄, -cycloalkyl-Y₁₄, -heterocyclic-Y₁₄, or -adamantyl-Y₁₄;
Y₃ and Y₄ are each independently —F, —Cl, or —OH; or Y3 and Y4 taken together form a ketone;
Y₅ is —F, —CONH₂, —SO₂NH₂, —COOH, —COOMe, —COOEt, —CF₃, —C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₆ and Y₇ are each independently —F, —Cl, or —OH;
Y₈ is NH, O, or heterocycle;
Y₉ is —OY₁₀, —N(Y₁₁)Y₁₂, or heterocycle;
Y₁₀ is alkyl, aryl, benzyl, difluorophenyl, fluorophenyl, heteroaryl, cycloalkyl, adamantyl, heterocyclic, —C₁₋₅-alkyl-Y₁₄; -aryl-Y₁₄, -heteroaryl-Y₁₄; -cycloalkyl-Y₁₄, -adamantyl-Y₁₄, or -heterocyclic-Y₁₄;
Y₁₁ is —H, -alkyl, -aryl, or -alkyl-aryl;
Y₁₂ is alkyl, aryl, heteroaryl, cycloalkyl, adamantyl, heterocyclic, —C₁₋₅-alkyl-Y₁₄, —C₁₋₅-alkyl-aryl, —C₁₋₅-alkyl-heteroaryl, -aryl-(Y₁₄)₁₋₄, -heteroaryl-Y₁₄, -cycloalkyl-Y₁₄, -adamantyl-Y₁₄; or -heterocyclic-Y₁₄; or Y₁₁ and Y₁₂ when taken together along with the N to which they are bonded form a 5- or 6-membered saturated heterocylic ring, the ring containing up to one additional heteroatom selected from the group consisting of N, O, and S;
Y₁₃ is —H, —OH, —SH, —NH₂, —CN, —N₃, —NCS, —NCO, —CONH₂, —SO₂NH₂, —COOH, —COOMe, —COOEt, —NO₂, —CF₃, —SO₃H, —P(O)(OH)₂, —CH₂—C≡CH, —CH═CH₂, fluoroalkyl, —C₁₋₆-alkyl, aryl, heteroaryl, cycloalkyl, adamantyl, heterocyclic, —C₁₋₆-alkyl-Y₁₄, -aryl-Y₁₄, -heteroaryl-Y₁₄, -cycloalkyl-Y₁₄, -adamantyl-Y₁₄, or -heterocyclic-Y₁₀;
Y₁₄ is —H, —F, —Cl, Br, —I, —OH, —OMe, —OEt, —OPh, —OBn, —SH, —NH₂, —CN, —N₃, —NCS, —NCO, —CONH₂, —SO₂NH₂, —COOH, —COOMe, —COOEt, —NO₂, -alkyl, —CF₃, —SO₃H, —P(O)(OH)₂, —CH₂—C≡CH, —CH═CH₂, or —NHCOCH₃, or —CH₂OH;
W₁ is CH or N if Y₁₃ is not bonded to W₁, or W₁ is C if Y₁₃ is bonded to W₁;
W₂ is CH or N if W₂ is not bonded to Y₁₃, or W₂ is C if W₂ is bonded to Y₁₃; if W₂ is N then it can occupy position 4, 5, 6, or 7 in VIII;
Q₁ is —CH₂, —O, —S, or —NH if Q₁ is not bonded to Y₁₃; Q₁ is —CH or —N if Q₁ is bonded to Y₁₃;
Q₂ is —SO₂, —C(O), or —S(O);
wherein:
X is —(CH₂)ₙ—, —(CH₂)ⱼ-A-(CH₂)ₖ—, cycloalkyl, or heterocycle, wherein:
A is —CH═CH—, —C≡C—, C═O, O, S, or NH;
n is an integer from 0 to 15;
j is an integer from 0 to 10;
k is an integer from 0 to 10;
wherein:
R is selected from the group consisting of: 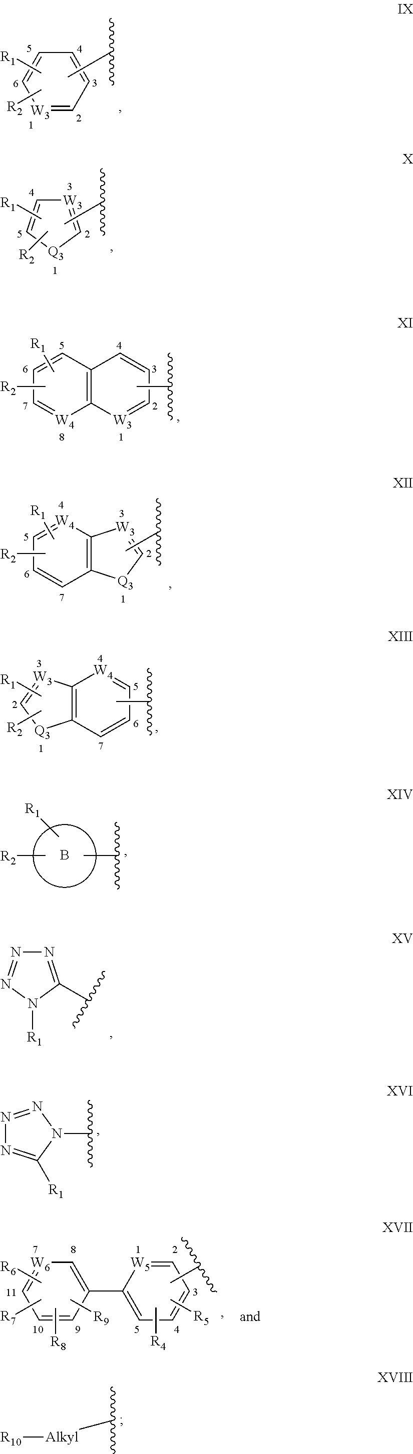
wherein:
W₃ is CH, O, or N if W₃ is not bonded to X or R₁ or R₂; W₃ is C if W₃ is bonded to X or R₁ or R₂; if W₃ is N then it can occupy position 1, 2, 3, 4, 5 or 6 in IX, position 2, 3, 4 or 5 in X, position 1, 2, 3 or 4 in XI, position 2 or 3 in XII, and position 2 or 3 in XIII;
W₄ is CH or N if W₄ is not bonded to X or R₁ or R₂; W₄ is C if W₄ is bonded to X or R₁ or R₂; if W₄ is N then it can occupy position 5, 6, 7 or 8 in XI, position 4, 5, 6 or 7 in XII and position 4, 5, 6 or 7 in XIII;
W₅ is CH or N if W₅ is not bonded to X or R₄ or R₅; W₅ is C if W₅ is bonded to X or R₄ or R₅; if W₅ is N then it can occupy position 1, 2, 3, 4 or 5 in XVII;
W₆ is CH or N if W₆ is not bonded to R₆ or R₇ or R₈ or R₉; W₆ is C if W₆ is bonded to R₆ or R₇ or R₈ or R₉; if W₆ is N then it can occupy position 7, 8, 9, 10 or 11 in XVII;
Q₃ is CH₂, O, S or NH if Q₃ is not bonded to X or R₁ or R₂; Q₃ is CH or N if Q₃ is bonded to X or R₁ or R₂;
B is adamantyl or heteroadamantyl;
R₁ and R₂ are each independently —H, —F, —Cl, —Br, —I, —OH, —SH, —NH₂, —CN, —N₃, —NCS, —NCO, —CONH₂, —SO₂NH₂, —COOH, —NO₂, —CHO, —CF₃, —SO₃H, —SO₂Cl, —SO₂F, —O—P(O)(OH)₂, —O—P(O)(O-alkyl)₂, —O—P(O)(OH)(O-alkyl), —P(O)(O-alkyl)₂, —P(O)(OH)(O-alkyl), —Sn(alkyl)₃, —Si(alkyl)₃, —C≡CH, —CH₂—C≡CH, —CH═CH₂, -alkyl-R₃, -cycloalkyl-R₃, -heterocyclic-R₃, -aryl-R₃, -heteroaryl-R₃, -alkyl-cycloalkyl-R₃, -alkyl-heterocyclic-R₃, -alkyl-aryl-R₃, -alkyl-heteroaryl-R₃, —Z-alkyl-R₃, —Z-cycloalkyl-R₃, —Z-heterocyclic-R₃, —Z-aryl-R₃, —Z-heteroaryl-R₃, —Z-alkyl-cycloalkyl-R₃, —Z-alkyl-heterocyclic-R₃, —Z-alkyl-aryl-R₃, —Z-alkyl-heteroaryl-R₃, -aryl-Z-alkyl-R₃, -aryl-Z-cycloalkyl-R₃, -aryl-Z-heterocyclic-R₃, -aryl-Z-aryl-R₃, -aryl-Z-heteroaryl-R₃, -aryl-Z-alkyl-cycloalkyl-R₃, -aryl-Z-alkyl-heterocyclic-R₃, -aryl-Z-alkyl-aryl-R₃, -aryl-Z-alkyl-heteroaryl-R₃, —CH(alkyl-R₃)₂, —C(alkyl-R₃)₃, —N(alkyl-R₃)₂, —C(O)N(alkyl-R₃)₂, —SO₂N(alkyl-R₃)₂, or adamantyl;
Z is —O, —S, —NH, —C(O), —C(O)O, —OC(O), —C(O)NH, —NHC(O), —SO, —SO₂, —SO₂NH, —NHSO₂, —SO₂O, or —OSO₂;
R₃ is —H, —F, —Cl, —Br, —I, -Me, -Et, —OH, —OAc, —SH, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, —C≡CH, —CH₂—C≡CH, or —CH═CH₂;
R₄, R₅, R₆, R₇, R₈, and R₉ are each independently —H, —F, —Cl, —Br, —I, —OH, —OMe, —OEt, —OCH₂OCH₃, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, -alkyl, or -alkyl-R₃; and
R₁₀ is —H, —F, —Cl, —Br, —I, —OH, —OMe, —OEt, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —O Si(alkyl)₃, —C≡CH, —CH₂—C≡CH, or —CH═CH₂;
wherein:
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0, then R can not be IX, X, XI, XII, XIII, or XVIII when one of R₁ or R₂ is H;
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0-3, and R is XVII, then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂;
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0; then R can not be IX, X, XI, XII, XIII, or XVIII when one of R₁ or R₂ is H; and
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0-3, and R is XVII; then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂; and
(b) detecting a decrease in a symptom of the neuropathy,
the administration of the compound treating the neuropathy of the subject.

10. The method of claim 9, wherein the neuropathy is inflammation, pain, neuropathic pain, neuropathic low back pain, complex regional pain syndrome, post trigeminal neuralgia, causalgia, toxic neuropathy, reflex sympathetic dystrophy, diabetic neuropathy, chronic neuropathy caused by chemotherapeutic agents, central pain, peripheral pain, pellagric neuropathy, alcoholic neuropathy, Beriberi neuropathy, or burning feet syndrome.

11. The method of claim 9 wherein the neuropathy is a neurodegenerative disease.

12. The method of claim 11, wherein the neurodegenerative disease is multiple sclerosis, Parkinson's disease, Huntington's chorea, Alzheimer's disease, amyotrophic lateral sclerosis, memory disorder, mood disorder, sleep disorder, gastrointestinal motility disorder, irritable bowel syndrome, diarrhea, cardiovascular disease, hypertension, osteoporosis, osteoarthritis, emesis, epilepsy, a mental disorder, schizophrenia, depression, glaucoma, cachexia, insomnia, traumatic brain injury, spinal cord injury, seizures, excitotoxin exposure, ischemia, or AIDS wasting syndrome.

13. The method of claim 9, wherein the compound having formula R—X—Y is a compound listed in Table 2.

14. A method of treating an anxiety disorder in a subject, comprising:
(a) administering to the subject a therapeutically effective amount of a compound having formula R—X—Y,
wherein:
Y is selected from the group consisting of: 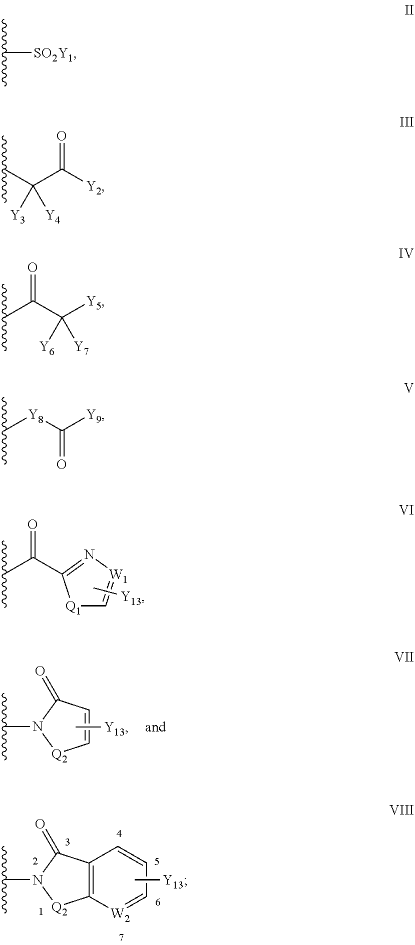
wherein:
Y₁ is —F, —Cl, —O-alkyl, —O-cycloalkyl, —O-heterocyclic, —O-aryl, —O-heteroaryl, or —O-adamantyl;
Y₂ is —H, —OH, —NH₂, —OMe, —OEt, —CF₃, —C≡CH, —CH₂—C≡CH, —CH═CH₂, fluoroalkyl, —C₁₋₅-alkyl, -aryl, -alkyl-aryl, -aryl-alkyl, -aryl-alkyl-Y₁₄, -aryl-heteroaryl, -aryl-aryl, -heteroaryl, -heteroaryl-alkyl, -heteroaryl-alkyl-Y₁₄, -heteroaryl-aryl, -heteroaryl-heteroaryl, -cycloalkyl, -cycloalkyl-alkyl, -cycloalkyl-alkyl-Y₁₄, -heterocyclic, -heterocyclic-alkyl, -heterocyclic-alkyl-Y₁₄, -adamantyl, —C₁₋₅-alkyl-Y₁₄, -aryl-Y₁₄, -heteroaryl-Y₁₄, -cycloalkyl-Y₁₄, -heterocyclic-Y₁₄, or -adamantyl-Y₁₄;
Y₃ and Y₄ are each independently —F, —Cl, or —OH; or Y3 and Y4 taken together form a ketone;
Y₅ is —F, —CONH₂, —SO₂NH₂, —COOH, —COOMe, —COOEt, —CF₃, —C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₆ and Y₇ are each independently —F, —Cl, or —OH;
Y₈ is NH, O, or heterocycle;
Y₉ is —OY₁₀, —N(Y₁₁)Y₁₂, or heterocycle;
Y₁₀ is alkyl, aryl, benzyl, difluorophenyl, fluorophenyl, heteroaryl, cycloalkyl, adamantyl, heterocyclic, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₁₁ is —H, -alkyl, -aryl, or -alkyl-aryl;
Y₁₂ is alkyl, aryl, heteroaryl, cycloalkyl, adamantyl, heterocyclic, —C₁₋₅-alkyl-Y₁₄, —C₁₋₅-alkyl-aryl, —C₁₋₅-alkyl-heteroaryl, -aryl-(Y₁₄)₁₋₄, -heteroaryl-Y₁₄, -cycloalkyl-Y₁₄, -adamantyl-Y₁₄, or -heterocyclic-Y₁₄; or Y₁₁ and Y₁₂ when taken together along with the N to which they are bonded form a 5- or 6-membered saturated heterocylic ring, the ring containing up to one additional heteroatom selected from the group consisting of N, O, and S;
Y₁₃ is —H, —OH, —SH, —NH₂, —CN, —N₃, —NCS, —NCO, —CONH₂, —SO₂NH₂; —COOH, —COOMe, —COOEt, —NO₂, —CF₃, —SO₃H, —P(O)(OH)₂, —CH₂—C≡CH, —CH═CH₂, fluoroalkyl, —C₁₋₆-alkyl, aryl, heteroaryl, cycloalkyl, adamantyl, heterocyclic, —C₁₋₆-alkyl-Y₁₄; aryl Y₁₄ heteroaryl Y₁₄ cycloalkyl-Y₁₄, -adamantyl-Y₁₄, or -heterocyclic-Y₁₀;
Y₁₄ is —H, —F, —Cl, Br, —I, —OH, —OMe, —OEt, —OPh, —OBn, —SH, —NH₂, —CN, —N3, —NCS, —NCO, —CONH₂, —SO₂NH₂, —COOH, —COOMe, —COOEt, —NO₂, -alkyl, —CF₃, —SO₃H, —P(O)(OH)₂, —CH₂—C≡CH, —CH═CH₂, or —NHCOCH₃, or —CH₂OH;
W₁ is CH or N if Y₁₃ is not bonded to W₁, or W₁ is C if Y₁₃ is bonded to W₁;
W₂ is CH or N if W₂ is not bonded to Y₁₃, or W₂ is C if W₂ is bonded to Y₁₃; if W₂ is N then it can occupy position 4, 5, 6, or 7 in VIII;
Q₁ is —CH₂, —O, —S, or —NH if Q₁ is not bonded to Y₁₃; Q₁ is —CH or —N if Q₁ is bonded to Y₁₃;
Q₂ is —SO₂, —C(O), or —S(O);
wherein:
X is —(CH₂)ₙ—, —(CH₂)ⱼ-A-(CH₂)ₖ—, cycloalkyl, or heterocycle, wherein:
A is —CH═CH—, C═O, O, S, or NH;
n is an integer from 0 to 15;
j is an integer from 0 to 10;
k is an integer from 0 to 10;
wherein:
R is selected from the group consisting of: 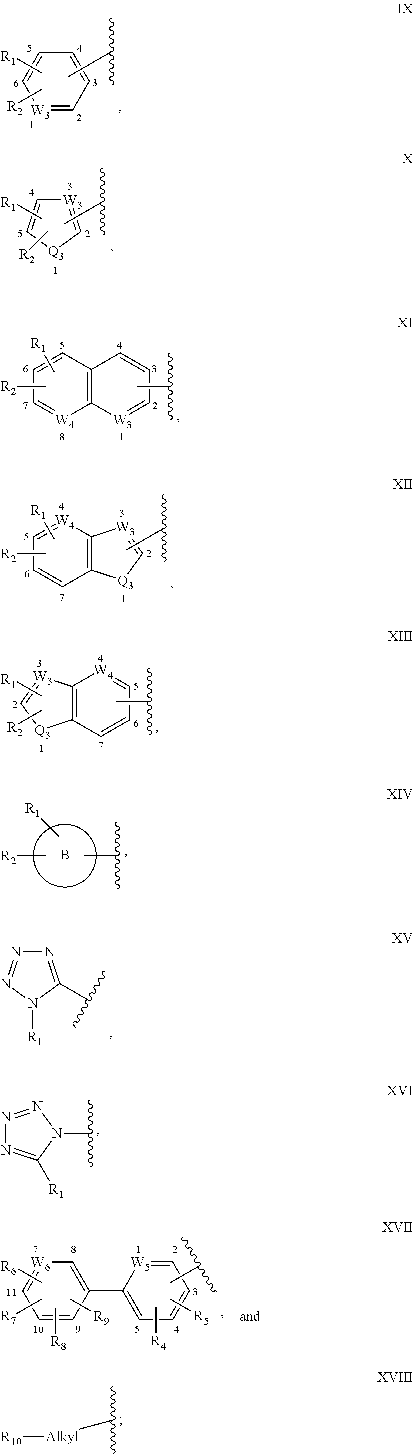
wherein:
W₃ is CH, O, or N if W₃ is not bonded to X or R₁ or R₂; W₃ is C if W₃ is bonded to X or R₁ or R₂; if W₃ is N then it can occupy position 1, 2, 3, 4, 5 or 6 in IX, position 2, 3, 4 or 5 in X, position 1, 2, 3 or 4 in XI, position 2 or 3 in XII, and position 2 or 3 in XIII;
W₄ is CH or N if W₄ is not bonded to X or R₁ or R₂; W₄ is C if W₄ is bonded to X or R₁ or R₂; if W₄ is N then it can occupy position 5, 6, 7 or 8 in XI, position 4, 5, 6 or 7 in XII and position 4, 5, 6 or 7 in XIII;
W₅ is CH or N if W₅ is not bonded to X or R₄ or R₅; W₅ is C if W₅ is bonded to X or R₄ or R₅; if W₅ is N then it can occupy position 1, 2, 3, 4 or 5 in XVII;
W₆ is CH or N if W₆ is not bonded to R₆ or R₇ or R₈ or R₉; W₆ is C if W₆ is bonded to R₆ or R₇ or R₈ or R₉; if W₆ is N then it can occupy position 7, 8, 9, 10 or 11 in XVII;
Q₃ is CH₂, O, S or NH if Q₃ is not bonded to X or R₁ or R₂; Q₃ is CH or N if Q₃ is bonded to X or R₁ or R₂;
B is adamantyl or heteroadamantyl;
R₁ and R₂ are each independently —H, —F, —Cl, —Br, —I, —OH, —SH, —NH₂, —CN, —N₃, —NCS, —NCO, —CONH₂, —SO₂NH₂, —COOH, —NO₂, —CHO, —CF₃, —SO₃H, —SO₂Cl, —SO₂F, —O—P(O)(OH)₂, —O—P(O)(O-alkyl)₂, —O—P(O)(OH)(O-alkyl), —P(O)(O-alkyl)₂, —P(O)(OH)(O-alkyl), —Sn(alkyl)₃, —Si(alkyl)₃, —C≡CH, —CH₂—C≡CH, —CH═CH₂, -alkyl-R₃, -cycloalkyl-R₃, -heterocyclic-R₃, -aryl-R₃, -heteroaryl-R₃, -alkyl-cycloalkyl-R₃, -alkyl-heterocyclic-R₃, -alkyl-aryl-R₃, -alkyl-heteroaryl-R₃, —Z-alkyl-R₃, —Z-cycloalkyl-R₃, —Z-heterocyclic-R₃, —Z-aryl-R₃, —Z-heteroaryl-R₃, —Z-alkyl-cycloalkyl-R₃, —Z-alkyl-heterocyclic-R₃, —Z-alkyl-aryl-R₃, —Z-alkyl-heteroaryl-R₃, -aryl-Z-alkyl-R₃, -aryl-Z-cycloalkyl-R₃, -aryl-Z-heterocyclic-R₃, -aryl-Z-aryl-R₃, -aryl-Z-heteroaryl-R₃, -aryl-Z-alkyl-cycloalkyl-R₃, -aryl-Z-alkyl-heterocyclic-R₃, -aryl-Z-alkyl-aryl-R₃, -aryl-Z-alkyl-heteroaryl-R₃, —CH(alkyl-R₃)₂, —C(alkyl-R₃)₃, —N(alkyl-R₃)₂, —C(O)N(alkyl-R₃)₂, —SO₂N(alkyl-R₃)₂, or adamantyl;
Z is —O, —S, —NH, —C(O), —C(O)O, —OC(O), —C(O)NH, —NHC(O), —SO, —SO₂, —SO₂NH, —NHSO₂, —SO₂O, or —OSO₂;
R₃ is —H, —F, —Cl, —Br, —I, -Me, -Et, —OH, —OAc, —SH, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, —C≡CH, —CH₂—C≡CH, or —CH═CH₂;
R₄, R₅, R₆, R₇, R₈, and R₉ are each independently —H, —F, —Cl, —Br, —I, —OH, —OMe, —OEt, —OCH₂OCH₃, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, -alkyl, or -alkyl-R₃; and
R₁₀ is —H, —F, —Cl, —Br, —I, —OH, —OMe, —OEt, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —O Si(alkyl)₃, —C≡CH, —CH₂—C≡CH, or —CH═CH₂;
wherein:
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0, then R can not be IX, X, XI, XII, XIII, or XVIII when one of R₁ or R₂ is H;
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0-3, and R is XVII, then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂;
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0; then R can not be IX, X, XI, XII, XIII, or XVIII when one of R₁ or R₂ is H; and
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0-3, and R is XVII; then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂; and
(b) detecting a decrease in a symptom of the anxiety disorder,
the administration of the compound treating the anxiety disorder of the subject.

15. The method of claim 14 wherein the anxiety disorder is panic disorder, acute stress disorder, post-traumatic stress disorder, substance-induced anxiety disorder, obsessive compulsive disorder, agoraphobia, specific phobia, or social phobia.

16. The method of claim 14, wherein the compound having formula R—X—Y is a compound listed in Table 2.

17. A method of treating a motor function disorder in a subject, comprising:
(a) administering to the subject a therapeutically effective amount of a compound having formula R—X—Y,
wherein:
Y is selected from the group consisting of: 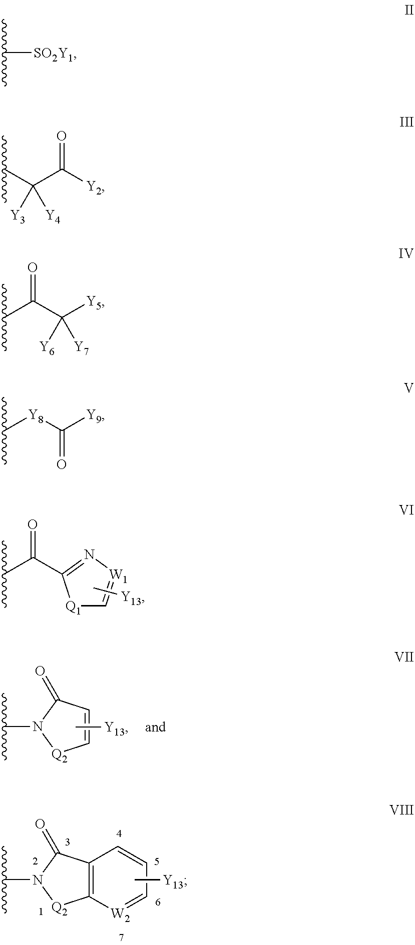
wherein:
Y₁ is —F, —Cl, —O-alkyl, —O-cycloalkyl, —O-heterocyclic, —O-aryl, —O-heteroaryl, or —O-adamantyl;
Y₂ is —H, —OH, —NH₂, —OMe, —OEt, —CF₃, —C≡CH, —CH₂—C≡CH, —CH═CH₂, fluoroalkyl, —C₁₋₅-alkyl, -aryl, -alkyl-aryl, -aryl-alkyl, -aryl-alkyl-Y₁₄, -aryl-heteroaryl, -aryl-aryl, -heteroaryl, -heteroaryl-alkyl, -heteroaryl-alkyl-Y₁₄, -heteroaryl-aryl, -heteroaryl-heteroaryl, -cycloalkyl, -cycloalkyl-alkyl, -cycloalkyl-alkyl-Y₁₄, -heterocyclic, -heterocyclic-alkyl, -heterocyclic-alkyl-Y₁₄, -adamantyl, —C₁₋₅-alkyl-Y₁₄, -aryl-Y₁₄, -heteroaryl-Y₁₄, -cycloalkyl-Y₁₄, -heterocyclic-Y₁₄, or -adamantyl-Y₁₄;
Y₃ and Y₄ are each independently —F, —Cl, or —OH; or Y3 and Y4 taken together form a ketone;
Y₅ is —F, —CONH₂, —SO₂NH₂, —COOH, —COOMe, —COOEt, —CF₃, —C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄; -aryl-Y₁₄, heteroaryl Y₁₄ cycloalkyl-Y₁₄, -adamantyl-Y₁₄, or -heterocyclic-Y₁₄;
Y₆ and Y₇ are each independently —F, —Cl, or —OH;
Y₈ is NH, O, or heterocycle;
Y₉ is —OY₁₀, —N(Y₁₁)Y₁₂, or heterocycle;
Y₁₀ is alkyl, aryl, benzyl, difluorophenyl, fluorophenyl, heteroaryl, cycloalkyl, adamantyl, heterocyclic, —C₁₋₅-alkyl-Y₁₄; -aryl-Y₁₄, -heteroaryl-Y₁₄, -cycloalkyl-Y₁₄; -adamantyl-Y₁₄, or -heterocyclic-Y₁₄;
Y₁₁ is —H, -alkyl, -aryl, or -alkyl-aryl;
Y₁₂ is alkyl, aryl, heteroaryl, cycloalkyl, adamantyl, heterocyclic, —C₁₋₅-alkyl-Y₁₄, —C₁₋₅-alkyl-aryl, —C₁₋₅-alkyl-heteroaryl, -aryl-(Y₁₄)₁₋₄; -heteroaryl-Y₁₄, -cycloalkyl-Y₁₄, -adamantyl-Y₁₄; or -heterocyclic-Y₁₄; or Y₁₁ and Y₁₂ when taken together along with the N to which they are bonded form a 5- or 6-membered saturated heterocylic ring, the ring containing up to one additional heteroatom selected from the group consisting of N, O, and S;
Y₁₃ is —H, —OH, —SH, —NH₂, —CN, —N₃, —NCS, —NCO, —CONH₂, —SO₂NH₂; —COOH, —COOMe, —COOEt, —NO₂, —CF₃, —SO₃H, —P(O)(OH)₂, —C≡CH, —CH₂—C≡CH, —CH═CH₂, fluoroalkyl, —C₁₋₆-alkyl, aryl, heteroaryl, cycloalkyl, adamantyl, heterocyclic, —C₁₋₆-alkyl-Y₁₄; -aryl-Y₁₄, -heteroaryl-Y₁₄, -cycloalkyl-Y₁₄, -adamantyl-Y₁₄, or -heterocyclic-Y₁₀;
Y₁₄ is —H, —F, —Cl, Br, —I, —OH, —OMe, —OEt, —OPh, —OBn, —SH, —NH₂, —CN, —N₃, —NCS, —NCO, —CONH₂, —SO₂NH₂, —COOH, —COOMe, —COOEt, —NO₂, -alkyl, —CF₃, —SO₃H, —P(O)(OH)₂, —C≡CH, —CH₂—C≡CH, —CH═CH₂, or —NHCOCH₃, or —CH₂OH;
W₁ is CH or N if Y₁₃ is not bonded to W₁, or W₁ is C if Y₁₃ is bonded to W₁;
W₂ is CH or N if W₂ is not bonded to Y₁₃, or W₂ is C if W₂ is bonded to Y₁₃; if W₂ is N then it can occupy position 4, 5, 6, or 7 in VIII;
Q₁ is —CH₂, —O, —S, or —NH if Q₁ is not bonded to Y₁₃; Q₁ is —CH or —N if Qᵢ is bonded to Y₁₃;
Q₂ is —SO₂, —C(O), or —S(O);
wherein:
X is —(CH₂)ₙ—, —(CH₂)ⱼ-A-(CH₂)ₖ—, cycloalkyl, or heterocycle, wherein:
A is —CH═CH—, C═O, O, S, or NH;
n is an integer from 0 to 15;
j is an integer from 0 to 10;
k is an integer from 0 to 10;
wherein:
R is selected from the group consisting of: 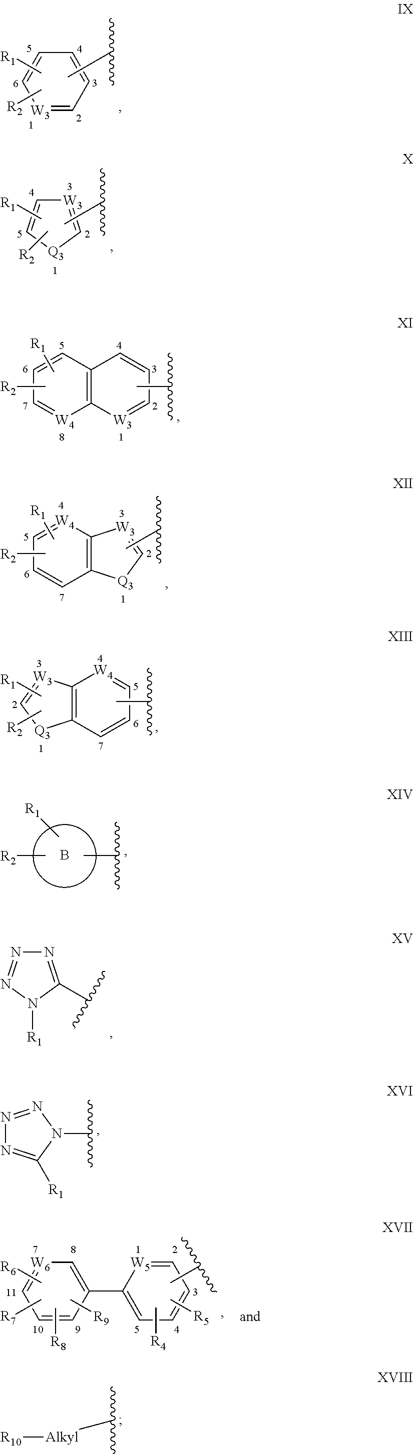
wherein:
W₃ is CH, O, or N if W₃ is not bonded to X or R₁ or R₂; W₃ is C if W₃ is bonded to X or R₁ or R₂; if W₃ is N then it can occupy position 1, 2, 3, 4, 5 or 6 in IX, position 2, 3, 4 or 5 in X, position 1, 2, 3 or 4 in XI, position 2 or 3 in XII, and position 2 or 3 in XIII;
W₄ is CH or N if W₄ is not bonded to X or R₁ or R₂; W₄ is C if W₄ is bonded to X or R₁ or R₂; if W₄ is N then it can occupy position 5, 6, 7 or 8 in XI, position 4, 5, 6 or 7 in XII and position 4, 5, 6 or 7 in XIII;
W₅ is CH or N if W₅ is not bonded to X or R₄ or R₅; W₅ is C if W₅ is bonded to X or R₄ or R₅; if W₅ is N then it can occupy position 1, 2, 3, 4 or 5 in XVII;
W₆ is CH or N if W₆ is not bonded to R₆ or R₇ or R₈ or R₉; W₆ is C if W₆ is bonded to R₆ or R₇ or R₈ or R₉; if W₆ is N then it can occupy position 7, 8, 9, 10 or 11 in XVII;
Q₃ is CH₂, O, S or NH if Q₃ is not bonded to X or R₁ or R₂; Q₃ is CH or N if Q₃ is bonded to X or R₁ or R₂;
B is adamantyl or heteroadamantyl;
R₁ and R₂ are each independently —H, —F, —Cl, —Br, —I, —OH, —SH, —NH₂, —CN, —N₃, —NCS, —NCO, —CONH₂, —SO₂NH₂, —COOH, —NO₂, —CHO, —CF₃, —SO₃H, —SO₂Cl, —SO₂F, —O—P(O)(OH)₂, —O—P(O)(O-alkyl)₂, —O—P(O)(OH)(O-alkyl), —P(O)(O-alkyl)₂, —P(O)(OH)(O-alkyl), —Sn(alkyl)₃, —Si(alkyl)₃, —C≡CH, —CH₂—C≡CH, —CH═CH₂, -alkyl-R₃, -cycloalkyl-R₃, -heterocyclic-R₃, -aryl-R₃, -heteroaryl-R₃, -alkyl-cycloalkyl-R₃, -alkyl-heterocyclic-R₃, -alkyl-aryl-R₃, -alkyl-heteroaryl-R₃, —Z-alkyl-R₃, —Z-cycloalkyl-R₃, —Z-heterocyclic-R₃, —Z-aryl-R₃, —Z-heteroaryl-R₃, —Z-alkyl-cycloalkyl-R₃, —Z-alkyl-heterocyclic-R₃, —Z-alkyl-aryl-R₃, —Z-alkyl-heteroaryl-R₃, -aryl-Z-alkyl-R₃, -aryl-Z-cycloalkyl-R₃, -aryl-Z-heterocyclic-R₃, -aryl-Z-aryl-R₃, -aryl-Z-heteroaryl-R₃, -aryl-Z-alkyl-cycloalkyl-R₃, -aryl-Z-alkyl-heterocyclic-R₃, -aryl-Z-alkyl-aryl-R₃, -aryl-Z-alkyl-heteroaryl-R₃, —CH(alkyl-R₃)₂, —C(alkyl-R₃)₃, —N(alkyl-R₃)₂, —C(O)N(alkyl-R₃)₂, —SO₂N(alkyl-R₃)₂, or adamantyl;
Z is —O, —S, —NH, —C(O), —C(O)O, —OC(O), —C(O)NH, —NHC(O), —SO, —SO₂, —SO₂NH, —NHSO₂, —SO₂O, or —OSO₂;
R₃ is —H, —F, —Cl, —Br, —I, -Me, -Et, —OH, —OAc, —SH, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, —C≡CH, —CH₂—C≡CH, or —CH═CH₂;
R₄, R₅, R₆, R₇, R₈, and R₉ are each independently —H, —F, —Cl, —Br, —I, —OH, —OMe, —OEt, —OCH₂OCH₃, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, -alkyl, or -alkyl-R₃; and
R₁₀ is —H, —F, —Cl, —Br, —I, —OH, —OMe, —OEt, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —O Si(alkyl)₃, —C≡CH, —CH₂—C≡CH, or —CH═CH₂;
wherein:
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0, then R can not be IX, X, XI, XII, XIII, or XVIII when one of R₁ or R₂ is H;
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0-3, and R is XVII, then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂;
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0; then R can not be IX, X, XI, XII, XIII, or XVIII when one of R₁ or R₂ is H; and
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0-3, and R is XVII; then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂; and
(b) detecting a decrease in a symptom of the motor function disorder,
the administration of the compound treating the motor function disorder of the subject.

18. The method of claim 17 wherein the motor function disorder is Tourette's syndrome.

19. The method of claim 17, wherein the compound having formula R—X—Y is a compound listed in Table 2.

20. A method of treating a fertility disorder in a subject, comprising:
(a) administering to the subject a therapeutically effective amount of a compound having formula R—X—Y,
wherein:
Y is selected from the group consisting of: 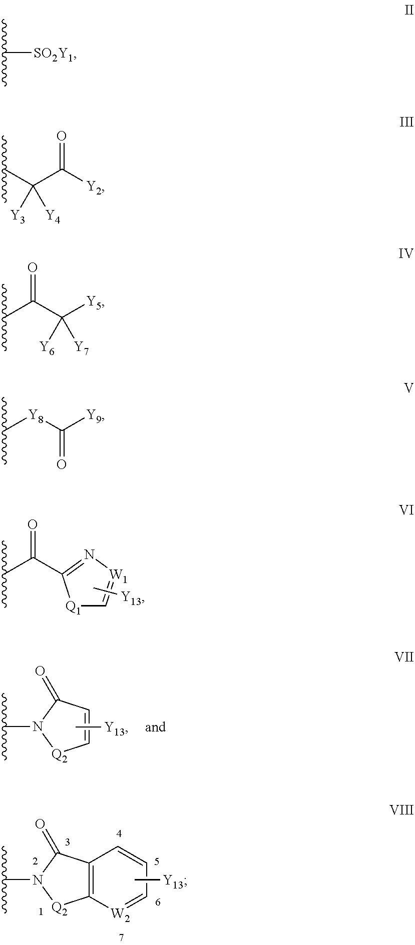
wherein:
Y₁ is —F, —Cl, —O-alkyl, —O-cycloalkyl, —O-heterocyclic, —O-aryl, —O-heteroaryl, or —O-adamantyl;
Y₂ is —H, —OH, —NH₂, —OMe, —OEt, —CF₃, —C≡CH, —CH₂—C≡CH, —CH═CH₂, fluoroalkyl, —C₁₋₅-alkyl, -aryl, -alkyl-aryl, -aryl-alkyl, -aryl-alkyl-Y₁₄, -aryl-heteroaryl, -aryl-aryl, -heteroaryl, -heteroaryl-alkyl, -heteroaryl-alkyl-Y₁₄, -heteroaryl-aryl, -heteroaryl-heteroaryl, -cycloalkyl, -cycloalkyl-alkyl, -cycloalkyl-alkyl-Y₁₄, -heterocyclic, -heterocyclic-alkyl, -heterocyclic-alkyl-Y₁₄, -adamantyl, —C₁₋₅-alkyl-Y₁₄, -aryl-Y₁₄, -heteroaryl-Y₁₄, -cycloalkyl-Y₁₄, -heterocyclic-Y₁₄, or -adamantyl-Y₁₄;
Y₃ and Y₄ are each independently —F, —Cl, or —OH; or Y3 and Y4 taken together form a ketone;
Y₅ is —F, —CONH₂, —SO₂NH₂, —COOH, —COOMe, —COOEt, —CF₃, —C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₆ and Y₇ are each independently —F, —Cl, or —OH;
Y₈ is NH, O, or heterocycle;
Y₉ is —OY₁₀, —N(Y₁₁)Y₁₂, or heterocycle;
Y₁₀ is alkyl, aryl, benzyl, difluorophenyl, fluorophenyl, heteroaryl, cycloalkyl, adamantyl, heterocyclic, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₁₁ is —H, -alkyl, -aryl, or -alkyl-aryl;
Y₁₂ is alkyl, aryl, heteroaryl, cycloalkyl, adamantyl, heterocyclic, —C₁₋₅-alkyl-Y₁₄, —C₁₋₅-alkyl-aryl, —C₁₋₅-alkyl-heteroaryl, -aryl-(Y₁₄)₁₋₄, -heteroaryl-Y₁₄, -cycloalkyl-Y₁₄, -adamantyl-Y₁₄, or -heterocyclic-Y₁₄; or Y₁₁ and Y₁₂ when taken together along with the N to which they are bonded form a 5- or 6-membered saturated heterocylic ring, the ring containing up to one additional heteroatom selected from the group consisting of N, O, and S;
Y₁₃ is —H, —OH, —SH, —NH₂, —CN, —N₃, —NCS, —NCO, —CONH₂, —SO₂NH₂, —COOH, —COOMe, —COOEt, —NO₂, —CF₃, —SO₃H, —P(O)(OH)₂, —C≡CH, —CH₂—C≡CH, —CH═CH₂, fluoroalkyl, —C₁₋₆-alkyl, aryl, heteroaryl, cycloalkyl, adamantyl, heterocyclic, —C₁₋₆-alkyl-Y₁₄; aryl Y₁₄-heteroaryl-Y₁₄, -cycloalkyl-Y₁₄, -adamantyl-Y₁₄, or -heterocyclic-Y₁₀;
Y₁₄ is —H, —F, —Cl, Br, —I, —OH, —OMe, —OEt, —OPh, —OBn, —SH, —NH₂, —CN, —N₃, —NCS, —NCO, —CONH₂, —SO₂NH₂, —COOH, —COOMe, —COOEt, —NO₂, -alkyl, —CF₃, —SO₃H, —P(O)(OH)₂, —CH₂—C≡CH, —CH═CH₂, or —NHCOCH₃, or —CH₂OH;
W₁ is CH or N if Y₁₃ is not bonded to W₁, or W₁ is C if Y₁₃ is bonded to W₁;
W₂ is CH or N if W₂ is not bonded to Y₁₃, or W₂ is C if W₂ is bonded to Y₁₃; if W₂ is N then it can occupy position 4, 5, 6, or 7 in VIII;
Q₁ is —CH₂, —O, —S, or —NH if Q₁ is not bonded to Y₁₃; Q₁ is —CH or —N if Q₁ is bonded to Y₁₃;
Q₂ is —SO₂, —C(O), or —S(O);
wherein:
X is —(CH₂)ₙ—, —(CH₂)ⱼ-A-(CH₂)ₖ—, cycloalkyl, or heterocycle, wherein:
A is —CH═CH—, C═O, O, S, or NH;
n is an integer from 0 to 15;
j is an integer from 0 to 10;
k is an integer from 0 to 10;
wherein:
R is selected from the group consisting of: 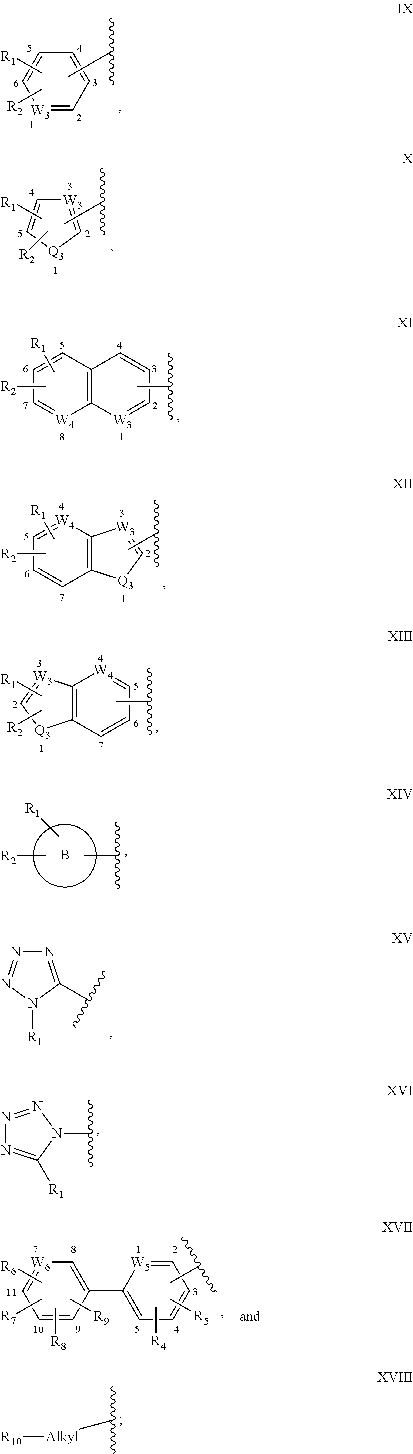
wherein:
W₃ is CH, O, or N if W₃ is not bonded to X or R₁ or R₂; W₃ is C if W₃ is bonded to X or R₁ or R₂; if W₃ is N then it can occupy position 1, 2, 3, 4, 5 or 6 in IX, position 2, 3, 4 or 5 in X, position 1, 2, 3 or 4 in XI, position 2 or 3 in XII, and position 2 or 3 in XIII;
W₄ is CH or N if W₄ is not bonded to X or R₁ or R₂; W₄ is C if W₄ is bonded to X or R₁ or R₂; if W₄ is N then it can occupy position 5, 6, 7 or 8 in XI, position 4, 5, 6 or 7 in XII and position 4, 5, 6 or 7 in XIII;
W₅ is CH or N if W₅ is not bonded to X or R₄ or R₅; W₅ is C if W₅ is bonded to X or R₄ or R₅; if W₅ is N then it can occupy position 1, 2, 3, 4 or 5 in XVII;
W₆ is CH or N if W₆ is not bonded to R₆ or R₇ or R₈ or R₉; W₆ is C if W₆ is bonded to R₆ or R₇ or R₈ or R₉; if W₆ is N then it can occupy position 7, 8, 9, 10 or 11 in XVII;
Q₃ is CH₂, O, S or NH if Q₃ is not bonded to X or R₁ or R₂; Q₃ is CH or N if Q₃ is bonded to X or R₁ or R₂;
B is adamantyl or heteroadamantyl;
R₁ and R₂ are each independently —H, —F, —Cl, —Br, —I, —OH, —SH, —NH₂, —CN, —N3, —NCS, —NCO, —CONH₂, —SO₂NH₂, —COOH, —NO₂, —CHO, —CF₃, —SO₃H, —SO₂Cl, —SO₂F, —O—P(O)(OH)₂, —O—P(O)(O-alkyl)₂, —O—P(O)(OH)(O-alkyl), —P(O)(O-alkyl)₂, —P(O)(OH)(O-alkyl), —Sn(alkyl)₃, —Si(alkyl)₃, —C≡CH, —CH₂—C≡CH, —CH═CH₂, -alkyl-R₃, -cycloalkyl-R₃, -heterocyclic-R₃, -aryl-R₃, -heteroaryl-R₃, -alkyl-cycloalkyl-R₃, -alkyl-heterocyclic-R₃, -alkyl-aryl-R₃, -alkyl-heteroaryl-R₃, —Z-alkyl-R₃, —Z-cycloalkyl-R₃, —Z-heterocyclic-R₃, —Z-aryl-R₃, —Z-heteroaryl-R₃, —Z-alkyl-cycloalkyl-R₃, —Z-alkyl-heterocyclic-R₃, —Z-alkyl-aryl-R₃, —Z-alkyl-heteroaryl-R₃, -aryl-Z-alkyl-R₃, -aryl-Z-cycloalkyl-R₃, -aryl-Z-heterocyclic-R₃, -aryl-Z-aryl-R₃, -aryl-Z-heteroaryl-R₃, -aryl-Z-alkyl-cycloalkyl-R₃, -aryl-Z-alkyl-heterocyclic-R₃, -aryl-Z-alkyl-aryl-R₃, -aryl-Z-alkyl-heteroaryl-R₃, —CH(alkyl-R₃)₂, —C(alkyl-R₃)₃, —N(alkyl-R₃)₂, —C(O)N(alkyl-R₃)₂, —SO₂N(alkyl-R₃)₂, or adamantyl;
Z is —O, —S, —NH, —C(O), —C(O)O, —OC(O), —C(O)NH, —NHC(O), —SO, —SO₂, —SO₂NH, —NHSO₂, —SO₂O, or —OSO₂;
R₃ is —H, —F, —Cl, —Br, —I, -Me, -Et, —OH, —OAc, —SH, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, —C≡CH, —CH₂—C≡CH, or —CH═CH₂;
R₄, R₅, R₆, R₇, R₈, and R₉ are each independently —H, —F, —Cl, —Br, —I, —OH, —OMe, —OEt, —OCH₂OCH₃, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, -alkyl, or -alkyl-R₃; and
R₁₀ is —H, —F, —Cl, —Br, —I, —OH, —OMe, —OEt, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —O Si(alkyl)₃, —C≡CH, —CH₂—C≡CH, or —CH═CH₂;
wherein:
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0, then R can not be IX, X, XI, XII, XIII, or XVIII when one of R₁ or R₂ is H;
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0-3, and R is XVII, then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂;
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0; then R can not be IX, X, XI, XII, XIII, or XVIII when one of R₁ or R₂ is H; and
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0-3, and R is XVII; then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂; and
(b) detecting a decrease in a symptom of the fertility disorder,
the administration of the compound treating the fertility disorder of the subject.

21. The method of claim 20, wherein the compound having formula R—X—Y is a compound listed in Table 2.

22. A method of treating an appetite disorder in a subject, comprising:
(a) administering to the subject a therapeutically effective amount of a compound having formula R—X—Y,
wherein:
Y is selected from the group consisting of: 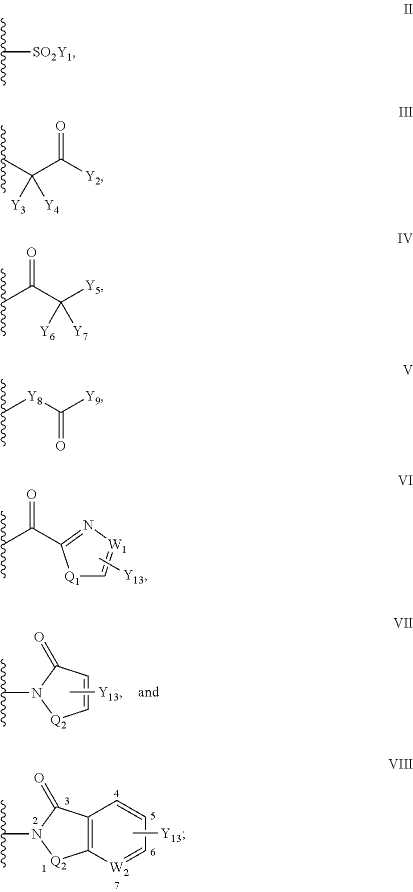
wherein:
Y₁ is —F, —Cl, —O-alkyl, —O-cycloalkyl, —O-heterocyclic, —O-aryl, —O-heteroaryl, or —O-adamantyl;
Y₂ is —H, —OH, —NH₂, —OMe, —OEt, —CF₃, —C≡CH, —CH₂—C≡CH, —CH═CH₂, fluoroalkyl, —C₁₋₅-alkyl, -aryl, -alkyl-aryl, -aryl-alkyl, -aryl-alkyl-Y₁₄, -aryl-heteroaryl, -aryl-aryl, -heteroaryl, -heteroaryl-alkyl, -heteroaryl-alkyl-Y₁₄, -heteroaryl-aryl, -heteroaryl-heteroaryl, -cycloalkyl, -cycloalkyl-alkyl, -cycloalkyl-alkyl-Y₁₄, -heterocyclic, -heterocyclic-alkyl, -heterocyclic-alkyl-Y₁₄, -adamantyl, —C₁₋₅-alkyl-Y₁₄, -aryl-Y₁₄, -heteroaryl-Y₁₄, -cycloalkyl-Y₁₄, -heterocyclic-Y₁₄, or -adamantyl-Y₁₄;
Y₃ and Y₄ are each independently —F, —Cl, or —OH; or Y3 and Y4 taken together form a ketone;
Y₅ is —F, —CONH₂, —SO₂NH₂, —COOH, —COOMe, —COOEt, —CF₃, —C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₆ and Y₇ are each independently —F, —Cl, or —OH;
Y₈ is NH, O, or heterocycle;
Y₉ is —OY₁₀, —N(Y₁₁)Y₁₂, or heterocycle;
Y₁₀ is alkyl, aryl, benzyl, difluorophenyl, fluorophenyl, heteroaryl, cycloalkyl, adamantyl, heterocyclic, —C₁₋₅-alkyl-Y₁₄; -aryl-Y₁₄, -heteroaryl-Y₁₄; -cycloalkyl-Y₁₄; -adamantyl-Y₁₄, or -heterocyclic-Y₁₄;
Y₁₁ is —H, -alkyl, -aryl, or -alkyl-aryl;
Y₁₂ is alkyl, aryl, heteroaryl, cycloalkyl, adamantyl, heterocyclic, C₁₋₅ alkyl Y₁₄—C₁₋₅-alkyl-aryl, —C₁₋₅-alkyl-heteroaryl, -aryl-(Y₁₄)₁₋₄, -heteroaryl-Y₁₄, -cycloalkyl-Y₁₄, -adamantyl-Y₁₄; or -heterocyclic-Y₁₄; or Y₁₁ and Y₁₂ when taken together along with the N to which they are bonded form a 5- or 6-membered saturated heterocylic ring, the ring containing up to one additional heteroatom selected from the group consisting of N, O, and S;
Y₁₃ is —H, —OH, —SH, —NH₂, —CN, —N₃, —NCS, —NCO, —CONH₂, —SO₂NH₂, —COOH, —COOMe, —COOEt, —NO₂, —CF₃, —SO₃H, —P(O)(OH)₂, —C≡CH, —CH₂—C≡CH, —CH═CH₂, fluoroalkyl, —C₁₋₆-alkyl, aryl, heteroaryl, cycloalkyl, adamantyl, heterocyclic, —C₁₋₆-alkyl-Y₁₄, -aryl-Y₁₄, -heteroaryl-Y₁₄, -cycloalkyl-Y₁₄, -adamantyl-Y₁₄, or -heterocyclic-Y₁₀;
Y₁₄ is —H, —F, —Cl, Br, —I, —OH, —OMe, —OEt, —OPh, —OBn, —SH, —NH₂, —CN, —N₃, —NCS, —NCO, —CONH₂, —SO₂NH₂, —COOH, —COOMe, —COOEt, —NO₂, -alkyl, —CF₃, —SO₃H, —P(O)(OH)₂, —C≡CH, —CH₂—C≡CH, —CH═CH₂, or —NHCOCH₃, or —CH₂OH;
W₁ is CH or N if Y₁₃ is not bonded to W₁, or W₁ is C if Y₁₃ is bonded to W₁;
W₂ is CH or N if W₂ is not bonded to Y₁₃, or W₂ is C if W₂ is bonded to Y₁₃; if W₂ is N then it can occupy position 4, 5, 6, or 7 in VIII;
Q₁ is —CH₂, —O, —S, or —NH if Q₁ is not bonded to Y₁₃; Q₁ is —CH or —N if Q₁ is bonded to Y₁₃;
Q₂ is —SO₂, —C(O), or —S(O);
wherein:
X is —(CH₂)ₙ—, —(CH₂)ⱼ-A-(CH₂)ₖ—, cycloalkyl, or heterocycle, wherein:
A is —CH═CH—, —C≡C—, C═O, O, S, or NH;
n is an integer from 0 to 15;
j is an integer from 0 to 10;
k is an integer from 0 to 10;
wherein:
R is selected from the group consisting of: 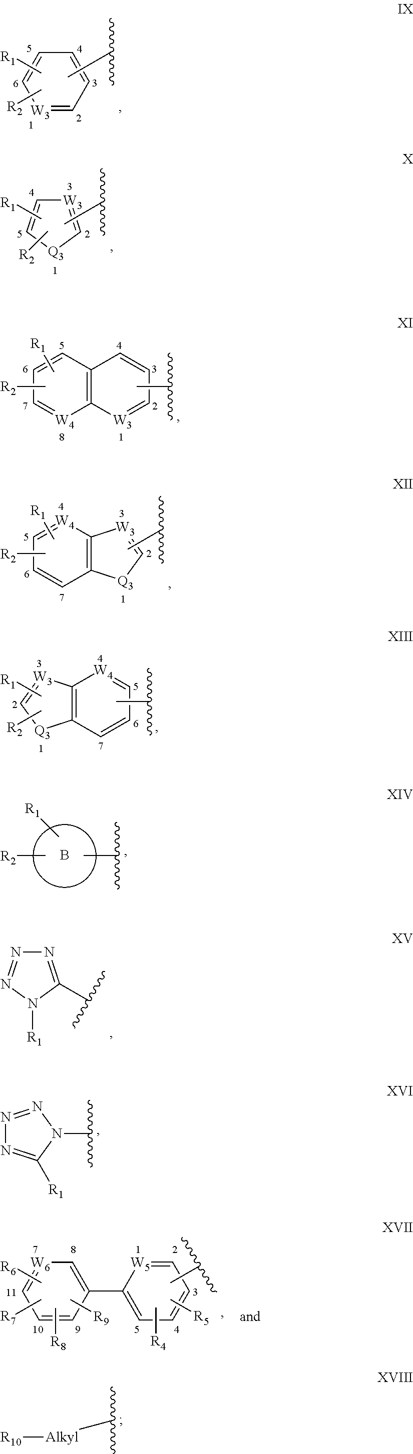
wherein:
W₃ is CH, O, or N if W₃ is not bonded to X or R₁ or R₂; W₃ is C if W₃ is bonded to X or R₁ or R₂; if W₃ is N then it can occupy position 1, 2, 3, 4, 5 or 6 in IX, position 2, 3, 4 or 5 in X, position 1, 2, 3 or 4 in XI, position 2 or 3 in XII, and position 2 or 3 in XIII;
W₄ is CH or N if W₄ is not bonded to X or R₁ or R₂; W₄ is C if W₄ is bonded to X or R₁ or R₂; if W₄ is N then it can occupy position 5, 6, 7 or 8 in XI, position 4, 5, 6 or 7 in XII and position 4, 5, 6 or 7 in XIII;
W₅ is CH or N if W₅ is not bonded to X or R₄ or R₅; W₅ is C if W₅ is bonded to X or R₄ or R₅; if W₅ is N then it can occupy position 1, 2, 3, 4 or 5 in XVII;
W₆ is CH or N if W₆ is not bonded to R₆ or R₇ or R₈ or R₉; W₆ is C if W₆ is bonded to R₆ or R₇ or R₈ or R₉; if W₆ is N then it can occupy position 7, 8, 9, 10 or 11 in XVII;
Q₃ is CH₂, O, S or NH if Q₃ is not bonded to X or R₁ or R₂; Q₃ is CH or N if Q₃ is bonded to X or R₁ or R₂;
B is adamantyl or heteroadamantyl;
R₁ and R₂ are each independently —H, —F, —Cl, —Br, —I, —OH, —SH, —NH₂, —CN, —N₃, —NCS, —NCO, —CONH₂, —SO₂NH₂, —COOH, —NO₂, —CHO, —CF₃, —SO₃H, —SO₂Cl, —SO₂F, —O—P(O)(OH)₂, —O—P(O)(O-alkyl)₂, —O—P(O)(OH)(O-alkyl), —P(O)(O-alkyl)₂, —P(O)(OH)(O-alkyl), —Sn(alkyl)₃, —Si(alkyl)₃, —C≡CH, —CH₂—C≡CH, —CH═CH₂, -alkyl-R₃, -cycloalkyl-R₃, -heterocyclic-R₃, -aryl-R₃, -heteroaryl-R₃, -alkyl-cycloalkyl-R₃, -alkyl-heterocyclic-R₃, -alkyl-aryl-R₃, -alkyl-heteroaryl-R₃, —Z-alkyl-R₃, —Z-cycloalkyl-R₃, —Z-heterocyclic-R₃, —Z-aryl-R₃, —Z-heteroaryl-R₃, —Z-alkyl-cycloalkyl-R₃, —Z-alkyl-heterocyclic-R₃, —Z-alkyl-aryl-R₃, —Z-alkyl-heteroaryl-R₃, -aryl-Z-alkyl-R₃, -aryl-Z-cycloalkyl-R₃, -aryl-Z-heterocyclic-R₃, -aryl-Z-aryl-R₃, -aryl-Z-heteroaryl-R₃, -aryl-Z-alkyl-cycloalkyl-R₃, -aryl-Z-alkyl-heterocyclic-R₃, -aryl-Z-alkyl-aryl-R₃, -aryl-Z-alkyl-heteroaryl-R₃, —CH(alkyl-R₃)₂, —C(alkyl-R₃)₃, —N(alkyl-R₃)₂, —C(O)N(alkyl-R₃)₂, —SO₂N(alkyl-R₃)₂, or adamantyl;
Z is —O, —S, —NH, —C(O), —C(O)O, —OC(O), —C(O)NH, —NHC(O), —SO, —SO₂, —SO₂NH, —NHSO₂, —SO₂O, or —OSO₂;
R₃ is —H, —F, —Cl, —Br, —I, -Me, -Et, —OH, —OAc, —SH, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, —C≡CH, —CH₂—C≡CH, or —CH═CH₂;
R₄, R₅, R₆, R₇, R₈, and R₉ are each independently —H, —F, —Cl, —Br, —I, —OH, —OMe, —OEt, —OCH₂OCH₃, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, -alkyl, or -alkyl-R₃; and
R₁₀ is —H, —F, —Cl, —Br, —I, —OH, —OMe, —OEt, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —O Si(alkyl)₃, —C≡CH, —CH₂—C≡CH, or —CH═CH₂;
wherein:
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0, then R can not be IX, X, XI, XII, XIII, or XVIII when one of R₁ or R₂ is H;
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0-3, and R is XVII, then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂;
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0; then R can not be IX, X, XI, XII, XIII, or XVIII when one of R₁ or R₂ is H; and
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0-3, and R is XVII; then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂; and
(b) detecting a decrease in a symptom of the appetite disorder,
the administration of the compound treating the appetite disorder of the subject.

23. The method of claim 22, wherein the compound having formula R—X—Y is a compound listed in Table 1.

24. A method of treating a metabolic disorder in a subject, comprising:
(a) administering to the subject a therapeutically effective amount of a compound having formula R—X—Y,
wherein:
Y is selected from the group consisting of: 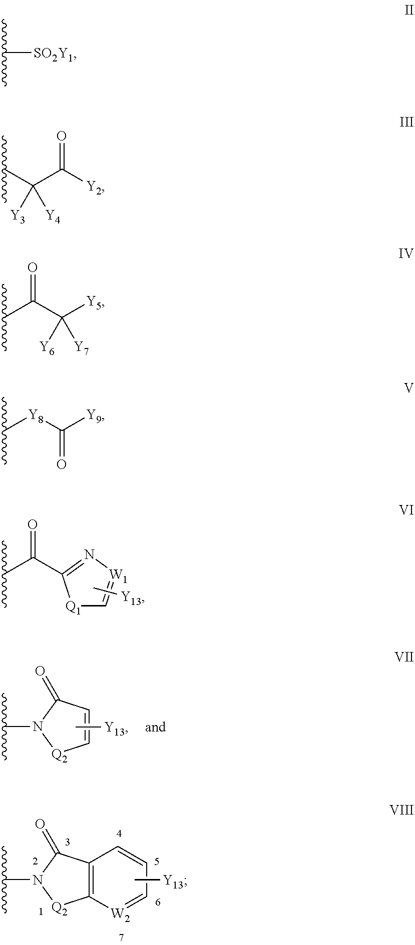
wherein:
Y₁ is —F, —Cl, —O-alkyl, —O-cycloalkyl, —O-heterocyclic, —O-aryl, —O-heteroaryl, or —O-adamantyl;
Y₂ is —H, —OH, —NH₂, —OMe, —OEt, —CF₃, —C≡CH, —CH₂—C≡CH, —CH═CH₂, fluoroalkyl, —C₁₋₅-alkyl, -aryl, -alkyl-aryl, -aryl-alkyl, -aryl-alkyl-Y₁₄, -aryl-heteroaryl, -aryl-aryl, -heteroaryl, -heteroaryl-alkyl, -heteroaryl-alkyl-Y₁₄, -heteroaryl-aryl, -heteroaryl-heteroaryl, -cycloalkyl, -cycloalkyl-alkyl, -cycloalkyl-alkyl-Y₁₄, -heterocyclic, -heterocyclic-alkyl, -heterocyclic-alkyl-Y₁₄, -adamantyl, —C₁₋₅-alkyl-Y₁₄, -aryl-Y₁₄, -heteroaryl-Y₁₄, -cycloalkyl-Y₁₄, -heterocyclic-Y₁₄, or -adamantyl-Y₁₄;
Y₃ and Y₄ are each independently —F, —Cl, or —OH; or Y3 and Y4 taken together form a ketone;
Y₅ is —F, —CONH₂, —SO₂NH₂, —COOH, —COOMe, —COOEt, —CF₃, —C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄; -aryl-Y₁₄, heteroaryl Y₁₄ cycloalkyl-Y₁₄, -adamantyl-Y₁₄, or -heterocyclic-Y₁₄;
Y₆ and Y₇ are each independently —F, —Cl, or —OH;
Y₈ is NH, O, or heterocycle;
Y₉ is —OY₁₀, —N(Y₁₁)Y₁₂, or heterocycle;
Y₁₀ is alkyl, aryl, benzyl, difluorophenyl, fluorophenyl, heteroaryl, cycloalkyl, adamantyl, heterocyclic, —C₁₋₅-alkyl-Y₁₄; -aryl-Y₁₄, -heteroaryl-Y₁₄, -cycloalkyl-Y₁₄; -adamantyl-Y₁₄, or -heterocyclic-Y₁₄;
Y₁₁ is —H, -alkyl, -aryl, or -alkyl-aryl;
Y₁₂ is alkyl, aryl, heteroaryl, cycloalkyl, adamantyl, heterocyclic, —C₁₋₅-alkyl-Y₁₄, —C₁₋₅-alkyl-aryl, —C₁₋₅-alkyl-heteroaryl, -aryl-(Y₁₄)₁₋₄; -heteroaryl-Y₁₄, -cycloalkyl-Y₁₄, -adamantyl-Y₁₄; or -heterocyclic-Y₁₄; or Y₁₁ and Y₁₂ when taken together along with the N to which they are bonded form a 5- or 6-membered saturated heterocylic ring, the ring containing up to one additional heteroatom selected from the group consisting of N, O, and S;
Y₁₃ is —H, —OH, —SH, —NH₂, —CN, —N₃, —NCS, —NCO, —CONH₂, —SO₂NH₂; —COOH, —COOMe, —COOEt, —NO₂, —CF₃, —SO₃H, —P(O)(OH)₂, —C≡CH, —CH₂—C≡CH, —CH═CH₂, fluoroalkyl, —C₁₋₆-alkyl, aryl, heteroaryl, cycloalkyl, adamantyl, heterocyclic, —C₁₋₆-alkyl-Y₁₄; -aryl-Y₁₄, -heteroaryl-Y₁₄, -cycloalkyl-Y₁₄; -adamantyl-Y₁₄, or -heterocyclic-Y₁₀;
Y₁₄ is —H, —F, —Cl, Br, —I, —OH, —OMe, —OEt, —OPh, —OBn, —SH, —NH₂, —CN, —N₃, —NCS, —NCO, —CONH₂, —SO₂NH₂, —COOH, —COOMe, —COOEt, —NO₂, -alkyl, —CF₃, —SO₃H, —P(O)(OH)₂, —C≡CH, —CH₂—C≡CH, —CH═CH₂, or —NHCOCH₃, or —CH₂OH;
W₁ is CH or N if Y₁₃ is not bonded to W₁, or W₁ is C if Y₁₃ is bonded to W₁;
W₂ is CH or N if W₂ is not bonded to Y₁₃, or W₂ is C if W₂ is bonded to Y₁₃; if W₂ is N then it can occupy position 4, 5, 6, or 7 in VIII;
Q₁ is —CH₂, —O, —S, or —NH if Q₁ is not bonded to Y₁₃; Q₁ is —CH or —N if Q₁ is bonded to Y₁₃;
Q₂ is —SO₂, —C(O), or —S(O);
wherein:
X is —(CH₂)ₙ—, —(CH₂)ⱼ-A-(CH₂)ₖ—, cycloalkyl, or heterocycle, wherein:
A is —CH═CH—, C═O, O, S, or NH;
n is an integer from 0 to 15;
j is an integer from 0 to 10;
k is an integer from 0 to 10;
wherein:
R is selected from the group consisting of: 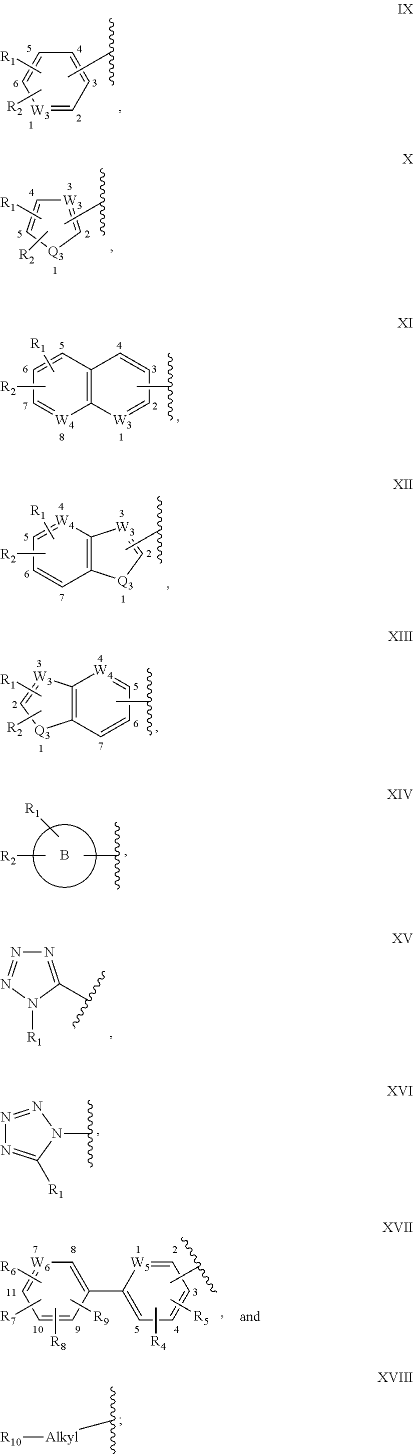
wherein:
W₃ is CH, O, or N if W₃ is not bonded to X or R₁ or R₂; W₃ is C if W₃ is bonded to X or R₁ or R₂; if W₃ is N then it can occupy position 1, 2, 3, 4, 5 or 6 in IX, position 2, 3, 4 or 5 in X, position 1, 2, 3 or 4 in XI, position 2 or 3 in XII, and position 2 or 3 in XIII;
W₄ is CH or N if W₄ is not bonded to X or R₁ or R₂; W₄ is C if W₄ is bonded to X or R₁ or R₂; if W₄ is N then it can occupy position 5, 6, 7 or 8 in XI, position 4, 5, 6 or 7 in XII and position 4, 5, 6 or 7 in XIII;
W₅ is CH or N if W₅ is not bonded to X or R₄ or R₅; W₅ is C if W₅ is bonded to X or R₄ or R₅; if W₅ is N then it can occupy position 1, 2, 3, 4 or 5 in XVII;
W₆ is CH or N if W₆ is not bonded to R₆ or R₇ or R₈ or R₉; W₆ is C if W₆ is bonded to R₆ or R₇ or R₈ or R₉; if W₆ is N then it can occupy position 7, 8, 9, 10 or 11 in XVII;
Q₃ is CH₂, O, S or NH if Q₃ is not bonded to X or R₁ or R₂; Q₃ is CH or N if Q₃ is bonded to X or R₁ or R₂;
B is adamantyl or heteroadamantyl;
R₁ and R₂ are each independently —H, —F, —Cl, —Br, —I, —OH, —SH, —NH₂, —CN, —N₃, —NCS, —NCO, —CONH₂, —SO₂NH₂, —COOH, —NO₂, —CHO, —CF₃, —SO₃H, —SO₂Cl, —SO₂F, —O—P(O)(OH)₂, —O—P(O)(O-alkyl)₂, —O—P(O)(OH)(O-alkyl), —P(O)(O-alkyl)₂, —P(O)(OH)(O-alkyl), —Sn(alkyl)₃, —Si(alkyl)₃, —C≡CH, —CH₂—C≡CH, —CH═CH₂, -alkyl-R₃, -cycloalkyl-R₃, -heterocyclic-R₃, -aryl-R₃, -heteroaryl-R₃, -alkyl-cycloalkyl-R₃, -alkyl-heterocyclic-R₃, -alkyl-aryl-R₃, -alkyl-heteroaryl-R₃, —Z-alkyl-R₃, —Z-cycloalkyl-R₃, —Z-heterocyclic-R₃, —Z-aryl-R₃, —Z-heteroaryl-R₃, —Z-alkyl-cycloalkyl-R₃, —Z-alkyl-heterocyclic-R₃, —Z-alkyl-aryl-R₃, —Z-alkyl-heteroaryl-R₃, -aryl-Z-alkyl-R₃, -aryl-Z-cycloalkyl-R₃, -aryl-Z-heterocyclic-R₃, -aryl-Z-aryl-R₃, -aryl-Z-heteroaryl-R₃, -aryl-Z-alkyl-cycloalkyl-R₃, -aryl-Z-alkyl-heterocyclic-R₃, -aryl-Z-alkyl-aryl-R₃, -aryl-Z-alkyl-heteroaryl-R₃, —CH(alkyl-R₃)₂, —C(alkyl-R₃)₃, —N(alkyl-R₃)₂, —C(O)N(alkyl-R₃)₂, —SO₂N(alkyl-R₃)₂, or adamantyl;
Z is —O, —S, —NH, —C(O), —C(O)O, —OC(O), —C(O)NH, —NHC(O), —SO, —SO₂, —SO₂NH, —NHSO₂, —SO₂O, or —OSO₂;
R₃ is —H, —F, —Cl, —Br, —I, -Me, -Et, —OH, —OAc, —SH, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, —C≡CH, —CH₂—C≡CH, or —CH═CH₂;
R₄, R₅, R₆, R₇, R₈, and R₉ are each independently —H, —F, —Cl, —Br, —I, —OH, —OMe, —OEt, —OCH₂OCH₃, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, -alkyl, or -alkyl-R₃; and
R₁₀ is —H, —F, —Cl, —Br, —I, —OH, —OMe, —OEt, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —O Si(alkyl)₃, —C≡CH, —CH₂—C≡CH, or —CH═CH₂;
wherein:
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0, then R can not be IX, X, XI, XII, XIII, or XVIII when one of R₁ or R₂ is H;
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0-3, and R is XVII, then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂;
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0; then R can not be IX, X, XI, XII, XIII, or XVIII when one of R₁ or R₂ is H; and
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0-3, and R is XVII; then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂; and
(b) detecting a decrease in a symptom of the metabolic disorder,
the administration of the compound treating the metabolic disorder of the subject.

25. The method of claim 24, wherein the compound having formula R—X—Y is a compound listed in Table 1.

26. A method of treating a movement disorder in a subject, comprising:
(a) administering to the subject a therapeutically effective amount of a compound having formula R—X—Y,
wherein:
Y is selected from the group consisting of: 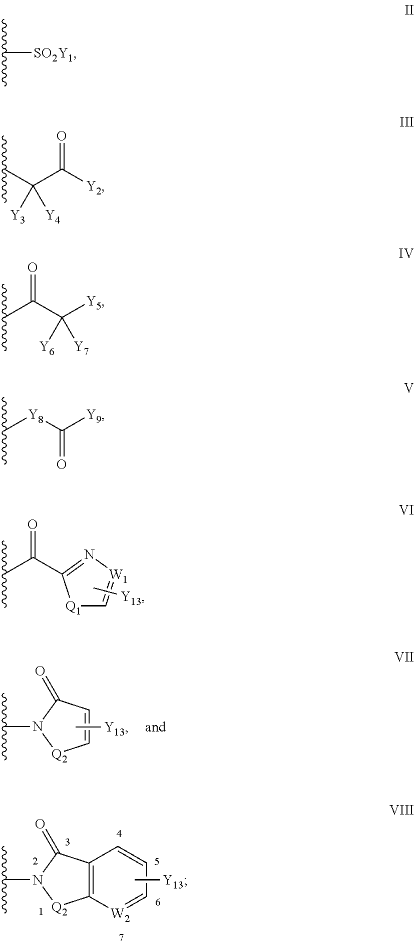
wherein:
Y₁ is —F, —Cl, —O-alkyl, —O-cycloalkyl, —O-heterocyclic, —O-aryl, —O-heteroaryl, or —O-adamantyl;
Y₂ is —H, —OH, —NH₂, —OMe, —OEt, —CF₃, —C≡CH, —CH₂—C≡CH, —CH═CH₂, fluoroalkyl, —C₁₋₅-alkyl, -aryl, -alkyl-aryl, -aryl-alkyl, -aryl-alkyl-Y₁₄, -aryl-heteroaryl, -aryl-aryl, -heteroaryl, -heteroaryl-alkyl, -heteroaryl-alkyl-Y₁₄, -heteroaryl-aryl, heteroaryl-heteroaryl, -cycloalkyl, -cycloalkyl-alkyl, -cycloalkyl-alkyl-Y₁₄; -heterocyclic, -heterocyclic-alkyl, -heterocyclic-alkyl-Y₁₄, -adamantyl, —C₁₋₅-alkyl-Y₁₄, -aryl-Y₁₄, -heteroaryl-Y₁₄, -cycloalkyl-Y₁₄, -heterocyclic-Y₁₄, or -adamantyl-Y₁₄;
Y₃ and Y₄ are each independently —F, —Cl, or —OH; or Y3 and Y4 taken together form a ketone;
Y₅ is —F, —CONH₂, —SO₂NH₂, —COOH, —COOMe, —COOEt, —CF₃, —C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₆ and Y₇ are each independently —F, —Cl, or —OH;
Y₈ is NH, O, or heterocycle;
Y₉ is —OY₁₀, —N(Y₁₁)Y₁₂, or heterocycle;
Y₁₀ is alkyl, aryl, benzyl, difluorophenyl, fluorophenyl, heteroaryl, cycloalkyl, adamantyl, heterocyclic, —C₁₋₅-alkyl-Y₁₄; -aryl-Y₁₄, -heteroaryl-Y₁₄; -cycloalkyl-Y₁₄; -adamantyl-Y₁₄, or -heterocyclic-Y₁₄;
Y₁₁ is —H, -alkyl, -aryl, or -alkyl-aryl;
Y₁₂ is alkyl, aryl, heteroaryl, cycloalkyl, adamantyl, heterocyclic, —C₁₋₅-alkyl-Y₁₄, —C₁₋₅-alkyl-aryl, —C₁₋₅-alkyl-heteroaryl, -aryl-(Y₁₄)₁₋₄; -heteroaryl-Y₁₄, -cycloalkyl-Y₁₄, -adamantyl-Y₁₄; or -heterocyclic-Y₁₄; or Y₁₁ and Y₁₂ when taken together along with the N to which they are bonded form a 5- or 6-membered saturated heterocylic ring, the ring containing up to one additional heteroatom selected from the group consisting of N, O, and S;
Y₁₃ is —H, —OH, —SH, —NH₂, —CN, —N₃, —NCS, —NCO, —CONH₂, —SO₂NH₂; —COOH, —COOMe, —COOEt, —NO₂, —CF₃, —SO₃H, —P(O)(OH)₂, —C≡CH, —CH₂—C≡CH, —CH═CH₂, fluoroalkyl, —C₁₋₆-alkyl, aryl, heteroaryl, cycloalkyl, adamantyl, heterocyclic, —C₁₋₆-alkyl-Y₁₄; -aryl-Y₁₄, heteroaryl Y₁₄ cycloalkyl-Y₁₄, -adamantyl-Y₁₄, or -heterocyclic-Y₁₀;
Y₁₄ is —H, —F, —Cl, Br, —I, —OH, —OMe, —OEt, —OPh, —OBn, —SH, —NH₂, —CN, —N₃, —NCS, —NCO, —CONH₂, —SO₂NH₂, —COOH, —COOMe, —COOEt, —NO₂, -alkyl, —CF₃, —SO₃H, —P(O)(OH)₂, —C≡CH, —CH₂—C≡CH, —CH═CH₂, or —NHCOCH₃, or —CH₂OH;
W₁ is CH or N if Y₁₃ is not bonded to W₁, or W₁ is C if Y₁₃ is bonded to W₁;
W₂ is CH or N if W₂ is not bonded to Y₁₃, or W₂ is C if W₂ is bonded to Y₁₃; if W₂ is N then it can occupy position 4, 5, 6, or 7 in VIII;
Q₁ is —CH₂, —O, —S, or —NH if Q₁ is not bonded to Y₁₃; Q₁ is —CH or —N if Q₁ is bonded to Y₁₃;
Q₂ is —SO₂, —C(O), or —S(O);
wherein:
X is —(CH₂)ₙ—, —(CH₂)ⱼ-A-(CH₂)ₖ—, cycloalkyl, or heterocycle, wherein:
A is —CH═CH—, C═O, O, S, or NH;
n is an integer from 0 to 15;
j is an integer from 0 to 10;
k is an integer from 0 to 10;
wherein:
R is selected from the group consisting of: 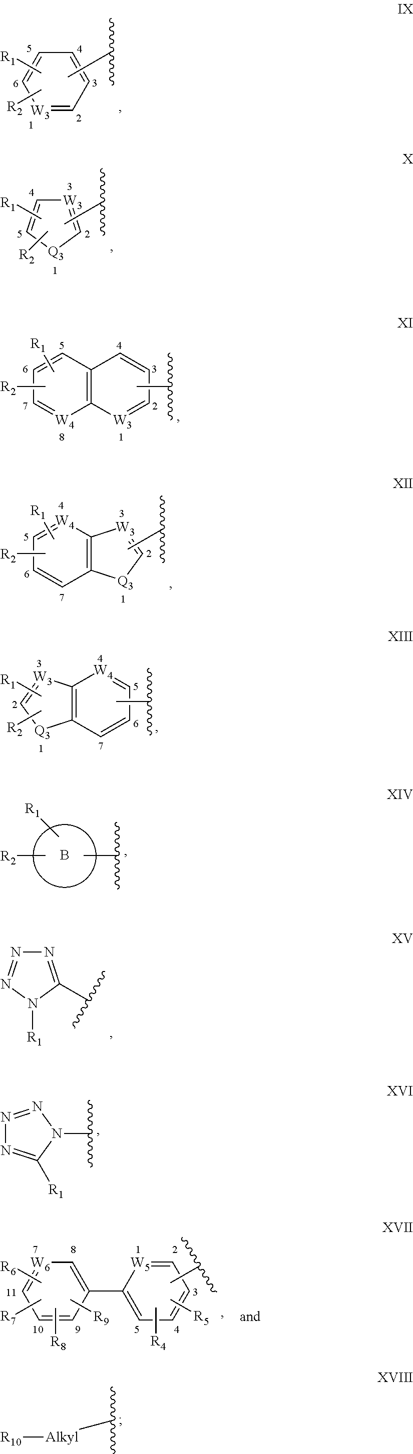
wherein:
W₃ is CH, O, or N if W₃ is not bonded to X or R₁ or R₂; W₃ is C if W₃ is bonded to X or R₁ or R₂; if W₃ is N then it can occupy position 1, 2, 3, 4, 5 or 6 in IX, position 2, 3, 4 or 5 in X, position 1, 2, 3 or 4 in XI, position 2 or 3 in XII, and position 2 or 3 in XIII;
W₄ is CH or N if W₄ is not bonded to X or R₁ or R₂; W₄ is C if W₄ is bonded to X or R₁ or R₂; if W₄ is N then it can occupy position 5, 6, 7 or 8 in XI, position 4, 5, 6 or 7 in XII and position 4, 5, 6 or 7 in XIII;
W₅ is CH or N if W₅ is not bonded to X or R₄ or R₅; W₅ is C if W₅ is bonded to X or R₄ or R₅; if W₅ is N then it can occupy position 1, 2, 3, 4 or 5 in XVII;
W₆ is CH or N if W₆ is not bonded to R₆ or R₇ or R₈ or R₉; W₆ is C if W₆ is bonded to R₆ or R₇ or R₈ or R₉; if W₆ is N then it can occupy position 7, 8, 9, 10 or 11 in XVII;
Q₃ is CH₂, O, S or NH if Q₃ is not bonded to X or R₁ or R₂; Q₃ is CH or N if Q₃ is bonded to X or R₁ or R₂;
B is adamantyl or heteroadamantyl;
R₁ and R₂ are each independently —H, —F, —Cl, —Br, —I, —OH, —SH, —NH₂, —CN, —N3, —NCS, —NCO, —CONH₂, —SO₂NH₂, —COOH, —NO₂, —CHO, —CF₃, —SO₃H, —SO₂Cl, —SO₂F, —O—P(O)(OH)₂, —O—P(O)(O-alkyl)₂, —O—P(O)(OH)(O-alkyl), —P(O)(O-alkyl)₂, —P(O)(OH)(O-alkyl), —Sn(alkyl)₃, —Si(alkyl)₃, —C≡CH, —CH₂—C≡CH, —CH═CH₂, -alkyl-R₃, -cycloalkyl-R₃, -heterocyclic-R₃, -aryl-R₃, -heteroaryl-R₃, -alkyl-cycloalkyl-R₃, -alkyl-heterocyclic-R₃, -alkyl-aryl-R₃, -alkyl-heteroaryl-R₃, —Z-alkyl-R₃, —Z-cycloalkyl-R₃, —Z-heterocyclic-R₃, —Z-aryl-R₃, —Z-heteroaryl-R₃, —Z-alkyl-cycloalkyl-R₃, —Z-alkyl-heterocyclic-R₃, —Z-alkyl-aryl-R₃, —Z-alkyl-heteroaryl-R₃, -aryl-Z-alkyl-R₃, -aryl-Z-cycloalkyl-R₃, -aryl-Z-heterocyclic-R₃, -aryl-Z-aryl-R₃, -aryl-Z-heteroaryl-R₃, -aryl-Z-alkyl-cycloalkyl-R₃, -aryl-Z-alkyl-heterocyclic-R₃, -aryl-Z-alkyl-aryl-R₃, -aryl-Z-alkyl-heteroaryl-R₃, —CH(alkyl-R₃)₂, —C(alkyl-R₃)₃, —N(alkyl-R₃)₂, —C(O)N(alkyl-R₃)₂, —SO₂N(alkyl-R₃)₂, or adamantyl;
Z is —O, —S, —NH, —C(O), —C(O)O, —OC(O), —C(O)NH, —NHC(O), —SO, —SO₂, —SO₂NH, —NHSO₂, —SO₂O, or —OSO₂;
R₃ is —H, —F, —Cl, —Br, —I, -Me, -Et, —OH, —OAc, —SH, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, —C≡CH, —CH₂—C≡CH, or —CH═CH₂;
R₄, R₅, R₆, R₇, R₈, and R₉ are each independently —H, —F, —Cl, —Br, —I, —OH, —OMe, —OEt, —OCH₂OCH₃, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, -alkyl, or -alkyl-R₃; and
R₁₀ is —H, —F, —Cl, —Br, —I, —OH, —OMe, —OEt, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —O Si(alkyl)₃, —C≡CH, —CH₂—C≡CH, or —CH═CH₂;
wherein:
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0, then R can not be IX, X, XI, XII, XIII, or XVIII when one of R₁ or R₂ is H;
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0-3, and R is XVII, then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂;
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0; then R can not be IX, X, XI, XII, XIII, or XVIII when one of R₁ or R₂ is H; and
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0-3, and R is XVII; then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂; and
(b) detecting a decrease in a symptom of the movement function disorder,
the administration of the compound treating the movement disorder of the subject.

27. The method of claim 26, wherein the compound having formula R—X—Y is a compound listed in Table 1.

28. A method of treating cancer in a subject, comprising:
(a) administering to the subject a therapeutically effective amount of a compound having formula R—X—Y,
wherein:
Y is selected from the group consisting of: 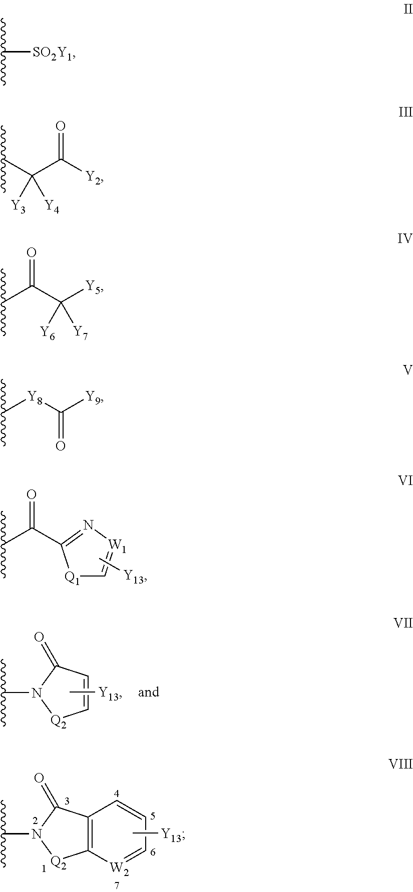
wherein:
Y₁ is —F, —Cl, —O-alkyl, —O-cycloalkyl, —O-heterocyclic, —O-aryl, —O-heteroaryl, or —O-adamantyl;
Y₂ is —H, —OH, —NH₂, —OMe, —OEt, —CF₃, —C≡CH, —CH₂—C≡CH, —CH═CH₂, fluoroalkyl, —C₁₋₅-alkyl, -aryl, -alkyl-aryl, -aryl-alkyl, -aryl-alkyl-Y₁₄, -aryl-heteroaryl, -aryl-aryl, -heteroaryl, -heteroaryl-alkyl, -heteroaryl-alkyl-Y₁₄, -heteroaryl-aryl, -heteroaryl-heteroaryl, -cycloalkyl, -cycloalkyl-alkyl, -cycloalkyl-alkyl-Y₁₄, -heterocyclic, -heterocyclic-alkyl, -heterocyclic-alkyl-Y₁₄, -adamantyl, —C₁₋₅-alkyl-Y₁₄, -aryl-Y₁₄, -heteroaryl-Y₁₄, -cycloalkyl-Y₁₄, -heterocyclic-Y₁₄, or -adamantyl-Y₁₄;
Y₃ and Y₄ are each independently —F, —Cl, or —OH; or Y3 and Y4 taken together form a ketone;
Y₅ is —F, —CONH₂, —SO₂NH₂, —COOH, —COOMe, —COOEt, —CF₃, —C≡CH, —CH₂—C≡CH, —CH═CH₂, fluoroalkyl, —C₁₋₅-alkyl, aryl, heteroaryl, cycloalkyl, heterocyclic, adamantyl, —C₁₋₅-alkyl-Y₁₄, -aryl-Y₁₄, -heteroaryl-Y₁₄, -cycloalkyl-Y₁₄, -adamantyl-Y₁₄, or -heterocyclic-Y₁₄;
Y₆ and Y₇ are each independently —F, —Cl, or —OH;
Y₈ is NH, O, or heterocycle;
Y₉ is —OY₁₀, —N(Y₁₁)Y₁₂, or heterocycle;
Y₁₀ is alkyl, aryl, benzyl, difluorophenyl, fluorophenyl, heteroaryl, cycloalkyl, adamantyl, heterocyclic, —C₁₋₅-alkyl-Y₁₄; -aryl-Y₁₄, -heteroaryl-Y₁₄; -cycloalkyl-Y₁₄; -adamantyl-Y₁₄, or -heterocyclic-Y₁₄;
Y₁₁ is —H, -alkyl, -aryl, or -alkyl-aryl;
Y₁₂ is alkyl, aryl, heteroaryl, cycloalkyl, adamantyl, heterocyclic, C₁₋₅ alkyl Y₁₄—C₁₋₅-alkyl-aryl, —C₁₋₅-alkyl-heteroaryl, -aryl-(Y₁₄)₁₋₄, -heteroaryl-Y₁₄, -cycloalkyl-Y₁₄, -adamantyl-Y₁₄; or -heterocyclic-Y₁₄; or Y₁₁ and Y₁₂ when taken together along with the N to which they are bonded form a 5- or 6-membered saturated heterocylic ring, the ring containing up to one additional heteroatom selected from the group consisting of N, O, and S;
Y₁₃ is —H, —OH, —SH, —NH₂, —CN, —N₃, —NCS, —NCO, —CONH₂, —SO₂NH₂, —COOH, —COOMe, —COOEt, —NO₂, —CF₃, —SO₃H, —P(O)(OH)₂, —C≡CH, —CH₂—C≡CH, —CH═CH₂, fluoroalkyl, —C₁₋₆-alkyl, aryl, heteroaryl, cycloalkyl, adamantyl, heterocyclic, —C₁₋₆-alkyl-Y₁₄, -aryl-Y₁₄, -heteroaryl-Y₁₄, -cycloalkyl-Y₁₄, -adamantyl-Y₁₄, or -heterocyclic-Y₁₀;
Y₁₄ is —H, —F, —Cl, Br, —I, —OH, —OMe, —OEt, —OPh, —OBn, —SH, —NH₂, —CN, —N₃, —NCS, —NCO, —CONH₂, —SO₂NH₂, —COOH, —COOMe, —COOEt, —NO₂, -alkyl, —CF₃, —SO₃H, —P(O)(OH)₂, —C≡CH, —CH₂—C≡CH, —CH═CH₂, or —NHCOCH₃, or —CH₂OH;
W₁ is CH or N if Y₁₃ is not bonded to W₁, or W₁ is C if Y₁₃ is bonded to W₁;
W₂ is CH or N if W₂ is not bonded to Y₁₃, or W₂ is C if W₂ is bonded to Y₁₃; if W₂ is N then it can occupy position 4, 5, 6, or 7 in VIII;
Q₁ is —CH₂, —O, —S, or —NH if Q₁ is not bonded to Y₁₃; Q₁ is —CH or —N if Q₁ is bonded to Y₁₃;
Q₂ is —SO₂, —C(O), or —S(O);
wherein:
X is —(CH₂)ₙ—, —(CH₂)ⱼ-A-(CH₂)ₖ—, cycloalkyl, or heterocycle, wherein:
A is —CH═CH—, —C≡C—, C═O, O, S, or NH;
n is an integer from 0 to 15;
j is an integer from 0 to 10;
k is an integer from 0 to 10;
wherein:
R is selected from the group consisting of: 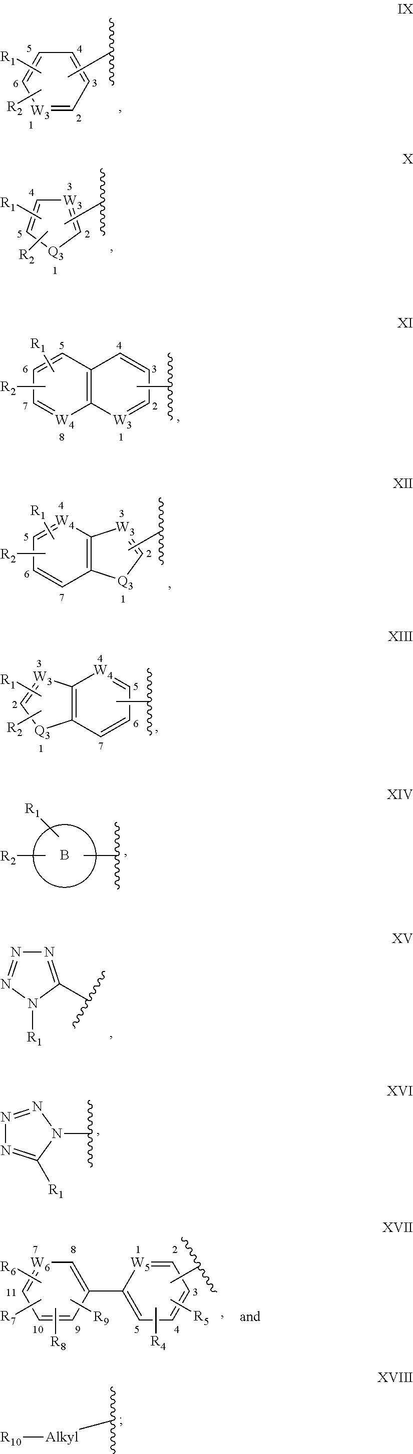
wherein:
W₃ is CH, O, or N if W₃ is not bonded to X or R₁ or R₂; W₃ is C if W₃ is bonded to X or R₁ or R₂; if W₃ is N then it can occupy position 1, 2, 3, 4, 5 or 6 in IX, position 2, 3, 4 or 5 in X, position 1, 2, 3 or 4 in XI, position 2 or 3 in XII, and position 2 or 3 in XIII;
W₄ is CH or N if W₄ is not bonded to X or R₁ or R₂; W₄ is C if W₄ is bonded to X or R₁ or R₂; if W₄ is N then it can occupy position 5, 6, 7 or 8 in XI, position 4, 5, 6 or 7 in XII and position 4, 5, 6 or 7 in XIII;
W₅ is CH or N if W₅ is not bonded to X or R₄ or R₅; W₅ is C if W₅ is bonded to X or R₄ or R₅; if W₅ is N then it can occupy position 1, 2, 3, 4 or 5 in XVII;
W₆ is CH or N if W₆ is not bonded to R₆ or R₇ or R₈ or R₉; W₆ is C if W₆ is bonded to R₆ or R₇ or R₈ or R₉; if W₆ is N then it can occupy position 7, 8, 9, 10 or 11 in XVII;
Q₃ is CH₂, O, S or NH if Q₃ is not bonded to X or R₁ or R₂; Q₃ is CH or N if Q₃ is bonded to X or R₁ or R₂;
B is adamantyl or heteroadamantyl;
R₁ and R₂ are each independently —H, —F, —Cl, —Br, —I, —OH, —SH, —NH₂, —CN, —N₃, —NCS, —NCO, —CONH₂, —SO₂NH₂, —COOH, —NO₂, —CHO, —CF₃, —SO₃H, —SO₂Cl, —SO₂F, —O—P(O)(OH)₂, —O—P(O)(O-alkyl)₂, —O—P(O)(OH)(O-alkyl), —P(O)(O-alkyl)₂, —P(O)(OH)(O-alkyl), —Sn(alkyl)₃, —Si(alkyl)₃, —C≡CH, —CH₂—C≡CH, —CH═CH₂, -alkyl-R₃, -cycloalkyl-R₃, -heterocyclic-R₃, -aryl-R₃, -heteroaryl-R₃, -alkyl-cycloalkyl-R₃, -alkyl-heterocyclic-R₃, -alkyl-aryl-R₃, -alkyl-heteroaryl-R₃, —Z-alkyl-R₃, —Z-cycloalkyl-R₃, —Z-heterocyclic-R₃, —Z-aryl-R₃, —Z-heteroaryl-R₃, —Z-alkyl-cycloalkyl-R₃, —Z-alkyl-heterocyclic-R₃, —Z-alkyl-aryl-R₃, —Z-alkyl-heteroaryl-R₃, -aryl-Z-alkyl-R₃, -aryl-Z-cycloalkyl-R₃, -aryl-Z-heterocyclic-R₃, -aryl-Z-aryl-R₃, -aryl-Z-heteroaryl-R₃, -aryl-Z-alkyl-cycloalkyl-R₃, -aryl-Z-alkyl-heterocyclic-R₃, -aryl-Z-alkyl-aryl-R₃, -aryl-Z-alkyl-heteroaryl-R₃, —CH(alkyl-R₃)₂, —C(alkyl-R₃)₃, —N(alkyl-R₃)₂, —C(O)N(alkyl-R₃)₂, —SO₂N(alkyl-R₃)₂, or adamantyl;
Z is —O, —S, —NH, —C(O), —C(O)O, —OC(O), —C(O)NH, —NHC(O), —SO, —SO₂, —SO₂NH, —NHSO₂, —SO₂O, or —OSO₂;
R₃ is —H, —F, —Cl, —Br, —I, -Me, -Et, —OH, —OAc, —SH, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, —C≡CH, —CH₂—C≡CH, or —CH═CH₂;
R₄, R₅, R₆, R₇, R₈, and R₉ are each independently —H, —F, —Cl, —Br, —I, —OH, —OMe, —OEt, —OCH₂OCH₃, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —OSi(alkyl)₃, -alkyl, or -alkyl-R₃; and
R₁₀ is —H, —F, —Cl, —Br, —I, —OH, —OMe, —OEt, —OAc, —SH, —SMe, —SEt, —NH₂, —CN, —N₃, —NCS, —NCO, —CONH₂, —SO₂NH₂, —COOH, —NO₂, —CHO, —CF₃, —SO₃H, —SO₂F, —O—P(O)(OH)₂, —Sn(alkyl)₃, —Si(alkyl)₃, —O Si(alkyl)₃, —C≡CH, —CH₂—C≡CH, or —CH═CH₂;
wherein:
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0, then R can not be IX, X, XI, XII, XIII, or XVIII when one of R₁ or R₂ is H;
if Y is V, Y₈ is O or NH, Y₉ is OY₁₀ where Y₁₀ is alkyl, cycloalkyl, heterocyclic, aryl, phenyl, or heteroaryl, and X is —(CH₂)n- where n=0-3, and R is XVII, then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂;
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0; then R can not be IX, X, XI, XII, XIII, or XVIII when one of R₁ or R₂ is H; and
if Y is V, Y₈ is O or NH, Y₉ is N(Y₁₁)Y₁₂ where Y₁₁ is H and Y₁₂ is alkyl, cycloalkyl, heterocyclic, aryl, phenyl, or heteroaryl, or where Y₁₁ and Y₁₂ when taken together along with the N to which they are bonded form a 5- or 6-membered saturated heterocylic ring, X is —(CH₂)n- where n=0-3, and R is XVII; then each of R₄, R₅, R₆, R₇, R₈, and R₉ can not be H, alkyl, OMe, OEt, F, Cl, Br, I, CN, OH, NO₂, NH₂, SH, SMe, SEt, CONH₂, or SO₂NH₂; and
(b) detecting a decrease in a symptom of the cancer,
the administration of the compound treating the cancer of the subject.

29. The method of claim 28, wherein the compound having formula R—X—Y is a compound listed in Table 2.

30. A compound selected from the group consisting of: 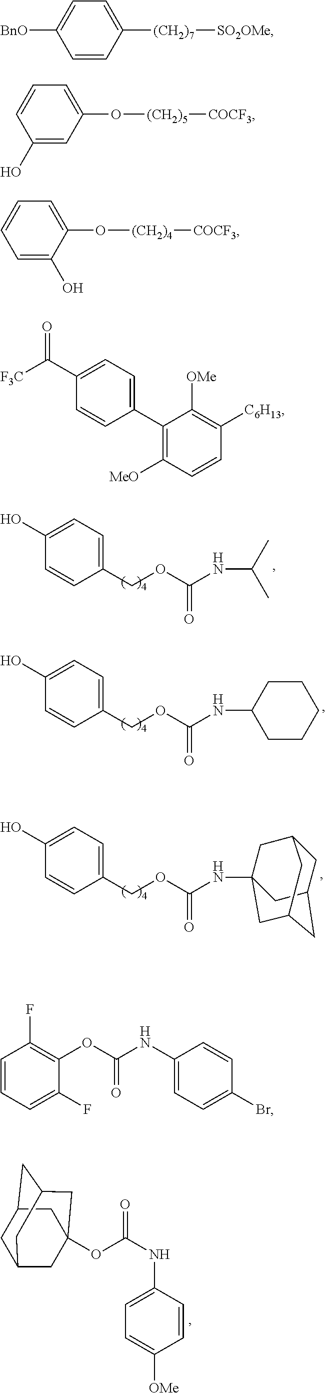 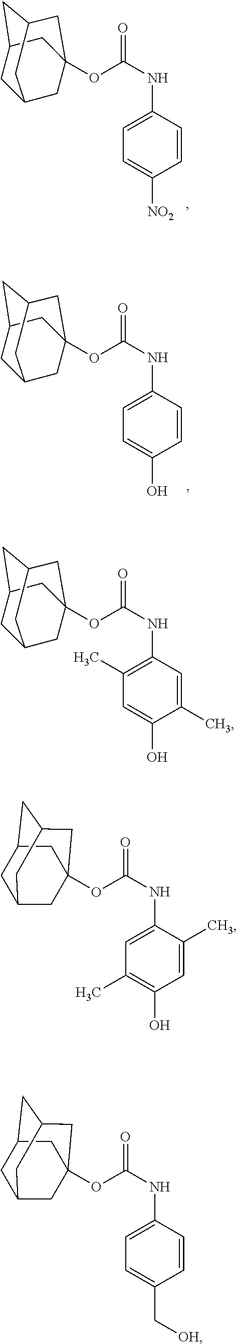 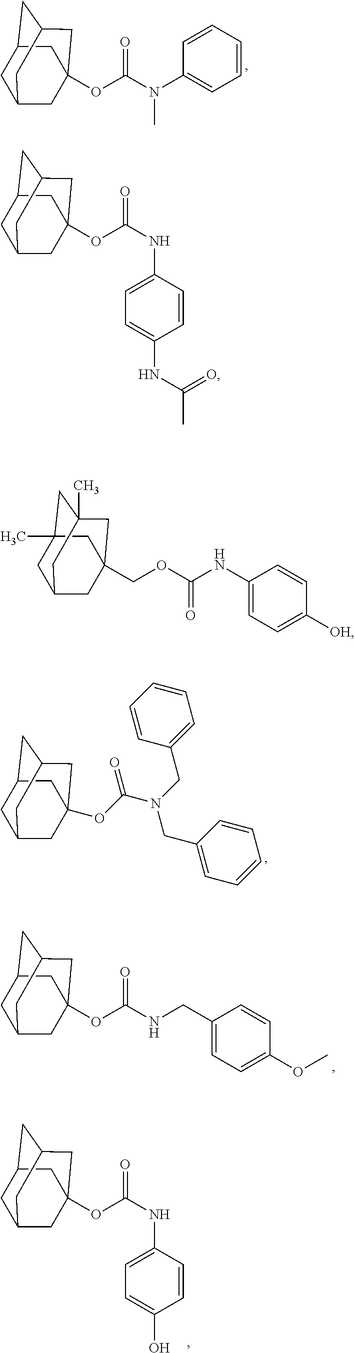 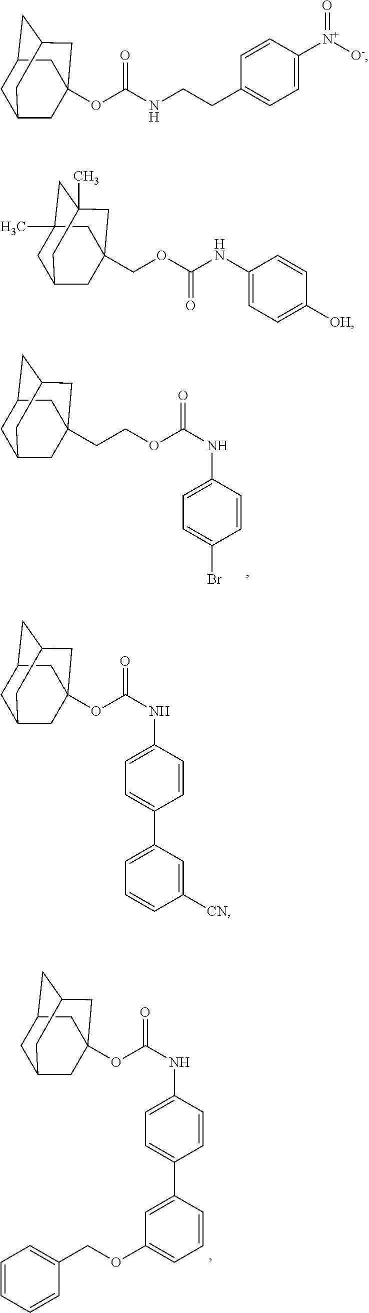 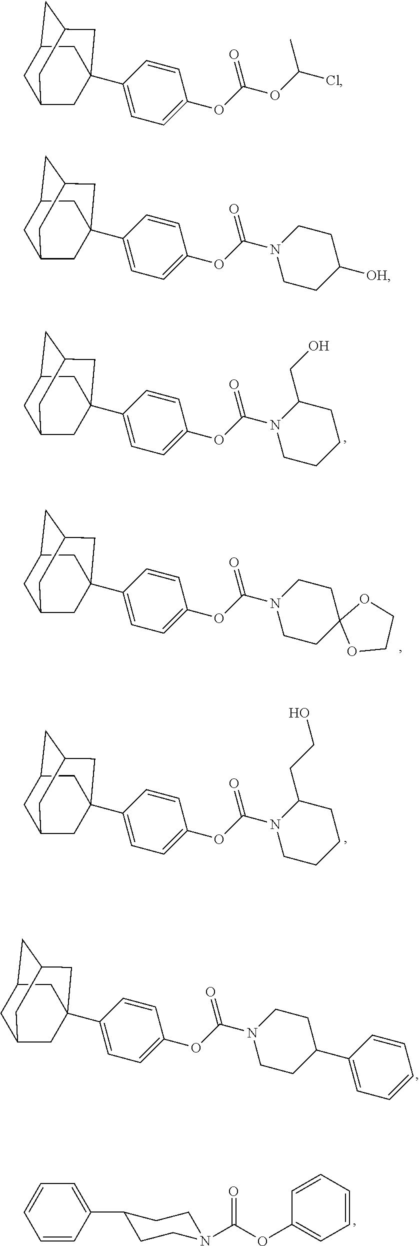 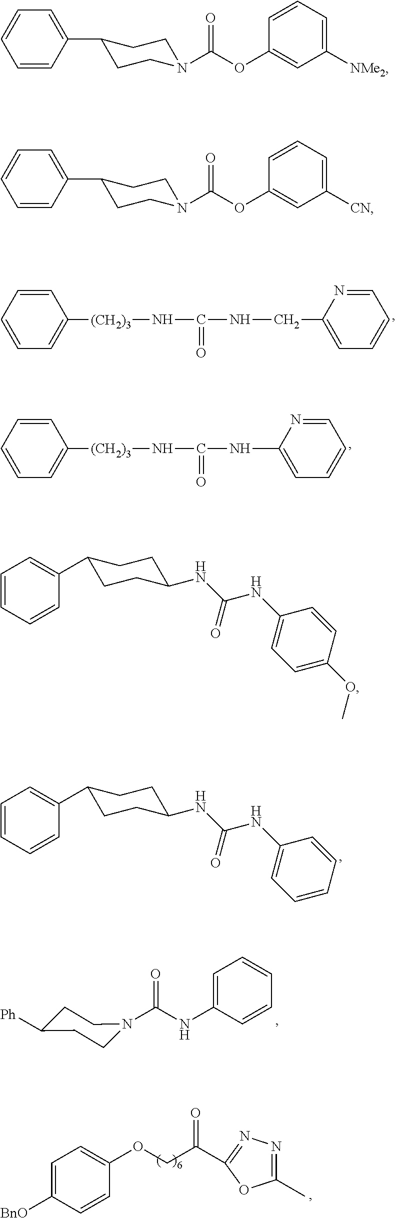 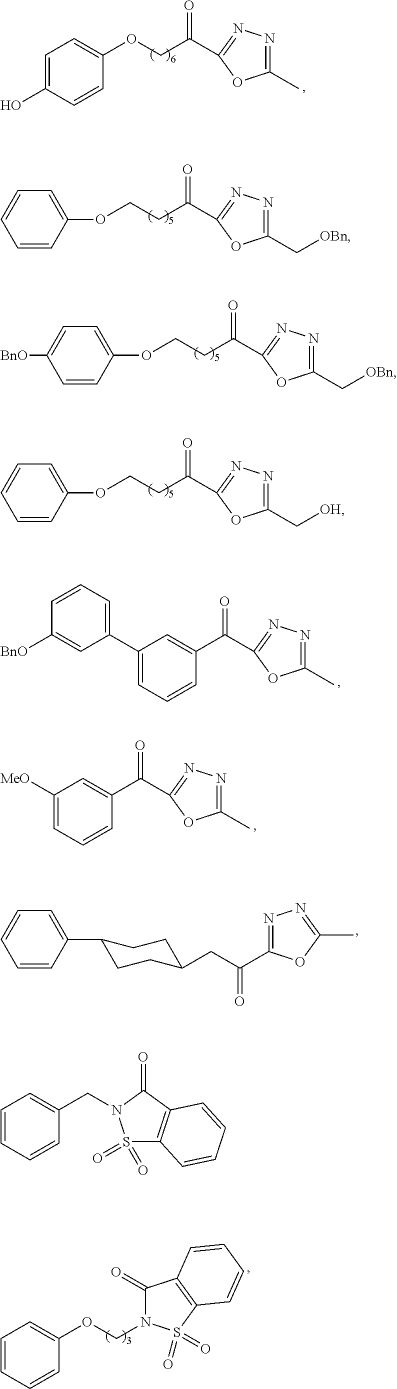 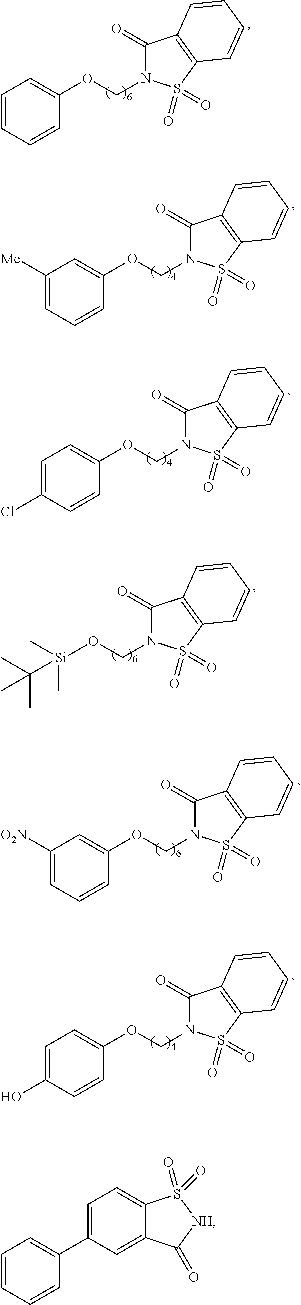 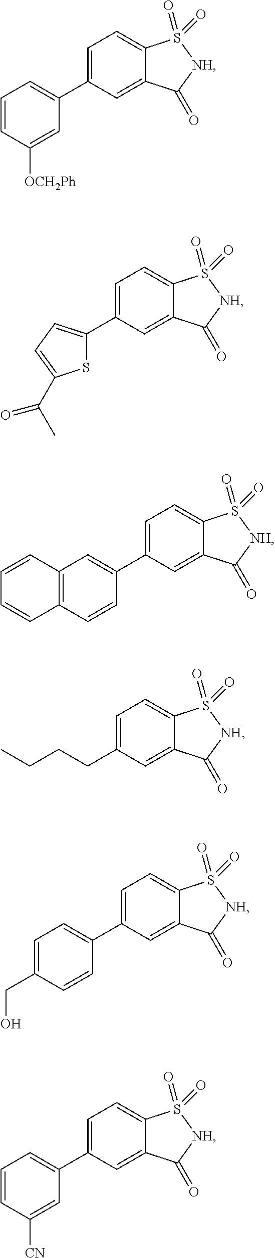 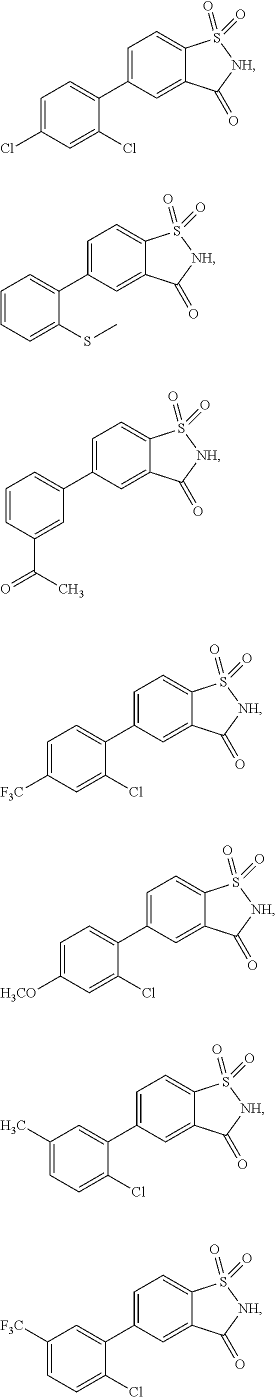 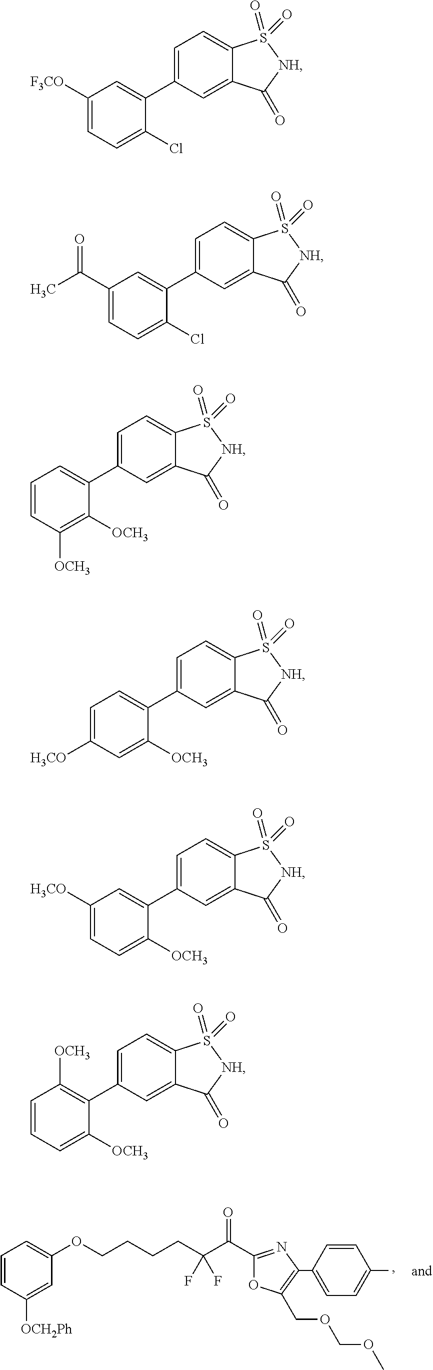 [chemical structure]
